SEQUENCE LISTING (99 sequences)

SEQ ID NO: 1 - DNA, 4938 bp, Oryza sativa
ataactcaaccttttgatctcgctacgaagctcctccgctccacccaggcacgagcgccgccgcggccgactccaccggacaccacccaacgccgccagccgcctcctcccatcctccctcgatccgtacggatagcctccccgctcgctcggtctgcgcccgtcgtcgtcgtcccgccgtcttatccctcccgcgactcggcgacgcgcgaaggcctccggcgcggggctgaaggtacgtccttttcccgagctcgttctactgttccaactctgccatggctgcagttttccattttgggctgggtctgccactgttctatggggtatgcaactgttgtcgacgtcgcgtgaagcccctgaatggccagtgtgcgatgcttgcaaggtgtttgttaaaatgtctcggtagaatatcttggaccgtttgcatattttatccatgcaaagtttagtccagtaggctgttaatttttctgtgattgcctatttagattttaggtggtgttaattcagcctcctgaagagggtggttattggatactacgatcctagaatgccgctgcagcttagctaggcggatcagccgcttaggcaggtagagcagcttccttgtcttggccggatgtcgtccctgaggttccaacggaatggagctgctgtagctgaataagaccataagttgtttttgtgcaggaaagaataagcagaactgtcatggaactcctctgcactgttgagacaaacctgggatgcctcatcataaagaataagtgtctcccacgtgataggccatcctgatgcaccttatttcctcatgtagttttttccttgtagaaggtcacactgaacaaatgcttttgagaatagaactgctaccaggaaaatgttgaatgaagttgacgactgtgatgtttatgttgttttgaaatccattaaaggaataggaaaaaaatatatgttctgatgattatttttatgattttattatttaacgtttttcctcaatgtgcgtagatttaccatgaattctctcagcaatggattactgaagggcttgaggagggtcttagaacagcagagaaaaccaattggtgggttgtttttcatggttgtttcaagatttctactcagtttttactgagtaagttttgattaaatatttgtctgtgtagatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggtatttaagtataatatgaaaaggatacttcatgttttggttttcaagcttaacttggtattaattgattgtttcaactttctaggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaagtacgtggtttcagtgagaagatcaccctttttcttttctccatacaagtatacaactatataccatttcctagttctatggcttgaaattcttttttactgtggttggtgctgtctttgttcttgccttatttcagttctgcagtttatgttgagggtgatattgaaaccagagtatacaatgacagcattaatgatcaagtgaaaaatataccagagatttgtcttcgacgcgatggtatgttgatctgccattccactagaaactttgtggtagtaatattttatagcaagaataaataacgtttgaagtttgaacaattttctcggggtaactccataactctgttaccaaactggacacagaagcctgctgctttgtttaattatactgcatttgagtaatgttgatagctattcttttgtacttatttattttgattgtctttgttgttgtaaaagcttatccttggaaaaatatttatatgtacaggtaagattcggctgattaaatctggcgagagtgctgctagcatttcattagatgagctaagtaagttcactgaaaaccttaatattctgatttatgagaatcttgcattacttgtatttgttgtgattatgcaactatgattggtctccacaattatctgcatgcagatgtccagtgttttagattttagtatttaccatttaatcatgacaacctttgtatgcaggagaaggattgttctagtttgaaagatctcagcacaagcaggccaggagtacaggcatgtttcaactcttttgttgattgttttaacagtattaggattttgacttctaagaagtgtccatgcaccctgttagctgtgggtcatgtgttttttattaatttgaacttgaaatgtgcatattgtgaattatctgtagaacaccttcagctttcaatcaagcatttccctgccaatacttatctgtcattgattggtgcctcgtgctatctttcagtagaaagtgatattttgatgcctttaccaggctcatttcttcataagtttctcatagcgtggtattttacttgtatcgcgtcttcatcttaatgaaatatgttgttacaacttcagcttgttacataagggtgattacaaattataagaaatgtcatagattcacggatcttgcgaatcaaatattacaaacctggaatggtagagtacctaattctatttatatttcttcactctatttgtacatgttgctgatagattctccctggggtatttgtacatgaatagttgcacagtgacaatcttaggccaatttgttcaatgattttaagaatgtaagacactgaaccataactagtctctagttggtggaataaaagtgatcacttgtcattattaattttcatcatagatcatcaaaatcaattaggtgagaaacaaacctctgagtaaatattctcgagtggcgaagaagaacacaggataacctgcatgttcacacaaaagcatagtatctcttgtcaacgaagtttgtagatcttcatgctgaaatgttgtgatcttgaaacttattcatatgaccaaattgtatttacacttgacatgttgccagggaatataaaaggtaatactagtaaattaactgttgagtaggttgagagaaatcttacatcagtcacacccttatctaaaagcataattctgatgttcttcgctttggaaccttgtcaattgtcaccaagtaagaatgggtgtgcttttgatggtatttcaggctttaccttatcctttgacctctttctgagcatctccttcatgccatccgccaccttgactgcagggcttgatagtgccatcctgcagaatgccatatgagctttgatagcatcttcgatgccatcctgttctcttctgtctttactgagctcatcccctaccgcctctgagcataagccacaaagccatttccctccgaagtttgccttgacattctctatgtattcctgggtgcaatcctcttccaggccacagcacgcacacctcactgattcaatgtccatcttcaatgctgatgcctgatggaaagaggactaaaagaacaatagtagatgcatgggagtagagaaggagaaagaaggtgggagaaggcaaatagaaggaaacagtatggagttcaggactaggttgctgcttgtgtggataatttttgtagacctctccttccttttggattggcttgtttagcttggtatgttattgaaattttgagatttgcccagacccatagtatcttcaacagtgtactacctcgtgaaagaataagaaagataatatgatttttttgatgcctaccagtttcctaggtaaaaatataggaaaccgtagttcacatgactgcaactgtgttgaaggtaaaggcaggagcaggttcccatagcctgaattagctagtacagttttgtttttcctgtgtgaagacgcacgatggatgacaaataaacatgctataagatccaaatgaaaatgcaaaagaaaagttattcccatatctaaaaattgtatacttgatcacatgcggtcaattagccatctacatatgcacattacaaagaacacaacacaagcttatctacctttagtgaaggagtgggggcatatcaccatcacattagcttatccttttacgacagtataaatttcaatgattttgttttatataagtctttacatgatgatgtctttaaaggttgttacatgcctaatatgtgtgatatatactagaaatgtcagacacctggtttctaacgcaacaaccttgtttaggttgattactgaaatgtcgcactcctaatattagtcgcatcgccttcgtttgtacgtatatacttttctgggcaggtcgtaacctgtttatctgtgcattattaggttcaaaagataaagtggatacgtgcaggctcaacgatgatgctggcctctcaaagaaaagaaaaacgacgatgctgcttcaccgaagcgaaatttgttgtagcaggcatgtgaaccttgtcctggccgcccttccagtcgtattttcattatcaacatcctttaatgtactcttgattaacatcttagcagtcaaatttacagtagtgtcatggtgtgtatgtgttgggaacgtattgctttggtggctatacgtcttgtcacgacaacacgacgatgtaacgatgaattctcgttgttttggtacacggggtagatgcgctcagcctaactgctcgtgtcgcaactcgcaaatgcagcgtctttttctgtcgagttgtcaga

SEQ ID NO: 2 - DNA, 621 bp, Oryza sativa
atgaattctctcagcaatggattactgaagggcttgaggagggtcttagaacagcagagaaaaccaattgatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaattctgcagtttatgttgagggtgatattgaaaccagagtatacaatgacagcattaatgatcaagtgaaaaatataccagagatttgtcttcgacgcgatggtaagattcggctgattaaatctggcgagagtgctgctagcatttcattagatgagctaagagaaggattgttctag

SEQ ID NO: 3 - protein, 206 aa, Oryza sativa
MNSLSNGLLKGLRRVLEQQRKPIDFYRKSQAWSSTVSFSDIDEKSEMGGDDDYTDSRRELEPQSVDPKKGWGFRGVHRAIICGKVGQVPVQKILRNGRTVTVFTVGTGGMFDQRVVGDADLPKPAQWHRIAIHNDQLGAFAVQKLVKNSAVYVEGDIETRVYNDSINDQVKNIPEICLRRDGKIRLIKSGESAASISLDELREGLF

SEQ ID NO: 4 - DNA, 4938 bp, Oryza sativa
ataactcaaccttttgatctcgctacgaagctcctccgctccacccaggcacgagcgccgccgcggccgactccaccggacaccacccaacgccgccagccgcctcctcccatcctccctcgatccgtacggatagcctccccgctcgctcggtctgcgcccgtcgtcgtcgtcccgccgtcttatccctcccgcgactcggcgacgcgcgaaggcctccggcgcggggctgaaggtacgtccttttcccgagctcgttctactgttccaactctgccatggctgcagttttccattttgggctgggtctgccactgttctatggggtatgcaactgttgtcgacgtcgcgtgaagcccctgaatggccagtgtgcgatgcttgcaaggtgtttgttaaaatgtctcggtagaatatcttggaccgtttgcatattttatccatgcaaagtttagtccagtaggctgttaatttttctgtgattgcctatttagattttaggtggtgttaattcagcctcctgaagagggtggttattggatactacgatcctagaatgccgctgcagcttagctaggcggatcagccgcttaggcaggtagagcagcttccttgtcttggccggatgtcgtccctgaggttccaacggaatggagctgctgtagctgaataagaccataagttgtttttgtgcaggaaagaataagcagaactgtcatggaactcctctgcactgttgagacaaacctgggatgcctcatcataaagaataagtgtctcccacgtgataggccatcctgatgcaccttatttcctcatgtagttttttccttgtagaaggtcacactgaacaaatgcttttgagaatagaactgctaccaggaaaatgttgaatgaagttgacgactgtgatgtttatgttgttttgaaatccattaaaggaataggaaaaaaatatatgttctgatgattatttttatgattttattatttaacgtttttcctcaatgtgcgtagatttaccatgaattctctcagcaatggattactgaagggcttgaggagggtcttagaacagcagagaaaaccaattggtgggttgtttttcatggttgtttcaagatttctactcagtttttactgagtaagttttgattaaatatttgtctgtgtagatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggtatttaagtataatatgaaaaggatacttcatgttttggttttcaagcttaacttggtattaattgattgtttcaactttctaggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaagtacgtggtttcagtgagaagatcaccctttttcttttctccatacaagtatacaactatataccatttcctagttctatggcttgaaattcttttttactgtggttggtgctgtctttgttcttgccttatttcagttctgcagtttatgttgagggtgatattgaaaccagagtatacaatgacagcattaatgatcaagtgaaaaatataccagagatttgtcttcgacgcgatggtatgttgatctgccattccactagaaactttgtggtagtaatattttatagcaagaataaataacgtttgaagtttgaacaattttctcggggtaactccataactctgttaccaaactggacacagaagcctgctgctttgtttaattatactgcatttgagtaatgttgatagctattcttttgtacttatttattttgattgtctttgttgttgtaaaagcttatccttggaaaaatatttatatgtacaagtaagattcggctgattaaatctggcgagagtgctgctagcatttcattagatgagctaagtaagttcactgaaaaccttaatattctgatttatgagaatcttgcattacttgtatttgttgtgattatgcaactatgattggtctccacaattatctgcatgcagatgtccagtgttttagattttagtatttaccatttaatcatgacaacctttgtatgcaggagaaggattgttctagtttgaaagatctcagcacaagcaggccaggagtacaggcatgtttcaactcttttgttgattgttttaacagtattaggattttgacttctaagaagtgtccatgcaccctgttagctgtgggtcatgtgttttttattaatttgaacttgaaatgtgcatattgtgaattatctgtagaacaccttcagctttcaatcaagcatttccctgccaatacttatctgtcattgattggtgcctcgtgctatctttcagtagaaagtgatattttgatgcctttaccaggctcatttcttcataagtttctcatagcgtggtattttacttgtatcgcgtcttcatcttaatgaaatatgttgttacaacttcagcttgttacataagggtgattacaaattataagaaatgtcatagattcacggatcttgcgaatcaaatattacaaacctggaatggtagagtacctaattctatttatatttcttcactctatttgtacatgttgctgatagattctccctggggtatttgtacatgaatagttgcacagtgacaatcttaggccaatttgttcaatgattttaagaatgtaagacactgaaccataactagtctctagttggtggaataaaagtgatcacttgtcattattaattttcatcatagatcatcaaaatcaattaggtgagaaacaaacctctgagtaaatattctcgagtggcgaagaagaacacaggataacctgcatgttcacacaaaagcatagtatctcttgtcaacgaagtttgtagatcttcatgctgaaatgttgtgatcttgaaacttattcatatgaccaaattgtatttacacttgacatgttgccagggaatataaaaggtaatactagtaaattaactgttgagtaggttgagagaaatcttacatcagtcacacccttatctaaaagcataattctgatgttcttcgctttggaaccttgtcaattgtcaccaagtaagaatgggtgtgcttttgatggtatttcaggctttaccttatcctttgacctctttctgagcatctccttcatgccatccgccaccttgactgcagggcttgatagtgccatcctgcagaatgccatatgagctttgatagcatcttcgatgccatcctgttctcttctgtctttactgagctcatcccctaccgcctctgagcataagccacaaagccatttccctccgaagtttgccttgacattctctatgtattcctgggtgcaatcctcttccaggccacagcacgcacacctcactgattcaatgtccatcttcaatgctgatgcctgatggaaagaggactaaaagaacaatagtagatgcatgggagtagagaaggagaaagaaggtgggagaaggcaaatagaaggaaacagtatggagttcaggactaggttgctgcttgtgtggataatttttgtagacctctccttccttttggattggcttgtttagcttggtatgttattgaaattttgagatttgcccagacccatagtatcttcaacagtgtactacctcgtgaaagaataagaaagataatatgatttttttgatgcctaccagtttcctaggtaaaaatataggaaaccgtagttcacatgactgcaactgtgttgaaggtaaaggcaggagcaggttcccatagcctgaattagctagtacagttttgtttttcctgtgtgaagacgcacgatggatgacaaataaacatgctataagatccaaatgaaaatgcaaaagaaaagttattcccatatctaaaaattgtatacttgatcacatgcggtcaattagccatctacatatgcacattacaaagaacacaacacaagcttatctacctttagtgaaggagtgggggcatatcaccatcacattagcttatccttttacgacagtataaatttcaatgattttgttttatataagtctttacatgatgatgtctttaaaggttgttacatgcctaatatgtgtgatatatactagaaatgtcagacacctggtttctaacgcaacaaccttgtttaggttgattactgaaatgtcgcactcctaatattagtcgcatcgccttcgtttgtacgtatatacttttctgggcaggtcgtaacctgtttatctgtgcattattaggttcaaaagataaagtggatacgtgcaggctcaacgatgatgctggcctctcaaagaaaagaaaaacgacgatgctgcttcaccgaagcgaaatttgttgtagcaggcatgtgaaccttgtcctggccgcccttccagtcgtattttcattatcaacatcctttaatgtactcttgattaacatcttagcagtcaaatttacagtagtgtcatggtgtgtatgtgttgggaacgtattgctttggtggctatacgtcttgtcacgacaacacgacgatgtaacgatgaattctcgttgttttggtacacggggtagatgcgctcagcctaactgctcgtgtcgcaactcgcaaatgcagcgtctttttctgtcgagttgtcaga

SEQ ID NO: 5 - DNA, 652 bp, Oryza sativa
atgaattctctcagcaatggattactgaagggcttgaggagggtcttagaacagcagagaaaaccaattgatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaattctgcagtttatgttgagggtgatattgaaaccagagtatacaatgacagcattaatgatcaagtgaaaaatataccagagatttgtcttcgacgcgatgcttatccttggaaaaatatttatatgtacaagtaagattcggctgattaaatctggcgagagtgctgctagcatttcattagatgagctaagagaaggattgttctag

SEQ ID NO: 6 - DNA, 561 bp, Oryza sativa
atgaattctctcagcaatggattactgaagggcttgaggagggtcttagaacagcagagaaaaccaattgatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaattctgcagtttatgttgagggtgatattgaaaccagagtatacaatgacagcattaatgatcaagtgaaaaatataccagagatttgtcttcgacgcgatggagaaggattgttctag

SEQ ID NO: 7 - DNA, 620 bp, Oryza sativa
atgaattctctcagcaatggattactgaagggcttgaggagggtcttagaacagcagagaaaaccaattgatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaattctgcagtttatgttgagggtgatattgaaaccagagtatacaatgacagcattaatgatcaagtgaaaaatataccagagatttgtcttcgacgcgatgtaagattcggctgattaaatctggcgagagtgctgctagcatttcattagatgagctaagagaaggattgttctag

SEQ ID NO: 8 - protein, 192 aa, Oryza sativa
MNSLSNGLLKGLRRVLEQQRKPIDFYRKSQAWSSTVSFSDIDEKSEMGGDDDYTDSRRELEPQSVDPKKGWGFRGVHRAIICGKVGQVPVQKILRNGRTVTVFTVGTGGMFDQRVVGDADLPKPAQWHRIAIHNDQLGAFAVQKLVKNSAVYVEGDIETRVYNDSINDQVKNIPEICLRRDAYPWKNIYMYK

SEQ ID NO: 9 - protein, 186 aa, Oryza sativa
MNSLSNGLLKGLRRVLEQQRKPIDFYRKSQAWSSTVSFSDIDEKSEMGGDDDYTDSRRELEPQSVDPKKGWGFRGVHRAIICGKVGQVPVQKILRNGRTVTVFTVGTGGMFDQRVVGDADLPKPAQWHRIAIHNDQLGAFAVQKLVKNSAVYVEGDIETRVYNDSINDQVKNIPEICLRRDGEGLF

SEQ ID NO: 10 - protein, 185 aa, Oryza sativa
MNSLSNGLLKGLRRVLEQQRKPIDFYRKSQAWSSTVSFSDIDEKSEMGGDDDYTDSRRELEPQSVDPKKGWGFRGVHRAIICGKVGQVPVQKILRNGRTVTVFTVGTGGMFDQRVVGDADLPKPAQWHRIAIHNDQLGAFAVQKLVKNSAVYVEGDIETRVYNDSINDQVKNIPEICLRRDVRFG

SEQ ID NO: 11 - DNA, 141 bp, Oryza sativa
gtatacaatgacagcattaatgatcaagtgaaaaatataccagagatttgtcttcgacgcgatggtaagattcggctgattaaatctggcgagagtgctgctagcatttcattagatgagctaagagaaggattgttctag

SEQ ID NO: 12 - DNA, 172 bp, Oryza sativa
gtatacaatgacagcattaatgatcaagtgaaaaatataccagagatttgtcttcgacgcgatgcttatccttggaaaaatatttatatgtacaagtaagattcggctgattaaatctggcgagagtgctgctagcatttcattagatgagctaagagaaggattgttctag

SEQ ID NO: 13 - DNA, 81 bp, Oryza sativa
gtatacaatgacagcattaatgatcaagtgaaaaatataccagagatttgtcttcgacgcgatggagaaggattgttctag

SEQ ID NO: 14 - DNA, 140 bp, Oryza sativa
gtatacaatgacagcattaatgatcaagtgaaaaatataccagagatttgtcttcgacgcgatgtaagattcggctgattaaatctggcgagagtgctgctagcatttcattagatgagctaagagaaggattgttctag

SEQ ID NO: 15 - protein, 201 aa, Arabidopsis thaliana
MNSLAIRVSKVLRSSSISPLAISAERGSKSWFSTGPIDEGVEEDFEENVTERPELQPHGVDPRKGWGFRGVHRAIICGKVGQAPLQKILRNGRTVTIFTVGTGGMFDQRLVGATNQPKPAQWHRIAVHNEVLGSYAVQKLAKNSSVYVEGDIETRVYNDSISSEVKSIPEICVRRDGKIRMIKYGESISKISFDELKEGLI

SEQ ID NO: 16 - protein, 206 aa, Zea mays
MTGVSTGLLTGLRRVMEQQRISTAFCRQSRVSSSTVSFSDLDEKGDMEYDDNSPNSKRELRPQGVDPNKGWEFRGVHRAIICGKVGQVPVQKILRNGHTVTVFTVGTGGMFDQRVIGPKDLPKPAQWHRIAVHNDYLSAYAVQKLVKNSAVYVEGDIETRVYNDSVNDQVKNIPEICVRRDGKIQLVKSGDSAANISLDELREGLF

SEQ ID NO: 17 - protein, 206 aa, Sorghum bicolor
MTRVSTGLLKGLRRVMEQQRISTAFCRQSQVWSSTVSFSDFDEKGDMEYDDNSPDSKRELRPQSVDPKKGWEFRGVHRAIICGKVGQVPVQKILRNGHTVTVFTVGTGGMFDQRVIGPKDLPKPAQWHRIAVHNDQLGAYAVQKLVKNSAVYVEGDIETRVYNDSINDQVKNIPEICVRRDGKIRLVKSGDSAANISLDELRDGLF

SEQ ID NO: 18 - protein, 206 aa, Hordeum vulgare
MGSVGTGLLKGLRRVLEQQRKSVDLCRTSRAWSSTVSFSDIDEKDDMGDDGDLKDSRRELEPQSVDPKKGWGFRGVHRAIICGKVGQAPVQKILRNGRTVTIFTIGTGGMFDQRVIGSADTPKPAQWHRIAVHSEHLGAYAVQKLVKNSAVYIEGDIETRIYNDGIDGQVRNIPEICIRGDGKIRLVKSGESAANISLDELRDGLF

SEQ ID NO: 19 - protein, 206 aa, Triticum aestivum
MGSVGTGLLKGLRRVLEQQRKSVDLCRTSRAWSSTVSFSDIDEKDDMGDDGDLKDSRRELEPQSVDPKKGWGFRGVHRAIICGKVGQAPVQKILRNGRTITIFTIGTGGMFDQRVIGSADTPKPAQWHRIAVHSEHLGAYAVQKLVKNSAVYVEGEIETRVYNDGVDGQVRNISEICIRGDGKIRLVKSGESAASISLDELRDGLF

SEQ ID NO: 20 - protein, 205 aa, Brachypodium distachyon
MGSGGTGLMKGLRRFLEQQRKSVDLCRKSRAWSSTVSFSDIDDKSDMGDDGDYNSRRELEPQTVDPKKGWGFRGVHRAILCGKVGQVPVQKILRNGRTITVFTIGTGGMFDQRVVESADVPKPAQWHRIAVHNEQLGAYAVQKLVKNSAVYIEGDIETRVYNDSINDQVKNIPEICIRRDGKIRLVKSGESASSISLDELREGLF

SEQ ID NO: 21 - protein, 212 aa, Populus trichocarpa
MSSLVLRFAKLLRVPSPVIMPTSSLGVGLQRSLRSCYSTVSFNSDNEEGKNDKVEEEFDDLLGDRRESRFQGVDPRKGWEFRGVHRAIICGKVGQAPVQKILRNGRTVTIFTVGTGGMFDQRIIGSKDLPKPAQWHRIAVHNDSLGAYAVQQLTKNSSVYVEGDIEIRVYNDSISGEVKNICVRRDGKIRLIRSGENISNISFEDLREGLFS

SEQ ID NO: 22 - protein, 206 aa, Triticum aestivum
MGSVGTGLLKGLRRVLEKQRKPVDLCRTSRAWSSTVSFSDIDEKDDMGDDSDLKDSRRELEPQSVDPKKGWGFRGVHRAIICGKVGQAPVQKILRNGRTITIFTIGTGGMFDQRVIGSADTPKPAQWHRIAVHSEHLGAYAVQKLVKNSAVYIEGEIETRVYNDGIDGQVRNIPEICIRGDGKIRLVKSGESAASISLDELRDGLF

SEQ ID NO: 23 - protein, 206 aa, Setaria italica
MSHASTGLLKGLRRVLEQHRISTVFCRQSRAWSSTVSFSDLDEKGDMDIDDDYTDSKRELQPHSVDPKKGWAFRGVHRAIICGKVGQVPVQKILRNGRTVTVFTVGTGGMFDQRVIGPEDLPKPAQWHRIAVHNDRLGAYVVQKLVKNSAVYVEGDIETRVYNDSINDQVRNIPEICVRRDGKIRLVKSGDSAANISLDGLREGLF

SEQ ID NO: 24 - protein, 197 aa, Oryza sativa
MMSAKLLRSMSVRRLLGQQRNSVEFYRKSQAWNSTASFSDVDEKNRKGGDAEDDFTHSRPDHVFRGVHRAIICGKVGQVPVQKILRNGHTVTVFTVGTGGMFDQRTVGAENLPMPAQWHRISVHNEQLGAFAVQKLVKNSAVYVEGDIETRVYNDRINDQVKNIPEICVRRDGKIQLMQSGDSNVGKSLEELREGLF

SEQ ID NO: 25 - protein, 196 aa, Setaria italica
MGTRLLKGLSLKRLLGQCSSADLYIKSRAFSSTVSFSDLNEKFGMGGKDDDDFKGPRKNKEYQFRGVYRAIICGKVGQVPVQKKLRNGHTVTVFTVGTAGMFDQRIVADNLPMPAQWHRIAVHNEELGAYAVQKLVKNSAVFVEGDIETRVYSDSVNDQVKNIPEICLRRDGKIRLLQSGEGDVSKSLEELREGLF

SEQ ID NO: 26 - protein, 197 aa, Hordeum vulgare
MSTKLLNSLSLRRLLGQQRKPIDLYTASRAWSSSASFSDVHEKKNGMGVEADGDLADSWKDADFRGVYRAIICGSVGQVPVQKKLRNGHTVTVFTVGTGGMFDQRRLGAENLPMPAQWHRIAVHNEHLGTYAVRKLVKNAAVYVEGDIETRVYNDDINNLVKIVPEICVRFDGKIHLVQSGGTDVSKSLEELREGLF

SEQ ID NO: 27 - protein, 196 aa, Triticum aestivum
MSTKLLNSLSLTRLLGLRRNPIDLYTASRAWISSTSFSGVHEKNGMGVEADGDLADSWKDADFRGVYRAIICGSVGQVPVQKKLRNGHTVTVFTVGTGGMFDQRRVGAENLPMPAQWHRIAVHNAQLGAYAVQKLVKNAAVYVEGEIETRVYNDDINNLVKIVPEICVRYDGKIHLVQSGGSDVSKSLEELREGLF

SEQ ID NO: 28 - protein, 196 aa, Triticum aestivum
MSTKLLNSLSLRRLLGQQRNPIDLYTASRAWSSSTSFSGVHEKNGMGVEADGDVADSWKDADFRGVYRAIICGSVGQVPVQKKLRNGHIVTVFTVGTGGMFDQRRAGAENLPMPAQWHRIAVHNEQLGTYAVQKLVKNAAVYVEGDIETRVYNDDINNLVKIVPEICVRFDGKIHLVQSGGSDVSKSLEELREGLF

SEQ ID NO: 29 - protein, 196 aa, Triticum aestivum
MSTKLLNSLSLRRLLGQQRNPIDLYTASRAWSSSTSFSGAHEKNGMGVEADGDLADSWKDADFRGVYRAIICGSVGQVPVQKKLRNGHTVTVFTVGTGGMFDQRRAGAENLPMPAQWHRIAVHNEQLGTYAVQKLVKNAAVYVEGDIETRAYNDNINNQVKIVPEICVRFDGKIHLVQSGGSDVSKSLEELREGLF

SEQ ID NO: 30 - protein, 224 aa, Oryza sativa
MAATASSFLARRLLLTRRVLSSPLRPFSTTDSSSSSSSSSSSDDSRAGSDAGPDPEQQQPPPAGQDQQAAARPRAGDTRPLENGLDPGIYKAIMVGKVGQEPIQKRLRSGRTVVLFSLGTGGIRNNRRPLDREEPHQYAERCSVQWHRVCIYPERLGSLALKHVKTGSVLYLEGNLETKVFSDPITGLVRRIREIAVRSNGRLLFLGNDCNAPKLGEAKGVGYF

SEQ ID NO: 31 - protein, 217 aa, Arabidopsis thaliana
MANSMATLSRRLYRSLLSNPRISQASMSFCTNNITSPEDSDFDELESPIEPKASDPVSRFSGEERVMEERPLENGLDSGIFKAILVGQVGQLPLQKKLKSGRTVTLFSVGTGGIRNNRRPLINEDPREYASRSAVQWHRVSVYPERLADLVLKNVEPGTVIYLEGNLETKIFTDPVTGLVRRIREVAIRRNGRVVFLGKAGDMQQPSSAELRGVGYY

SEQ ID NO: 32 - protein, 221 aa, Sorghum bicolor
MAASSASLLARRLILSRRFLSSRLSSFSTTTTPSSSTPSFNGSDAESDPEHEHYQPRADQDRQQTSNRPRPPDTTRPLENGLDPGIYKAILVGKVGQEPMQKRLRNGRTVVLFSLGTGGIRNNRRPLDREEPHQYADRCSVQWHRVCVYPERLGTLALKNVKTGTVLYLEGNLETKVFCDPITGLVRRIREIAVRSTGRLLFLGNDTNAPKLGEVKGVGYF

SEQ ID NO: 33 - protein, 224 aa, Zea mays
MAASSASLLARRLIMSRRFLSSPLGSLSTTTATRSFSISSPGFKSFVVESDLENEHDQPPADQNRQQTSNSPRPPDTTRPLENGLDPGIYKAILVGKVGQEPMQKRLRSGKTVVLFSLGTGGIRNNRRPLDREEPHQYADRCSVQWHRVCVYPDRLGTVALNNVKTGTILYLEGNLETKVFCDPITGLVRRIREIAVRSSGRLLFLGNDANAPKLGEVKGVGYF

SEQ ID NO: 34 - protein, 225 aa, Setaria italica
MAASSASLLARRLLLSRRFLSSPLRPFSTTTTPSSSSSISSPSFNGSDTESDPELENDQAPGEQDRQQAQNRPRPPNTTRPLENGLDPGIYKAIMVGKVGQEPMQKRLRNGRTIVLFSLGTGGIRNNRRPLDREEPHQYADRCSVQWHRVCVYPERLGTLALNHVKTGTVLYLEGNLETKVFCDPITGLVRRIREIAVRANGRLLFLGNDANGPKLGEVKGVGYF

SEQ ID NO: 35 - protein, 217 aa, Hordeum vulgare
MAAATSSSLLGRRFLLLSRRFVSSPLRPLSTTSPPFSHSPEGSDAEPDPDPAPPADQGNQYRDQPRAPHTTRPLENGLDHGIYKAIMVGKVGQEPVQKRLRSGKTVVLFSLGTGGIRNNRRPLDNEEPHQYAERSSVQWHRVCIYPDRLGSLALKHVKTGSVLYLEGNLETKVFSDPITGLVRRIREIAVRGNGRFLFLGNDGNGPKIGEVKGVGYF

SEQ ID NO: 36 - protein, 215 aa, Triticum aestivum
MAAATSSSLLGRRFLLLSRRFVASPLRSLSTTAPPSSHSAAGSDAEPDPEPPADQGNQYRSQPRPPITTRPLENGLDHGIYKAIMVGKVGQEPVQKRLRSGKTVVLFSLGTGGIRNNRRPLDNEEPHQYAERSSVQWHRVCIYPDRLGSLALKHVKTGSVLYLEGNIETKVFSDPITGLVRRIREIAVRGNGRFLFLGNDGNGPKIGEVKGVGYF

SEQ ID NO: 37 - protein, 214 aa, Triticum aestivum
MAAATSSSLLGRRFLLSRRFVSSPFRPLSSTAPPSSHSAAVSDAEPDPEPPADQGNQYRSQPRPPNTTRTLENGLDHGIYKAIMVGKVGQEPMQKRLRSGKTVVLLSLGTGGIRNNRRPLDNEEPHQYAERSSVQWHRVCIYPDRLGSLALKHVKTGSLLYLEGNIETKVFSDPITGLVRRIREIAVRGNGRLLFLGNDGNGPKIGEVKGVGYF

SEQ ID NO: 38 - protein, 214 aa, Triticum aestivum
MAAATSSLLGRRLLLLSRRFVSSPLRPLSTTGPPSSHSAAGSDVEPDPEPPADQGNQYRSQPRPPITTRPLENGLDHGIYKAIMVGKVGQEPVQKRLRSGKTVVLFSLGTGGIRNNRRPLDNEEPHQYAERSSVQWHRVCIYPDRLGSLALKHVKTGSVLYLEGNIETKVFSDPITGLVRRIREIAVRGNGRFLFLGNDGNGPKIGEVKGVGYF

SEQ ID NO: 39 - protein, 238 aa, Populus trichocarpa
MANSMAVLSRRLYRSLLSSNPKISQLSMPFCTSSSTTTTTTTTNNLSFREEESELDGSDTHSVPNSTTSSSPSSSTTTSEGSSGSRMVQDRPLENGLDAGVYKAILVGQVGQNPLQKKLRSGREITIFSMGTGGIRNNRRPLQNEDPREYANRCAVQWHRVSVYPERLGRVVMQNVLPGSILYVEGNLETKVFTDPITGLVRRIREIAVRQNGRLVFLGKGANDQQASSLELKGLGYY

SEQ ID NO: 40 - protein, 152 aa, Mus musculus
MFRRPVLQVFRQFVRHESEVASSLVLERSLNRVQLLGRVGQDPVMRQVEGKNPVTIFSLATNEMWRSGDSEVYQMGDVSQKTTWHRISVFRPGLRDVAYQYVKKGARIFVEGKVDYGEYMDKNNVRRQATTIIAGKKLVVHSVSGCSLEGLA

SEQ ID NO: 41 - protein, 148 aa, Homo sapiens
MFRRPVLQVLRQFVRHESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 42 - protein, 146 aa, Drosophila melanogaster
MQHTRRMLNPLLTGLRNLPARGATTTTAAAPAKVEKTVNTVTILGRVGADPQLRGSQEHPVVTFSVATHTNYKYENGDWAQRTDWHRVVVFKPNLRDTVLEYLKKGQRTMVQGKITYGEITDQQGNQKTSTSIIADDVLFFRDANN

SEQ ID NO: 43 - protein, 135 aa, Saccharomyces cerevisiae
MFLRTQARFFHATTKKMDFSKMSIVGRIGSEFTEHTSANNNRYLKYSIASQPRRDGQTNWYNITVFNEPQINFLTEYVRKGALVYVEADAANYVFERDDGSKGTTLSLVQKDINLLKNGKKLEDAEGQENAASSE

SEQ ID NO: 44 - protein, 184 aa, Escherichia coli
MTVRGINKVIIVGNLGQDPEVRYMPNGGAVASLSLATSESWRDKQTGEIRENTEWHRVVLFGKLAEIASEYLRKGAQVYVEGQLRTRNWQDDSGVTRYVTEIVVGQNGTMQMLGGRRDGGQPQNTAPQQPAQPQTPATPAQPAGTPEKSPKKGGRKGRQSAAPSQQVPQPLPDDYPPMDDDAPF

SEQ ID NO: 45 - protein, 42 aa, Artificial Sequence
  mtSSB consensus motif
  X (9)..(9): I or L
  X (16)..(16): V or A
  X (18)..(18): V or L
  X (26)..(26): R or H
  X (28)..(28): V or I
  X (30)..(30): V or I
  X (33)..(33): V or I
FRGVHRAIXCGKVGQXPXQKILRNGXTXTXFTXGTGGMFDQR

SEQ ID NO: 46 - protein, 14 aa, Artificial Sequence
  mtSSB consensus motif
  X (2)..(2): K or M
  X (10)..(10): A or S
  X (11)..(11): V or I
  X (13)..(13): N or S
  X (14)..(14): D or E
PXPAQWHRIXXHXX

SEQ ID NO: 47 - protein, 24 aa, Artificial Sequence
  mtSSB consensus motif
  X (4)..(4): K or Q
  X (6)..(6): V or T
  X (10)..(10): A or S
  X (13)..(13): V or I
  X (16)..(16): D or E
  X (19)..(19): T or I
  X (21)..(21): V or I
AVQXLXKNSXVYXEGXIEXRXYND

SEQ ID NO: 48 - protein, 9 aa, Artificial Sequence
  mtSSB consensus motif
  X (3)..(3): L or V or I
  X (5)..(5): R or G
ICXRXDGKI

SEQ ID NO: 49 - DNA, 4550 bp, Oryza sativa
cagtccttcattggctatcttacattaccgtaattttaaagcattctgatttgtggttttgttccctacttgaaggtaattgaggatcacagaatgagcctgataccgttgcctggcatggttggtctcataccttctggttctaaggtgctttcattttccgaaatgaagattgagatttcctttttgttcttctgttatcattaatgatatagatcaacattcttaataactctaggtgtttaatttctgtggtggatgttatttcaagttaatagagtgctatctaggtagtaaggatctgcatacatgttttctgatttctctctgattatggcttataaaatgagttcagtacttgatttcctctacttttttttttatctttaccatgttgccaaatgccaatcatgtggtcggcctcttcatcttgaactactcatcatcagttaaagtttctcataacacatttaattttgtgctcttcaggagaagaagaagcaaacaccaaccaaaccatctgatgcaaaaagaatacgtagggatcgcagggaactggaaaatattatattcaagctttttgaaagacagcccaattgggcactaaaggcgctggtgcaagaaactgaccagccagaggtatattcttgatttatttggcgtctctatttttaagtgaaaaaaacattggttaagatactttatttgtggtgttcctatgaacaacaaatattttattcattactctcaatggttgctttgtgattgtttgtgcttcgttttatgcagcaattcctgaaggagattctgaatgatctgtgtttttacaacaaacgaggaccaaaccagggaacgcatgagctcaagcctgagtacaagaaatctacaggggacactgatgcttcttgaatatgctagttcatttaactgctttcggataaccaacatactcaacctgttgactctgaagtcacgaacattgagttgtgcagacatctcatatttcagtaacattcgttctgctgctaaatgctaagaggcataggcgaatagttgtaaggatagtttgatacatttattagagaaaagaatagaaaagaaaaatctggaatctatgttcaggtaaaataatgtgcctggtgggagtcgcagcgtagagcatagtcctattcataattttcgttgtttatatgctgtttttatgtctcatttcatcatttgaaatgcaggatttatctgattctttgaagatgttttttttattaccaagaatattatgtcttcatattcttataccccgtgtttccgtatttctttatcaattgaatcatatagttttattaattaagcaatgttaccaacgcgtgacaaggtgttagcctctcttatcagggaagaataatttttttttagagaagggtattttctacccggtttccatatccaaccggatatatacggccattgagatagggaatttagccccgcaaacaacccaatctaaaattcgctccataggagatttgaacttaggacctttgggtactacttaggtcactgtaaccactaggctatatgcgctttcgttactgagttgccaacgcgagcatgagactactaattcccggtggtaatgaaatggtgctttggcttaacaccaaccgtaaaagattgatttactagagtgaattgatcaatgcaccactctttggattggcaccctaacgttttccatctctaactcagtgtcagatttgttactttcgttactcaacaagagttggatcaaattttggatacttgtggcaatcttttaaaactgttaaactgtgtattttgtatgaaaactttcaacaagattaatccattttttcaagtttgtaataattaagattcaattaatcacacgttattatcacatcgttttacgtaaaacactcgatctttatcttcatcttcagaagattcaaacaccacctaattcaccttttgcatttatcattttttttcctatttcaagacaagaggagggggttctcaagtttgaagtttcaacccagaaccatgatgagaagcagaagtctcgactctcaaggatagcagcaacccactaactttggtgcaggttcggcccgttacgccaggagaagcagaaatatccaggcccggcccaaagttttcacaataactcaaccttttgatctcgctacgaagctcctccgctccacccaggcacgagcgccgccgcggccgactccaccggacaccacccaacgccgccagccgcctcctcccatcctccctcgatccgtacggatagcctccccgctcgctcggtctgcgcccgtcgtcgtcgtcccgccgtcttatccctcccgcgactcggcgacgcgcgaaggcctccggcgcggggctgaaggtacgtccttttcccgagctcgttctactgttccaactctgccatggctgcagttttccattttgggctgggtctgccactgttctatggggtatgcaactgttgtcgacgtcgcgtgaagcccctgaatggccagtgtgcgatgcttgcaaggtgtttgttaaaatgtctcggtagaatatcttggaccgtttgcatattttatccatgcaaagtttagtccagtaggctgttaatttttctgtgattgcctatttagattttaggtggtgttaattcagcctcctgaagagggtggttattggatactacgatcctagaatgccgctgcagcttagctaggcggatcagccgcttaggcaggtagagcagcttccttgtcttggccggatgtcgtccctgaggttccaacggaatggagctgctgtagctgaataagaccataagttgtttttgtgcaggaaagaataagcagaactgtcatggaactcctctgcactgttgagacaaacctgggatgcctcatcataaagaataagtgtctcccacgtgataggccatcctgatgcaccttatttcctcatgtagttttttccttgtagaaggtcacactgaacaaatgcttttgagaatagaactgctaccaggaaaatgttgaatgaagttgacgactgtgatgtttatgttgttttgaaatccattaaaggaataggaaaaaaatatatgttctgatgattatttttatgattttattatttaacgtttttcctcaatgtgcgtagatttaccatgaattctctcagcaatggattactgaagggcttgaggagggtcttagaacagcagagaaaaccaattggtgggttgtttttcatggttgtttcaagatttctactcagtttttactgagtaagttttgattaaatatttgtctgtgtagatttctacagaaaatctcaagcttggagttctactgtttccttttctgatattgatgagaagagtgaaatgggaggtgatgatgattatacagattcaagacgagagttagaaccccaaagcgtagaccccaagaagggctggggattccgtggtgttcacagggtatttaagtataatatgaaaaggatacttcatgttttggttttcaagcttaacttggtattaattgattgtttcaactttctaggctataatatgcggaaaagtcggacaggttcctgtgcagaaaattttaaggaatggtcgtacagtgactgtttttacagttggaactggtggcatgtttgaccagagggtagtaggggatgcagatctgccaaagccagctcagtggcatcggatagccattcataatgaccagctaggtgcttttgctgtccagaagctggtgaagaagtacgtggtttcagtgagaagatcaccctttttcttttctccatacaagtatacaactatataccatttcctagttctatggcttgaaattcttttttactgtggttggtgctgtctttgttcttgccttatttcagttctgcagtttatgttgagggtgatattgaaaccagagtatacaatgacagcattaatgatcaagtgaaaaatataccagagatttgtcttcgacgcgatggtatgttgatctgccattccactagaaactttgtggtagtaatattttatagcaagaataaataacgtttgaagtttgaacaattttctcggggtaactccataactctgttaccaaactggacacagaagcctgctgctttgtttaattatactgcatttgagtaatgttgatagctattcttttgtacttatttattttgattgtctttgttgttgtaaaagcttatccttggaaaaatatttatatgtacaggtaagattcggctgattaaatctggcgagagtgctgctagcatttcattagatgagctaagtaagttcactgaaaaccttaatattctgatttatgagaatcttgcattacttgtatttgttgtgattatgcaactatgattggtctccacaattatctgcatgcagatgtccagtgttttagattttagtatttaccatttaatcatgacaacctttgtatgcaggagaaggattgttc

SEQ ID NO: 50 - DNA, 24 bp, Artificial Sequence
  Oligonucleotide primer
ttatcagcttctcggttcatccaa

SEQ ID NO: 51 - DNA, 24 bp, Artificial Sequence
  Oligonucleotide primer
aaacagggttagcaatttcgtttt

SEQ ID NO: 52 - DNA, 21 bp, Artificial Sequence
  Oligonucleotide primer
tctgtcccatatatacaaccg

SEQ ID NO: 53 - DNA, 22 bp, Artificial Sequence
  Oligonucleotide primer
atttgtgtttcaatcctatata

SEQ ID NO: 54 - DNA, 21 bp, Artificial Sequence
  Oligonucleotide primer
ggtcctttcaaacactccaca

SEQ ID NO: 55 - DNA, 21 bp, Artificial Sequence
  Oligonucleotide primer
ccaactttgccttagcattca

SEQ ID NO: 56 - DNA, 25 bp, Artificial Sequence
  Oligonucleotide primer
actactccctatttctgtattcact

SEQ ID NO: 57 - DNA, 21 bp, Artificial Sequence
  Oligonucleotide primer
gagcgtcatggttcacctctt

SEQ ID NO: 58 - DNA, 26 bp, Artificial Sequence
  Oligonucleotide primer
taccagagatttgtcttcgacgcgat

SEQ ID NO: 59 - DNA, 17 bp, Artificial Sequence
  Oligonucleotide primer
ctagaacaatccttctc

SEQ ID NO: 60 - DNA, 45 bp, Artificial Sequence
  Oligonucleotide primer
actccctctcccttgcggattcgaatggtagtacattctcttcga

SEQ ID NO: 61 - protein, 429 aa, Oryza sativa
MANLLRRASLRRAVAAVAAAAAPCPESYKQGICGSTFHCRYFSSKAKKKTKSSGTDSGEENLSKKDLALHQAIDQITSAFGKGAIMWLGRSEGRREVPVVSTGSFSLDLALGTGGLPKGRVIEVYGPEASGKTTLALHVIAEAQKHGGYCAFVDAEHALDPALAESIGVNTSNLLLSQPDCGEQALSLVDTLIRSGSVDVVVVDSVAALVPKSELDGEMGDAHVALQARLMSQALRKLSHSLSLSQTILLFINQIRSKVTTFGGFGGPMEVTSGGNALKFYASVRMNIKRIGLVKKGEETIGSQVLVKIVKNKHAPPFKTAQFELEFGKGICRSSELIELGLKHKLVKKLGGAFYSFNEESYRGKDALKSYLNENESIAKELETNLRKLMETQAPKKQEDEGDFLSDLPEESLATETSSEEELAAVIEA

SEQ ID NO: 62 - DNA, 5131 bp, Oryza sativa
ccgagaattaaaagggggcaaaaaccaccgcaaaaaaagggggcaaaaaccctaaaccctagcaatccccaccctcttcctcgccgcacggttccaggggtcgccgctgccgtcgccgccgccgccgccgacgacgacgacatggcgaacctcctgaggcgcgcgtcgctgcggcgcgccgtcgccgccgtcgcggcagcagccgcgccctgccccgaggtcgaaactcccctcctctctccccgccgtatttgactgtgttttttttgccccccttggtggccatggagtgttcctactggcagcttgtgccatatgtgattgcgatttggcatccttgctggggtttgatcgcttgcgtttggtgaaccgcgcggcttatatgccctcggcgatttgctctgtgccaccgaatgacccccattttgatcactgagggtacccctccccttattttgtactacaaacctagaatccgggattttggagaaatgcctattaaggctatgtgcaatctgtgtgcttggacagatgcttaggggagtaaactgtgtctatttttgccacgctggtgctgcgtgaggtgcttgccgccaagtgaaacagtcgatgcttgcgagcgcctgcatgaaaaatggagtggagaagcacctgaaataactaaagcgtccgtaaaataggtgttctctattggacatcaaggctcttgaacagatgctaagggatttttttcctaagcatctgtactatttgtacatgccctttaatgcgccataaatatattttttgcttacaatagattaacctgcaacaccaaaggtgatttaggttattagattagtgtagcctccaggggttttcttgaggttgcaacacatctccgtgtcatctttatcctgacctcacgatttgtttgcaccttcagacttgtaggtatttgtatgcacttaagacaaatactggatcatttgtgacacacacagtatgcacaatgaaattgatgagaatggaagagattcactcatagtgtgcacttcaaaacaaatactgaaatcatttgtgatacgcacagtatgcataatgaaattaatgcggctggaagatatgtattcataattagctttaaatgatgccggttgagaatgaaacaatgctgtttaatctttcacataaaaaccttgatatggttttgctaatgcatcaatctttgtgcagagctataagcaagggatctgtggctctactttccactgccggtatttctcgtctaaaggtagtagacacttattccttttatgatttattatgtgcataccttaccttagatatactaaatactgcatcccatggctcagttgctatcatcttaaatcttgatattacctgtattgacacataagttacattgtgaatttgtgatgaaattgaactgcatgattttttttcttttgatttatgttttattgctgccatctactgaatcttatatttattctttttgaatcatctacttagccaaaaagaagacaaagtccagtggaactgactctggtgaagagaatctgtcaaagaaagacttggctctacaccaggctattgatcaaataacatctgcatttgggaagggcgcaataatgtggcttggtcgttctgaaggtcgtagagaagtacctgttgtgtcaactgggtctttctctttggatttggcattgggaactggtggacttccaaaggtaaggattcctatgtagttacaaaatatcttgtaagtagaatctcacttgtatcaatgtttaatttgtattgcatgatattctttgcaaatctaagttctgctgagtgttaactaccctaaaaattagtcttctctcatcatatgaagtgtgcaaatgttctttcatggacaaaacatactttgttttgtatgaatgtcatgctattgttctctgtagggtcgtgttatagaggtgtatggtccagaagcttcaggcaagacaactcttgcacttcacgttattgcagaagcacaaaagcatggcggtaatatcagcacttcgaacctaatctatatattttagcaatagcatatattttacatgagaaaaggtacagattattctgtgttctcctttaatccaaagatttaggctaacaacatctaatacgtataaaaattattaccatgtgcttaatgctgtgaacttgtgatggtatagtcagtcagatgctttttatgtagtatgcataagggttttgctggcttacaactgcttgacagtgaaagtgatcttgctaattatgatgtgaacatagttcttaaacttgtctgttcaaaatattgtaccattgagtattttgttgacatcatcagcaggggaggcccccacggtgcgctatgtattttaccaagcagcttggtatctgtgtaatagccaatagcattatagtgtctttatatttggagtttggagaatggaatatgacgaaaccagttttagtgttttagaacatggaatatgctgacattgtgtagcccaatattagctcaaagcccagcaaccatgggataagctacatatgcaaagtacctgtcgtgggcttttgtcctcgttcgctcagaggggttagggaatgctatgttttgagctcacagaggttatatagtttaattcagtgtaaacatttaaatatgaaggtgttatttgctccttcatacttctagattactagcttcaccaaataatgttggtcttcaaacacgcacagaacctactcgggaaaatctgcctgtaatgcaatttaatggagcatttcaaaaaaccatgaaccatttggtctggaaatcatgagcactgacaactgcaagttcatctaaaatagaatttggacgaacctaatatatacctcaccgagtttttgtttttgctatttttctaggttattgtgccttcgtagatgcagagcatgccttagatccagctctggctgaatcaattggggtcaacaccagtaatttacttctctctcagccagactgtggcgagcaggcactcagtcttgtggacacactgattcgaagcggctctgttgatgttgttgttgttgatagtgtaagaacctgaccatctatccccctcccatttttatttttcttttcggagcattgccttctttcaaattgataatagtgcactttctgcttaaaattttaataacaatgtccaattagcagttggaacatgcttatcgacactcaacgttaataaatacaacttcatttatatgagaagtttgctaagcagtgcatcttattttgtgacataggtagcagctcttgtccctaagagtgagcttgacggggagatgggtgatgcacatgttgctctccaggctagattgatgagccaagctcttcgcaagctgagccactctctctcactttcccagacaattttgttattcattaaccaggtattaaacatgtgcactgggcactgctcttagtgtttgttgaatttgcttttttcaatttatatatcccatgtttttgtttggtgaagcgaaggaacaaatttaactagtcttaaggctgttaattccatgatgattagcttattagctgaaaagctccaaaatacgtgacactacagctaaataaggatggtattttcagaaacattattgtacaattcagacatttgtttacagtgatgcaactgattctacttgaatcttgttcgtcgatcatgagttctcacagagtgatcatttgaacactcgttcaatacattctgtcccatcattactatagacttatattcaacctgtcccagaaaaaaattaaatactttgtaccccactaatcaatgcagtctattcgcttcatttctctcagttttatattatttgcatcctttgtatgtgttatagatcaggtctaaagtgaccacatttggaggatttggaggtccaatggaggttacttctggtggaaatgcccttaaattctatgcttccgttagaatgaacatcaagcgtattggtttggtaaagaaaggcgaagaggtaattaaccaagtcatatccttgacatttaccttatttagacagttagtcaatacttgacatatatgcactgctatgcccatatcaaggtttctttggaacctagtttttcactcctgcgtcatatctatttggatagcatctccaaaacactgttagacacgtggcacatgcatgctttgtatttagccatatgattttataacctcatcaatcaaaattacagacctagtaaccttacagatttagcttcaacatattgtagtgcttcttgaaatgttactgacaatttttacttctgtttgcagactataggtagtcaagtgcttgtgaagattgtgaaaaacaagcatgccccaccgttcaagacagcacagtttgagctggaatttggtaaagggatatgccgcagttcagagcttattgaacttggattaaagcacaagcttgtcaaaaagttgggtggtgcattttattctttcaacgaagagtcatatcgtggtaaagatgcccttaaatcttacctcaacgaaaatgaaagtatcgcaaaagaactggagacgaatttaaggaaattgatggaaactcaagcacctaaaaagcaggaggatgaaggtgatttcctgagtgatctgcctgaagagagtctcgccactgaaacatcctccgaggaggaactggcagctgtaatagaagcttaatcagtgatgcttaatcagtgatgatagatcaacaagaaggaatatgcgctgagcagttattccactgaaccatacatattttggttcttggagatgtcattttggctgcaccgggcatatagaatgtgtggaattcgtatttgaacatccttcagttgtcaaatgcctggagcgaagtgaggtagagccggggaaggaccggggggcttacgtatggacttgtatggtggctatttagacattgaaagggttattagtagaatgtaaccatgtatatatgtcgcatatctgggattcctcgtcggttctggcttgagatacacttactaaatgcttctgatatttgattattccggttccattcc

SEQ ID NO: 63 - DNA, 1796 bp, Oryza sativa
ccgagaattaaaagggggcaaaaaccaccgcaaaaaaagggggcaaaaaccctaaaccctagcaatccccaccctcttcctcgccgcacggttccaggggtcgccgctgccgtcgccgccgccgccgccgacgacgacgacatggcgaacctcctgaggcgcgcgtcgctgcggcgcgccgtcgccgccgtcgcggcagcagccgcgccctgccccgagagctataagcaagggatctgtggctctactttccactgccggtatttctcgtctaaagccaaaaagaagacaaagtccagtggaactgactctggtgaagagaatctgtcaaagaaagacttggctctacaccaggctattgatcaaataacatctgcatttgggaagggcgcaataatgtggcttggtcgttctgaaggtcgtagagaagtacctgttgtgtcaactgggtctttctctttggatttggcattgggaactggtggacttccaaagggtcgtgttatagaggtgtatggtccagaagcttcaggcaagacaactcttgcacttcacgttattgcagaagcacaaaagcatggcggttattgtgccttcgtagatgcagagcatgccttagatccagctctggctgaatcaattggggtcaacaccagtaatttacttctctctcagccagactgtggcgagcaggcactcagtcttgtggacacactgattcgaagcggctctgttgatgttgttgttgttgatagtgtagcagctcttgtccctaagagtgagcttgacggggagatgggtgatgcacatgttgctctccaggctagattgatgagccaagctcttcgcaagctgagccactctctctcactttcccagacaattttgttattcattaaccagatcaggtctaaagtgaccacatttggaggatttggaggtccaatggaggttacttctggtggaaatgcccttaaattctatgcttccgttagaatgaacatcaagcgtattggtttggtaaagaaaggcgaagagactataggtagtcaagtgcttgtgaagattgtgaaaaacaagcatgccccaccgttcaagacagcacagtttgagctggaatttggtaaagggatatgccgcagttcagagcttattgaacttggattaaagcacaagcttgtcaaaaagttgggtggtgcattttattctttcaacgaagagtcatatcgtggtaaagatgcccttaaatcttacctcaacgaaaatgaaagtatcgcaaaagaactggagacgaatttaaggaaattgatggaaactcaagcacctaaaaagcaggaggatgaaggtgatttcctgagtgatctgcctgaagagagtctcgccactgaaacatcctccgaggaggaactggcagctgtaatagaagcttaatcagtgatgcttaatcagtgatgatagatcaacaagaaggaatatgcgctgagcagttattccactgaaccatacatattttggttcttggagatgtcattttggctgcaccgggcatatagaatgtgtggaattcgtatttgaacatccttcagttgtcaaatgcctggagcgaagtgaggtagagccggggaaggaccggggggcttacgtatggacttgtatggtggctatttagacattgaaagggttattagtagaatgtaaccatgtatatatgtcgcatatctgggattcctcgtcggttctggcttgagatacacttactaaatgcttctgatatttgattattccggttccattcc

SEQ ID NO: 64 - protein, 723 aa, Oryza sativa
MPPRPSLQSLLLMAASAAAGGDSGLLLAARRRLPAAALAAGGHRIRLLHAFSAASRRGRHEVACCVRTEPAPRPASPVAVRSRSVHSTENYKKGCEQRLGQLIERLKKEGISPKQWRLGTYQRMMCPKCNGGSTEEPSLSVMIRMDGKNAAWQCFRANCGWKGFVEPDGVLKLSQAKNNTECETDQDGEANLAVNKVYRKICEEDLHLEPLCDELVTYFSERMISPETLRRNSVMQRNWSNKIVIAFTYRRDGVLVGCKYREVSKKFSQEANTEKILYGLDDIKRARDIIIVEGEIDKLSMEEAGYRNCVSVPDGAPPKVSSKLPDKDQDKKYQYLWNCKEYLDPASRIILATDADPPGQALAEELARRLGKERCWRVNWPKKNENEICKDANEVLMFLGPQALRKVIEDAELYPIRGLFSFKDFFPEIDNYYLGIRGDELGVPTGWKSMDELYKVVPGELTVVTGVPNSGKSEWIDALLCNINDQVGWKFVLCSMENKVREHARKLLEKRIKKPFFDARYGGSAERMSLDEFEEGKQWLNETFHLIRCEDDCLPSVNWVLELAKAAVLRYGVRGLVIDPYNELDHQRPSNQTETEYVSQMLTKIKRFAQHHSCHVWFVAHPRQLHNWNGGPPNMYDISGSAHFINKCDNGIVIHRNRDPNSGPLDVVQVCVRKVRNKVIGQIGDAFLSYERVSGEFRDADKDTAKKAAVAAANVAKAPQRKG

SEQ ID NO: 65 - DNA, 7684 bp, Oryza sativa
aaaccctaaacctccgcgacccaatccaaactccatcctctcctccctcctccttcttcttcttcttcttccccaagcccctctcccctcccctctcgcctcctctgctccgcgccccgcatgccccgcgcggcgaaaccctagacctcgtgatgccaccgcgcccctccctccagtcgctcctcctcatggccgcctccgctgccgccggcggggactccggcctcctcctcgccgcgcgccgccgcctccccgccgccgccctcgccgcggggggccaccgtatccggctgctccacgccttctccgccgcgagccggcgggggaggcacgaggtggcctgctgcgtcaggactgaacccgcgccgcggcccgccagccccgtcgccgttcgctccaggagtggtatggaacaactcgctttcgagctccgaacatgcgcgatactgttttcgccatgttttactaggagatcggtctctgggaatacgcttctcaatggcgtgtctccgattaatttcgtggctcgttactactgtacttttaggcgtttagctgttaatcctcccaaatgctgtgttcttaacttcttatcgccaggaactccttgaggatttcaatcaatcgtgctgggtaaatcgttttgattgattgattctactctttcaaactcctttagttgaaattgtcacattaatttggagcgcttttactgttttgtgacagtttgagtgaactgttcgtccaaggcattatgtctgccttgttttcgcagttctggtgcccacttgttcttcttaggcggcgtacatgtcatagtttgagcatttgcaagtgatctataacaattggatgtagtagcggtgatactttccgttgatgtcgtgtctctttggataacagttcattcaacggaaaactacaaaaagggttgcgagcagcgactaggacagttaattgagagactgaagaaagaaggaataagtccaaagcaatggaggcttggtacttatcaacgaatgatgtgcccaaaggtattgtgcgtttctttttaattactctaattattggtcatgtttcccttttttacatgaagagccccatcgtttggcttattcgtggaataagccaaatggcatatttgcaaacgaaaaataatttgtgaataaaacttttatatacgtgttcttagcgatctaaaagcaaaggctgaagaataaactttgatgaaaaaaaccccaaaatcaacttcaaatttaaggttgaaaattcaaattttggctgataagcataagcttaagcggaaagatgaggctagaagtgtctagctagataagaagcaattgttttgtggtttaagacatcaagagaaatgagagacattgtggcatcaaatccattgttttctactagtatctgatttgattagcctacccctgtatcataaagtatcatcgttttggtggctggttcataaaacccactagtttctacttatagcatgctcacctgctcatgatcctttggtgatccatttactttacatgctgagctatcagacagcaatgatgagtattactttccatatgttcactagtgcaatgggggatctactgaggagccgagcctcagtgttatgatccgaatggatgggtgagattttatatttacccttcttcaacttttacttcttcagatgtagtacggcctgtctcatctgatgtttctcctatttttgttcttcaggaaaaatgctgcatggcaatgttttagagctaattgtggttggaagggttttgtcgaggtgcctttaccaaataaatataggaataaagattgaagttaataaaatgtcctgatatcactattgttctctctgcagcctgatggagttctcaaactatcacaagcaaaaaataatacagaatgtgaaacggaccaggatggtgaagctaatctggcagtcaataaggtttacagaaaaatttgtgaggaggacctccatcttgagccactttgcgatgaggtaagaaattgttttttaggctggaatattaccaattactgtacaaatatcatcaaaagtttcttgttgtttttattcttttcccattcttctttgatgaatcctaatagaagtttttgcacatgggtgttctacaaaacaatcacattttggcagcttgtaacatacttctcagagagaatgatatcgccagaaacacttcggagaaacagcgtaatgcagcgtaactggagtaataaggtatagatggatgcatttccgtagatctgctttggatacaacatctttgtgttagcattgtaagtacattttgtttgcagatagttattgctttcacctacaggcgtgatggggtcctcgttggctgcaaatacagggaagtctctaagaaattttcacaggtttgtggtgctactcgtttttagggtgtatgagcttatgctatatttcttttgttcttatttttgcacttatataccaggaagcaaacacagagaaaattttatatggtctggacgatataaagcgagcacgagatattatcattgtatgttcaaaatctgttacttgatattctttttgcatattgctcctgaagatttgtgccaatgttttcatattcccagatttttagttgtttgttctccttactgacatggcaggttgagggtgaaattgataagctctcaatggaagaagctggctaccgtaattgtgtcagtgtaccagatggtgcaccaccgaaagtatccagcaagctcccagataaagatcaggttagcgctactttctgctgttgccaaagtttttgtccctcaagattcacgacaacttatgtcttcttgtcactattatattcttcagtttgagtgttgatctctggtacagttttagctaatctgcgacctaacagttgcttatgttccctgtaaagctaggataagaagtaccaatatctgtggaattgcaaagaatatcttgacccggtgggcattttattatctcagaataatttgtttatagaactgtgtagctgagatacacaggtatcattttaattcctttatttgccatggaaacaggcgtctaggataatactggcaacagatgctgatcctccagggcaggctctggctgaggaacttgctcgtcgtcttggtaaagaaaggtttgtgtatcagtgtgttgctatcataagcttcgtgatgaataagaatgattataaaagccctcatgtattgtatttaacttgatgcaatcagatgctggagggtcaattggccaaagaagaacgagaacgaaatttgcaaagacgcaaatgaggtttgtcatggcatacatgggttcctctgacttgcacattatatcttacccctaaagttggactgattttctcattgtagttacaacttgattaaacagtaaacatgttgttccttttgtttgtgtatatgctatccttaatggctgttcctggctgaagtatttgacatatgaggctagtgtggcagttaataaacatgagttggctacctcgggtgtaaataatagttgtgaatttagctaaatcaatgtacaagttaaggctgttgatatgtgcatgatactgaacttacatttatatattatttttaattaattgattgaggctatcaactgcttagtgcgtaccaaattatcatattgatctgtaacttcacaggaagttagtttatatactatgataaatatgcagtcaaactccttgtccttttaggtcttgtattttatgcagaaatgtatactagaatttacaggcggtctggccctgcaaggtagtgtgtaagacttgaagtgtgaggaaatagtctatttctagaatatggtttccaagatgagtttttattcaacttcagaatttctattgaggcataagcttcctaaccctaaattatggtattagtattagtagtgctccctataagcttcagcatgtccatatatctgtgcagatattcttattaaaaatctataagtttttttccgtcaagaaaaaaatctataatgatttttgtgtgctttgcatctacgtcctgatggaccatctgaaacatatgatcatgtatgaattgagttgttgctgccattggctccattttgttgaaatgagaatatgacatcacatcattttgcaagttagagtttgagaacttctgacactggacctcgtaatgcccttttctactacaggtactgatgtttttaggaccacaagcattgagaaaggttatagaagacgcggaactgtatcccataagaggtcttttctccttcaaagatttcttccctgagatcgataattattatcttggaatccgtggggatgagcttggtgttcctactggatggaaatccatggatgagctttacaaggtatttcttagttgcattcagttatgctcatacttattgctgtatgttggacctgaaatctggcttgcctattttttttgtattcgcttgttttctttcgctatatttatttgttggagctacccatcttgtttgcatcactctgttctcgatggcatcacttaactttttttttattttgggcgtagaatgtaaacaattattgcccactgtttttaccccatgaatctgcatgcatttccaatataggcatgcagaaattatttgctgacttgactaagagcaacttagagaagtgcatattttttcccttgagtgtaactatcacctaaatctgattgtgacttgaacctagatgatctacacttcctagaagcacgggaaattgtgctgtcaaaccttttttatcattcttccatcataaacaattcatcatggttctgttgttttctgttttgtaggttgttcctggggagttgactgtagtaacaggagtgccgaattctggtaaaagtgagtggattgatgctttactgtgcaatataaatgaccaggttgggtggaaattcgttctgtgctcaatggaaaacaaggcaagcaattactgatggtactaaattaactattaatgtcattggggagattcaaataatggttattttggtatttaggtcagggagcatgctagaaagctcttagagaagcgcattaagaaaccattctttgatgctaggtaagtaatatatttgtgtttaatctcatgataatccctgtcttttgagcattttgagaagcacacagcaaaagcctctgtgattatgcctggcaagttcaatgtgaaatcctctcgggataaatggttttatgaatcatatgtgctgagaagttggacttaagttggctgtttgcagtatttgttttgaaggttatgttgttatgtgtaattggtttagcaatccttgtttaatacagatatgggggctctgcagaaagaatgagtctagacgaatttgaagaaggcaagcaatggctgaatgaaacattccatctgatccggtatattttatatcttttgataacataattgtcacaatgggttagccaatttcactaaagtcagctttagttcatttttttgctggggtgttcagcttctccaactatatctgcatactccacatttgctggttgggtgcaaaccattttcctttttccgtcaaccctagccttgtattagtactcttattgtagtaatatgctggaatgcaataagatctcctggcttttcttggataatatgcctctcctaatgatgatttgtatacacaggtttacattcttgtttgtgacactgaatctgcttaattaatcaggtgtgaagatgattgccttccatctgttaattgggttctcgagcttgctaaagctgcagttctaagatatggagtgcgtggtcttgtgattgatccctacaatgaacttgatcaccaacgcccctcaaatcagtaagcattgcctaagcctctttttaagattgaatttgtcattagtgatacatggtgttatcatatgctgcattcgaactggctggtttggaacacctctctctatcatggaaaacggagcgactcattagcatgtgattaattaagtgttagctaaaaaaaacttgaaaaatggattaatatgattttctaaagcaactttccttgaaacttccttttcccaacttctactcccttttccatgcgcacgcttcctgaactctgaactactaaacagtgtgttttttgcaaaaaaaaaattataggaaagttgcttttaaaaatcatattaattccttttttttcagatatttttagctaatattcaattagtcattcactaatttgctcctccgttttgtgtgccgaggttgaatggttcccaaccctcaccaccgaacacagccttggcctcaaccatactcggctctcaagcacttaacttcatgtttgcttttccttactgtattgtgccattgacagaaccgaaacagaatatgtcagtcagatgcttaccaagattaagcggtttgctcagcaccattcttgtcatgtttggtttgttgcccaccctagacaggtaagaagattgaacatagttgcatattaatctttgtaacattattgaatacacaattagccttacatgctgttactgctttgctagcttcataactggaatggtggtcctcctaatatgtatgacatcagtggaagcgcacatttcataaacaaatgtgataatggaattgtcatccatcgaaacagggatccaaacagtggcccacttgatgttgtgcaggttacttttgccctctttattaccttataagttaaaatcttaatgcttcaatttagggcctcgcctaactcgtgtatttttttatattatgttggggttggattgggttatcacaggtttgtgtgaggaaggtgcgcaacaaagtgattggacaaataggagatgctttcttgtcatatgaacggtatgtctgtttatggagcatgcttttataaccattccatatgtcagttttctgtctacttttgtatgtatcactgtaaataagtactatatctagttcatatttaccaagtcttcaagctgcttgctaactaaatcaccaaatcaatcgttttgcccccacagggttagtggtgagttcagggatgctgataaagatactgcaaagaaggctgctgttgcagccgctaatgtagcaaaagccccacagaggaaaggatagatccgctgatttgatttactcactcactgaccgatcgcacaaggtggagcgatattgttcttgatgctgcacactactggagaccgtggccaacttcatatgctggagctgactggtcagtattttttttgcgacaagaccctttggaggtcacctgtctgctgcagaatttggtgggaagatggttaaccttgttacatggcctacaagacattatttgggcgatgtatatcaaagcatcaattgtagttaattttacaccaggtgctatgaacatattggtggattctatcagtagggaaacttcatcagattattatgtactatgcccttccaaaagttgtagttgctggaagggcgtttattaatccatcagatgtatttattccaggcttccacatctatgatcagaatctaatgattttttattacccaaattgtggagtcttaagatt

SEQ ID NO: 66 - DNA, 2779 bp, Oryza sativa
aaaccctaaacctccgcgacccaatccaaactccatcctctcctccctcctccttcttcttcttcttcttccccaagcccctctcccctcccctctcgcctcctctgctccgcgccccgcatgccccgcgcggcgaaaccctagacctcgtgatgccaccgcgcccctccctccagtcgctcctcctcatggccgcctccgctgccgccggcggggactccggcctcctcctcgccgcgcgccgccgcctccccgccgccgccctcgccgcggggggccaccgtatccggctgctccacgccttctccgccgcgagccggcgggggaggcacgaggtggcctgctgcgtcaggactgaacccgcgccgcggcccgccagccccgtcgccgttcgctccaggagtgttcattcaacggaaaactacaaaaagggttgcgagcagcgactaggacagttaattgagagactgaagaaagaaggaataagtccaaagcaatggaggcttggtacttatcaacgaatgatgtgcccaaagtgcaatgggggatctactgaggagccgagcctcagtgttatgatccgaatggatgggaaaaatgctgcatggcaatgttttagagctaattgtggttggaagggttttgtcgagcctgatggagttctcaaactatcacaagcaaaaaataatacagaatgtgaaacggaccaggatggtgaagctaatctggcagtcaataaggtttacagaaaaatttgtgaggaggacctccatcttgagccactttgcgatgagcttgtaacatacttctcagagagaatgatatcgccagaaacacttcggagaaacagcgtaatgcagcgtaactggagtaataagatagttattgctttcacctacaggcgtgatggggtcctcgttggctgcaaatacagggaagtctctaagaaattttcacaggaagcaaacacagagaaaattttatatggtctggacgatataaagcgagcacgagatattatcattgttgagggtgaaattgataagctctcaatggaagaagctggctaccgtaattgtgtcagtgtaccagatggtgcaccaccgaaagtatccagcaagctcccagataaagatcaggataagaagtaccaatatctgtggaattgcaaagaatatcttgacccggcgtctaggataatactggcaacagatgctgatcctccagggcaggctctggctgaggaacttgctcgtcgtcttggtaaagaaagatgctggagggtcaattggccaaagaagaacgagaacgaaatttgcaaagacgcaaatgaggtactgatgtttttaggaccacaagcattgagaaaggttatagaagacgcggaactgtatcccataagaggtcttttctccttcaaagatttcttccctgagatcgataattattatcttggaatccgtggggatgagcttggtgttcctactggatggaaatccatggatgagctttacaaggttgttcctggggagttgactgtagtaacaggagtgccgaattctggtaaaagtgagtggattgatgctttactgtgcaatataaatgaccaggttgggtggaaattcgttctgtgctcaatggaaaacaaggtcagggagcatgctagaaagctcttagagaagcgcattaagaaaccattctttgatgctagatatgggggctctgcagaaagaatgagtctagacgaatttgaagaaggcaagcaatggctgaatgaaacattccatctgatccggtgtgaagatgattgccttccatctgttaattgggttctcgagcttgctaaagctgcagttctaagatatggagtgcgtggtcttgtgattgatccctacaatgaacttgatcaccaacgcccctcaaatcaaaccgaaacagaatatgtcagtcagatgcttaccaagattaagcggtttgctcagcaccattcttgtcatgtttggtttgttgcccaccctagacagcttcataactggaatggtggtcctcctaatatgtatgacatcagtggaagcgcacatttcataaacaaatgtgataatggaattgtcatccatcgaaacagggatccaaacagtggcccacttgatgttgtgcaggtttgtgtgaggaaggtgcgcaacaaagtgattggacaaataggagatgctttcttgtcatatgaacgggttagtggtgagttcagggatgctgataaagatactgcaaagaaggctgctgttgcagccgctaatgtagcaaaagccccacagaggaaaggatagatccgctgatttgatttactcactcactgaccgatcgcacaaggtggagcgatattgttcttgatgctgcacactactggagaccgtggccaacttcatatgctggagctgactggtcagtattttttttgcgacaagaccctttggaggtcacctgtctgctgcagaatttggtgggaagatggttaaccttgttacatggcctacaagacattatttgggcgatgtatatcaaagcatcaattgtagttaattttacaccaggtgctatgaacatattggtggattctatcagtagggaaacttcatcagattattatgtactatgcccttccaaaagttgtagttgctggaagggcgtttattaatccatcagatgtatttattccaggcttccacatctatgatcagaatctaatgattttttattacccaaattgtggagtcttaagatt

SEQ ID NO: 67 - protein, 429 aa, Brachypodium distachyon
MATLLRRASVRRAIAAVASSPCPESYRQVICGSTFHCRDFASKAKKKTKSSGTDSGEENMSKKDLALHQAIDQITSSFGKGAIMWLGRSEGHREVPVVSTGSFSLDLALGIGGLPKGRVVEVYGPEASGKTTLALHVIAEAQKHGGGGYCAFIDAEHALDPSLAESIGVDTSNLLLSQPDSAEQALSLVDTLIRSGSVDVVVVDSVAALVPKTELDGEMGDAHVALQARLMSQALRKLSHSLSLSQTILLFINQIRAKVSTFGGFGGPTEVTSGGNALKFYASVRLNIRRVGLVKKGEETIGSQVNVKIVKNKHAPPFRTAQFELEFGKGISRSSELVELGLKHKLVKKTGGAYYSFNEKAFHGKDALKSFLTGNESIAKELEMELRRLIETEAPIKQEAEDDLLSDLPEETVRSDTSSEEDLAAVIEA

SEQ ID NO: 68 - DNA, 1800 bp, Brachypodium distachyon
ccatggtgctggctagtgggccacacgctcgctcccctcttcccggcgcccgagaacggggagagagaccggaggcgcaaaacggggtggaagcggccgaaaatataagcgcaaaaccctaaaccctagtccccttccatcttctccacgcggctccagggggtcggagacgaagccgcaatcgccgccgacgccatggcgaccctcctgaggcgcgcgtcggtacggcgcgccatcgccgccgtcgcctcttccccctgccctgagagctataggcaggtgatctgtggctcaacttttcattgccgagatttcgcatctaaagccaaaaagaagacaaagtcaagtggcactgactctggtgaggagaatatgtcaaaaaaggacttggccttacatcaggccatcgatcaaataacatcttcgtttgggaagggagcaataatgtggcttggccgctctgaaggtcatagagaagtacctgttgtctctactgggtctttctctttggacctggcactgggaattggtggccttccaaagggacgtgttgtagaggtgtatggcccagaggcttcaggcaagacaactcttgctctccatgttattgcagaagcacaaaagcatggcggtggcggttattgtgcatttatagatgcagagcatgctttggatccatctttggcagaatccatcggtgttgacaccagtaatttacttctctctcagccagactctgctgagcaagcactcagtcttgtagacacactgattcgaagtggctctgttgatgttgttgttgttgacagtgtagcagctctcgtccctaagactgagcttgatggagagatgggtgatgcacatgtggctctccaggctagactgatgagccaagctcttcgcaaactgagccattctctttcactttcccagacaattttgctatttattaatcagatcagggctaaggtaagcacatttggaggatttggaggaccaacggaggtcacttctggtggaaatgcccttaaattttatgcctctgttcgcttgaacatcaggcgtgttggtttggtaaagaaaggcgaagagactattggcagtcaggttaatgtcaaaattgtgaaaaacaagcatgccccaccgttcaggactgcgcaatttgaactagaatttggaaaagggatatcccgcagctcagagcttgttgaacttgggttgaagcacaagcttgttaaaaagactgggggtgcatattattctttcaacgaaaaggcatttcatggtaaagatgcccttaaatctttcctaactggaaatgagagtattgcaaaagaactggagatggagttaaggagattgattgaaactgaagcacctattaagcaggaggctgaagacgatttactgagtgacttgcctgaagagactgtcagatctgatacatcgtcagaagaggatctggcagctgtaatcgaagcttaaccagtaatgataattcagctaggagcgacacttgcagggcaattagttttggatctccagccttcggttatcactgggaaatgtgacaggatgctcaggaggccaagttatgatggttgtttagacttgacattttgggagtcagggattcagtagcatgtaatgatgtatatgtgttgcatatcttaacgattcctcgtgtaagttaagtccaaccgagcctgagccaccctcaagagcagcccattagcagctttttgctggaaagaacttaagcccagcgaattcgttctaaatttaaatctccttctatac

SEQ ID NO: 69 - protein, 427 aa, Sorghum bicolor
MATLLRRASLRRVVAAAASASSSQPESYKQVICGSTFHCREFASKAKKKKSSGTDSGEENMSKKDLALHQAIDQITSAFGKGAIMWLGRSQGHRDVPVVSTGSFALDMALGTGGLPKGRVIEVYGPEASGKTTLALHVIAEAQKNGGYCAFVDAEHALDPALAEKIGVDTNNLLLSQPDCAEQALSLVDTLIRSGSVDVVVVDSVAALVPKTELDGEMGDAHVALQARLMSQALRKLSHSLSLSQTILLFINQIRAKVATFGFGGPTEVTSGGNALKFYASVRLNIRRIGLVKKGEETIGSQIAVKIVKNKHAPPFKTAQFELEFGKGICRSSELFELGLKHKLIKKTGGAFYSFNDMSFCGKNNLKSYLDENKSVANDLEMELRRLMATEAPKEQEAEDSSPSDLPEEIVTPEVSSEEDLGAVIEG

SEQ ID NO: 70 - DNA, 1981 bp, Sorghum bicolor
gggtccacgggaggtccatcggagtgtatggcgagcgccggcccgcggggcgctcttcccgacacccggagtgggagcccggagaggcggagaccggactcaaaaggggacgaggcggagcgcagccaaaaaccccaagccccgcaaaacccctaaaccctagcacctccataccccgtccccggggttctccgcgcggcttccaacggttggaggcgaagccgccgccatggcgacactcctcaggcgcgcgtcgctgcggcgcgtcgtcgccgccgccgcctccgcctcttcttctcaacctgagagctataagcaagtgatctgtggctccacgtttcattgccgagagttcgcatcaaaagctaaaaaaaagaagtcaagtggaacagactctggtgaggagaatatgtcaaagaaagacttggctttacaccaggctattgatcaaataacgtctgcatttgggaagggggcgataatgtggcttgggcgttcacaaggccatagagatgtacctgtcgtgtctactggctctttcgctttggatatggctctaggaactggtggtcttccaaaggggcgtgtcatagaggtctatggtccagaggcttcaggcaagacaacgcttgctctacatgtcattgcagaagcacaaaagaatgggggttactgtgcctttgtagatgcagagcacgctttggatccagctctcgcagagaaaattggtgttgacactaacaatttgctcctttctcagccagactgtgctgagcaggcactcagtcttgtggatacactgattcgaagtggatctgttgatgttgttgtagtagacagtgtagctgcgcttgttccaaagactgagctcgatggtgagatgggtgatgcacatgttgctcttcaggctaggttgatgagccaagctcttcgcaagcttagccactccctttcgctttcgcagacaattttgttatttattaatcagatcagggccaaggtagccacatttggatttggaggaccaactgaggtcacttctggtggcaacgccttgaagttttatgcttctgttcgcttgaacatcaggcgtattggtttggtaaagaaaggtgaagagacaataggtagtcagattgctgtgaagattgtaaaaaacaagcatgctccacccttcaagaccgcacagtttgagcttgaatttggaaaggggatatgccgcagttctgagcttttcgaactcggattgaagcacaagcttatcaaaaagactggtggtgcattttatagtttcaatgatatgtctttctgtggtaaaaataaccttaaatcttaccttgatgaaaacaagagtgttgcaaatgatctggagatggaactaaggagattgatggcaactgaagcacctaaagagcaggaggcagaagatagttcaccgagtgatttgcctgaagagattgtcacacctgaagtatcgtcagaagaggatctgggagctgtaatcgaaggttagccagtgatttgatgtgctggcaagtgggaagagaatttctggagcagttgttcccgtattttgggccctgaagtcaccaccgtttggatcaacatgacctgtggtgttctggtgatctcattgtaacagacttcagttcttactgtgcttatacggtagtgtagcatgaagagaattcgcattgtgtgaaatcatgctgataactgagaggtggaagttgaagtaagccgggaatggtgaggtactgtggtccgtgtggcaatttgaactttattaagcgatggtaaaatgtagtggcgtggtcgtgtatatatgatgtgcacattttgagctcttgttgtcacgtcgaagtcaatttcaaaagcattctttattgagtgctgctagaattacattgagctccatccacttccggtacttcagtacttgccttgggttttgcttggagtagcgagttttggtttttga

SEQ ID NO: 71 - protein, 425 aa, Zea mays
MTTLLRRASLRRVIAAAAASSFHPESYKQGICGSTFHCREFASKAKKKSSGTDSGEENMSKKDLALHQAIDQITSAFGKGAIMWLGRSQGHRDVPVVSTGSLDLDMALGTGGLPKGRVVEVYGPEASGKTTLALHVIAEAQKNGGYCAFVDAEHALDPALAESIGVDTNNLLLSQPDCAEQALSLVDTLIRSGSVDVVVVDSVAALVPKTELDGEMGDAHIALQARLMSQALRKLSHSLSLSQTILLFINQIRAKVATFGFGGPTEVTSGGNALKFYASVRLNIRRIGLVKKGEETIGSQIAVKIVKNKHAPPFKTAQFELEFGKGICRSSELFELGLKHKLIRKSGGSYYSFNGKAFNGKSNLKSYLTENKSVANDLEMELKRLMGTDASKEQEAGDSSQSDFPEESVTPEASSEEDLGAIIEG

SEQ ID NO: 72 - DNA, 1711 bp, Zea mays
cctctcgcctccgccctcatccgcacgacttgcagcggttggaggcgaagccggctccatgacgaccctcctcaggcgcgcgtcgctgcggcgcgtcattgccgccgccgccgcctcttcttttcaccctgagagctataagcaagggatctgtggctccacatttcattgccgagagttcgcatcaaaagcaaaaaagaagtcaagtggaacagactctggggaggagaacatgtcaaagaaagacttggctttacaccaggctattgatcagataacgtctgcatttgggaagggggcaataatgtggcttgggcgttcacaaggccatagagatgtaccagtcgtgtctactgggtctttggatttggatatggctctaggaactggtggtcttccaaaggggcgtgttgtagaggtatatggtccagaggcttcaggcaagacaacgcttgctctacatgtcattgcagaagcacaaaagaatggaggttactgtgcctttgtagatgcagagcacgctttggatccagctctcgccgagtcaattggtgttgacactaacaatttactcctttctcagcccgattgtgctgagcaggcactcagtcttgtggacacactgattcgaagtggatctgttgatgttgttgtagtagacagtgttgctgcgcttgttccaaagactgagcttgatggtgagatgggtgatgcacatattgctcttcaggctaggttgatgagtcaagcccttcgcaagcttagccactccctttcactttcacaaacaattttgttatttattaatcagatcagggccaaggtagccacatttggatttggaggaccaactgaggttacttctggtggcaacgccttgaagttttatgcatctgttcgcttgaacatcaggcgtattggtttggtaaagaaaggcgaagagacaataggtagtcagattgctgtgaagattgtaaaaaacaagcatgccccacccttcaagactgcacagtttgagcttgaatttggaaaggggatatgccgcagttctgagctttttgaacttggattgaagcacaagcttatccgaaagagtggtggttcgtattatagtttcaatggtaaggctttcaatggtaaaagtaaccttaaatcttaccttactgaaaacaagagtgttgccaatgatctggagatggaactaaagagattgatgggaactgatgcgtctaaagagcaggaggcaggagacagttcgcagagtgattttcctgaagagagtgtcacacctgaagcatcgtcagaagaggatctgggagccataattgaaggttagccagtgatctgatgtgctggcaagtgggaacagaatttctggagcagttgttcctgtaaaacaatatcttgggtcctgaagtcaccaccgtttggatcgacatgcctgtggtgttctggtgatctcatcatagcagacttcagtctttactgtgcttatacagtagtgcagcaggaagagaattcacattgtgtgaaatcatggtgatatctgagaggtggaagttgtagtaagccgggaagggtgaggtattgtggtccgtggcatttaaacttcagtaagcaaacgccgtgtatatatgatgtgcacattttgaaaccgggcattctttattgactgctgctagatttacattgagctccctccacttccaat

SEQ ID NO: 73 - protein, 429 aa, Hordeum vulgare
MATLLRRASLRRAIASAAASCPESYRQVICGSSFHCRDFASKAKKKSKSSGTDSGEENMSKKDLALHQAIDQITTSFGKGAIMWLGRSEGHREVPVVSTGSFSLDLALGIGGLPKGRVIEVYGPEASGKTTLALHVIAEAQKLSGGGYCAFVDAEHALDPTLAESIGVDTSNLLLSQPDSAEQALSLVDTLIRSGSVDVVVVDSVAALVPKTELDGEMGDAHVALQARLMSQALRKLSHSLSLSQTVLLFINQIRSKVQTFGGGFGGPQEVTSGGNALKFYASVRLNIRRIGMVKKGEEIIGSQVTVKIVKNKHAPPFRTAQFELEFGKGICRSSELFELGLKHKLVKKTGGAYYSFNEQQFHGKDAVKSFLTKNEGAAKELEMELRRLIQTEPPRKQEAEDDMLGDLPEETVRPETSEEEDLAAIIEA

SEQ ID NO: 74 - DNA, 1622 bp, Hordeum vulgare
gactgagcagagaagaaaaaaccgagcgcgtaaaaccctaaaccctagcccccctccctccccaaatcctcccctcgcggctgcaggggacggagaaccaccgagcgcgccgacggcagccatggcgaccctcctgaggcgcgcgtcgctgcggcgggccatcgcctccgccgccgcctcctgccccgagagctataggcaggtgatctgtggttcttcctttcattgccgagatttcgcatctaaagccaaaaagaagtcaaagtcaagtggaaccgactccggtgaggagaacatgtcaaagaaagacttggctttacatcaggccattgatcaaataacaacttcgtttggaaaaggagcaataatgtggcttggccgctctgaaggtcatagagaagtacctgttgtgtctaccggatctttctctttggatctggcactgggaattggtgggcttccaaagggacgtgttatagaggtgtacggtccagaggcttcaggaaagacaactcttgctcttcacgttattgcagaagcacaaaagcttagcggtggtggttattgtgcatttgtagatgcagagcatgctttggatccaactctggcagagtcaattggtgtcgacaccagtaacttacttctctctcagccagactctgctgagcaggcactcagtcttgtagacacgctgattcgaagtggctctgttgatgttgttgttgttgacagtgtagcagctcttgtccccaagacagagcttgatggagagatgggtgatgcacatgtcgctctccaagccagactgatgagtcaagctcttcgcaagctgagccattctctttcactgtcccagacagttttgttatttattaatcagatcaggtctaaggtacagacgtttgggggaggatttggagggccacaagaggtcacttccggtggaaatgcccttaaattttatgcctcggttcgcctgaatatcaggcgaattggcatggtaaagaaaggtgaagagattataggaagtcaggttactgtgaagattgtgaaaaataagcacgccccaccattcaggaccgcacagtttgagctggaatttggaaaaggaatatgccgcagttcagagcttttcgagcttggattaaagcacaagcttgttaaaaagactggaggcgcatattattctttcaacgaacagcagttccatggtaaagatgccgttaaatctttccttactaaaaacgagggtgctgcgaaggaactggagatggagctaaggagattgatccagactgaaccaccgagaaagcaagaggctgaagacgacatgctgggcgatctgcctgaggagactgtcaggcctgaaacatcagaagaagaagatctggctgctataatcgaggcttgaaccagcgatgataggttagctaggaatgttacttgtggcacgaactgtgttctggacttggacattgagttcttactgcactggaaaacgaggaggtggcttcttagagtagacactgggtgtgttttgggaggagggggaagggctcaggattcagtagcatgtaatcatgtactgtatatatgttgcatgccttgactattcttgactg

SEQ ID NO: 75 - protein, 435 aa, Triticum aestivum
MATLLRRASLRRAIASAASAATASASCPESYRQVICGSTFHCRDFASKAKKKSKSSGTDSGEENMSKKDLALHQAIDQITTSFGKGAIMWLGRSEGHREVPVVSTGSFSLDLALGIGGLPKGRVIEVYGPEASGKTTLALHVIAEAQKLSGGGYCAFVDAEHALDPTLAESIGVDTGNLLLSQPDSAEQALSLVDTLIRSGSVDVVVVDSVAALVPKTELDGEMGDAHVALQARLMSQALRKLSHSLSLSQTVLLFINQIRSKVQTFGGGFGGPQEVTSGGNALKFYASVRLNIRRIGMVKKGEEIIGSQVTVKIVKNKHAPPFRTAQFELEFGKGICRSSELFDLGLKHKLVKKTGGAYYSFNEQQFHGKDAVKAFLTKNESVAEELEMELRRLIQTEPPRKPEAEDDLLDDSPEEIVRPETSSEEDMAAIIRA

SEQ ID NO: 76 - DNA, 1715 bp, Triticum aestivum
gcggagcaaaaaagaaaaaaccgagcgcgcaaaaccctaaaccctagccccccccctccctccccaaatcctcccctcgcggctgcaggggacggagaccaccgagccgccatggcgaccctcctgaggcgcgcgtcgctgcggcgggccatcgcctccgccgcctcggccgccaccgcctccgcctcctgccccgagagctataggcaggtgatctgtggctctacctttcattgccgagatttcgcatctaaagccaaaaagaagtcaaagtcaagtggcaccgactccggtgaggagaatatgtcaaagaaagacttggctttacatcaggccattgatcaaataacaacttcgtttgggaaaggagcaataatgtggcttggccgctctgaaggtcatagagaagtacctgttgtgtctaccggatctttctctttggatctggcactgggaattggtgggcttccaaagggacgtgttatagaggtgtacggcccagaggcttcaggaaagacaactcttgcacttcacgttattgcagaagcacaaaagcttagcggcggtggttattgtgcatttgtagatgcagagcatgctttggatccaactctggcagagtcaattggtgtcgacaccggtaacttacttctctctcagccagactctgctgagcaggcactcagtcttgtagacacgctgattcgaagtggctctgttgatgttgttgttgttgacagtgtagcagctcttgtccccaagaccgagcttgatggggagatgggtgatgcacatgtggctctccaagccagactgatgagtcaagctcttcgcaagctgagccattctctttcactatcccagacagttttgctatttattaatcagatcaggtctaaggtacagacatttgggggaggatttggagggccacaagaggtcacttccggtggaaatgcccttaaattttatgcctcggtccgcctgaacatcaggcgaattggtatggtaaagaaaggtgaagagattataggaagtcaggttactgtgaagattgtgaaaaacaagcatgccccaccgttcaggactgcacagtttgagctggaatttggaaaaggaatatgccgcagctcagagcttttcgatcttgggttaaagcacaagcttgttaagaagaccgggggcgcgtactattctttcaacgaacagcagttccatggtaaagatgccgttaaagctttccttactaaaaatgagagtgttgcggaagaactggagatggagctaaggagattgatccaaactgaaccgcctagaaagccggaggctgaggacgatctgctggacgattcgcctgaagagattgtcagacctgaaacgtcatcggaagaggatatggccgctataatcagggcttaaccagcgatgataggtgagctaggaatgttacttgcggtgcggattgtattttgtacttgggaccttgtgttcttactgtattggaaaatgaggaaggcaaaattatggtagctgcttagggtaggcattggggtggatttggggaggggcgaggggagggatcaggattcagtagcatgtaatcatgtatatatgttgcatgccttgactatttcctcttgtctaagtaaggcccagcaacattcttgactgagcagctaacttgccgctgctactggaaaaaaaaaaaaaaacga

SEQ ID NO: 77 - protein, 433 aa, Aegilops tauschii
MATLLRRASLRRAIASAASAATASCPESYRQVICGSTFHCRDFASKAKKKSKSSGTDSGEENMSKKDLALHQAIDQITTSFGKGAIMWLGRSEGHREVPVVSTGSFSLDLALGIGGLPKGRVVEVYGPEASGKTTLALHVIAEAQKLSGGGYCAFVDAEHALDPTLAESIGVDTSNLLLSQPDSAEQALSLVDTLIRSGSVDVVVVDSVAALVPKTELDGEMGDAHVALQARLMSQALRKLSHSLSLSQTVLVFINQIRSKVQTFGGGFGGPQEVTSGGNALKFYASVRLNIRRIGMVKKGEEIIGSQVTVKIVKNKHAPPFRTAQFELEFGKGICRSSELFDLGVKHKLVKKTGGAYYSFNEQQFHGKDAVKAFLTKNESVAKELEMELRRLIQTEPPRKPEAEDDLLDDSPEEIVRPETSSEEDLAAIIGA

SEQ ID NO: 78 - DNA, 1785 bp, Aegilops tauschii
aggcggccgaaaacactgagcaaaaagaaaaaagaaaaaaccgagcgcgcaaaaccctaaaccctagcccccctccctccccaaatcctcccctcgcggctgcaggggacggagaccaccgagccgccatggcgaccctcctgaggcgcgcgtcgctgcggcgggccatcgcctcggccgcctcggccgccaccgcctcctgccccgagagctataggcaggtgatctgtggctctacctttcattgccgagatttcgcatctaaagccaaaaagaagtcaaagtcaagtggcactgactccggtgaggagaatatgtcaaagaaagacttggctttacatcaggccatcgatcaaataacaacttcgtttgggaaaggagcaataatgtggcttggccgctctgaaggtcatcgagaagtacctgttgtgtctactggatctttctctttggatctggcactgggaattggtgggcttccaaagggacgtgttgtagaggtttacggcccagaggcttcaggaaagacaactcttgctcttcacgttattgcagaagcacaaaagcttagcggcggtggttattgtgcatttgtagatgcagagcatgctttggatccaactctggcagagtcgattggtgtcgacactagtaatttacttctctctcagccagactctgctgagcaggcgctcagtcttgtagacactctgattcgaagtggctctgttgatgttgttgttgttgacagtgtagcagctcttgtccccaagaccgagcttgatggagagatgggtgatgcacatgtggctctccaagctagactgatgagtcaagctcttcgcaagctgagccattctctttcactatcccagacagttttggtatttattaatcagatcaggtctaaggtacagacatttgggggaggatttggaggaccacaggaggtcacttccggtggaaatgcccttaaattttatgcctcggttcggctgaacatcaggcgaattggcatggtaaagaaaggtgaagagattataggaagtcaggttactgtgaagattgtgaaaaacaagcatgccccaccgttcaggactgcacagtttgagctggaatttggaaaaggaatatgccgcagctcagagcttttcgatcttggggtaaagcacaagcttgttaagaagaccgggggcgcgtactactctttcaacgaacagcagttccatggtaaagatgctgttaaagctttccttacaaaaaatgagagtgttgcgaaagaactggagatggagctaaggagattgatccaaactgagccgcctagaaagccggaggctgaggacgatctgctggacgattcgcctgaagagattgtcagacctgaaacgtcatcggaagaggacctggccgctataatcggggcttaaccagcgatgataggtgagctaggaacgttacttgcggcgcggattgtattttggacttgggaccttgtgtgctcactgtactggaaaatgaggaagggtgctcgagaggcaaaattatggtagctgcttagggtaggcattggggtggatttggggaggggagggatcaggattcagtagcatgtaatcatgtatatatgttgcatgccttgactattcctcttgtctaagtaaggtccagcaacattcttgactgagcagctaacttgccgctgctaccggaaagaatttgattctcctgctccagaaacattctccggtcttgcctgctagtagttgttttgagtggcagcac

SEQ ID NO: 79 - protein, 435 aa, Triticum turgidum
MATLLRRASLRRAIASAASAATASASCPESYRQVICGSTFHCRDFASKAKKKSKSSGTDSGEENMSKKDLALHQAIDQITTSFGKGAIMWLGRSEGHREVPVVSTGSFSLDLALGIGGLPKGRVIEVYGPEASGKTTLALHVIAEAQKLSGGGYCAFVDAEHALDPTLAESIGVDTGNLLLSQPDSAEQALSLVDTLIRSGSVDVVVVDSVAALVPKTELDGEMGDAHVALQARLMSQALRKLSHSLSLSQTVLLFINQIRSKVQTFGGGFGGPQEVTSGGNALKFYASVRLNIRRIGMVKKGEEIIGSQVTVKIVKNKHAPPFRTAQFELEFGKGICRSSELFDLGLKHKLVKKTGGAYYSFNEQQFHGKDAVKAFLTKNESVAKELEMELRRLIQTEPPRKPEAEDDLLDDSPEEIVRPETSSEEDMAAIIRA

SEQ ID NO: 80 - protein, 723 aa, Brachypodium distachyon
MPPRPSLQSLLLMAASSTSAAGGSGLLLAARRRRFPAASAAAAGAHRIRLLHSFSAGTRLPRRPELVCCFGTTPVAAPVADPVVPQPRNVRSKEDKKACEERIGILVEKMKKEGINSERWKLDIYDKLLCPVCNGGSTEERSLSVFIRQDGLSASWKCFRATCGWKGSAKPDGVSEVSQAKYVRGKENNQVVKANQAVKVYRKLHEEDLPLEPLCDELVTYFSERMISAGTLQRNNVMQRKWNKKIVIAFTYKRGKVLVGCKYREVNKKFSQEPNTEKILYGLDDIKQARDVIIVEGEIDKLSMEEAGYRNCVSVPDGAPSQVSNKLPDKDEDKKYQYLWNCKEYLDKASRIILATDADPPGQALAEELARRLGKERCWRVIWPKKNETDICKDANEVLMFLGTQALKKVIEGAELYPIRGLFNFKDFFPDIDNYYLGIRGDELGIPTGWKCMDELYKVVPGELTIVTGVPNSGKSEWIDALLCNINDQCGWKFVLCSMENKVRDHARKLLEKHIKKPFFGARYGGSVERMSPDEFEEGKQWLNETFHLIRCDDDCLPSIDWVLNLAKAAVLRHGVRGLVIDPYNELDHQRPSNQTETEYVSQILTKIKRFAQHHSCHVWFVAHPKQLQNWNGAPPNMYDISGSAHFINKCDNGIVIHRNRDPDAGPIDVVQVSMKKVRNKVIGTIGDAFLTYDRVTGEYKDADKEIVAKVVKQQSKKTAIRR

SEQ ID NO: 81 - DNA, 2824 bp, Brachypodium distachyon
aaaatcctctgcttcctcgtcgcctctcctctttcttcctccgcaagacccccaaaacgaagccccacctcctcctcctcccgtccatgccccgctccccgccctgaagccctaggccccccgtcctcgtgatgccaccgcggccgtctctccaatcgctcctcctcatggcggcctcctccacctccgccgccgggggctccggcctcctcctcgccgcgcgccgccgccgcttccccgccgcctccgctgccgccgcgggggcccaccgcatccgcttgctgcactccttctccgccgggacccgcctgcccaggcggcccgagctggtctgctgcttcggaaccaccccagtggcggcgccagtcgccgacccagtggttccgcaaccgaggaatgttcgttcgaaggaggacaagaaggcttgtgaagaacgcatagggatactagttgagaagatgaaaaaagaaggaataaattcggagcgatggaagcttgacatttatgacaagctgctttgcccagtgtgcaatgggggatcaactgaggagcgaagcctcagtgtcttcatccgacaggatggcttaagtgcatcatggaaatgttttagggcgacttgtggttggaaaggcagtgccaagcccgatggagtttccgaagtctcccaagcaaaatatgtcagaggaaaagaaaacaatcaggttgtcaaagctaatcaggcagtcaaggtttacaggaaacttcatgaggaggacctccctcttgagccactttgcgatgagctcgtaacatacttttctgaaagaatgatatcggcaggaacacttcaaaggaacaatgtaatgcaacgtaaatggaataaaaagatagttattgctttcacctataaacgtggtaaagtccttgttggctgcaaatacagggaagtcaataagaaattttcacaggagccgaacacagagaaaattttgtatggtctggacgatataaagcaagcacgtgatgttatcattgttgagggtgaaattgataaactttccatggaggaggctggctatcgtaactgtgtgagtgtgccagatggtgcaccatcgcaagtatccaacaaactcccagacaaagatgaggataagaagtatcaatatctgtggaactgcaaagaatatcttgacaaggcatctaggataatactggcaacagatgctgatcctccagggcaggctcttgctgaggaacttgctcgtcggcttggtaaagaaaggtgttggagggtcatctggccaaagaagaacgagacagatatttgcaaagatgcaaatgaggtactgatgtttttagggacacaggcattgaaaaaggttatagaaggtgcagaactgtatcctataagaggtcttttcaacttcaaagatttcttccctgacattgataattattatcttggaatccgcggggatgagcttggtattcctactggttggaaatgtatggatgagctatacaaggttgttcctggggagttgactatagtaactggagtcccaaattctggcaaaagtgagtggattgatgcattattgtgcaatataaacgatcagtgtgggtggaaatttgtactatgctcaatggagaacaaggtcagagaccatgctaggaagctattggagaagcacattaagaaaccattctttggtgcaagatatgggggttccgtggagcggatgagtccagatgagtttgaagaaggcaagcaatggttaaacgagacattccatctgattcggtgtgatgatgattgccttccatccattgactgggttcttaatcttgcaaaagctgcagttctaagacatggagttcgcggtcttgtgattgatccttataatgaacttgaccatcaacgcccctcaaatcaaaccgagacagaatatgtcagccagatacttaccaaaattaagcgctttgctcagcatcattcatgtcacgtttggtttgttgcccaccctaagcagcttcaaaactggaatggtgcaccgcctaatatgtatgacatcagtggaagtgcacatttcatcaacaaatgtgataatgggattgtcatccaccgaaacagggatccagatgctggtcctattgatgtcgtgcaggtttctatgaagaaggtgcggaacaaagtgatagggacaataggggatgctttcttgacatatgaccgggttactggtgaatacaaggatgccgataaagaaattgtggcaaaggtcgtcaagcagcagtcaaagaagacggcgattcgacgctgatttgctttactatctccctggatagttaacaaacacgagatgatatggttgcttctatggcatgtcgcagtttgctgtatgatcagcactattggcggagatggtcgtacattgtagctgactgctgcctaattcttcagcaacatcgtcctcctgaacggtatgcacgccctcaggtgtctcccagactcgtggtcaaaatgtggtttgaaggctaaccttgctaagataggcacatgacattgcttggttgtgacgtatagcagtaagcttatattgtaattaattaatcgtcgcaccagctgggtcatgtcagcagcaaaaagctcatatcatccttaggttattttgtatgcccttccaacggttgtagcagttggaagtccatcaattagcagtccatcagaaatatctattccagattcaagtattcataccagaattctggaacctagtgctttcttttttgttctaccaccacacacatgcaatgcaatccatcggccatgtggttagagcaa

SEQ ID NO: 82 - protein, 761 aa, Sorghum bicolor
MPPRPSLHSLLLMAASASSSAAAAGGDSGLLLAARRRLAATAAAGGHRIRLLHSFSPGRVPRRPEVACCVRGAPDARRSAPAPTPLRSRNVHSANNYAAASEERLGQLIQKLKNEGINPKQWRLGNFQRMLCPQCNGGSSEELSLSVYIRKDGMNATWNCFRSKCGWRGFIQADGVTNISQGKTDIESETDQEVESKKTANKVYRKVIEEDLNLEPLCDELVEYFSTRMISAETLRRNKVMQRNWNNKISIAFTYRRDGVLVGCKYRAVDKTFSQEPNTEKIFYGLDDIKRAHDVIIVEGEIDKLSMDEAGYRNCVSVPDGAPPKVSSKIPDKEQDKKYNYLWNCKDYLDSASRIILATDDDGPGQALAEELARRLGKERCWRVKWPKKNDTDTCKDANEVLMFLGPQALRKVIEDAELYPIRGLFSFKDFFPEIDNYFLGIHGDELGIHTGWKSLDDLYKVVPGELTVVTGVPNSGKSEWIDALLCNINTQCGWKFVLCSMENKVKEHARKLLEKRIGKPFFDARYGGDAQRMTPDDFEAGKEWLNETFHLIRCEDDSLPSINWVLDLAKAAVLRHGVRGLVIDPYNELDHQRPSNQTETEYVSQILTKVKRFAQHHSCHVWFVAHPRQLHNWNGGPPNMYDISGSAHFINKCDNGIVIHRNRDQNAGPLDVVQVCVRKVRNKVIGQIGDAFLTYDRVTGQYKDAGKSTIAAVTAVQNRQNSYAKSKKDNVAYEMPFPHPVEDDSVSAEDDGNSFGLNSA

SEQ ID NO: 83 - DNA, 3035 bp, Sorghum bicolor
ctatcctctccgctccgcccctctttcttcttcaccaaggcccccaccccccaaacggccaaacccctcttcctcccctgctccgctccggccggcccatgcgcccgcgcccctgaccaaccaggctgccggaaccctacccggcgccgcgtgatgcccccgcggccgtctctccactcgctgctcctcatggccgcctccgcctcgtcgtccgccgccgcggccggcggggactcgggcctcctcctcgccgcgcgccgccgcctcgcggcgacggccgccgcgggaggacaccgcatccgcctgctccactcgttctcgccgggacgcgtccccaggcggcccgaggtggcctgctgcgtgcgcggcgcaccggacgcgcggcggtcggcgcccgcccccactcccctgcgctcaaggaacgttcattcagcaaacaactatgcagctgcttctgaggagagacttgggcagctcattcagaagttgaaaaatgaaggtattaatcccaagcagtggagacttggaaatttccagcgcatgttgtgcccacagtgcaatggtggatctagcgaggagttgagtctcagtgtctacatccgaaaggacggtatgaatgcaacatggaactgttttagatctaaatgtggctggagaggttttatccaggctgatggagttaccaacatatcccaaggaaagactgacatagaaagtgaaactgaccaagaggttgaatctaagaagacggcaaataaggtatacagaaaagtcattgaagaggacctcaatcttgagccactttgcgacgagcttgtagaatacttctctacgagaatgatttcagcggaaacacttcgaaggaataaagtaatgcaacgtaactggaataataagatatccattgcttttacctatagacgtgatggagttcttgttggctgcaaatacagggcagtcgataagacattttcacaggagccaaacacggagaaaattttttatggtctggatgatataaagcgtgcacatgatgttatcattgtggagggtgaaattgataagctgtccatggatgaagctgggtatcgtaattgtgtcagtgtgccagatggtgcaccacccaaagtgtccagtaaaatcccagacaaagaacaggataagaagtataactatctttggaactgcaaagactatcttgattcggcatcaaggataatattagcaactgatgatgatggtccaggtcaggctctggccgaggaacttgctcgtcgacttggtaaagaaagatgttggagagtcaagtggccaaagaagaatgatacagatacttgtaaggatgcaaatgaggtactgatgtttttaggtccacaagcattaagaaaggttatagaagatgcagaactgtatccaataagaggccttttttcattcaaagatttcttcccagagattgataattactttcttggaatacatggtgatgagcttggtattcatactggatggaaatctttggatgatctttacaaggttgttcctggggaattgactgtagtaactggagtaccgaattctggtaagagcgagtggatagatgctttattatgcaatataaacacacaatgtgggtggaaatttgttctgtgctcgatggaaaacaaggtaaaggagcatgctaggaaactcttagagaagcgcattggaaaaccattctttgatgctaggtatggtggggatgcgcagcggatgactcctgatgattttgaagcaggaaaagaatggttaaatgaaactttccatcttatccggtgtgaagatgatagccttccatctattaattgggttcttgaccttgcaaaagctgcagttctaagacatggagtgcgtggtcttgtgattgatccttataatgaacttgaccatcagcgcccctcaaaccagacagaaacagaatacgtcagccagattcttaccaaggttaagcgctttgctcagcaccattcatgtcatgtatggtttgttgcgcatcctagacagcttcataactggaatggaggcccacctaatatgtatgacataagtggaagcgcacatttcataaacaaatgtgataatggaattgtcatccacagaaacagggatcaaaatgctggcccccttgatgttgtgcaggtttgtgtgaggaaggtgcggaataaagtgattggacaaataggagatgctttcttgacatatgaccgggttactggtcaatataaagatgctggtaaatccactatagcagccgtaacagcagtgcaaaatcggcaaaacagttatgcaaagagtaaaaaggacaatgtggcatatgagatgccatttccacatcctgttgaagacgactcagtttctgctgaagacgatgggaatagttttggtctgaacagtgcataagaaagacatgttcctgctgatttgctgtactcttgtatcaatcaacgcaagtgagattacacgattgtttctggggatggcctgttggagctcaggtagtcctcattgatgaactggattggaaaccccaggtttcctctgattcctgctgcaaatgaggttcaaagctaaaacttagatgcacacaagacatttgcggggtggtgtatagcagtaagcttatgtagattaattgccacaccaggtgctatcctatgaacatttgggtgggttatatcagtagctaaactttagtgctagggtagtaaggttaatcatgcaggcctttgccaaaaaaaaagtagcatttggaagggtttttctgttcacacttctccacacctgacgaggaacctactatgtctttaatgtgccccagccgcgaattcaggtgttcgtttgacacatgcaaagcaatctaccgaccatgtgccgagcattttagattggggaccgtctccgtcaatctgttttgtaacaggagccgcgctgctgtgctgtgcagctggaacaattagcatgaatgaatgcatccaaaatctcagatcatttgcacc

SEQ ID NO: 84 - protein, 736 aa, Zea mays
MPPRPSLHSLLLMAASAAAASGDSGPLLAARRRLAATAAAGGHRIRLLHSFSPGRVPRRPEVVCCVRSAPDSRRSAPAPPCSRNVLSAKNYSTASEERLGKIIQKLKNEGINPKQWRLGNFQRMLCPQCNGGSSEELSLSVYIRKDGINATWNCFRSKCGWRGFVQADGVTNISQGKSGIESETDQEVEAKKAANKVYRKISDEDLNLEPLCDELVEYFATRMISAETLRRNKVMQRNWNNKISIAFTYRRDGALVGCKYRAVDKTFSQEANTEKIFYGLDDIKRAHDVIIVEGEIDKLSMDEAGYRNCVSVPDGAPPKVSSKIPDQEQDKKYSYLWNCKDYLDSASRIILATDNDRPGQALAEELARRLGKERCWRVNWPKKNDTDTCKDANEVLMFLGPQALRKVIEDAELYPIRGLFSFEQFFPEIDNYFLGIHGDELGIHTGWKSMDDLYKVVPGELTVVTGVPNSGKSEWIDALLCNINKQSGWKFVLCSMENKVKEHARKLLEKHIQKPFFNARYGGSAQRMTPDEFEAGKQWLNKTFHLIRCEDDSLPSINWVLDLAKAAVLRHGVRGLVIDPYNELDHQRPSNQTETEYVSQILTKIKRFAQHHSCHVWFVAHPRQLQNWNGGPPNIYDISGSAHFINKCDNGIVIHRNRDPNAGPLDTVQVCVRKVRNKVVGQIGDAFLTYDRVTGEFKDAGKATTAPSAKAAKQSRKEAYKMPFQHAAEDGENSGL

SEQ ID NO: 85 - DNA, 2809 bp, Zea mays
caatctatcttcccccgccccgcctctccctttcttctttaccaagccccccacacccaaacccctcctcccctgctctgcctgccccatgtccccgcgcctctgaccaaccaggctgccggaaccctacccggtgccccgtgatgcccccgcggccgtcgctccactctctgctcctcatggccgcctccgccgccgcggccagcggggactcgggcccgctcctcgctgcgcgccgccgtctagctgcgacggccgccgcaggggggcaccgcatccggctgctccactccttctcgccgggacgcgtccccaggcggcctgaggtggtctgctgcgtgcgcagcgcccccgactcgcggcggtcggcccccgcccccccgtgctccaggaacgttctttcggcaaaaaactactcaactgcttctgaggagaggcttgggaagatcattcagaagttgaaaaacgaaggtattaatcccaagcagtggagacttggaaattttcagcgcatgttgtgtccacagtgcaatggtggatctagtgaggaattgagtctcagtgtctacatccgaaaggatggcataaatgcaacatggaactgttttaggtctaagtgtggctggagaggttttgtccaggctgatggagttaccaacatatcccaaggaaagagtggcatagaaagtgaaactgaccaagaggttgaagctaagaaggcagcaaataaggtttatagaaaaatcagtgacgaggacctcaatcttgagccactctgtgatgagcttgtagagtactttgctacgagaatgatttcggcggaaacacttcggaggaataaagtcatgcaacgtaactggaataataagatatccattgcttttacctatagacgtgatggagctcttgttggctgcaaatacagggcagtcgataagacattttcacaggaggcaaacacggagaaaattttttatggtctggacgatataaagcgtgcacatgatgttatcattgtggagggtgaaattgataagctgtccatggatgaagctggttatcgtaattgtgtcagtgtgccagatggtgcaccacccaaagtgtccagtaagattccagaccaagaacaggataagaagtatagctatctttggaactgcaaagactatcttgactcggcatctaggataatattagcaaccgataatgatcgtccaggtcaggctctggctgaggaacttgctcgtcggcttggtaaagaaagatgttggagagtcaattggccgaagaagaatgatacagatacttgcaaagatgcaaatgaggttctaatgtttttaggtccacaagcattgagaaaggttatagaagatgcagaactgtatcctataagaggccttttttcattcgaacagttcttcccagagattgacaattactttcttggaatacatggtgatgagcttggtattcatactggatggaaatctatggatgatctttacaaggttgttcctggtgaattgactgttgttactggagtaccaaattctggtaaaagcgagtggatagatgctttattgtgcaatataaacaaacaatctgggtggaaatttgttctgtgctcaatggaaaacaaggtaaaggagcatgctaggaaactcttagagaagcacattcagaagccattctttaatgctaggtatggtgggtctgcacagcggatgactcctgatgaatttgaagcaggaaaacaatggttaaataaaactttccatcttatccggtgtgaagatgatagccttccatctattaattgggttcttgaccttgcgaaagctgcagttctaagacatggagtgcgtggtctggtgatagatccttataatgaacttgaccatcagcgcccctcaaaccagacggaaacagaatatgtcagccagattctgaccaagattaagcgctttgctcagcaccattcatgtcatgtatggtttgttgcacatcctagacagcttcaaaactggaatggaggcccaccaaatatatacgacataagtggaagcgcacattttataaacaaatgtgataatggaattgtcatccacagaaacagggacccaaatgctggcccccttgataccgtgcaggtttgtgtgaggaaggtgcggaataaagtggttgggcaaataggagatgctttcctgacatatgaccgggttactggtgaattcaaggatgctggtaaggccactacagcacccagtgcaaaagcggcaaaacagtcacgcaaagaggcatataagatgccatttcaacatgctgctgaagacggtgagaatagtggtctgtgactgcataagaaagacatgttcctgctgatttgatgtatctgtatcattcaacgaaagtgagatttcacgattgtttctggggatggcctgctggagctcaggcagtacctcattgaactggatttgaaaccccaggatttcaccgattcctgctgcaaatgaagttgaaagctaaacttgctcagatgcgcacaagacattggcggggtggtgtatagtagtaagattaatgtaaatcaattgccacaccaggtgctaatctatgaacatttgggtgggttatatcagtagctaaactttagcagctaaggtagtacgattaatcatgcaggctttcccaaaaaattgtagcagttggaagggtttttatgttcacccttctcaacgcctgaccaggaaccaatgcctttaatgtggccgtattttttaaaaaagatgcctttaatgtgacccaa

SEQ ID NO: 86 - protein, 726 aa, Aegilops tauschii
MPPRPSLQSLLLMAASTSAGAGGSGLLLAARRRFPAAFAAAGGHRVGLLHSFPSRTRLPRRPELACCFGTAPAEAVPAAAPATPRSRNVRSMEERRACEERLGQVVEKMKKEGIDMSQWRLGTFQRTICPQCEGGSTEERSLSVFIREDGTHGNWTCFRANCGWKGYTQPDGVPKAYQAKKDSGNENESDQEVKANQPVKVIRKLREEDLRLEPLCDELVTYFSERMISAKTLRRNNVSQRKRNNKIVIAFTYRRDKVLVGCKYREVSKKFSQEANTEKILYGLDDIKQARDIIIVEGEIDKLSMEEAGYCNCVSVPDGAPAQVSNKLPDKDHDKKYSYLWNCKEYLDPASRIILATDADPPGQALAEELARRLGKERCWRVKWPKKNETEFCKDANEVLMFLGPRALKEVIEGAELYPIRGLFNFKDFFPEIDSYYLGIHGDELGIPTGWKCMDGLYKVVPGELTIVTGVPNSGKSEWIDALLCNINNQCGWKFVLCSMENKVRDHARKLLEKHIKKPFFDARYGGSVERMSKDEFEEGKEWLNESFHLIRCEDDCLPSIDWVLNLAKAAVLRHGVRGLVIDPYNELDHQRPPNQTETEYVSQILTKIKRFAQHHSCHVWFVAHPRQLHNWTGAPPNMYDISGSAHFINKCDNGIVIHRNRDPDAGPVDVVQVCMKKVRNKVIGQIGDAFLTYDRITGEYKEADEAIVAKVVKQQIRQKSTSYQR

SEQ ID NO: 87 - DNA, 2773 bp, Aegilops tauschii
cccccccccccccgcccgcctccgccccgccatgccgccgcggccgtccctccagtcgctgctcctcatggccgcctccacctccgcgggcgccgggggctcgggcctcctcctcgccgcgcgccgccgcttccccgccgccttcgccgccgcggggggccaccgcgtcggcctgctccactccttcccctcccggacccgcctcccccggcggcccgagctcgcctgctgcttcgggaccgcccccgccgaggccgtgcccgccgccgcccccgcgaccccgcgctccaggaatgttcgttccatggaggagagaagggcttgtgaagaacgactagggcaggtagttgagaagatgaaaaaggaaggaatagatatgagccaatggaggcttggcacttttcaacggacgatttgcccacagtgcgaagggggctcaactgaggaacgaagcctcagcgtattcatccgggaagatggtacacatggaaactggacttgttttagagcgaattgtggatggaaaggctatacccagcctgatggagttccgaaggcgtaccaagcaaagaaggactcgggaaatgaaaatgaaagtgaccaggaagtcaaagcaaatcagccagtcaaggttatcagaaaacttcgtgaagaggacctgcgtctcgagcctctttgtgatgagctcgtgacatacttttcagaaagaatgatatcggcaaaaacacttagaaggaacaatgtatcgcaacgtaaaaggaataacaagatcgttattgctttcacctatagacgtgataaagtccttgttggctgcaaatacagggaagtatctaagaaattttcacaggaggcgaacacagagaaaattttgtacggtctggatgatataaagcaagcacgcgatattatcattgttgagggtgaaattgataaactttccatggaggaggctggctattgtaattgtgtgagtgtgccagatggtgcaccagcgcaagtatcaaacaaactcccggacaaagatcatgataaaaagtattcatatctgtggaactgcaaagagtatcttgacccggcatctaggataatactggcaacggatgctgatcctccagggcaggctcttgctgaggaacttgctcgacgtcttggtaaagaaaggtgttggagggtcaaatggccaaagaagaacgagacagaattttgcaaagatgctaacgaggtacttatgttcttaggaccacgagcattgaaagaggttatagaaggtgcagaactgtatcctataagaggtctattcaacttcaaagatttcttccctgagatcgatagttattatctaggaattcatggggatgagcttggaattcctactggttggaaatgtatggatgggctctacaaggttgttcctggggaattgaccatagtaacaggagtaccaaattccggcaaaagtgagtggatcgatgcattactgtgtaatataaacaatcagtgtggatggaaatttgtactatgctcgatggaaaacaaggtcagagaccatgcccggaaactattggagaagcacattaagaaacctttctttgatgctagatatgggggctctgtggaacggatgagtaaagatgaatttgaagaaggcaaagaatggttgaatgagtcattccatctgattaggtgtgaagatgattgccttccatccatcgattgggttcttaatcttgctaaagccgcagttctaagacatggagtacgtggtcttgtgattgatccttataacgagcttgaccatcagcgccccccaaatcaaaccgagacagaatatgtcagccagatccttacgaagattaagcgttttgcacaacatcattcatgtcatgtatggtttgttgcgcaccctaggcagcttcacaactggactggcgcaccgcctaatatgtacgacatcagtggaagtgcgcatttcataaacaaatgtgataatggtatcgtcatacaccgaaacagggatccagatgctggccctgtggatgtcgtgcaggtctgtatgaagaaggttcggaacaaagtgattgggcaaataggggatgctttcttgacatacgaccggattactggtgaatacaaggaagccgatgaggccattgtagcaaaggtggtgaagcagcagattcgtcagaaatcgacaagttaccaacgctgatttactttgctcgctctcccggacagttgccacggttgagatattatggccctcgcttctgtggatgtcacagttgacagtttgccgtgtgatcgtggcagattggctaacttgatcgttcatgcatctgagctgattcctggtgcgaaaaatgtggtcgaaggctactaaccttactaagacggccacatggcattgctggggtgtggtggtgtatagcaagctagatcatattattgtaattaatcgtcgcaccaggcggaggtcgtgtcagcagggaaagctcatctcaaccgctagtaggttattttttgtatgcccttccgacggttgtagcagttggaagtccatgacactcacacgtaccgttggcatcagtttgcatgcatcagcagaagcccatgatttgctcatcggaggccccatgttttgaaccttcctaccacccagcacatgccatcgaccatgtggtgaccaaatctatttggatctgagctggaacgacaaatgcttccaaaatctatgatcattagatcattaaccagcatttttctttcccgc

SEQ ID NO: 88 - protein, 748 aa, Panicum hallii
MPPRPSLHSLLLMAASASSSSAAAGGDSGLLLAARRRLPAAAGHRIRLLHSFSGPRVPRRAEVACCVRSDPDARPAGPVTVRSRNVHSANNYKATSEGRLGQLVQKLKNEGINPKQWKLGNFQRMMCPKCNGGSNEELSLSVYIRMDGMNASWNCFRSTCGWRGFIQPDGVPKLSQAKTGIKSETDQEVEPNKAANKVYRKISEEDLNLEPLCDELVTYFSERRISAETLRRNKVMQRKWKNKISIAFTYRRDGVVVGCKYREVDKKFSQEANTEKIFYGLDDIKRTQDIIIVEGEIDKLSMDEAGYRNCVSVPDGAPPKVSSKIPDRDQDKKYQYLWNCKDYLDSASRIILATDADPPGQALAEELARRLGKERCWRVKWPKKNETDTCKDANEVLMFLGPQALRKVIKDAELYPIRGLFAFKDFFPEIDNYYLGIHGDELGIRTGWKSMDDLYKVVPGELTVVTGVPNSGKSEWIDALLCNINKECGWKFVLCSMENKVREHARKLLEKRIKKPFFDARYGASAERMTPDEFEAGKQWLNETFHLIRCEDDSLPSINWVLDLAKAAVLRHGVRGLVIDPYNELDHQRPSNQTETEYVSQILTKIKRFAQHHSCHVWFVAHPRQLHNWNGGPPNMYDISGSAHFINKCDNGIVIHRNRDKNAGPLDVVQVCVRKVRNKVIGQIGDAFLTYDRVTGQFKDAGKATIAATTAATVQKQKNSYVKSTKDNVAYEMPLPHVEPADSVDGKG

SEQ ID NO: 89 - DNA, 2888 bp, Panicum hallii
accccccttccgccttgccccctcctccgctccctctccgccccatgcgcccccgcccccgcgcctgaccagcaagcaaggctgccgagaccccccgtgatgcccccgcggccgtccctccactcgctgctcctcatggccgcctccgcctcctcctcctccgccgcggccggcggggactcgggcctcctcctcgccgcgcgccgccgcctccccgccgcggcgggccaccgcatccggctgctccactccttctcggggccccgcgtccccaggcgggccgaggtggcctgctgcgtgcggagcgacccggacgcgcggcccgcgggacccgtcaccgtgcgctccaggaacgttcattcagcgaacaactacaaggctacttctgaggggagacttgggcagctcgttcagaagttgaaaaacgaaggtattaatcccaagcagtggaagcttggaaattttcagcgtatgatgtgcccgaagtgcaatggtggatctaatgaggagttgagccttagtgtatacatccgaatggatggtatgaacgcatcatggaattgttttagatcgacgtgtggttggagaggttttatccagcctgatggagttccaaagctatcacaagcaaaaactggcataaaaagtgaaacggatcaagaggtcgaacctaacaaggcagcaaataaggtttacagaaaaatcagtgaagaggacctcaaccttgagccactttgtgatgagcttgtaacatacttctctgagagaaggatatccgctgaaacacttcgaaggaataaagtaatgcaacgtaaatggaagaataagatatccattgctttcacctatagacgtgatggtgtcgttgttggctgcaaatacagggaagttgataagaaattttcacaggaggcaaacacggagaaaattttttatggtctggatgatataaagcgcacacaagatattatcattgttgagggtgaaattgataagctgtcgatggatgaagctggctatcgtaattgtgtcagcgtaccagatggtgcaccaccaaaagtatccagtaaaatcccagacagagaccaggataagaagtatcaatatctgtggaactgcaaagactatcttgactcggcatctaggataatactggccaccgatgctgatcctccaggccaggctctggctgaagaacttgctcgtcgacttggtaaagaaagatgctggagagtcaagtggccaaagaagaatgagacagatacttgcaaagatgcaaatgaggtactgatgtttttaggtccacaagcattgagaaaggttataaaagatgcagaactgtatcccataagaggcctttttgcattcaaagatttcttccctgagattgataattactatcttggaatccatggggatgagctcggtattcgtactggatggaaatctatggatgacctttacaaggttgtgcctggggagttgactgtagtaacaggagtaccaaattctggtaagagtgagtggatagatgcgttgttgtgcaatataaacaaagaatgtgggtggaaatttgttctgtgctcaatggaaaacaaggtcagggagcatgctaggaaactcttagagaagcgcattaagaaaccattctttgatgctaggtatggtgcgtctgcagagcgaatgactcctgacgaatttgaagcaggaaaacaatggttgaatgaaactttccatcttatccggtgtgaagatgatagccttccgtctattaattgggttcttgaccttgcgaaagctgcagtgctaagacatggagtgcgcggtcttgtgattgatccttacaacgaacttgaccatcagcgcccctcaaaccaaacggagacagagtatgtcagccagattcttaccaagattaagcgctttgctcagcaccattcgtgtcatgtatggtttgttgcccatcctagacagctccataactggaatggaggcccaccgaatatgtatgacatcagtggaagtgcacatttcataaacaaatgtgacaatggaattgtcatccacagaaacagggataagaatgcaggccctcttgatgtcgtgcaggtttgtgtgaggaaggtgcggaacaaagtgattgggcaaataggagatgctttcttgacatatgaccgggttactggtcaattcaaggatgctggtaaagccaccatagcagccactacggcagcaacggtacaaaagcaaaagaacagctatgtaaagagtacgaaggacaatgtggcatatgagatgccattaccgcatgttgaaccagccgactcggttgatggtaaagggtaactgcctggcacagtagcacaccataatccttcgtcctgagttcagtttgctctggtttcggcggcgcagaagaaatagacatgtttttggtgtttttctctactcactggtatcatttgacgcaagtgagattgcaccattgtttctggggatgggcctgttgcagctaggttcactgttcacctcacacatagggtactgatctctagttcttccgtgatgtggcactcctgaattgggttggaaatcaagtgtctactgcttcctgctgcgaatggggttggaggctaacctcccttggatggatacaagacattggcggggtggtgtatagtaagcttatgtaattaattgccacaccaggcgctagctctatgagcagttgctgggtggatttttagcggctagggtcatcatcctttccaaaaattgtagcagttggaagggcatctgttgatctctgttcacacttatacacctgacctgggaaactatatttaatgtgccagaattgtgaaatcaggtgttgcgcttgtttcttc

SEQ ID NO: 90 - protein, 759 aa, Setaria viridis
MPPRLSLHSLLLMAASASSSAAAAGGDSGLLLAARRRLPVAAAAAAAGGHRIRLLHSFAGGRVTRRLEVACCVRSAPGARPAGPVTVRSRNVHSENNYKAASEERLGQLVQKLKSEGINPKQWRLGNFQRMMCPKCNGGSNEELSLSVYIRMDGTNATWTCFRSTCGWRGFIQPDGVPKVSQAKGDIESETDQEVEANKAAKKVYRKISEEDLNLEPLCDELVTYFSERRISAETLRRNKVMQRKWKNKISIAFTYRRDGVVVGCKYREVDKKFSQEANTEKIFYGLDDIKRAQDIIIVEGEIDKLSMDEAGYRNCVSVPDGAPPKVSSKIPDKEQDKKYQYLWNCKDYLDSTSRIILATDADPPGQALAEELARRLGKERCWRVKWPKKNETDTCKDANEVLMFLGPQALRKVIEDAELYPIRGLFAFKDFFPEIDNYYLGTHGDELGIHTGWKSMDDLYKVVPGELTVVTGVPNSGKSEWIDALLCNINKESGWKFVLCSMENKVREHARKLLEKRIKKPFFDARYGGSAERMTPDEFEAGKQWLNETFHLIRCEDDSLPSINWVLDLAKAAVLRHGVRGLVIDPYNELDHQRPSNQTETEYVSQILTKIKRFAQHHSCHVWFVAHPRQLHNWNGGPPNMYDISGSAHFINKCDNGIVIHRNRDKNAGPLDVVQVCVRKVRNKVIGQIGDAFLTYDRVTGQFRDAGKATIAAATAATAQTATRKNSYGKSTKDNVAYEMPVPHVAEDDSVSGLDSLF

SEQ ID NO: 91 - DNA, 2906 bp, Setaria viridis
cgctccgccccatgcgtccccgcccctgacccagcaaggctgccgagacccctacccgccgtccccgtgatgcccccgcggctgtccctccactcgctgctcctcatggccgcctccgcctcctcctccgccgccgcggccggcggggactcgggtctcctcctcgccgcgcgccgccgcctccccgtcgccgcggccgcggccgccgcggggggccaccgcatccggctgctccactccttcgcggggggccgcgtcaccaggcggctcgaggtggcctgctgcgtccggagcgccccgggcgcgcggcccgcgggacccgtcaccgtgcgctccaggaacgttcattcggagaacaactacaaggctgcatctgaggagagacttgggcagctcgttcagaagttgaaaagtgaaggtattaatcccaagcagtggaggcttggaaattttcagcgcatgatgtgccccaagtgcaatggtggatctaatgaggagttgagcctcagtgtctacatccgaatggatggtacgaacgcaacatggacttgttttagatcgacatgtgggtggagaggttttatccagcctgatggagttcccaaggtatcccaagcgaaaggtgacatagaaagtgaaacggatcaagaggtcgaagctaacaaggcagcaaaaaaggtttacagaaaaatcagcgaagaggacctcaatcttgagccactttgcgatgagcttgtaacgtacttttctgagagaaggatatccgcggaaacacttcgaaggaataaagtaatgcaacgtaaatggaaaaataagatatccattgctttcacctacagacgtgatggtgttgttgttggctgcaaatacagggaagttgataagaaattttcacaggaggcaaacacggagaaaattttttatggtctggatgatataaagcgtgcacaagatattatcattgttgagggtgaaattgacaagctgtcgatggatgaagctggctatcgtaattgtgttagcgtgcctgatggtgcaccaccaaaagtatccagtaaaatcccagacaaagagcaggataagaagtatcaatatctgtggaactgcaaagactatcttgactcgacatctaggataatacttgccaccgatgctgatcctccaggccaggctctggctgaagaacttgctcgtcgacttggtaaagaaagatgttggagagttaagtggccaaagaagaatgagacagatacttgcaaagatgcaaatgaggtactgatgtttttaggtccgcaagcattgagaaaggttatagaagacgcggaactgtatcctataagaggcctttttgcattcaaagatttcttccccgagattgataattactatcttggaacccatggggatgagcttggtattcatactggatggaaatctatggatgacctttacaaggttgtgcctggggagttgactgtagtaacaggagtaccaaattctggtaaaagtgagtggattgatgcgttgttgtgcaatataaacaaagaaagtgggtggaaatttgttctgtgctcaatggaaaacaaggtcagggagcatgctaggaaactcttagagaagcgcattaagaaaccattctttgatgctaggtatggtgggtctgcagagcgaatgactcctgatgaatttgaagcaggaaaacaatggttgaatgaaacttttcatcttatccggtgtgaagatgatagccttccatctattaattgggttcttgaccttgcgaaagctgcagttctaagacatggagtgcgtggtcttgtgattgacccttacaacgaacttgaccatcagcgcccctcaaaccaaacagagacagagtacgtcagccagattcttaccaagattaagcgctttgctcaacaccattcatgtcatgtatggtttgttgctcatcctagacagctccataactggaatggaggcccaccaaatatgtatgacataagtggaagtgcacatttcataaacaaatgtgataatggaattgtgatccacagaaacagggataaaaatgcaggccctcttgatgtcgtgcaggtttgtgtgaggaaggtgcggaacaaagtgattggacaaataggagatgctttcttgacatatgaccgggttactggtcaattcagggatgctggtaaagccaccatagcagccgcaacggcagcaacagcacaaaccgcaacgcgaaaaaacagttatggaaagagtacaaaggacaatgtggcatatgagatgccagttccgcatgtggctgaagatgactcggtttctggtctagactctctcttttaggtcggccttctgctgaagactaactgcctggcacaatccttaatcctgaattcagtttgctcgggtttcaatggttcagaagaaatggacatgttcttgggtgtttttctctacttgtgtcaattggcgcaagtgagatggcaccattgtttctggggatggcctgttgcagctaggttcactgttcacctcatacatagcgtacttatctctagttcttctgtgatatggcactcatgagttgggttggaaacctcaagtgcctactgttcctgctgcaaatggggttggaggctaacctcactcggatggatacaagaaattggcggggtggtgtatagtaagcttatgtaattaattgccacaccagaagctagcacaatgagcagctgggttatcatgtaggcctttacaaaaattgtagcagttggacgggcatcaattgatatctgttcacacttctatacacctgacctagtgacctgggaacctatatttaatgtgccagaattgtgaaatcaggtgttcgtttcttcgaccggacacatgccatgcaa

SEQ ID NO: 92 - protein, 699 aa, Nicotiana tabacum
MILLPYRRLVLNNSNNFAMGSKYFLHKPSITIHKTIIPVLSSKTHLFQNQRLIFSTFASKPISPIRGTSNLSYRPHRIPPPVSGVVLEDPVEGIAESEHVKALKEKLSQVGIDIGSCGPGQYSGLLCPMCTGGDSNEKSLSLFITQDGHAATWTCFRAKCGWRGGTRAFADVKTAFADMKRIGKVTKKYRQITEESLGLEPLCDVLLSYFSERMISRETLRRNAVMQRRYGDQIVIAFTYRRDGALVSCKYRDITKKFWQEADTLKIFYGLDDIKGASDIIIVEGEMDKLAMEEAGFRNCVSVPDGAPPVVSDKELPPVDKDTKYQYLWNCKEYLEKASRIILATDGDPPGQALAEELARRLGRERCWRVTWPKKSTKDRFKDANEVLMYLGPGALREVIEGAELYPIQGLFNFKDYFTEIDAYYHQTIGYELGVSTGWRSLNQLYNVVPGELTIVTGVPNSGKSEWIDALLCNLNQSVGWKFALCSMENKVRDHARKLLEKHIKKPFFDVRYGESVQRMSAQEFEEGKQWLSDTFFLIRCENDCLPNIDWVLSLAKAAVLRHGVNGLVIDPYNELDHQRPSSQTETEYVSQMLTKIKRFAQHHSCHVWFVAHPRQLHHWVGGPPNLYDISGSAHFINKCDNGIVIHRNRDPSAGPVDQVQVCVRKVRNKVSGTIGDAFLSYDRVTGEFMDLDEHPSKG

SEQ ID NO: 93 - DNA, 2802 bp, Nicotiana tabacum
ctactgtagaacccttccttaacccctcgcatttcgaagtttcttacaaaaacacaacactcacacaccatgactgcaaattctacattgtaaaatagcaggatgattcttctaccttatcgcagacttgtactaaacaactctaacaattttgccatgggctccaaatattttctccacaagccatccattaccatacacaagactattattcctgtcctttcttcaaaaacccacctttttcaaaaccaaagactcatcttttcaacatttgcttcaaaacccatctccccaattcgtggaacttcaaacttgtcttacaggccccaccgtattcctccaccagtttctggagtagttctggaagacccagtggagggaattgcggagtcagaacatgtgaaggcactgaaggagaaattaagccaggttggaatagatattggttcttgtggaccagggcaatactctggtttgctttgtccaatgtgcacaggtggggattcaaatgaaaagagcttatctctcttcattactcaggatgggcatgcagctacatggacatgctttcgagctaaatgtgggtggagaggtggtacccgggcctttgcagatgtgaagacagcttttgcagatatgaaaaggattggaaaagtgactaagaagtacagacagataactgaggagagtttggggctggaacctttatgtgatgtgttactctcatacttttctgagcgaatgatatcgagagaaacattgcggagaaatgcggttatgcaacggagatatggtgatcagattgttattgcatttacatatcggagggatggagcacttgtaagctgcaagtaccgggacatcaccaaaaagttttggcaggaagctgatacattaaagatattttatggtctagacgatataaaaggagcaagtgatatcatcattgttgagggtgagatggacaagcttgctatggaagaagctggctttcggaactgtgtgagcgttcccgatggggcacctccagttgtttcagataaagagttgccacctgtagataaggacacaaagtatcagtatctttggaactgcaaagagtatttagaaaaggcatctcgcataatactggcaactgatggggatccacctggtcaagctttagctgaagaacttgcccgccgcttggggcgagaaaggtgctggcgagtcacatggccgaaaaagagcactaaagaccgtttcaaagatgcaaatgaggtacttatgtatctggggcctggtgcactgagggaggttattgaaggtgcagagctatacccaatacaagggttattcaactttaaagattacttcactgagattgatgcatattatcaccaaacaattggctatgagcttggggtttcaactggatggagatctttaaatcaactatacaatgttgtgcctggagagttgactattgttacaggagtcccaaattcggggaagagtgaatggattgatgctcttttgtgcaatctcaatcagagtgttggctggaaatttgcactttgctctatggaaaataaggtgcgggaccatgctaggaaacttttggagaaacacataaagaaaccattctttgatgtgagatatggggaatctgttcaacggatgagtgctcaagagtttgaagagggaaagcaatggctcagtgatacttttttcctcataaggtgtgagaatgattgcctaccgaatattgactgggttctgtcacttgcaaaagctgcagttttaagacatggggtgaatggactcgtaattgatccatacaatgagcttgatcatcaacgtccttcaagccagactgaaacagagtatgtcagccagatgctgacaaagatcaaacggtttgcgcaacaccattcctgtcatgtttggtttgttgcacatccgagacagttgcatcattgggtaggaggtcctccaaatctgtacgatattagcggaagtgcacacttcataaacaaatgtgacaatggtattgttattcaccgtaacagagacccttcggctggccctgtggatcaagtgcaggtctgtgtaagaaaggtgcggaataaagttagtggaacaattggtgatgccttcttgtcatatgacagggttactggtgaattcatggaccttgacgagcacccaagcaaaggctagtaaattctggctgttcctccattttggaaggtaatgcgtgtgcgatacttgacttttgatggaagatcacagctcctacaagaagttggagccatactttgagtggaaaatcaggcggaaaaatctcagtgattgcccattggttggaaacatatatgtaacggagcattatgcagagttatgttctctgtagtggaggatcttttaagataaggtaacaagcgacatattaagctgcacgaaatctgttgcgccttaagagtatccatcatcttagggtatacccttacagggtgacacaagatcgaagtgttggatgccttaaaacttttccccagcttcagctattgtaaattatttacaaagtgcagataggcaaatagtcagtgcttaggtgagcagatatcatgttgtcaagtaacaaatgtacgtgtaattataaaaaacttatagtttcttttctattttggtggcttatttctgataattattggtatcaactgttgaggcggtttgtatttctgttacttcaccaatctgtaaaattagttgtgttgccaacatgttaatgattgtgacgtggtactgtctccgctgatc

SEQ ID NO: 94 - protein, 695 aa, Solanum tuberosum
MKFLPYRRIVVNNSNNFVMGSKYFLHKPSITLPTIYKSIPVLFQTQRLIFSAFASKPISPNRGTSSFSYRPQRIPPPVSGVMLEDPKEEIAESDHEKALKQKLSQVGIDIGSCGPGQYNGLLCPMCKGGGSNEKSLSLFITPDGHAATWTCFRAKCGWRGGTRAFADVRTAFADMKRIGKVKKKYRQITEESLGLEPLCDVLLTYFSERMISRETLRRNAVMQQRHGDQVVIAFTYRRDGALVSCKYRNMTKKFWQEADTLKIFYGLDDIKGASDIIIVEGEMDKLAMEEAGFRNCVSVPDGAPPSISDKDLPPVDKDTKYQYLWNCKEYLEKASRIILATDGDPPGQALAEELARRLGRERCWRVTWPKKSTIDHFKDANEVLMCLGPGALREVIEGAELYPIQGLFDFKNYFTEIDAYYHQTIGYELGVPTGWRSLNQLYNVVPGELTIVTGVPNSGKSEWIDALLCNLNHSVGWKFALCSMENRVREHARKLLEKHIKKPFFDVRYGESVERMSAQEFEEGKQWLSDTFFLIRCENDCLPNIDWVLSLAKAAVLRHGVNGLVIDPYNELDHQRPSSQTETEYVSQMLTKIKRFAQHHSCHVWFVAHPRQLHHWVGGPPNLYDISGSAHFINKCDNGIVIHRNRDPSAGPVDQVQVCVRKVRNKVSGTIGDAFLSYDRVTGEFMDIDEHPRKG

SEQ ID NO: 95 - DNA, 2748 bp, Solanum tuberosum
tgagaaaccataaaacccttccttaaccccctaacggatgagtgcagttttgtgttgttgaaaattagcagtagcagtagcagtagcaggaatgaaatttcttccttatcgcagaattgtagtgaataactctaacaactttgtgatgggctccaaatattttctccacaagccatccattactttaccaacaatatacaagtcgattcctgtactttttcaaactcaaagactcatcttttcagcatttgcttcaaaacccatatccccaaatcgcggaacttcaagcttctcatacaggcctcaacgaattcctccaccagtttctggagtaatgctggaagacccaaaggaggaaattgcagagtcagaccacgagaaggcactgaagcagaaattgagccaggttggaatagatattggttcttgtggaccaggccagtacaatgggttgctttgtccaatgtgcaaaggtgggggctcaaacgaaaagagcttatctctctttattactcctgatgggcatgcagctacatggacatgctttcgagctaaatgtggatggagaggtggtacacgggcctttgcagatgtgaggacagcttttgcggatatgaaaaggattggaaaagtgaaaaagaagtacagacagataactgaggagagtttggggctggaacctttatgtgatgtgttactcacatacttttctgagcgaatgatatcaagagaaacattgcggagaaatgctgttatgcaacaaagacatggtgatcaggttgttattgcatttacgtatcggagggatggagcacttgtgagctgtaagtaccggaacatgaccaaaaagttttggcaggaagctgatactttaaaaatattctatggtctcgacgatataaagggagcaagtgatatcatcattgttgagggtgagatggacaagcttgctatggaagaagcaggctttcggaattgtgtgagcgttccagatggcgctcctccatctatttcagataaagatttgccgcctgtagataaggacacaaagtatcagtatctttggaactgcaaagaatatttggaaaaggcatctcgcataatactggcaactgatggggatccacctggtcaagcattagctgaagagcttgcccgccggttagggagagaaaggtgctggcgagttacatggccaaaaaagagcactatagaccacttcaaagatgcaaatgaggtacttatgtgtctggggcctggtgctctgagggaggttattgaaggtgcagagctgtacccaatacaaggattattcgactttaaaaattactttactgagattgatgcatattatcaccaaacaattggctacgagcttggggttccaactggatggaggtccttaaatcaactatacaatgttgttcctggagagttgactattgttacaggagtcccaaactcggggaagagtgagtggattgatgctcttttatgcaatctcaatcacagtgttggctggaaatttgcactttgctctatggaaaacagggtacgggagcatgctaggaaacttttggagaagcacataaagaaacctttttttgatgtgagatatggggaatctgttgaacggatgagtgctcaggagtttgaagagggaaagcaatggctcagtgatacatttttcctcataaggtgtgagaatgattgcctaccgaatattgactgggttctctcgcttgcaaaagctgcagttttaagacatggggtgaatggacttgtaattgacccatacaatgaacttgatcatcaacgtccttcaagccagaccgaaacagagtatgtcagccagatgctgacgaagataaaacggtttgcacaacaccattcctgtcatgtttggtttgttgcacatccgagacagttgcatcattgggtaggaggtcctccaaatctgtatgatattagtggaagtgcacacttcataaacaaatgtgacaatggtattgttattcatcgaaacagagacccttcagctggccccgtggatcaagtgcaggtttgtgtaagaaaggtacggaataaagttagtggaacaattggtgatgccttcttatcatatgacagggttactggtgaattcatggacattgatgagcacccaaggaaaggctagctagctaattctggatattcttccattgtacataaggggaatgatactcgacttttgatgactgatcacagtgctcctagaagaagtttggagccatattgtgtggaaaatctggcagaaaaatctcacagtggttggcatacatatatgtggtggaggcgtggagcattgtgcctagtgaagttcttcgctttagtggctcttgtaagataaggtaacaagcaactattaagcttagaacatccattttcttagggcggcggtgttggaagctttaaaatttacctcttgttgaattgttcagtaagtgtgcttgattcctggcagattaaattagtctgatagtgcttaggtgatcaaatgtcatattctcaagttacatttgttttagtggtggtttattggttctgataagttgtaaaattaattgggttgatcccaataggaaatattaatgatttattgtaggcatccttctctcacagatattattggctttgcctatgtattcactatattaatattgtatttgtttttctagtctagccaattatgttgttgtaggttaa

SEQ ID NO: 96 - protein, 697 aa, Solanum lycopersicum
MILLPYRRIVVNNSNNFVMGSKYFLHKPSITLPTIYKSIPVLFKTQRLIFSAFASKPISPNRGTSSFSYRPQRIPPPVSVSGVMLEDPKEDITESDHEKALKQKLSQVGIDIGSCGPGQYNGLLCPMCKGGGSNEKSLSLFITPDGYAATWTCFRAKCGWRGGTRAFADVRTAFADMKRIGKVNKKYRQITEESLGLEPLCDVLLTYFSERMISRETLRRNAVMQQRHGDQVVIAFTYRRDGALVSCKYRNMTKKFWQEADTLKIFYGLDDIKGASDIIIVEGEMDKLAMEEAGFRNCVSVPDGAPPSISDKDLPPVEKDTKYQYLWNCKEYLEKTSRIILATDGDPPGQALAEELARRLGRERCWRVTWPKKSTIDHFKDANEVLMCLGPGALREVIEGAELYPIQGLFNFNNYFTEIDAYYHQTIGYELGVPTGWRSLNHLYNVVPGELTIVTGVPNSGKSEWIDALLCNLNYSVGWKFALCSMENRVREHARKLLEKHIKKPFFDVRYGESVERMSAQEFEEGKQWLSDTFFLIRCENDCLPNIDWVLSLAKAAVLRHGVNGLVIDPYNELDHQRPSSQTETEYVSQMLTKIKRFAQHHSCHVWFVAHPRQLHHWVGGPPNLYDISGSAHFINKCDNGIVIHRNRDPSAGPVDQVQVCVRKVRNKVSGTIGDAFLSYDRVTGEFMDIDEHPRKG

SEQ ID NO: 97 - DNA, 2905 bp, Solanum lycopersicum
tagtccatttccaatacattaaataccaatttgaagtaaatgagaaaccataaaacccttccttaaccccctaacggatgagtgtagttttgtgttgttgaaaattagcattagcattagcaggagcaggagcaggaatgattcttcttccttatcgcaggattgtagtgaataactccaacaactttgtaatgggctccaaatattttctccacaagccatccattactttaccaacaatatacaagtcgattcctgtactttttaaaacccaaagactcatcttttcagcatttgcttcaaaacccatatccccaaatcgcggaacttcaagcttctcatacagacctcaacgaattcctcctccagtttcagtttctggagtaatgctggaagatccgaaggaggatattacagagtcagaccacgagaaggcactgaagcagaaattaagccaggttgggatagatattggttcttgtggaccaggccagtacaatgggttgctttgtccaatgtgcaaaggtgggggctcaaatgaaaagagcttatctctctttattactccggatgggtatgcagctacatggacatgctttcgagctaaatgtggatggagaggtggtacacgggcctttgcagatgtgaggacagcttttgcggatatgaaaaggattgggaaagtgaataagaagtacagacagataactgaggagagtttggggctggaacctttatgtgatgtgttactcacatacttttctgagcgaatgatatcaagagaaacattgcggagaaatgctgttatgcaacaaagacatggtgatcaggttgttattgcatttacgtatcggagggatggagcacttgtaagctgtaagtaccggaacatgaccaaaaaattttggcaggaagctgatactttaaaaatattctatggtctcgacgatataaagggagcaagtgatatcatcattgttgagggtgagatggacaagcttgctatggaagaagcaggatttcggaattgtgtgagcgttccagatggcgctcctccatctatttcagataaagatttgccgcctgtagagaaggacacaaagtatcagtatctttggaactgcaaagaatatctggaaaagacatctcgcattatactggcaactgatggggatccacctggtcaagcattagctgaagagcttgcccgccggttagggagagaaaggtgctggcgagttacatggccaaaaaagagcactatagatcacttcaaagatgcaaatgaggtacttatgtgtctggggcctggtgcactgagagaggttattgaaggtgcagagctgtacccgatacaaggattattcaactttaataattactttactgagattgatgcatattatcatcaaacaattggctacgagcttggggttccaactggatggaggtccttaaatcatctatacaatgttgtaccgggagagttgactattgttacaggagtcccaaactccgggaagagtgaatggattgatgctcttctatgcaatctcaattacagtgttggctggaaatttgcactttgctctatggaaaacagggtacgggagcatgctagaaaacttttggagaagcacataaagaaacctttttttgatgtgagatatggggaatctgttgaacggatgagcgctcaggagtttgaagagggaaaacaatggctcagtgatacatttttcctcataaggtgtgagaatgattgcctgccgaatattgactgggttctctcgcttgcaaaagctgcagttttaagacatggggtgaatggacttgtaattgacccatacaatgaacttgatcatcaacgtccttcaagccagaccgaaacagagtatgtcagccagatgctgacgaagataaaacggtttgcacaacaccattcatgtcatgtttggtttgttgcacatccgagacagttgcatcattgggtaggaggtcctccaaatctgtacgatattagtggaagtgcacacttcataaacaaatgtgacaatggtattgttattcatcgtaacagagatccttcggctggccccgtggatcaagtgcaggtttgtgtaagaaaggtacggaataaagttagtggaacaattggtgatgccttcttatcatatgacagggttactggtgaattcatggacattgatgagcacccaaggaaaggctagttagcaaattctggatattcttccattgtacataacgggaatgatacttacttgacctgtgatgaatgatcacatcgctcctagaagaagtttgcagccatattatgtggaaatcaggcagaaaaatgtcgcagtggttggcatacatatatgtagtggagcattgtgcctagtgaagttcttcgctttagtggctcttgtaagataaggtaacaagcaactattaagcttagaacatcaattttcttagggcggcggtgttggaagctttaaaatttacctcttgttgaattgttcagtaagtgtgcttgattcctggcagattaaattagtctgatagtgcttaggtgatccaatgccatattctcaagttaacatttgttttggtcgtggcttattgttgtaaaattaattgggttgatcccaataggaaatattaatgattgattgtaggcatccttctttcacagatgttattggctttgcctatgtattcactatattactattgtatgtgttctctagtctagccaattgtgtggttgtaggttaatgttagctcctttttgctctgggcttcttactcaactacacaggtgaaaaaaagtttatgcctagtgtatttatgtggtataaatagctatataatgtatcgattcgaaattttatattaa

SEQ ID NO: 98 - DNA, 25 bp, Artificial Sequence
  Oligonucleotide primer
cattcataatgaccagctaggtgct

SEQ ID NO: 99 - DNA, 25 bp, Artificial Sequence
  Oligonucleotide primer
gagttgaaacatgcctgtactcctg

(19) United States
(12) Patent Application Publication
(10) Pub. No.: US 2024/0040982 A1
(43) Pub. Date: Feb. 8, 2024

(54) CEREAL GRAIN WITH THICKENED ALEURONE

(71) Applicants: Commonwealth Scientific and Industrial Research Organisation, Acton (AU); Institute of Botany, Chinese Academy of Sciences, Beijing (CN)

(72) Inventors: Dong-Qi LI, Beijing (CN); Chun-Ming LIU, Beijing (CN); Xiao-Ba WU, Florey (AU); Xue-Feng YAO, Beijing (CN); Jin-Xin LIU, Beijing (CN); Ronald Chun-Wai YU, Gungahlin (AU); Crispin Alexander HOWITT, Evatt (AU); Philip John LARKIN, Weston (AU)

(73) Assignees: Commonwealth Scientific and Industrial Research Organisation, Acton (AU); Institute of Botany, Chinese Academy of Sciences, Beijing (CN)

(21) Appl. No.: 18/255,184

(22) PCT Filed: Nov. 30, 2021
(86) PCT No.: PCT/AU2021/051425

§ 371 Date: May 31, 2023

(30) Foreign Application Priority Data
Dec. 1, 2020 (AU) 2020904452

(51) Int. Cl. A01H 1/00; A01H 5/10; A01H 6/46; C07K 14/415

(52) U.S. Cl. CPC A01H 1/121; A01H 5/10; A01H 6/46; C07K 14/415

(57) ABSTRACT

The present invention relates to cereal grain comprising an aleurone, an embryo, a starchy endosperm and a reduced level and/or activity of a mitochondrial single-stranded DNA binding (mtSSB) polypeptide, a RECA3 polypeptide or a TWINKLE polypeptide. Grain of the invention, or aleurone therefrom, has improved nutritional properties, and hence is particularly useful for human and animal feed products.

### FIELD OF THE INVENTION

The present invention relates to cereal grain comprising an aleurone, an embryo, a starchy endosperm and a reduced level and/or activity of a mitochondrial single-stranded DNA binding (mtSSB) polypeptide, a RECA3 polypeptide or a TWINKLE polypeptide. Grain of the invention, or aleurone therefrom, has improved nutritional properties, and hence is particularly useful for human and animal feed products.

### BACKGROUND OF THE INVENTION

Worldwide, cereal grains such as wheat, rice, maize and to a lesser extent barley, oats and rye are the major source of human caloric intake from the starch content of the grain. Cereal grain is also important in supplying other nutritional components such as protein, vitamins, minerals and dietary fibre. Different parts of the grain contribute differently for these nutritional components. Starch is stored in the starchy endosperm of cereal grains, whereas the other nutritional components are more concentrated in the embryo and bran (Buri et al., 2004). However, the bran is often removed before use in food, particularly in rice which is then eaten as white rice.

Cereal grain develops from double fertilisation events between maternal and paternal gametophytes. One of two sperm cells from the pollen tube fuses with an egg to produce a zygote that develops into an embryo, and the other sperm cell fuses with the diploid central cell of the megagametophyte to produce a primary endosperm nucleus, from which the genetically triploid endosperm develops. Thus, the endosperm including the aleurone is triploid, having two copies of the maternal haploid genome and one copy of the paternal haploid genome. In dicotyledonous seeds, the endosperm is consumed by the developing embryo whereas in monocotyledons such as rice the endosperm persists to make up the bulk of the mature grain.

The mature endosperm of cereals has four cell types with distinct characteristics, namely the starchy endosperm which is characterised by its abundant contents of starch granules and storage proteins, the epidermal-like aleurone which is most often one cell layer in thickness surrounding most of the starchy endosperm, transfer cells at the base of the seed over the main maternal vasculature, and a layer of embryo-surrounding cells which form a lining for the embryo early in grain development but later may only surround the suspensor which connects the embryo and starchy endosperm (Becraft et al., 2001a). The embryo forms within a cavity within the starchy endosperm. Cereal aleurone tissue therefore comprises the outermost layer(s) of the endosperm in cereal grains, and surrounds the starchy endosperm and part of the embryo.

Aleurone cells are distinguished from starchy endosperm cells by their morphology, biochemical composition and gene expression profiles (Becraft and Yi, 2011). Aleurone cells are generally oil and protein-rich and secrete enzymes allowing the mobilization of endosperm reserves during seed germination. Each aleurone cell is enclosed within a fibrous cell wall that is thicker than endosperm cell walls and that is composed mainly of arabinoxylans and beta glucans in various ratios and are highly autofluorescent. The aleurone layer is the only layer of the endosperm that in cereals is sometimes pigmented with anthocyanins.

Cereal aleurone is only one cell layer in thickness in wheat and wild-type maize (Buttrose 1963; Walbot, 1994), mostly one but up to three cell layers in the dorsal region of the endosperm in rice (Hoshikawa, 1993), and three cell layers in wild-type barley (Jones, 1969). In normal endosperm, the aleurone is extremely regular and the patterns of cell division are highly organised. Wild-type mature aleurone cells are nearly cuboid in section with a dense cytoplasm including granules, small vacuoles and inclusion bodies made of protein, lipid and phytin or of protein plus carbohydrate. In mature cereal grains, the aleurone is the only endosperm tissue that remains alive, although in a dormant, desiccated form. Upon imbibition, the embryo produces gibberellins which induce synthesis of amylases and other hydrolases by the aleurone which are released into the starchy endosperm to break down storage compounds to form sugars and amino acids for early growth of the embryo into a seedling.

The regulation of aleurone development in cereal grains has been reviewed by Becraft and Yi (2011). Multiple levels of genetic regulation control aleurone cell fate, differentiation and organisation, and many genes are involved in the processes, only some of which have been identified. For example, maize defective kernal1 (dek1) loss-of-function mutants have no aleurone layer indicating that the wild-type Dek1 polypeptide is required for specifying the outer cell layer as aleurone (Becraft et al., 2002). The Dek1 polypeptide is a large integral membrane protein with 21 membrane-spanning domains and a cytoplasmic domain containing an active calpain protease. Another gene in maize, CRINKLY4 (CR4) encodes a receptor kinase that functions as a positive regulator of aleurone fate, and cr4 mutants have reduced aleurone (Becraft et al., 2001b).

Several instances of thickened aleurones in cereal grain mutants have been reported in the literature, but none have proven useful because of pleiotrophic effects, or agronomic and production problems.

Shen et al. (2003) reported the identification of maize mutants in the supernumary aleurone layers1 (sal1) gene which in different mutants had 2-3 or up to seven layers of aleurone cells instead of the normal single layer. The SAL1 polypeptide was identified as a class E vacuolar sorting protein. Homozygous sail-1 mutant grain had defective embryos that failed to germinate and had much reduced starchy endosperm. A less complete mutant that was homozygous for the sail-2 allele exhibited a 2 cell-layer aleurone. However, the mutant plants grew to a height of only 30% of the wild-type, had a reduced root mass and were poor in seed setting (Shen et al. 2003). These plants were not agronomically useful.

Yi et al. (2011) reported the identification of a thick aleurone1 (thk1) mutant in maize. The mutant kernals showed a multilayer aleurone. However, the mutant kernals lacked well-developed embryos and did not germinate when sown. The wild-type Thk1 gene encoded a Thk1 polypeptide which acted downstream of the Dek1 polypeptide which was required for aleurone development in maize (Becraft et al., 2002).

A maize extra cell layer (Xcl) gene mutant was identified by its effect on leaf morphology. It produced a double aleurone layer as well as multilayered leaf epidermis (Kessler et al., 2002). The Xcl mutation was a semi-dominant mutation that disrupted cell division and differentiation patterns in maize, producing thick and narrow leaves with an abnormal shiny appearance.

Maize mutants in the disorgal1 and disorgal2 (dil1 and dil2) genes exhibited aleurones having a variable number of layers with cells of irregular shapes and sizes (Lid et al., 2004). However, homozygous dil1 and dil2 mutant grains were shrunken due to reduced accumulation of starch, and the mature mutant grains germinated at low rates and did not develop into viable plants.

In barley, elo2 mutants showed similarly disorganised cells and irregularities of the aleurone layers, resulting from aberrant periclinal cell division (Lewis et al., 2009). The plants also showed increased cell layers in the leaf epidermis, with bulging and distorted cells on the epidermis. Importantly, the homozygous mutant plants were dwarfed, producing grain weight of less than 60% of wild-type, and were not useful for grain production.

In rice, two transcription factors that control the expression of seed storage proteins also influence aleurone cell fate (Kawakatsu et al., 2009). Reduction in expression by co-suppression constructs of a gene encoding a rice prolamin box binding factor (RPBF) polypeptide, which is in the DOF zinc finger transcription factor class, resulted in a sporadic multilayered aleurone consisting of large, disordered cells. There was also a significant reduction in seed storage protein expression and accumulation, and starch and lipids were accumulated at substantially reduced levels. Expression of the rice homologs of the maize Dek1, CR4 and SAL1 genes was also reduced, showing that the RPBF and RISBZ1 factors operated in the same regulatory pathway as those genes.

More recently, it was determined a mutant ROS1a gene can result in thickened aleurone in rice (WO 2017/083920).

There is a need for cereal grain having thickened aleurone from plants, particularly rice plants that are also agronomically useful.

### SUMMARY OF THE INVENTION

The present inventors have identified numerous genes, and protein encoded thereby, which influence alureone development in cereal grain.

Thus, in an aspect the present invention provides cereal grain comprising an aleurone, an embryo, a starchy endosperm and a reduced level and/or activity of at least one mitochondrial polypeptide relative to a corresponding wild-type cereal grain, wherein the mitochondrial polypeptide which is reduced in its level and/or activity is at least one of a mitochondrial single-stranded DNA binding (mtSSB) polypeptide, a RECA3 polypeptide and a TWINKLE polypeptide.

In a preferred embodiment, the grain comprises a genetic variation which reduces the level and/or activity of the at least one mitochondrial polypeptide in the grain relative to the corresponding wild-type cereal grain. In an example of this embodiment, the grain comprises two or more genetic variations which reduce the level and/or activity of the at least one mitochondrial polypeptide in the grain relative to the corresponding wild-type cereal grain. In another example of this embodiment, the grain comprises two or more genetic variations which reduces the level and/or activity of two or three of the mitochondrial polypeptides in the grain relative to the corresponding wild-type cereal grain.

In an embodiment, the genetic variation comprises a mutation in an endogenous gene which encodes the mitochondrial polypeptide, whereby the mutation results in the reduced level and/or activity of the mitochondrial polypeptide.

In an embodiment, the genetic variation comprises an exogenous polynucleotide which encodes a silencing RNA molecule, wherein the silencing RNA molecule, and/or a processed RNA molecule produced from the silencing RNA molecule, reduces the expression of the endogenous gene, preferably wherein the exogenous polynucleotide comprises a DNA region encoding the silencing RNA molecule operably linked to a promoter which is expressed in developing grain of a cereal plant, wherein the promoter is preferably expressed at least at a time point between the time of pollination and 30 days post-pollination.

In an embodiment, the genetic variation comprises a splice site mutation which results in modified, preferably reduced, splicing of an RNA transcript of an endogenous gene which encodes the mitochondrial polypeptide, wherein the splice site mutation is preferably a single nucleotide substitution at a splice site, more preferably an adenine nucleotide at a position corresponding to nucleotide number 2126 of SEQ ID NO:4.

In an embodiment, the genetic variation comprises a deletion or an insertion within the endogenous gene which encodes the mitochondrial polypeptide, preferably a deletion which was introduced by mutagenesis of a progenitor cereal plant cell.

In an embodiment, the genetic variation comprises a premature translational stop codon in the protein coding region of an endogenous gene which encodes the mitochondrial polypeptide.

In an embodiment, the genetic variation comprises a mutation in an endogenous gene which encodes the mitochondrial polypeptide such that it encodes a polypeptide with reduced activity relative to the wild-type polypeptide, preferably wherein the mutation is a nucleotide substitution in the endogenous gene whereby the endogenous gene comprising the mutation encodes a polypeptide with an amino acid substitution relative to SEQ ID NO:3.

In an embodiment, the genetic variation comprises a mutation in an endogenous gene which encodes the mitochondrial polypeptide such that when the gene with the mutation is expressed it produces a reduced level of the polypeptide relative to the corresponding wild-type gene. For example, the genetic variation comprises a splice-site mutation that results in a reduced level of expression of the gene, or which gene comprises a mutation in its promoter which results in reduced expression of the gene.

In an embodiment, the genetic variation is an introduced genetic variation.

In an embodiment, the grain is heterozygous for the genetic variation. In an embodiment, the grain is homozygous for the genetic variation.

In a preferred embodiment, the grain has a thickened aleurone. In an embodiment, the grain has a thickened aleurone at day 20 after pollination. In an embodiment, the the thickened aleurone comprises at least two, at least three, at least four or at least five layers of cells, about 3, about 4, about 5 or about 6 layers of cells, or 2-8, 2-7, 2-6, 2-5 or 3-5 layers of cells, or a number of cell layers between 2 and 8, 2 and 7, or 2 and 6.

In an embodiment, the grain comprises, when compared to a corresponding wild-type cereal grain, one or more or all of the following, each on a weight basis,
i) a higher fat content,
ii) a higher ash content,
iii) a higher fiber content,
iv) a lower starch content,
v) a higher mineral content, preferably the mineral content is the content of one or more or all of calcium, iron, zinc, potassium, magnesium, phosphorus and sulphur,
vi) a higher antioxidant content,
vii) a higher phytate content,
viii) a higher content of one or more or all of vitamins B3, B6 and B9,
ix) a higher sucrose content,
x) a higher neutral non-starch polysaccharide content, and
xi) a higher monosaccharide content.

In an embodiment, the grain comprises, when compared to a corresponding wild-type cereal grain, a higher degree of chalkiness.

In an embodiment, the grain comprises, when compared to a corresponding wild-type cereal grain, a higher number of aleurone cells.

In an embodiment, the grain is whole grain or cracked grain.

In an embodiment, the grain has a germination rate which is between about 40 and about 100% relative to the germination rate of a corresponding wild-type cereal grain.

In an embodiment, the grain has an increased α-amylase activity during germination compared to the corresponding wild-type cereal grain.

In an embodiment, the grain has looser packing of irregular-shaped starch granules when compared to a corresponding wild-type cereal grain.

In an embodiment, the grain has one or more or all of about the same length, width, thickness, protein level and caryopsis morphology when compared to a corresponding wild-type cereal grain.

In an embodiment, the grain has been processed so that it is no longer able to germinate, preferably by heat treatment, more preferably which has been cooked.

In an embodiment, the polypeptide is expressed in one or more or all of the developing endosperm, testa, aleurone, and embryo of a developing grain.

In an embodiment, the grain pigmented in its outer layer(s).

Examples of cereal grain of the invention include, but are not limited to, rice grain, wheat grain, barley grain, maize grain, sorghum grain or oat grain. In an embodiment, the grain is rice grain. In an embodiment, the grain is brown rice grain or black rice grain.

In an embodiment, the wild-type grain comprises a mtSSB polypeptide which comprises an amino acid sequence as provided in any one of SEQ ID NOs: 3 or 15 to 39, or an amino acid sequence which is at least 75%, at least 80%, at least 90%, at least 91%, at least 92%, at least 93%, at least 94%, at least 95%, at least 96%, at least 97%, at least 98% or at least 99% identical to any one or more of SEQ ID NOs: 3 or 15 to 39.

In an embodiment, the mtSSB polypeptide is a mtSSB-1a polypeptide. In an embodiment, the wild-type grain comprises mtSSB-1a polypeptide which comprises an amino acid sequence as provided in any one of SEQ ID NOs: 3 or 15 to 23, or an amino acid sequence which is at least 75%, at least 80%, at least 90%, at least 91%, at least 92%, at least 93%, at least 94%, at least 95%, at least 96%, at least 97%, at least 98% or at least 99% identical to any one or more of SEQ ID NOs: 3 or 15 to 23.

In an embodiment, the wild-type grain comprises a mtSSB-1a polypeptide which comprises an amino acid sequence as provided in SEQ ID NO: 3, or an amino acid sequence which is at least 75%, at least 80%, at least 90%, at least 91%, at least 92%, at least 93%, at least 94%, at least 95%, at least 96%, at least 97%, at least 98% or at least 99% identical to SEQ ID NO: 3.

In an embodiment, the wild-type grain comprises a mtSSB polypeptide which comprises one or more or all of the following motifs:

(SEQ ID NO: 45)
FRGVHRAI(I/L)CGKVGQ(V/A)P(V/L)QKILRNG(R/H)T(V/I)T
(V/I)FT(V/I)GTGGMFDQR,
(SEQ ID NO: 46)
P(K/M)PAQWHRI(A/S)(V/I)H(N/S)(D/E),
(SEQ ID NO: 47)
AVQ(K/Q)L(V/T)KNS(A/S)VY(V/I)EG(D/E)IE(T/I)R(V/I)
YND
and
(SEQ ID NO: 48)
IC(L/V/I)R(R/G)DGKI.

In an embodiment, the grain comprises an endogenous gene which encodes a truncated mtSSB polypeptide, preferably a truncated mtSSB-1a polypeptide. In an embodiment, the truncated mtSSB polypeptide is C-terminally truncated. In an embodiment, the truncated mtSSB polypeptide is less than 200 amino acids in length. Examples of truncated mtSSB polypeptides of cereal grain of the invention include, but are not limited to, those consisting of an amino acid sequence selected from SEQ ID NO:8, SEQ ID NO:9, SEQ ID NO:10, or a truncated version of any one thereof.

In an embodiment, the reduced mtSSB activity is one or more or all of:
i) reduced ability to bind single stranded DNA,
ii) reduced ability to bind a RECA3 polypeptide, and
iii) reduced ability to bind a TWINKLE polypeptide.

In an embodiment, the wild-type grain comprises a RECA3 polypeptide which comprises an amino acid sequence as provided in any one of SEQ ID NOs: 61, 67, 69, 71, 73, 75, 77 or 79, or an amino acid sequence which is at least 75%, at least 80%, at least 90%, at least 91%, at least 92%, at least 93%, at least 94%, at least 95%, at least 96%, at least 97%, at least 98% or at least 99% identical to any one or more of SEQ ID NOs: 61, 67, 69, 71, 73, 75, 77 or 79.

In an embodiment, the wild-type grain comprises a RECA3 polypeptide which comprises an amino acid sequence as provided in SEQ ID NO: 61, or an amino acid sequence which is at least 75%, at least 80%, at least 90%, at least 91%, at least 92%, at least 93%, at least 94%, at least 95%, at least 96%, at least 97%, at least 98% or at least 99% identical to SEQ ID NO: 61.

In an embodiment, the reduced RECA3 polypeptide activity is one or both of:
i) reduced ability to bind a mtSSB polypeptide, and
ii) reduced recombinase activity.

In an embodiment, the wild-type grain comprises a TWINKLE polypeptide which comprises an amino acid sequence as provided in any one of SEQ ID NOs: 64, 80, 82, 84, 86, 88, 90, 92, 94 or 96, or an amino acid sequence which is at least 75%, at least 80%, at least 90%, at least 91%, at least 92%, at least 93%, at least 94%, at least 95%, at least 96%, at least 97%, at least 98% or at least 99% identical to any one or more of SEQ ID NOs: 64, 80, 82, 84, 86, 88, 90, 92, 94 or 96.

In an embodiment, the wild-type grain comprises a TWINKLE polypeptide which comprises an amino acid sequence as provided in SEQ ID NO: 64, or an amino acid sequence which is at least 75%, at least 80%, at least 90%, at least 91%, at least 92%, at least 93%, at least 94%, at least 95%, at least 96%, at least 97%, at least 98% or at least 99% identical to SEQ ID NO: 64.

In an embodiment, the reduced TWINKLE polypeptide activity is one or both of:
i) reduced ability to bind a mtSSB polypeptide, and
ii) reduced helicase activity.

Also provided is a mutant mitochondrial single-stranded DNA binding (mtSSB) polypeptide, a mutamt RECA3 polypeptide or a mutant TWINKLE polypeptide whose amino acid sequence is different to the amino acid sequence of a corresponding wild-type mitochondrial single-stranded DNA binding (mtSSB) polypeptide, RECA3 polypeptide or TWINKLE polypeptide, respectively, and which has reduced activity when compared to the corresponding wild-type polypeptide, preferably which is encoded by an endogenous gene of a cereal plant comprising a genetic variation of the invention.

In another aspect, the present invention provides a polynucleotide encoding mutant mitochondrial single-stranded DNA binding (mtSSB) polypeptide, RECA3 polypeptide or TWINKLE polypeptide of the invention, preferably an endogenous gene of a cereal plant comprising a genetic variation of the invention.

In an embodiment, the polynucleotide is operably linked to a promoter which is expressed in grain of a cereal plant.

In a further aspect, the present invention provides an isolated and/or exogenous polynucleotide which, when present in a grain of a cereal plant, reduces the expression of a gene encoding a mitochondrial single-stranded DNA binding (mtSSB) polypeptide, a RECA3 polypeptide or a TWINKLE polypeptide.

In an embodiment, the polynucleotide is operably linked to a promoter which is expressed in grain of a cereal plant.

Also provided is a polynucleotide of the above aspect when used for reducing the expression of the gene in developing grain of a cereal plant at least at a time point between the time of pollination and 30 days post-pollination.

The skilled person is well aware of different types of polynucleotides that can be used to reduce the expression of a target gene, and how these polynucleotides can be designed. Examples include, but are not limited to, a double stranded RNA (dsRNA) molecule or a processed RNA product thereof, a micro RNA, an antisense polynucleotide, a sense polynucleotide or a catalytic polynucleotide.

In an embodiment, the polynucleotide is a dsRNA molecule, or a processed RNA product thereof, comprising at least 19, at least 20 or at least 21 consecutive nucleotides which are at least 95% identical to the complement of a gene encoding a cereal mitochondrial single-stranded DNA binding (mtSSB) polypeptide, a RECA3 polypeptide or a TWINKLE polypeptide (where thymine (T) is uracil (U)), or at least 95% identical to the complement of an mRNA encoding a cereal mitochondrial single-stranded DNA binding (mtSSB) polypeptide, a RECA3 polypeptide or a TWINKLE polypeptide. For example, in an embodiment the polynucleotide is a dsRNA molecule, or a processed RNA product thereof, comprising at least 19 consecutive nucleotides which are at least 95% identical to the complement of a SEQ ID NO:1 (where thymine (T) is uracil (U)), or at least 95% identical to the complement of an mRNA encoding a mtSSB polypeptide comprising an amino acid sequence provided as SEQ ID NO:3.

In another embodiment, the dsRNA molecule is a microRNA (miRNA) precursor and/or wherein the processed RNA product thereof is a miRNA.

In another aspect, the present invention provides a nucleic acid construct and/or vector encoding a polynucleotide of the invention, wherein the nucleic acid construct or vector comprises a DNA region encoding the polynucleotide operably linked to a promoter which is expressed in developing grain of a cereal plant at least at a time point between the time of pollination and 30 days post-pollination.

In another aspect, the present invention provides a cell, tissue, organ, plant part or plant comprising a polynucleotide of the invention, or a nucleic acid construct and/or vector of the invention.

In an embodiment, the polynucleotide, nucleic acid construct or vector is integrated into the genome of the cell, tissue, organ, plant part or plant, preferably into the nuclear genome.

In another aspect, the present invention provides a cereal plant cell, seed or tissue therefrom, the cell, seed or tissue therefrom comprising a reduced level and/or activity of at least one mitochondrial polypeptide relative to a corresponding wild-type cereal plant cell, wherein the mitochondrial polypeptide which is reduced in its level and/or activity is at least one of a mitochondrial single-stranded DNA binding (mtSSB) polypeptide, a RECA3 polypeptide and a TWINKLE polypeptide.

In an embodiment, the cell, seed or tissue therefrom is or comprises an endosperm, testa, aleurone or embryo cell.

In an embodiment, the cell is an aleurone cell.

In another aspect, the present invention provides a cereal plant which produces grain of the invention, and/or which comprises one or more or all of a polypeptide of the invention, a polynucleotide of the invention, a nucleic acid construct and/or vector of the invention or a cell, seed or tissue of the invention.

In an embodiment, a plant of the invention has about the same height as a corresponding wild-type plant.

In an embodiment, a plant of the invention is male and female fertile.

In an embodiment, a plant of the invention exhibits delayed grain maturation.

Also provided is a population of at least 100 cereal plants of the invention growing in a field.

In another aspect, the present invention provides a method of producing the cell of the invention, the method comprising a step of introducing a genetic variation of the invention, a polynucleotide of the invention, or a nucleic acid construct and/or vector of the invention, into a cell, preferably a cereal plant cell.

In another aspect, the present invention provides a method of producing a cereal plant of the invention, or grain therefrom, the method comprising the steps of
i) introducing into a cereal plant cell, a genetic variation of the invention, a polynucleotide of the invention, or a nucleic acid construct and/or vector of the invention,
ii) obtaining a cereal plant comprising the genetic variation or polynucleotide from a cell obtained from step i), and
iii) optionally harvesting grain from the plant of step ii), the grain comprising the genetic variation or being transgenic for the polynucleotide, nucleic acid construct or vector, and
iv) optionally producing one or more generations of progeny plants from the grain, the progeny plants comprising the genetic variation or being transgenic for the polynucleotide, nucleic acid construct or vector, thereby producing the cereal plant or grain.

In another aspect, the present invention provides a method of producing a cereal plant of the invention, or grain therefrom, the method comprising the steps of
i) introducing into a cereal plant cell, a mutation to an endogenous gene which encodes a mitochondrial single-stranded DNA binding (mtSSB) polypeptide, a RECA3 polypeptide or a TWINKLE polypeptide, such that the cell has reduced level and/or activity of the mitochondrial single-stranded DNA binding (mtSSB) polypeptide, RECA3 polypeptide or TWINKLE polypeptide relative to a corresponding wild-type cereal plant cell,
ii) obtaining a cereal plant from a cell of step i), the cereal plant comprising the mutation of the endogenous gene, and
iii) optionally harvesting grain from the plant of step ii), the grain comprising the mutation, and
iv) optionally producing one or more generations of progeny plants from the grain, the progeny plants comprising the mutation, thereby producing the cereal plant or grain.

In another aspect, the present invention provides a method of selecting a cereal plant of the invention or grain of the invention, the method comprising the steps of
i) screening a population of cereal plants or grain each of which were obtained from a mutagenic treatment of progenitor cereal plant cells, grain or plants, for the production of grain of the invention or for the presence of a mutation in a gene encoding a mitochondrial single-stranded DNA binding (mtSSB) polypeptide, a RECA3 polypeptide or a TWINKLE polypeptide, and
ii) selecting from the population of step (i) a cereal plant which produces grain of the invention or which comprises a mutation in the gene, thereby selecting the cereal plant or grain.

In another aspect, the present invention provides a method of selecting a cereal plant of the invention, the method comprising the steps of
i) producing one or more progeny plants from cereal grain, the cereal grain having been derived from a cross of two parental cereal plants,
ii) screening the one or more progeny plants of step i) for the production of grain of the invention, and
iii) selecting a progeny plant which produces the grain, thereby selecting the cereal plant.

In an embodiment, step ii) comprises analysing a sample comprising DNA from a progeny plant for the genetic variation.

In an embodiment, step ii) comprises analysing the thickness of aleurone of grain obtained from a progeny plant.

In an embodiment, step ii) comprises analysing the nutritional content of the grain or a part thereof.

In an embodiment, step iii) comprises selecting a progeny plant which is homozygous for the genetic variation.

In an embodiment, step iii) selecting a progeny plant whose grain has an increased aleurone thickness compared to a corresponding wild-type cereal grain.

In an embodiment, step iii) selecting a progeny plant whose grain or a part thereof has an altered nutritional content compared to a corresponding wild-type cereal grain or part thereof.

In an embodiment, the method further comprises
i) crossing two parental cereal plants, preferably wherein one of the parental cereal plants produces grain of the invention, or
ii) backcrossing one or more progeny plants from step i) with plants of the same genotype as a first parental cereal plant which does not produce grain of the invention for a sufficient number of times to produce a plant with a majority of the genotype of the first parental cereal plant but which produces of the invention, and
iii) selecting a progeny plant which produces grain of the invention.

Also provided is a cereal plant produced using a method of the invention.

Also provided is the use of a polynucleotide of the invention, or a nucleic acid construct and/or vector of the invention, to produce a cell, a cereal plant or plant part such as cereal grain.

In an embodiment, the use is to produce grain of the invention.

In a further aspect, the present invention provides a method for identifying a cereal plant which produces grain of the invention, the method comprising the steps of
i) obtaining a nucleic acid sample from a cereal plant, and
ii) screening the sample for the presence or absence of a genetic variation which reduces the level of activity of a mitochondrial single-stranded DNA binding (mtSSB) polypeptide, a RECA3 polypeptide or a TWINKLE polypeptide in the plant when compared to a corresponding wild-type cereal plant.

In an embodiment, the genetic variation is one or both of
a) a nucleic acid construct expressing a polynucleotide, or the polynucleotide encoded thereby, which when present in a cereal plant reduces the expression of a gene encoding a mitochondrial single-stranded DNA binding (mtSSB) polypeptide, a RECA3 polypeptide or a TWINKLE polypeptide, and
b) a gene, or mRNA encoded thereby, which expresses a mutant, preferably truncated, mitochondrial single-stranded DNA binding (mtSSB) polypeptide, RECA3 polypeptide or TWINKLE polypeptide with reduced activity.

In an embodiment, the presence of the genetic variation indicates that grain of the cereal plant has a thickened aleurone when compared to a corresponding cereal plant lacking the genetic variation(s).

In another aspect, the present invention provides a method for identifying a cereal plant which produces grain of the invention, the method comprising the steps of
i) obtaining grain from a cereal plant, and
ii) screening the grain or a portion thereof for one or more of
   a) a thickened aleurone,
   b) the amount of mitochondrial single-stranded DNA binding (mtSSB) polypeptide, RECA3 polypeptide or TWINKLE polypeptide and/or activity in the grain, and
   c) the amount of mRNA encoded by a gene encoding a mitochondrial single-stranded DNA binding (mtSSB) polypeptide, a RECA3 polypeptide or a TWINKLE polypeptide in the grain.

In an embodiment, the method identifies a cereal plant of the invention.

In a further aspect, the present invention provides a method of producing a cereal plant part, the method comprising,
a) growing a cereal plant, or at least 100 such cereal plants in a field, of the invention, and
b) harvesting the cereal plant part from the cereal plant or cereal plants.

In a further aspect, the present invention provides a method of producing processed grain, or flour, bran, wholemeal, malt, starch or oil obtained from grain, the method comprising;
a) obtaining grain of the invention, and
b) processing the grain to produce the processed grain, flour, bran, wholemeal, malt, starch or oil.

Also provided is a product produced from grain of the invention, or a cereal plant of the invention, or from a part of said grain or cereal plant comprising the genetic variation.

In an embodiment, the product comprises one or more or all of the genetic variations, the polynucleotide, the nucleic acid construct or the vector, and the thickened aleurone.

In an embodiment, the part is cooked, boiled, parboiled, roasted, baked, polished, cracked, puffed, milled or flaked grain, or bran.

In an embodiment, the product is a food ingredient, beverage ingredient, food product or beverage product.

In an embodiment, the food ingredient or beverage ingredient is selected from the group consisting of roasted grain, polished grain, cracked grain, puffed grain, milled grain, flaked grain, wholemeal, flour, bran, starch, malt and oil.

In an embodiment, the food product is selected from the group consisting of processed grain, cooked grain, boiled grain, porridge, leavened or unleavened breads, pasta, noodles, animal fodder, breakfast cereals, snack foods, cakes, pastries and foods containing a flour-based sauce.

In an embodiment, the beverage product is a tea, a packaged beverage or a beverage comprising ethanol.

In a further aspect, the present invention provides a method of preparing a food or beverage ingredient of the invention, the method comprising processing grain of the invention, or bran, flour, wholemeal, malt, starch or oil from the grain, to produce the food or beverage ingredient.

In a further aspect, the present invention provides a method of preparing a food or beverage product of the invention, the method comprising processing grain, preferably by cooking, boiling, roasting or flaking grain or mixing grain of the invention, or bran, flour, wholemeal, malt, starch or oil from the grain, with another food or beverage ingredient.

Also provided is the use of grain of the invention or part thereof, or a cereal plant of the invention or part thereof, as animal feed or food, or to produce feed for animal consumption or food for human consumption.

In another aspect, the present invention provides a composition comprising one or more of a polypeptide of the invention, a polynucleotide of the invention a nucleic acid construct and/or vector of the invention, or a cell of the invention, and one or more acceptable carriers, preferably another food ingredient.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. ta1-1 mature caryopsis showed thick aleurone phenotype. Mature caryopsis of the wild-type (ZH11, A) and ta1-1 (B); transverse sections of ZH11 (C) and ta1-1 (D) mature caryopsis; Lugol's staining of ZH11 (E) and ta1-1 (F) mature caryopsis, arrows point to the aleurone; PAS and Coomassie brilliant blue (G-250) staining of ZH11 5 DAP (G), 10 DAP (I), 30 DAP (K) and ta1-1 5 DAP (H), 10 DAP (J), 30 DAP (L) caryopsis Semi-thin sections. Bar=2 mm in A and B, 1 mm in C, D, E and F, 20 μm in G, H, I, J, K and L. DAP: days after pollination.

FIG. 2. Average number of aleurone cell layers in mature grains of the wild-type and ta1-1 at different positions of the caryopsis. The values represent mean±SD (n=15), ** P<0.01.

FIG. 3. Analysis of grain dry weight changes in time. DAP: days after pollination. Data are shown as mean±SD (n=10), **P<0.01.

FIG. 4. Nutrient contents of ZH11 (wild-type) and ta1-1 mature caryopses. Total protein (A), crude lipids (B), dietary fiber (C), calcium (D), iron (E), zinc (F), vitamin B2 (G) and vitamin B3 (H) content in mature caryopses were measured. Data shown as mean±SEM, n=3, (*, P<0.05; **, P<0.01).

FIG. 5. α-amylase activity assay of ta1-1 endosperm and aleurone from germinated grains after imbibition. The darker bars are for ta1-1 grain. Differences between ZH11 and ta1 were significant as determined by Student t test: *P<0.05; **P<0.01. Each bar represented the mean of 3 samples.

FIG. 6. TA1 map-based cloning, showing Indel markers mapped to chromosome 5 of rice. Each linear map represents enlargement of a segment from the map immediately above. n shows the number of recombinants used in each stage of the mapping process. Indel=an insertion or deletion, identified by number. The number below each vertical line shows the number of recombinants between the Indel marker and the ta1-1 mutation. Mb, million basepairs, Kb, one thousand basepairs. The lowest line is a schematic diagram of the TA1 gene with filled rectangular boxes representing exons (Arabic numerals 1-6) within the protein coding region and lines representing introns (Roman numerals I-V) within the protein coding region. The arrow labelled G to A shows the position of the G to A point mutation in the tat-1 allele responsible for the thick aleurone phenotype.

FIG. 7. Alignment of three nucleotide sequences (SEQ ID NOs:12-14) of a region of the cDNAs produced from different ta1-1 RNAs, labelled ta1 I, ta1 II and ta1 III, compared to the nucleotide sequence (SEQ ID NO:11) of the corresponding region of wild-type TA1 cDNA (ZH11). This region was involving in the alternative splicing of the transcripts from the mutant tat-1 gene.

FIG. 8. Predicted amino acid sequences (SEQ ID NOs:8, 9 and 10) of the polypeptides translated from the three different RNAs produced in ta1-1 grain, compared to wild-type TA1 polypeptide sequence (ZH11, SEQ ID NO:3).

FIG. 9. Alignment of the amino acid sequences of wild-type OsTA1 polypeptide (SEQ ID NO:3) and homologs in Zea mays (SEQ ID NO:16), Sorghum bicolor (SEQ ID NO:17), barley Hordeum vulgare (SEQ ID NO:18), wheat Triticum aestivum (SEQ ID NO:19), Brachypodium distachyon (SEQ ID NO:20), Arabidopsis thaliana (SEQ ID NO:15) and poplar (SEQ ID NO:21). A related rice sequence (OsTA1L; SEQ ID NO:24) was also used in the alignment. Amino acids fully conserved in the 9 sequences are indicated by asterisks, mostly conserved amino acids are indicated with a semi-colon, and similar amino acids with a dot. The single-stranded DNA binding (SSB) domain is indicated by the solid bar, while the predicted cleavage site to remove the mitochondrial transit peptide (MTP) is indicated by the arrow.

FIG. 10. Relative expression level of TA1 gene in different rice tissues. SAM: shoot apical meristem, DAP: day after pollination. All expression levels are normalized to an actin gene. Three replicates for each sample were made and illustrated as mean±SD.

FIG. 11. 6×His-TA1 protein binding to ssDNA using a biotin-labelled, 45-nucleotide ssDNA molecule as a probe (lane 2). Adding non-labelled ssDNA at 2× (lane 3), 20× (lane 4), and 200× (lane 5) as a competitor ssDNA progressively diminished the amount of mobility shifted biotin-labelled ssDNA in the presence of the TA1 protein.

FIG. 12. 6×His-TA1 and its mutant form 6×His-ta1-1 polypeptide (Truncated TA1 polypeptide) were used to examine their binding to ssDNA by EMSA experiment using a biotin-labelled 45-nucleotide ssDNA molecule as a probe. 2 μg recombinant TA1 protein was used to perform the assay as for FIG. 11, and a gradient of 2 μg, 4 μg and 8 μg of recombinant 6×His-ta1-1 polypeptide was used under the same conditions.

FIG. 13. Expression of TA1 in aleurone and embryo of rice caryopsis in different development stages. Caryopses from pTA1:TA1-GUS transgenic plants were stained for GUS activity at 5 DAP (A and G), 7 DAP (B and H), 9 DAP (C and I), 11 DAP (D and J), 18 DAP (E and K), and 24 DAP (F and L). Arrows in C, D, I, and J indicate the aleurone cell layer. Bar=0.2 cm in A, B, C, D, E and F, bar=0.1 cm in G, H, 1, J, K and L. DAP: days after pollination.

FIG. 14. A. The numbers of mitochondria in each section of aleurone cells of ta1-1 developing grain compared to wild-type at 15 DAP (n=10). B. ATP contents in the wild-type and ta1-1 aleurone at 11 DAP. Data are shown as the mean±SD. n=3; ** indicates P<0.01.

FIG. 15. Down-regulation of RecA3 and TWINKLE genes in rice using RNAi. Upper panel shows schematic maps for the RecA3 and TWINKLE genes with exons as black boxes and introns as lines between the boxes. The regions of the genes selected (exons only) for the RNAi constructs are shown. b and c. Quantitative RT-PCR was used to measure mRNA levels in the grain of three transformed rice plants for each of the RNAi constructs, showing the reduced expression in each of the selected plants.

FIG. 16. Phylogenetic analysis of mtSSB proteins from different plant species, mostly cereals. Protein sequences were aligned with ClustalW and the phylogeny was produced with MEGA7 using the Neighbor-Joining method.

FIG. 17. Phylogenetic analysis of TWINKLE proteins from different plant species, including some cereals. Protein sequences were aligned with ClustalW and the phylogeny was produced with MEGA7 using the Neighbor-Joining method.

Key to the Sequence Listing

SEQ ID NO:1—Nucleotide sequence of the wild-type rice TA1 gene, locus Os05g43440. Nucleotides 1-1024, TA1 promoter and 5′UTR; 1025-1027, ATG translation start codon; protein coding region is nucleotides 1025-1094, 1176-1339, 1425-1633, 1771-1871, 2127-2186 and 2353-2369. Nucleotides 1095-1175, intron 1; 1340-1424, intron 2; 1634-1770, intron 3; 1872-2126, intron 4; 2187-2352, intron 5. Translation stop is nucleotides 2367-2369.

SEQ ID NO:2—Nucleotide sequence of the protein coding region of cDNA of wild-type TA1 gene including TAG stop codon.

SEQ ID NO:3—Amino acid sequence of wild-type TA1 (OsmtSSB) polypeptide.

SEQ ID NO:4—Nucleotide sequence of the rice ta1-1 (mutant) gene, locus Os05g43440. The ta1-1 mutation is at nucleotide 2126 (G2126A), the last nucleotide of intron 4. Nucleotides 1-1024, TA1 promoter and 5′UTR; 1025-1027, ATG translation start codon; protein coding region is nucleotides 1025-1094, 1176-1339, 1425-1633, 1771-1871, 2127-2186 and 2353-2369. Nucleotides 1095-1175, intron 1; 1340-1424, intron 2; 1634-1770, intron 3; 1872-2126, intron 4; 2187-2352, intron 5. Translation stop is nucleotides 2367-2369.

SEQ ID NO:5—Nucleotide sequence of the ta1-1 I cDNA, from the translation start ATG to the wild-type TA1 stop codon TAG.

SEQ ID NO:6—Nucleotide sequence of the ta1-1 II cDNA, from the translation start ATG to the wild-type TA1 stop codon TAG.

SEQ ID NO:7—Nucleotide sequence of the ta1-1 III cDNA, from the translation start ATG to the wild-type TA1 stop codon TAG.

SEQ ID NO:8—Predicted amino acid sequence of ta1-1 I polypeptide.

SEQ ID NO:9—Predicted amino acid sequence of ta1-1 II polypeptide.

SEQ ID NO:10—Predicted amino acid sequence of ta1-1 II polypeptide.

SEQ ID NO:11—Nucleotide sequence of a region of the cDNA from the wild-type TA1 gene.

SEQ ID NO:12—Nucleotide sequence of a region of cDNA I from the ta1-1 gene.

SEQ ID NO:13—Nucleotide sequence of a region of cDNA II from the ta1-1 gene.

SEQ ID NO:14—Nucleotide sequence of a region of cDNA III from the ta1-1 gene.

SEQ ID NO:15—Amino acid sequence of TA1 homologous protein in Arabidopsis thaliana.

SEQ ID NO:16—Amino acid sequence of TA1 homologous protein in Zea mays.

SEQ ID NO:17—Amino acid sequence of TA1 homologous protein in Sorghum bicolor.

SEQ ID NO:18—Amino acid sequence of TA1 homologous protein in Hordeum vulgare.

SEQ ID NO:19—Predicted amino acid sequence of TamtSSB-1a;D in Triticum aestivum.

SEQ ID NO:20—Amino acid sequence of TA1 homologous protein in Brachypodium distachyon.

SEQ ID NO:21—Amino acid sequence of TA1 homologous protein in poplar, Populus trichocarpa.

SEQ ID NO:22—Predicted amino acid sequence of TamtSSB-1a;B in Triticum aestivum.

SEQ ID NO:23—Predicted amino acid sequence of SimtSSB-1a in Setaria italica.

SEQ ID NO:24—Amino acid sequence of TA1-like (OsmtSSB-1b) protein in Oryza sativa.

SEQ ID NO:25—Predicted amino acid sequence of SimtSSB-1b in Setaria italica.

SEQ ID NO:26—Predicted amino acid sequence of HvmtSSB-1b in Hordeum vulgare sub sp. vulgare.

SEQ ID NO:27—Predicted amino acid sequence of TamtSSB-1b;A in Triticum aestivum.

SEQ ID NO:28—Predicted amino acid sequence of TamtSSB-1b;B in Triticum aestivum.

SEQ ID NO:29—Predicted amino acid sequence of TamtSSB-1b;D in Triticum aestivum.

SEQ ID NO:30—Predicted amino acid sequence of OsmtSSB-2 in Oryza saliva Japonica Group.

SEQ ID NO:31—Predicted amino acid sequence of AtmtSSB-2 in Arabidopsis thaliana.

SEQ ID NO:32—Predicted amino acid sequence of SbmtSSB-2 in Sorghum bicolor.

SEQ ID NO:33—Predicted amino acid sequence of ZmmtSSB-2 in Zea mays.

SEQ ID NO:34—Predicted amino acid sequence of SimtSSB-2 in Setaria italica.

SEQ ID NO:35—Predicted amino acid sequence of HvmtSSB-2 in Hordeum vulgare sub sp. vulgare.

SEQ ID NO:36—Predicted amino acid sequence of TamtSSB-2;A in Triticum aestivum.

SEQ ID NO:37—Predicted amino acid sequence of TamtSSB-2;B in Triticum aestivum.

SEQ ID NO:38—Predicted amino acid sequence of TamtSSB-2;D in Triticum aestivum.

SEQ ID NO:39—Predicted amino acid sequence of PtmtSSB-2 in Populus trichocarpa.

SEQ ID NO:40—Predicted amino acid sequence of MmmtSSB in Mus musculus.

SEQ ID NO:41—Predicted amino acid sequence of HsmtSSB in Homo sapiens.

SEQ ID NO:42—Predicted amino acid sequence of DmmtSSB in Drosophila melanogaster.

SEQ ID NO:43—Predicted amino acid sequence of ScmtSSB in Saccharomyces cerevisiae.

SEQ ID NO:44—Predicted amino acid sequence of EcmtSSB in Escherichia coli.

SEQ ID NO:45—Amino acid motif; 42aa.

SEQ ID NO:46—Amino acid motif; 14aa.

SEQ ID NO:47—Amino acid motif; 24aa.

SEQ ID NO:48—Amino acid motif; 9aa.

SEQ ID NO:49—Nucleotide sequence used to analyse TA1 expression pattern. Nucleotides 1-3208 contained the TA1 promoter and 5′UTR; 3209-3211, ATG translation start codon; protein coding region is nucleotides 3209-3278, 3360-3523, 3609-3817, 3955-4055, 4311-4370 and 4537-4553. Nucleotides 3279-3359, intron 1; 3524-3608, intron 2; 3818-3954, intron 3; 4056-4310, intron 4; 4371-4536, intron 5. A translation stop and the following GUS protein coding region are not included in this sequence.

SEQ ID NO:50—Nucleotide sequence of the forward primer for detecting molecular marker Indel 559 for ta1 mapping.

SEQ ID NO:51—Nucleotide sequence of the reverse primer for detecting molecular marker Indel 559 for ta1 mapping.

SEQ ID NO:52—Nucleotide sequence of the forward primer for detecting molecular marker Indel 548 for ta1 mapping.

SEQ ID NO:53—Nucleotide sequence of the reverse primer for detecting molecular marker Indel 548 for ta1 mapping.

SEQ ID NO:54—Nucleotide sequence of the forward primer for detecting molecular marker Indel 562 for ta1 mapping.

SEQ ID NO:55—Nucleotide sequence of the reverse primer for detecting molecular marker Indel 562 for ta1 mapping.

SEQ ID NO:56—Nucleotide sequence of the forward primer for detecting molecular marker Indel 583 for ta1 mapping.

SEQ ID NO:57—Nucleotide sequence of the reverse primer for detecting molecular marker Indel 583 for ta1 mapping.

SEQ ID NO:58—Nucleotide sequence of the forward primer for detecting transcripts of the TA1 and ta1-1 genes.

SEQ ID NO:59—Nucleotide sequence of the reverse primer for detecting transcripts of the TA1 and ta1-1 genes.

SEQ ID NO:60—Nucleotide sequence of probe used in EMSA experiment.

SEQ ID NO:61—Amino acid sequence of rice RECA3 DNA repair protein, Oryza sativa Japonica Group.

SEQ ID NO:62—Nucleotide sequence of rice RECA3 gene, Oryza sativa Japonica Group cultivar Nipponbare chromosome 1.

SEQ ID NO:63—Nucleotide sequence of the cDNA for the rice RECA3 gene, mitochondrial RECA3, Oryza saliva Japonica Group cultivar Nipponbare.

SEQ ID NO:64—Amino acid sequence of rice TWINKLE chloroplastic/mitochondrial DNA repair protein, Oryza sativa Japonica Group.

SEQ ID NO:65—Nucleotide sequence of rice TWINKLE gene, Oryza sativa Japonica Group cultivar Nipponbare chromosome 6.

SEQ ID NO:66—Nucleotide sequence of the cDNA for the rice TWINKLE gene, chloroplastic/mitochondrial, from Oryza sativa Japonica Group cultivar Nipponbare. The protein coding region is nucleotides 153-2321.

SEQ ID NO:67—Amino acid sequence of RECA3 DNA repair protein, mitochondrial, Brachypodium distachyon.

SEQ ID NO:68—Nucleotide sequence of the cDNA for the RECA3 gene, mitochondrial RECA3, Brachypodium distachyon. The protein coding region is nucleotides 196-1482.

SEQ ID NO:69—Amino acid sequence of RECA3 DNA repair protein, mitochondrial, Sorghum bicolor.

SEQ ID NO:70—Nucleotide sequence of the cDNA for the sorghum RECA3 gene, mitochondrial RECA3, Sorghum bicolor. The protein coding region is nucleotides 230-1510.

SEQ ID NO:71—Amino acid sequence of maize RECA3 DNA repair protein, mitochondrial, Zea mays.

SEQ ID NO:72—Nucleotide sequence of the cDNA for the maize RECA3 gene, mitochondrial RECA3, Zea mays. The protein coding region is nucleotides 59-1333. SEQ ID NO:73—Amino acid sequence of barley RECA3 DNA repair protein, mitochondrial, Hordeum vulgare subsp. vulgare.

SEQ ID NO:74—Nucleotide sequence of the cDNA for the barley RECA3 gene, mitochondrial RECA3, Hordeum vulgare subsp. vulgare. The protein coding region is nucleotides 122-1408.

SEQ ID NO:75—Amino acid sequence of wheat RECA3 DNA repair protein, mitochondrial, Triticum aestivum cultivar.

SEQ ID NO:76—Nucleotide sequence of the cDNA for the wheat RECA3 gene from Triticum aestivum, cultivar Chinese Spring. The protein coding region is nucleotides 112-1416.

SEQ ID NO:77—Amino acid sequence of RECA3 DNA repair protein, mitochondrial, from Aegilops tauschii subsp. tauschii.

SEQ ID NO:78—Nucleotide sequence of the cDNA for a RECA3 gene, encoding a mitochondrial RECA3, from Aegilops tauschii subsp. tauschii. The protein coding region is nucleotides 129-1427.

SEQ ID NO:79—Amino acid sequence of RECA3 DNA repair protein, mitochondrial, from Triticum turgidum subsp. durum.

SEQ ID NO:80—Amino acid sequence of TWINKLE homologous protein, chloroplastic/mitochondrial from Brachypodium distachyon.

SEQ ID NO:81—Nucleotide sequence of the cDNA for a TWINKLE gene, encoding a chloroplastic/mitochondrial TWINKLE protein, from Brachypodium distachyon. The protein coding region is nucleotides 132-2300.

SEQ ID NO:82—Amino acid sequence of TWINKLE homologous protein, chloroplastic/mitochondrial from Sorghum bicolor.

SEQ ID NO:83—Nucleotide sequence of the cDNA for a TWINKLE gene, encoding a chloroplastic/mitochondrial TWINKLE protein, from Sorghum bicolor. The protein coding region is nucleotides 154-2436.

SEQ ID NO:84—Amino acid sequence of TWINKLE homologous protein, chloroplastic/mitochondrial from Zea mays.

SEQ ID NO:85—Nucleotide sequence of the cDNA for a TWINKLE gene, encoding a chloroplastic/mitochondrial TWINKLE protein, from Zea mays. The protein coding region is nucleotides 144-2351.

SEQ ID NO:86—Amino acid sequence of TWINKLE homologous protein, chloroplastic/mitochondrial from Aegilops tauschii subsp. tauschii.

SEQ ID NO:87—Nucleotide sequence of a cDNA for a TWINKLE gene, encoding a chloroplastic/mitochondrial TWINKLE protein, from Aegilops tauschii subsp. tauschii, transcript variant X1. The protein coding region is nucleotides 32-2209.

SEQ ID NO:88—Amino acid sequence of TWINKLE homologous protein, chloroplastic/mitochondrial from Panicum hallii, isoform X1.

SEQ ID NO:89—Nucleotide sequence of the cDNA for a TWINKLE gene, encoding a chloroplastic/mitochondrial TWINKLE protein, from Panicum hallii, transcript variant X1. The protein coding region is nucleotides 100-2343.

SEQ ID NO:90—Amino acid sequence of TWINKLE homologous protein, chloroplastic/mitochondrial from Setaria viridis, isoform X1.

SEQ ID NO:91—Nucleotide sequence of a cDNA for a TWINKLE gene, encoding a chloroplastic/mitochondrial TWINKLE protein from Setaria viridis, transcript variant X1. The protein coding region is nucleotides 70-2346.

SEQ ID NO:92—Amino acid sequence of TWINKLE homologous protein, chloroplastic/mitochondrial from Nicotiana tabacum.

SEQ ID NO:93—Nucleotide sequence of the cDNA for a TWINKLE gene, encoding a chloroplastic/mitochondrial TWINKLE protein, from Nicotiana tabacum. The protein coding region is nucleotides 103-2199.

SEQ ID NO:94—Amino acid sequence of TWINKLE homologous protein, chloroplastic/mitochondrial from Solanum tuberosum.

SEQ ID NO:95—Nucleotide sequence of a cDNA for a TWINKLE gene, encoding a chloroplastic/mitochondrial TWINKLE protein from Solanum tuberosum. The protein coding region is nucleotides 92-2176.

SEQ ID NO:96—Amino acid sequence of TWINKLE homologous protein, chloroplastic/mitochondrial from Solanum lycopersicum, isoform X1.

SEQ ID NO:97—Nucleotide sequence of a cDNA for the TWINKLE gene, encoding a chloroplastic/mitochondrial TWINKLE protein from Solanum lycopersicum, transcript variant X1. The protein coding region is nucleotides 138-2228.

SEQ ID NO:98—TA1-1F primer used in TILLING assay.

SEQ ID NO:99—TA1-1R primer used in TILLING assay.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, more preferably +/−2.5%, even more preferably +/−1%, of the designated value. The term “about” includes the exact designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

The terms “aleurone” and “aleurone layer” are used interchangeably herein. The aleurone layer is the outermost layer of the endosperm of cereal grain, distinct from the inner starchy endosperm, and surrounds the starchy endosperm and part of the embryo. The cells that make up the aleurone layer are therefore the outermost cells of the endosperm, the starchy component of the grain. While it is technically part of the endosperm, sometimes referred to as the peripheral endosperm, the aleurone is considered part of the bran from a practical standpoint as it is removed with the pericarp and testa layers of the bran when the grain is polished e.g. when rice grain is milled to produce “white rice”. Unlike cells of the starchy endosperm, aleurone cells remain alive at grain maturity. The aleurone layer is an important part of the nutritional value of cereal grain comprising minerals, vitamins such as vitamin A and B group vitamins, phytochemicals, and fiber.

Embodiments of the invention relate to a range of number of “layers of cells”, at least in part because at any one cross sectional point of grain of the invention, the layers of cells observed at any single point within the cross section, or between cross sections, may vary to some extent. More specifically, an aleurone with, for example, seven layers of cells may not have the seven layers surrounding the entire inner starchy endosperm but has seven layers surrounding at least part of the inner starchy endosperm, preferably at least half of the starchy endosperm.

The term “thickened” when used in relation to aleurone of grain of the invention is a relative term used when comparing grain of the invention to a corresponding wild-type cereal grain. In this context, grain of the invention comprises a genetic modification which results in the thickened aleurone relative to the wild-type grain which lacks the genetic modification. The aleurone of grain of the invention has an increased number of cells and/or increased number of layers of cells, preferably both, when compared to the aleurone of corresponding wild-type cereal grain. The aleurone is thereby increased in thickness as measured in μm, for instance as determined by microscopy. In an embodiment, the thickness is increased by at least 50%, preferably by at least 100%, and may be increased by as much as 500% or 600%, each percentage being relative to the thickness of the aleurone of a corresponding wild-type cereal grain. In an embodiment, the increase is between 50% and 100%, or between 100% and 200%, or between 200% and 300%, or between 300% and 400% or 500%. In an embodiment, each percentage increase or range of percentages of increase is the average increase over at least the ventral side of the grain and preferably over the whole grain. In a preferred embodiment, the thickness of the aleurone layer is determined across an entire cross section of the grain. In an embodiment, the thickness of the aleurone is determined by analysis on at least the ventral side of the grain. In another embodiment, thickened aleurone of grain of the invention comprises cells of varying size and irregular orientation compared to that of corresponding wild-type cereal grain where the aleurone generally has regularly oriented rectangular cells. All of the combinations of these features of “thickened aleurone” are contemplated.

### Polypeptides

The terms “polypeptide” and “protein” are generally used interchangeably herein and mean a polymer of amino acids linked with peptide bonds.

As used herein, the term “reduced level”, or similar phrases, of at least one mitochondrial polypeptide relative to a corresponding wild-type cereal grain is a relative term where the the total amount of protein is reduced. This can be achieved by standard techniques in the art, for example, by a deletion of part or the whole of the gene encoding the polypeptide, which results in the grain lacking the polypeptide. Such a mutation would be a null mutation. In an embodiment, the level/amount of the protein is reduced by at least 30%, at least 40%, at least 50%, at least 60%, at least 70%, at least 80%, at least 90%, at least 95%, at least 99%, or completely lacking, when compared to the corresponding wild-type cereal grain.

As used herein, the terms “reduced activity”, or similar phrases, of at least one mitochondrial polypeptide relative to a corresponding polypeptide in a corresponding wild-type cereal grain is a relative term where the the total amount of the specific protein activity is reduced. In an embodiment, the activity of the protein is reduced by at least 30%, at least 40%, at least 50%, at least 60%, at least 70%, at least 80%, at least 90%, at least 95%, at least 99%, or completely abolished, when compared to the corresponding wild-type cereal grain. In this instance, the total amount of the protein can be about the same in grain of the invention when compared to corresponding wild type grain, but the polypeptide in grain of the invention has at least one amino acid difference when compared to the corresponding polypeptide in the corresponding wild type grain, such that protein function in grain of the invention is reduced.

As used herein, the term “mitochondrial single-stranded DNA binding polypeptide” or “mtSSB” refers to a member of a family of proteins which bind single stranded DNA and can be found in the mitochondria of cereal grain, and homologous proteins which are modified whereby they have reduced binding activity or no binding activity. The wild-type proteins play a role in DNA replication, recombination and repair through binding to single-stranded DNA, as may the modified proteins. The mitochondria of cereal grain typically have three different sub-families of mtSSBs designated mtSSB-1a, mtSSB-1b and mtSSB-2, although some cereals have only two sub-families, namely mtSSB-1 and mtSSB-2. The present inventors have found that reducing mtSSB-1a, or mtSSB-1, binding activity in cereal grain relative to the wild-type results in an increase in aleurone thickness. mtSSB polypeptides include such polypeptides found in wild-type cereal plants as well as variants thereof produced either artificially or found in nature, and may have or do not have single stranded DNA binding activity DNA activity, including mtSSB polypeptides which have some single stranded DNA binding activity but at a reduced level compared to a corresponding wild-type mtSSB polypeptide. As demonstrated herein, the wild-type mtSSB-1a polypeptide not only binds ssDNA but also two other mitochondrial polypeptides, namely TWINKLE and RECA3. In an embodiment, a mtSSB polypeptide of the invention, including a mtSSB variant with reduced activity, bind a TWINKLE polypeptide. In an embodiment, a mtSSB polypeptide of the invention, including a mtSSB variant with reduced activity, binds a RECA3 polypeptide. In an embodiment, a mtSSB polypeptide of the invention is capable of forming an oligomer of mtSSB's, in particular a homodimer. In an embodiment, a wild-type grain mtSSB polypeptide comprises an amino acid sequence as provided in any one of SEQ ID NOs: 3 or 15 to 39, or an amino acid sequence which is at least 75% identical to any one or more of SEQ ID NOs: 3 or 15 to 39. In a preferred embodiment, a wild-type grain mtSSB polypeptide comprises an amino acid sequence as provided in any one of SEQ ID NOs: 3, 16 to 20 or 23, or an amino acid sequence which is at least 75% identical to any one or more of SEQ ID NOs: 3, 16 to 20 or 23 along the full length of the reference sequence(s).

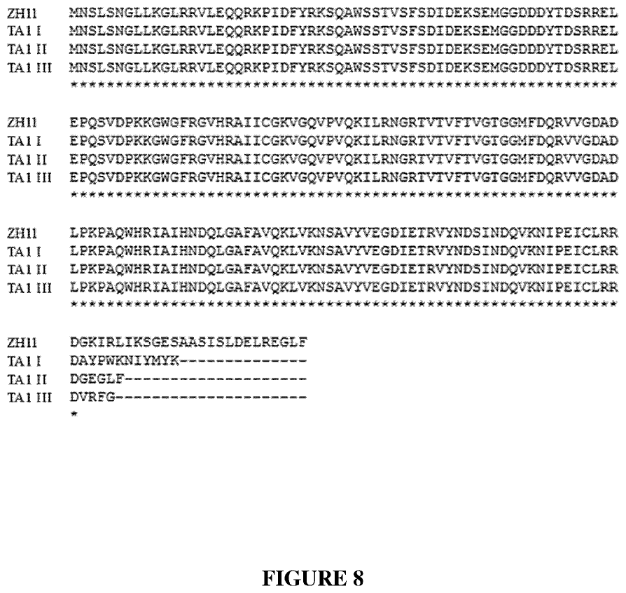

The wild-type mtSSB disclosed herein typically has a length of between 200-215 amino acid residues when including the mitochondrial targeting peptide (MTP) at the N-terminus, and a length of between 170-190 residues after cleavage by MTP. Mutant mtSSB polypeptides of the invention may be up to 215 amino acid residues in length with the MTP, in the absence of any insertion in the mtSSB gene, but may be shorter, even much shorter, if they are C-terminally truncated or encoded by a mutant gene comprising a premature translation termination mutation. In an embodiment, the mtSSB polypeptide of the invention lacks at least part of the DNA binding domain, for example lacks the amino acids GKI at the C-terminal end of the DNA binding domain (see, for example, FIG. 8).

In an embodiment, grain of the invention has at least one mtSSB polypeptide which has a sequence which is different to the amino acid sequence of a corresponding wild-type mtSSB polypeptide, or lacks the wild-type mtSSB polypeptide. In a related embodiment, grain of the invention has an endogenous gene that encodes at least one (mutant) mtSSB polypeptide which has a sequence which is different to the amino acid sequence of a corresponding wild-type mtSSB polypeptide, even if that mutant polypeptide is not expressed in the grain or is unstable and does not accumulate in the grain.

As used herein, the term “which has a sequence which is different to the amino acid sequence of the corresponding wild-type mtSSB polypeptide”, or similar phrases, are comparative terms where the amino acid sequence of a mtSSB polypeptide of the invention, and/or in grain of the invention, is different to the amino acid sequence of the protein from which it is derived and/or most closely related that exists in nature. In an embodiment, the amino acid sequence of the mtSSB polypeptide may have one or more insertions, deletions or amino acid substitutions (or a combination of these) relative to the corresponding wild-type amino acid sequence. The mtSSB polypeptide may have 2, 3, 4, 5 or 6-10 amino acid substitutions relative to the corresponding wild-type mtSSB polypeptide. In a preferred embodiment, the mtSSB polypeptide is C-terminally truncated, for example lacking the C-terminal 14 amino acids, or 20 amino acids or 21 amino acids, or more, of a wild-type mtSSB polypeptide (see, for example, Table 4). Such a truncated mtSSB polypeptide may be encoded by, for example, a mtSSB gene which comprises a premature translational stop codon in the protein open reading frame relative to the wild-type mtSSB gene from which it is derived or encoded by a mutant gene which produces an RNA transcript that is spliced differently to the wild-type RNA transcript, for example a gene comprising a splice-site mutation. In another embodiment, a mtSSB polypeptide with reduced activity has only a single insertion, single deletion or single amino acid substitution relative to the corresponding wild-type polypeptide. In this context, the “single insertion” and “single deletion” includes where multiple (e.g., 2, 3, 4, 5, 6, 7, 8, 9, 10 or more), contiguous amino acids are inserted or deleted, respectively and the “corresponding wild-type polypeptide” means the wild-type polypeptide from which the variant is derived and/or the natural polypeptide to which the variant is most closely related. As demonstrated in Example 13, such mutations can be anywhere within the coding region of the protein, preferably within the region of the gene encoding the DNA binding domain of the mtSSB polypeptide.

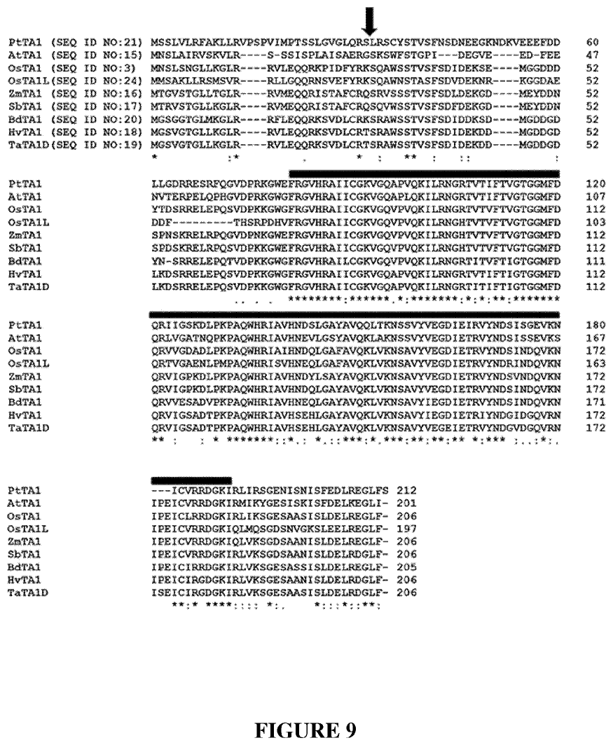

As used herein, the term “mtSSB polypeptide activity”, or variations thereof, refers to at least one biological action of a wild-type mtSSB polypeptide or a variant thereof. In one example, this term refers to the strength of binding, or lack thereof, of the polypeptide, or an oligomer thereof, to single stranded DNA. Thus, in one embodiment, a mtSSB polypeptide of the invention, such as a truncated mtSSB polypeptide, has reduced, or no, single stranded DNA binding activity when compared to a wildtype mtSSB polypeptide such as one comprising an amino acid sequence as provided in any one of SEQ ID NOs: 3 or 15 to 39. In this context, the comparison is with the wild-type mtSSB polypeptide that is closest in sequence to the mtSSB polypeptide of the invention, as determined by an amino acid alignment with known mtSSB polypeptide sequences, or with the wild-type mtSSB polypeptide from which the mtSSB polypeptide of the invention is derived by mutation. In an embodiment, a mtSSB polypeptide of the invention lacks at least part of the DNA binding domain (see FIG. 9), or has a variant (such as a C-terminally truncated variant) DNA binding domain that binds single stranded DNA more weakly than a corresponding wild-type mtSSB polypeptide (for example such as provided in SEQ ID NO:3). In one example, this term refers to the strength of binding, or lack thereof, of the polypeptide, or an oligomer thereof, to a TWINKLE polypeptide of the invention. In one example, this term refers to the strength of binding, or lack thereof, of the polypeptide, or an oligomer thereof, to a RECA3 polypeptide of the invention.

Reduced single stranded DNA binding activity can readily be determined by using standard assays in the art. For example, single stranded DNA binding can be measured using electrophoretic mobility shift assays (also known as gel mobility shift assays) such as described by Farr et al. (2004) and Ciesielski et al. (2016), or preferably as described in Examples 1 and 9 herein. In such assays, a single stranded DNA substrate and a polypeptide are added to a reaction mixture and gel electrophoresis used to determine if they assemble into protein-DNA complexes.

As used herein, the terms “mitochondrial single-stranded DNA binding polypeptide-1a”, “mtSSB-1a” or “TA1”, and variants thereof, refer to a sub-family of mtSSBs whose wild-type protein comprises, for example, an amino acid sequence as provided in any one of SEQ ID NOs: 3 or 15 to 23, or an amino acid sequence which is at least 75% identical to any one or more of SEQ ID NOs: 3 or 15 to 23 along the full length of the reference sequence(s). The variants thereof include mtSSB-1a polypeptides that have reduced ssDNA binding activity, or lack ssDNA binding activity, as a result of the mutation(s) in the endogenous gene that encodes the polypeptide. In an embodiment, a wild-type OsmtSSB-1a polypeptide has one or more of all of the following motifs: FRGVHRAI(I/L)CGKVGQ(V/A)P(V/L)QKILRNG(R/H)T(V/I)T(V/I)FT(V/I)GTGG MFDQR (Motif I, SEQ ID NO:45) corresponding to amino acids 73-114 of SEQ ID NO:3, P(K/M)PAQWHRI(A/S)(V/I)H(N/S)(D/E) (Motif II, SEQ ID NO:46) corresponding to amino acids 122-135 of SEQ ID NO:3, AVQ(K/Q)L(V/T)KNS(A/S)VY(V/I)EG(D/E)IE(T/I)R(V/I)YND (Motif III, SEQ ID NO:47) corresponding to amino acids 141-164 of SEQ ID NO:3 and 184 of SEQ ID NO:3. Examples of polypeptides with reduced OsmtSSB-1a polypeptide activity include those which have an amino acid sequence as set forth as SEQ ID NO:8, SEQ ID NO:9 or SEQ ID NO:10, or a truncated version thereof, or which have an amino acid sequence which is at least 75% identical to one or more of the amino acid sequences set forth as SEQ ID NO:8, SEQ ID NO:9 or SEQ ID NO:10 along the full length of the reference sequence(s).

As used herein, the terms “mitochondrial single-stranded DNA binding polypeptide-1b” or “mtSSB-1b”, and variants thereof, refer to a sub-family of mtSSBs whose wild-type protein comprises, for example, an amino acid sequence as provided in any one of SEQ ID NOs: 24 to 29, or an amino acid sequence which is at least 75% identical to any one or more of SEQ ID NOs: 24 to 29 along the full length of the reference sequence(s). mtSSB-1a polypeptides are not included in the set of mtSSB-1b polypeptides.

As used herein, the terms “mitochondrial single-stranded DNA binding polypeptide-2” or “mtSSB-2”, and variants thereof, refer to a sub-family of mtSSBs whose wild-type protein comprises, for example, an amino acid sequence as provided in any one of SEQ ID NOs: 30 to 39, or an amino acid sequence which is at least 75% identical to any one or more of SEQ ID NOs: 30 to 39 along the full length of the reference sequence(s). mtSSB-1a polypeptides are not included in the set of mtSSB-2 polypeptides.

As used herein, the term “TWINKLE” refers to a protein family which can be found in the mitochondria of wild-type cereal grain and which has helicase activity, and mutant variants thereof. The wild-type TWINKLE proteins play a role in mitochondrial DNA replication. Helicases utilize the energy from nucleotide triphosphate (NTP) hydrolysis to catalyze the unwinding of duplex DNA. The present inventors have found that reducing (but not eliminating) wild-type TWINKLE activity in cereal grain, preferably in rice grain, results in an increase in aleurone thickness (see Example 15). TWINKLE polypeptides include such polypeptides found in wild-type cereal plants as well as variants thereof produced either artificially or found in nature, and either have or do not have helicase activity, including TWINKLE polypeptides which have some helicase activity but at a reduced level compared to a corresponding wild-type TWINKLE polypeptide. In this context, the comparison is with the wild-type TWINKLE polypeptide that is closest in sequence to the TWINKLE polypeptide of the invention, as determined by an amino acid alignment, or with the wild-type TWINKLE polypeptide from which the TWINKLE polypeptide of the invention is derived by mutation. In an embodiment, a TWINKLE polypeptide binds a mtSSB polypeptide. In an embodiment, a TWINKLE polypeptide has helicase activity. In an embodiment, a TWINKLE polypeptide is capable of forming a multimer of TWINKLE polypeptides. TWINKLE typically has five conserved helicase motifs: (1) the H1/Walker A motif, which stabilizes the NTP phosphate; (2) H1a, which is involved in NTP binding/hydrolysis; (3) the H2/Walker B motif, which contains an arginine finger and a base stack residue required for positioning and stabilizing the bound NTP as well as stabilizing a bound Mg2+ ion; (4) H3, which with H1a is involved in NTP binding/hydrolysis; and (5) H4, which contributes to DNA binding. Furthermore, the C-terminal region of many TWINKLE helicases is required for interactions with other factors in the replisome. In an embodiment, a TWINKLE polypeptide comprises an amino acid sequence as provided in any one of SEQ ID NOs: 64, 80, 82, 84, 86, 88, 90, 92, 94 or 96, or an amino acid sequence which is at least 75% identical to any one or more of SEQ ID NOs: 64, 80, 82, 84, 86, 88, 90, 92, 94 or 96 along the full length of the reference sequence(s).

Reduced helicase activity can readily be determined by using standard assays in the art. For example, helicase activity can be determined by measuring the amount of helicase reaction products, such as ADP, inorganic phosphonate, and/or single stranded DNA. In these assays, fluorescently labelled proteins detect a target molecule, for example ADP, inorganic phosphonate, or single stranded DNA (Toseland et al., 2010). In one example, double stranded DNA unwinding can be measured to determine helicase activity. This measurement uses a fluorophore (for example Cy3) and quencher (for example Dabcyl) pair positioned at the end of a duplex, one on each strand of DNA. Upon helicase induced DNA unwinding, the fluorophore and quencher are separated from one another resulting in an increase in fluorescence (Toseland et al., 2010). In another example, a helicase substrate is generated which consists of a partially double stranded DNA substrate with a short 5′ single stranded DNA overhang (Korhonen et al., 2003). A helicase reaction is performed in a reaction buffer containing the helicase substrate and polypeptide. The fractional amounts of base-paired substrate and single stranded DNA product can then be isolated and analysed by gel electrophoresis.

As used herein, the term “RECA3” refers to a sub-family of plant recombinant associated proteins which can be found in the mitochondria of wild-type cereal grain and which help reduce the occurrence of improper recombination events in mitochondrial DNA, and mutant variants thereof. Without wishing to be limited by theory, current evidence suggests that RECA3 is involved in a surveillance mechanism that directs conversion events between short repeats, while allowing recombination-dependent replication to be initiated at long repeats (Shedge et al., 2007). The present inventors have found that reducing (but not eliminating) wild-type RECA3 activity in cereal grain, preferably in rice grain, results in an increase in aleurone thickness. RECA3 polypeptides include such polypeptides found in wild-type cereal plants as well as variants thereof produced either artificially or found in nature, which either have or do not have recombinase activity, including RECA3 polypeptides which have some recombinase activity but at a reduced level compared to a corresponding wild-type RECA3 polypeptide. In an embodiment, a RECA3 polypeptide binds a mtSSB polypeptide. In an embodiment, a RECA3 polypeptide has recombinase activity. RECA3 polypeptides typically have a RecA/RAD51 domain which comprises two highly conserved consensus motifs, Walker A and Walker B, which are present in ATPases and confer ATP binding and hydrolysis activities. In an embodiment, a RECA3 polypeptide comprises an amino acid sequence as set forth as SEQ ID NOs: 61, 67, 69, 71, 73, 75, 77 or 79, or an amino acid sequence which is at least 75% identical to any one or more of SEQ ID NOs: 61, 67, 69, 71, 73, 75, 77 or 79 along the full length of the reference sequence(s).

Reduced recombinase activity can readily be determined by using standard assays in the art. In one example, fluorophore-labelled single stranded DNA substrates and polypeptides are incubated in a reaction mixture (Silva et al., 2017). A fluorescence quenching effect occurs upon recombinase binding. Analysis of fluorescence can be performed using fluorescence spectroscopy. In another example, recombinase activity can be measured using strand assimilation (or “D-loop”) assays (Jayathilaka et al., 2008). In this assay, the polypeptide is first allowed to assemble on a radio-labelled single stranded DNA oligonucleotide. A homology-containing supercoiled target plasmid is then added, and the two are allowed to form a homology-dependent joint molecule referred to as a “D-loop”. D-loop proteins can be isolated and analysed by gel electrophoresis and phosphorimaging.

As used herein, the terms “substantially purified polypeptide” or “purified polypeptide” means a polypeptide that has been separated at least in part from the lipids, nucleic acids, other peptides, and other contaminating molecules with which it is associated in its native state. Preferably, the substantially purified polypeptide is at least 90% free from other components with which it is naturally associated. In an embodiment, the polypeptide of the invention has an amino acid sequence which is different to a naturally occurring cereal mtSSB, TWINKLE or RECA3 polypeptide i.e. is an amino acid sequence variant, as defined above.

Grain, plants and host cells of the invention may comprise an endogenous gene comprising a mutation which encodes a variant polypeptide of the invention or an exogenous polynucleotide encoding a polypeptide of the invention. In these instances, the grain, plants and cells produce a mutant or recombinant polypeptide. The term “mutant” in this context means a polypeptide which is different in amino acid sequence to the wild-type polypeptide to which it is most closely related to, or derived from by mutation. The mutant polypeptide may be expressed at a different level or in a different cell or at a different timing compared to the wild-type polypeptide to which it is most closely related and/or derived from. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. In an embodiment, the recombinant polypeptide is identical in amino acid sequence to a wild-type polypeptide, although the recombinant polypeptide may be expressed at a different level or in a different cell or at a different timing compared to the wild-type polypeptide. In an alternative embodiment, the recombinant polypeptide is different in amino acid sequence to the wild-type polypeptide to which it is most closely related or derived from. Again, this recombinant polypeptide may be expressed at a different level or in a different cell or at a different timing compared to the wild-type polypeptide. Typically, the cell comprises a non-endogenous gene that causes an altered amount of the polypeptide to be produced. In an embodiment, a “recombinant polypeptide” is a polypeptide made by the expression of an exogenous (recombinant) polynucleotide in a plant cell.

The % identity of a polypeptide is determined by GAP (Needleman and Wunsch, 1970) analysis (GCG program) with a gap creation penalty=5, and a gap extension penalty=0.3. In an embodiment, the query sequence is at least 150 amino acids in length, and the GAP analysis aligns the two sequences over a region of at least 150 amino acids. In an embodiment, the query sequence is at least 175 amino acids in length, and the GAP analysis aligns the two sequences over a region of at least 175 amino acids. In an embodiment, the query sequence is at least 200 amino acids in length, and the GAP analysis aligns the two sequences over a region of at least 200 amino acids. In an embodiment, the query sequence is at least 300 amino acids in length, and the GAP analysis aligns the two sequences over a region of at least 300 amino acids. In an embodiment, the query sequence is at least 400 amino acids in length, and the GAP analysis aligns the two sequences over a region of at least 400 amino acids. In an embodiment, the query sequence is at least 500 amino acids in length, and the GAP analysis aligns the two sequences over a region of at least 500 amino acids. In an embodiment, the query sequence is at least 600 amino acids in length, and the GAP analysis aligns the two sequences over a region of at least 600 amino acids. In an embodiment, the query sequence is at least 700 amino acids in length, and the GAP analysis aligns the two sequences over a region of at least 700 amino acids. Even more preferably, the GAP analysis aligns two sequences over their entire length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

As used herein, the phrase “at a position corresponding to amino acid number” or variations thereof refers to the relative position of the amino acid compared to surrounding amino acids, in the context of one or more specific amino acid sequences. In some embodiments a polypeptide of the invention may have a deletion or substitution mutation which alters the relative positioning of the amino acid when aligned against, for instance, SEQ ID NO: 3. Determining a corresponding amino acid position between two closely related proteins is well within the capability of the skilled person.

Conserved amino acids of the mtSSB, TWINKLE and RECA3 polypeptides can be readily identified by aligning the amino acid sequences for wild-type polypeptides, such as those described herein. For example, the rice OsmtSSB-1a (TA1) amino acid sequence (SEQ ID NO:3) can be aligned with mtSSB-1a polypeptides from other species, or even with mtSSB's from rice or oilier species, particularly plant species such as Arabidopsis thaliana (SEQ ID NO:15), Sorghum bicolor (SEQ ID NO:17), Hordeum vulgare (SEQ ID NO:18), Triticum aestivum (SEQ ID NO's 19 and 22) Brachypodium distachyon (SEQ ID NO:20) and Populus trichocarpa (SEQ ID NO:21) (see for example FIG. 9).

Amino acid sequence mutants of the polypeptides of the present invention can be prepared by mutagenesis of a progenitor cereal plant cell, for example mutagenesis of seeds of the cereal, either by random mutagenesis with a mutagenic agent followed by screening of a mutagenized population for mutants in an endogenous gene encoding the polypeptide, or by targeted mutagenesis such as, for example, with gene editing tools. Amino acid sequence mutants can also be prepared by introducing appropriate nucleotide changes into a nucleic acid of the present invention. Such mutants include, for example, deletions, insertions or substitutions of residues within the amino acid sequence which can occur anywhere along the length of the wild-type polypeptide. A combination of deletion, insertion and substitution can be made to arrive at the final construct, provided that the final peptide product possesses the desired characteristics. Preferred amino acid sequence mutants have only one, two, three, four or less than 10 amino acid changes relative to the reference wildtype polypeptide. Mutant polypeptides of the invention have “reduced activity” when compared to a corresponding wild-type naturally occurring polypeptide such as one whose amino acid sequence is set forth as, for example, SEQ ID NO:8, SEQ ID NO:9 or SEQ ID NO:10, or a truncated version thereof, or which has an amino acid sequence which is at least 75%, at least 80%, at least 85%, preferably at least 90%, or more preferably at least 95% identical to one or more of the amino acid sequences set forth as SEQ ID NO:8, SEQ ID NO:9 or SEQ ID NO:10 along the full length of the reference sequence(s).

Mutant (altered) polypeptides can be prepared using any technique known in the art, for example, using directed evolution or rational design strategies. Products derived from mutated/altered DNA can readily be screened using techniques described herein to determine if they have reduced activity, for example screening by TILLING (see Example 1). For instance, the method may comprise producing a transgenic plant expressing the mutated/altered DNA and determining i) the effect of the mutated/altered DNA on aleurone thickness and ii) whether a mtSSB, TWINKLE or RECA3 gene has been mutated/altered (for example, using a method as described in Example 13). The polypeptides and polynucleotides of the invention may therefore be used in a screening assay to determine their biological effect.

In designing amino acid sequence mutants, the location of the mutation site and the nature of the mutation will depend on characteristic(s) to be modified. The sites for mutation can be modified individually or in series, e.g., by (1) substituting with non-conservative amino acid choices, (2) deleting the target residue, or (3) inserting other residues adjacent to the located site.

Amino acid sequence deletions generally range from about 1 to 15 residues, more preferably about 1 to 10 residues and typically about 1 to 5 contiguous residues. Substitution mutants have at least one amino acid residue in the polypeptide molecule removed and a different residue inserted in its place. Where it is not desirable to maintain a certain activity, or to reduce a certain activity, it is preferable to make non-conservative substitutions, particularly at amino acid positions which are highly conserved in the relevant protein family. Examples of conservative substitutions are shown in Table 1, and thus non-conservative substitutions will be those not shown in Table 1.

TABLE 1
Conservative substitutions.

| Original | Conservative |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In an embodiment a mutant/variant polypeptide has one or two or three or four amino acid changes when compared to a naturally occurring polypeptide. In a preferred embodiment, the changes are in one or more of the motifs which are highly conserved between the different mtSSB, TWINKLE or RECA3 polypeptides provided herewith, particularly in known conserved structural domains. As the skilled person would be aware, such changes can reasonably be predicted to reduce the activity of the polypeptide when expressed in a cell or cereal grain.

The primary amino acid sequence of a polypeptide of the invention can be used to design variants/mutants thereof based on comparisons with closely related enzymes. As the skilled addressee will appreciate, alteration of residues that are highly conserved amongst closely related proteins are more likely to reduce activity than alteration of less conserved residues. In the present context, an alignment of the amino acid sequences of mtSSB, TWINKLE or RECA3 proteins from a range of sources is used to identify those amino acid residues that are more highly conserved and those less conserved.

### Polynucleotides and Genes

The present invention refers to various polynucleotides. As used herein, a “polynucleotide” or “nucleic acid” or “nucleic acid molecule” means a polymer of nucleotides, which may be DNA or RNA, and includes genomic DNA, mRNA, cRNA, dsRNA, and cDNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. A “hybridized polynucleotide” means the polynucleotide is actually basepaired to its complement. The term “polynucleotide” is used interchangeably herein with the term “nucleic acid”. Preferred polynucleotides of the invention encode a polypeptide of the invention.

By “isolated polynucleotide” we mean a polynucleotide which has been separated at least in part from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

The present invention involves reduction of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

An “allele” refers to one specific form of a genetic sequence (such as a gene) within a cell, an individual plant or within a population, the specific form differing from other forms of the same gene in the sequence of at least one, and frequently more than one, variant sites within the sequence of the gene. The sequences at these variant sites that differ between different alleles are termed “variances”, or “polymorphisms”. A “polymorphism” as used herein denotes a variation in the nucleotide sequence between alleles at a genetic locus of the invention, of different species, cultivars, strains or individuals of a plant. A “polymorphic position” is a preselected nucleotide position within the sequence of the gene at which the sequence difference occurs. In some cases, genetic polymorphisms cause an amino acid sequence variation within a polypeptide encoded by the gene, and thus a polymorphic position can result in the location of a polymorphism in the amino acid sequence at a predetermined position in the sequence of the polypeptide. In other instances, the polymorphic region may be in a non-polypeptide encoding region of the gene, for example in the promoter region and may thereby influence expression levels of the gene. Typical polymorphisms are deletions, insertions or substitutions. These can involve a single nucleotide (single nucleotide polymorphism or SNP) or two or more nucleotides.

As used herein, a “mutation” is a polymorphism which produces a phenotypic change in the plant or a part thereof. In this context, the primary phenotypic change is a thickening of the aleurone of the cereal grain relative to the wild-type (non-mutant) form. As known in the art, some polymorphisms are silent, for example a single nucleotide change in a protein coding region which does not change the amino acid sequence of the encoded polypeptide due to the redundancy of the genetic code. A diploid plant will typically have one or two different alleles of a single gene, but only one if both copies of the gene are identical i.e. the plant is homozygous for the allele. Polyploid plants generally have more than one homoeolog of any particular gene. For instance, hexaploid wheat has three subgenomes (often referred to as “genomes”) designated the A, B and D genomes, and therefore has three homoeologs of most of its genes, one in each of the A, B and D genomes.

The term “gene” refers to a nucleotide sequence which encodes a polypeptide as defined herein. The gene may be an endogenous naturally occurring gene, or comprise a genetic variation, preferably an introduced genetic variation, as defined herein. A gene encoding a polypeptide in grain of the invention may or may not have introns. In one example, the grain of the invention is from rice and at least one allele of an OsmtSSB-1a gene encodes a OsmtSSB-1a polypeptide with reduced OsmtSSB-1a polypeptide activity when compared to a OsmtSSB-1a polypeptide from a corresponding a wild-type rice plant (such as which comprises a sequence of amino acids as provided in SEQ ID NO: 3). An example of such a OsmtSSB-1a polypeptide with reduced OsmtSSB-1a polypeptide activity includes one or more of those which have an amino acid sequence which consist of the amino acids provided in SEQ ID NO:8, SEQ ID NO:9 or SEQ ID NO:10, or a truncated version thereof, or which have an amino acid sequence which is at least 75%, at least 90%, or preferably at least 95% identical to one or more of the amino acid sequences set forth in SEQ ID NO:8, SEQ ID NO:9 or SEQ ID NO:10 but are no longer in length.

In an embodiment, the genetic variation reduces expression of an endogenous gene encoding a wild-type mitochondrial polypeptide as described herein. As used herein, the phrase “reduces of expression of the endogenous gene” or variations thereof refers to any genetic variation which reduces (partially), or completely prevents, the expression of the gene encoding a functional polypeptide defined herein. Such genetic variations include mutations in the promoter region of the gene which reduce transcription of the gene, for example by using gene editing to delete or substitute nucleotides from the promoter of the gene, or intron splicing mutations which alter the amount or position of splicing to form mRNA.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region of the gene is operably linked to a promoter or polyadenylation/terminator region which is heterologous to the gene, thereby forming a chimeric gene. In an alternate embodiment, a gene encoding a polynucleotide which, when present in grain of a cereal plant, down regulates the production and/or activity of the polypeptide in the grain is operably linked to a promoter or polyadenylation/terminator region which is heterologous to the polynucleotide, thereby forming a chimeric gene.

The term “endogenous” is used herein to refer to a substance that is normally present or produced in an unmodified cereal plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA technology, including a nucleic acid molecule which is a modified endogenous gene that has been mutated by gene editing technology, for example by TALENS or CRISPR technology The terms “foreign polynucleotide” or “exogenous polynucleotide” or “heterologous polynucleotide” and the like refer to any nucleic acid which is introduced into the genome of a cell by experimental manipulations, including by gene editing technology.

Foreign or exogenous genes may be genes that are inserted into a non-native organism, native genes introduced into a new location within the native host, chimeric genes, or an endogenous gene which has been modified by gene editing technology. A “transgene” is a gene that has been introduced into the genome by a transformation procedure. The term “genetically modified” is broader and includes not only introducing genes into cells by transformation or transduction, but also mutating genes in cells, for example introducing an insertion, deletion or substitution in an endogenous gene by gene editing technology, and altering or modulating the regulation of a gene in a cell or organism to which these acts have been done or a progenitor cell or organism.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or it can comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. In an embodiment, the query sequence is at least 450 nucleotides in length, and the GAP analysis aligns the two sequences over a region of at least 450 nucleotides. In an embodiment, the query sequence is at least 525 nucleotides in length, and the GAP analysis aligns the two sequences over a region of at least 525 nucleotides. In an embodiment, the query sequence is at least 600 nucleotides in length, and the GAP analysis aligns the two sequences over a region of at least 600 nucleotides. In an embodiment, the query sequence is at least 900 nucleotides in length, and the GAP analysis aligns the two sequences over a region of at least 900 nucleotides. In an embodiment, the query sequence is at least 1,200 nucleotides in length, and the GAP analysis aligns the two sequences over a region of at least 1,200 nucleotides. In an embodiment, the query sequence is at least 1,500 nucleotides in length, and the GAP analysis aligns the two sequences over a region of at least 1,500 nucleotides. In an embodiment, the query sequence is at least 1,800 nucleotides in length, and the GAP analysis aligns the two sequences over a region of at least 1,800 nucleotides. In an embodiment, the query sequence is at least 2,100 nucleotides in length, and the GAP analysis aligns the two sequences over a region of at least 2,100 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 75%, more preferably at least 80%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. It is preferred that the % identity is calculated along the full length of the reference sequence. In an embodiment, the polynucleotide of the invention extends beyond a reference polynucleotide at the 5′ end, the 3′ end or both ends.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

The present invention includes oligonucleotides that can be used as, for example, probes to identify nucleic acid molecules, or primers to produce nucleic acid molecules, or as guide sequences for gene editing. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences provided as SEQ ID NO's 1, 2, 4 to 7, 11 to 14, 49 to 60, 62, 63, 65, 66, 68, 70, 72, 74, 76, 78, 81, 83, 85, 87, 89, 91, 93, 95 or 97. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide or an oligonucleotide of the invention includes molecules of varying sizes of, and/or are capable of hybridising to, the cereal genome close to that of the reference polynucleotide or oligonucleotide molecules defined herein, preferably the endogenous gene. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close to, for example to within 50 nucleotides, the region of the plant genome where the specific oligonucleotides defined herein hybridise. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

### Genetic Variations

As used herein, the term “genetic variation” refers to one or more cells of the grain, preferably cells in at least one or more or all of developing endosperm, testa, aleurone, and embryo of a developing grain, more preferably one or more or all of testa, aleurone, and embryo of the developing grain, or of a plant or part thereof of the invention, which have a genetic modification which may be introduced by man, or may be naturally occurring in cereal plant (for example, crossed to produce a plant of the invention). The grain may comprise two, three, four or more genetice variations in the same or different genes. In one example, a mtSSB gene comprises two genetic variations which reduce mtSSB polypeptide level and/or activity in the cereal grain. In another example, the cereal grain comprises a genetic variation in a mtSSB gene which reduces the mtSSB polypeptide level and/or activity in the cereal grain, and/or a genetic variation in a TWINKLE gene which reduces the TWINKLE polypeptide level and/or activity in the cereal grain, and/or a genetic variation in a RECA3 gene which reduces the RECA3 polypeptide level and/or activity in the cereal grain. In a preferred embodiment, every cell in the grain or the plant or part thereof comprises the introduced genetic variation(s). In an embodiment, the cell, grain or plant of the invention is homozygous for the one or more genetic variations. In an alternative embodiment, the cell, grain or plant of the invention is heterozygous for one or more genetic variations, or is heterozygous for one genetic variation and homozygous for another genetic variation. As the skilled person would understand, there are many different types of genetic modifications which can be made such as, but not limited to, a mutation in an endogenous gene encoding the polypeptide, which may be in the protein coding region of the gene or in an expression element such as a promoter, for example a mutation introduced by gene editing that reduces the activity of an endogenous gene, or using TILLING to introduce a mutation with selection for plants producing grain with reduced polypeptide activity. Alternately, the genetic modification comprises an introduced nucleic construct encoding an exogenous polynucleotide which reduces the expression of the gene, such as for example a dsRNA molecule or microRNA, or a nucleic construct encoding an exogenous polynucleotide which encodes the polypeptide whose amino acid sequence is different to the amino acid sequence of a corresponding wild-type polypeptide and which has reduced polypeptide activity when compared to the corresponding wild-type polypeptide.

### Mutagenesis

The plants of the invention can be produced and identified after mutagenesis. This may provide a plant which is non-transgenic, which is desirable in some markets.

Mutants can be either naturally occurring (that is to say, isolated from a natural source) or synthetic (for example, by performing mutagenesis on the nucleic acid) or induced. Generally, a progenitor cereal plant cell, tissue, seed or plant may be subjected to mutagenesis to produce single or multiple mutations, such as nucleotide substitutions, deletions, additions and/or codon modification. In the context of this application, an “induced mutation” is an artificially induced genetic variation which may be the result of chemical, radiation or biologically-based mutagenesis, for example transposon or T-DNA insertion. In some embodiments, mutations are null mutations such as nonsense mutations, frameshift mutations, insertional mutations or splice-site variants which completely inactivate the gene. Nucleotide insertional derivatives include 5′ and 3′ terminal fusions as well as intra-sequence insertions of single or multiple nucleotides. Insertional nucleotide sequence variants are those in which one or more nucleotides are introduced into the nucleotide sequence, which may be obtained by random insertion with suitable screening of the resulting products. Deletional variants are characterized by the removal of one or more nucleotides from the sequence. Preferably, a mutant gene has only a single insertion or deletion of a sequence of nucleotides relative to the wild-type gene. Substitutional nucleotide variants are those in which at least one nucleotide in the sequence has been removed and a different nucleotide inserted in its place. The preferred number of nucleotides affected by substitutions in a mutant gene relative to the wild-type gene is a maximum of ten nucleotides, more preferably a maximum of 9, 8, 7, 6, 5, 4, 3, or 2, or only one nucleotide. Such a substitution may be “silent” in that the substitution does not change the amino acid defined by the codon. Alternatively, conservative substitutions are designed to alter one amino acid for another similar acting amino acid. Typical conservative substitutions are those made in accordance with Table 1.

The term “mutation” as used herein does not include silent nucleotide substitutions which do not affect the activity of the gene, and therefore includes only alterations in the gene sequence which affect the gene activity. The term “polymorphism” refers to any change in the nucleotide sequence including such silent nucleotide substitutions.

In some embodiments, the cereal grain comprises non-conservative substitution deletion of at least part of a mtSSB gene, TWINKLE gene or RECA3 gene or a frameshift or splice site variation in such gene.

In some embodiments, one or more mutants are within a conserved region of the gene encoding the mtSSB polypeptide encoding conserved motifs, such as comprising the amino acid sequences set out in SEQ ID NOs: 45 to 48. In some embodiments, one or more mutations are within the region of OsmtSSB-1a encoding the DNA binding domain of OsmtSSB-1a.

Mutagenesis can be achieved by chemical or radiation means, for example EMS or sodium azide (Zwar and Chandler, 1995). Chemical mutagenesis tends to favour nucleotide substitutions rather than deletions. Heavy ion beam (HIB) irradiation is known as an effective technique for mutation breeding to produce new plant cultivars, see for example Hayashi et al. (2004) and Kazama et al. (2008). Ion beam irradiation has two physical factors, the dose (gy) and LET (linear energy transfer, keV/um) for biological effects that determine the amount of DNA damage and the size of DNA deletion, and these can be adjusted according to the desired extent of mutagenesis. RIB generates a collection of mutants, many of them comprising deletions, that may be screened for mutations in a mtSSB gene. Useful mutants which are identified may be backcrossed with non-mutated plants as recurrent parents in order to remove and therefore reduce the effect of unlinked mutations in the mutagenised genome.

Biological agents useful in producing site-specific mutants include enzymes that include double stranded breaks in DNA that stimulate endogenous repair mechanisms. These include endonucleases, zinc finger nucleases, transposases and site-specific recombinases. Zinc finger nuclease technology is reviewed in Le Provost et al. (2009), Durai et al. (2005) and Liu et al. (2010).

Isolation of mutants may be achieved by screening mutagenized plants or seed. For example, a mutagenized population of cereal plants may be screened for thick aleurone or for reduced expression of one of the genes by RT-PCR, or directly for mutation of one of the genes or by a PCR or heteroduplex based assay, or reduction of the protein by ELISA or Western blot analysis. Alternatively, the mutation may be identified using techniques such as TILLING in a population mutagenized with an agent such as EMS (Slade and Knauf, 2005) or by deep sequencing of mutagenized pools. Such mutations may then be introduced into desirable genetic backgrounds by crossing the mutant with a plant of the desired genetic background and performing a suitable number of backcrosses to cross out the originally undesired parent background.

The mutation may have been introduced into the plant directly by mutagenesis or indirectly by crossing of two parental plants, one of which comprised the introduced mutation. The modified plants may be transgenic or non-transgenic. Using mutagenesis, a non-transgenic plant having a reduced mtSSB-1a level or activity or essentially no mtSSB-1a of may be produced. The invention also extends to the grain or other plant parts produced from the plants and any propagating material of the plants that can be used to produce the plants with the desired characteristics, such as cultured tissue or cells. The invention clearly extends to methods of producing or identifying such plants or the grain produced by such plants.

### TILLING

Plants of the invention can be produced using the process known as TILLING (Targeting Induced Local Lesions IN Genomes), for example as described in Example 13 herein. In a first step, introduced mutations such as novel single base pair changes are induced in a population of plants by treating seeds (or pollen) with a chemical mutagen, and then advancing plants to a generation where mutations will be stably inherited. This is typically an M2 generation. DNA is extracted from individual plants or small pools of plants, and seeds are stored from all members of the population to create a resource that can be accessed repeatedly over time.

For a TILLING assay, PCR primers are designed to specifically amplify a single gene target of interest, for example a gene encoding mtSSB-1a, TWINKLE or RECA3. Specificity is especially important if a target is a member of a gene family or part of a polyploid genome such as in hexaploid or tetraploid wheat. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Ce/I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Genome Editing Using Site-Specific Nucleases

Genome editing can be used to modify a gene encoding a polypeptide defined herein. Genome editing uses engineered nucleases composed of sequence specific DNA binding domains fused to a non-specific DNA cleavage module. These chimeric nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs), transcription activator-like (TAL) effector nucleases (TALEN) and CRISPR-Cas9 or Cas12 type site-specific nucleases.

Typically nuclease encoded genes are delivered into cells by plasmid DNA, viral vectors or in vitro transcribed mRNA. The use of fluorescent surrogate reporter vectors also allows for enrichment of ZFN-, TALEN- or CRISPR-modified cells.

Complex genomes often contain multiple copies of sequences that are identical or highly homologous to the intended DNA target, potentially leading to off-target activity and cellular toxicity. To address this, structure (Miller et al., 2007; Szczepek et al., 2007) and selection based (Doyon et al., 2011; Guo et al., 2010) approaches can be used to generate improved ZFN and TALEN heterodimers with optimized cleavage specificity and reduced toxicity.

In order to target genetic recombination or mutation by ZFN according to a preferred embodiment of the present invention, two 9 bp zinc finger DNA recognition sequences must be identified in the host DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing (Gene Editing) Using CRISPR

Endonucleases can be used to generate single strand or double strand breaks in genomic DNA in a sequence-specific manner i.e. targeting a defined nucleotide sequence. The genomic DNA breaks in eukaryotic cells are repaired using non-homologous end joining (NHEJ) or homology directed repair (HDR) pathways. NHEJ may result in imperfect repair resulting in the production of mutations, often short deletions e.g. 1-30 basepairs. In contrast, HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into two major groups using various nucleases or combinations of nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by a synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a PolIII promoter such as a U3 or U6 small nuclear RNA promoter. The sgRNA generally comprises a CRISPR RNA (crRNA) spacer and repeat sequence, and Cas effector proteins together for a complex to recognise the protospacer adjacent motif (PAM) present in the target nucleotide sequence. The sgRNA recognises the specific gene and part of the gene for targeting by sequence homology. The protospacer adjacent motif (PAM) is adjacent to the target site constraining the number of potential CRISPR-Cas targets in a genome although the expansion of nucleases also increases the number of PAM's available. The Cas nuclease is activated by the base pairing between the crRNA space and the complementary target nucleotide sequence. There are numerous web tools available for designing gRNAs including CHOPCHOP (http.//chopchop.cbu.uib.no), CRISPR design https://omictools.com/crispr-design-tool, E-CRISP http://www.e-crisp.org/E-CRISP/, and Geneious or Benchling https://benchling.com/crispr.

In one example, guide RNA (gRNA) target sequences of about 17, 18, 19 or 20 nucleotides followed immediately by a 3-nucleotide PAM (protospacer adjacent motif) sequence are identified from the cDNA sequence of to a gene encoding a polypeptide defined herein, preferably one or more of an mtSSB-1 or mtSSB-1a polypeptide, a TWINKLE polypeptide or a RECA3 polypeptide.

CRISPR-Cas systems are the most frequently adopted in modifications of plant genes for gene editing, often using a Cas9 effector protein such as the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Modified Cas9 such as xCas9 are available for use where a variant PAM sequences is required (Hu et al., 2018). Due to the cell wall on plant cells the delivery of the CRISPR-Cas machinery into the cell and successful transgenic regenerations have typically used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, Accession number KR029109.1).

Alternative CRISPR-Cas systems are numerous and include effector enzymes that contain the nuclease RuvC domain but do not contain the HNH domain including Cas12 enzymes including Cas12a, Cas12b, Cas12f, Cpf1, C2c1, C2c3, and engineered derivatives. In plants Cpf1 from Prevotella and Francisella has been successfully used as a Cas9 alternative. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c. Alternative CRISPR-Cas systems include composite nuclease and effector systems designed and applied in the context of generating mutations in endogenous plant genes e.g. CRISPR TiD system (Osakabe et al., 2020); or composite nuclease and repair enzymes e.g. the prime editing system described by Anzalone et al, (2019), can tightly control the cell repair and sequence insertion that occurs following the DNA cutting.

### Sequence Insertion or Integration

The CRISPR-Cas system can be combined with the provision of a nucleic acid sequence to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains, namely a RuvC domain and a HNH domain. Mutations have been shown to alter the double strand cutting to single strand cutting and resulting in a technology variant referred to as a nickase or a nuclease-inactivated Cas9 The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art, for example S. pyogenes Cas9 nuclease with a D10A mutation or H840A mutation.

### Genome Base Editing or Modification

Base editors have been created by fusing a cytosine deaminase with a Cas9 domain (WO 2018/086623). By fusing the deaminase, the base editor uses the sequence targeting directed by the gRNA to make targeted cytidine (C) to uracil (U) substitutions by deamination of the cytidine in the DNA. The mismatch repair mechanisms of the cell then replace the U with a T. Suitable cytidine deaminases include APOBEC1 deaminase, activation-induced cytidine deaminase (AID), APOBEC3G and CDA1. Further, the Cas9-deaminase fusion may include a mutated Cas9 with nickase activity to generate a single strand break. It has been suggested that the nickase protein is potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of a Cas9 ribonucleoprotein (RNP) which is transformed into a cell such as a protoplast. It does not rely on the Cas9 encoding sequence being integrated into the host genome, thereby reducing the undesirable non-specific cleavages that may occur with integration of the Cas9 gene into sites at random. Only short flanking sequences are required to form a stable Cas9 and sgRNA stable ribonucleoprotein in vitro. Woo et al. (2015) produced pre-assembled Cas9/sgRNA protein/RNA complexes and introduced them into protoplasts of Arabidopsis, rice, lettuce and tobacco, and observed targeted mutagenesis frequencies of up to 45% in regenerated plants. In vitro introduction of RNP was demonstrated in several species including dicotyledonous plants (Woo et al., 2015), and the monocotyledonous plants maize (Svitashev et al., 2016) and wheat (Liang et al., 2017). Genome editing of plants using CRISPR-Cas 9 in vitro transcripts or ribonucleoproteins are described in Liang et al. (2018) and Liang et al. (2019).

### Method for Gene Insertion

Plant embryos may be bombarded with a Cas9 gene and sgRNA gene targeting the site of integration along with the DNA repair template. A DNA repair template may be a synthesised DNA fragment, a 127-mer polynucleotide or longer, each encoding the gene of interest to be inserted. For example, bombarded cells are grown on tissue culture medium. DNA is extracted from callus or leaf tissue of regenerated plants using CTAB DNA extraction method and analysed by PCR to confirm gene integration. T1 progeny plants can be selected from plants confirmed to contain the inserted gene of interest.

The method comprises introducing into a plant cell the DNA sequence of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site and the donor DNA is inserted into the plant genome.

Although homologous recombination occurs at low frequency in somatic cells of plants, the process appears to be increased/stimulated by the introduction of doublestrand breaks (DSBs) at selected endonuclease target sites.

### RNA Interference

RNA interference (RNAi) is particularly useful for specifically reducing the expression of a gene, which results in reduced production of a particular protein if the gene encodes a protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules is well within the capacity of a person skilled in the art, particularly considering Waterhouse et al. (1998), Smith et al (2000), WO 99/32619, WO 99/53050, WO 99/49029, WO 01/34815, WO19/051563 and WO20/024019.

In one example, a DNA is introduced that directs the synthesis of an at least partly double stranded RNA product(s) with homology to a gene encoding a polypeptide defined herein, preferably one or more of an mtSSB-1 or mtSSB-1a polypeptide, a TWINKLE polypeptide or a RECA3 polypeptide. The DNA therefore comprises both sense and antisense sequences that, when transcribed into RNA, can hybridize to form the double stranded RNA region. In one embodiment of the invention, the sense and antisense sequences are separated by a spacer region that comprises an intron which, when transcribed into RNA, is spliced out. This arrangement has been shown to result in a higher efficiency of gene silencing (Smith et al., 2000). The double stranded region may comprise one or two RNA molecules, transcribed from either one DNA region or two. The presence of the double stranded molecule is thought to trigger a response from an endogenous system that destroys both the double stranded RNA and also the homologous RNA transcript from the target gene, efficiently reducing or eliminating the activity of the target gene.

The length of the sense and antisense sequences that hybridize should each be at least 19 contiguous nucleotides, preferably at least 30, at least 32, at least 40 or at least 50 contiguous nucleotides, more preferably at least 100 or at least 200 contiguous nucleotides. Generally, a sequence of 100-1000 nucleotides corresponding to a region of the target gene mRNA is used. The full-length sequence corresponding to the entire gene transcript may be used. The degree of identity of the sense sequence to the targeted transcript (and therefore also the identity of the antisense sequence to the complement of the target transcript) should be at least 85%, at least 90%, or 95-100%, preferably is identical to the targeted sequence. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-25 contiguous nucleotides of the target mRNA. Preferably, the siRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the organism in which it is to be introduced, for example, as determined by standard BLAST search.

In an embodiment, the dsRNA comprises an “ledRNA” structure as described in WO2019/051563 and/or comprises G:U basepairs as described in WO2020/024019.

DsRNA's useful for the invention could readily be produced using routine procedures.

microRNA

MicroRNAs (abbreviated miRNAs) are non-coding RNA molecules having a length generally 19-25 nucleotides (commonly about 20-24 nucleotides in plants) that are derived from larger precursors that form imperfect stem-loop structures. The miRNA is typically fully complementary to a region of a target mRNA whose expression is to be reduced, but need not be fully complementary. miRNAs bind to complementary sequences in target messenger RNA transcripts (mRNAs), usually resulting in translational repression or target degradation and gene silencing. Artificial miRNAs (amiRNAs) can be designed based on natural miRNAs for reducing the expression of any gene of interest, as well known in the art.

In plant cells, miRNA precursor molecules are believed to be largely processed in the nucleus. The pri-miRNA (containing one or more local double-stranded or “hairpin” regions as well as the usual 5′ “cap” and polyadenylated tail of an mRNA) is processed to a shorter miRNA precursor molecule that also includes a stem-loop or fold-back structure and is termed the “pre-miRNA”. In plants, the pre-miRNAs are cleaved by distinct DICER-like (DCL) enzymes, yielding miRNA:miRNA*duplexes. Prior to transport out of the nucleus, these duplexes are methylated.

In the cytoplasm, the miRNA strand from the miRNA:miRNA duplex is selectively incorporated into an active RNA-induced silencing complex (RISC) for target recognition. The RISC-complexes contain a particular subset of Argonaute proteins that exert sequence-specific gene repression (see, for example, Millar and Waterhouse, 2005; Pasquinelli et al., 2005; Almeida and Allshire, 2005).

MicroRNA's useful for the invention could readily be produced using routine procedures. For example, the design of an OsmtSSB-1a amiRNA (artificial microRNA) construct may be based on the general method described by Fahim et al. (2012). WMD3 software (www.wmd3.weigelworld.org/) can be used to identify suitable amiRNA targets in an OsmtSSB-1a gene. The amiRNA targets are selected according to four criteria: 1) relative 5′ instability by using sequences which are AT rich at the 5′-end and GC rich at the 3′-end; 2) U at position 1 and A at the cleavage site (between positions 10 and 11); 3) maximum of 1 and 4 mismatches at positions 1 to 9, and 13 to 21, respectively; and 4) having a predicted free energy (ΔG) of less than −30 kcal mol⁻¹ when the amiRNA would hybridise to the target RNA (Ossowski et. al., 2008). For gene-specific reduction of expression, candidate amiRNA sequences are chosen in a region which shows the lowest homology upon the alignment of all the homologs of OsOsmtSSB-1a, thus reducing the potential for off-target reduction of the expression of OsmtSSB-1a homologs and homoeologs. The precursor of rice miR395 (Guddeti et al., 2005; Jones-Rhoades and Bartel, 2004; Kawashima et al., 2009) may be chosen as the amiRNA backbone for insertion of the amiRNA sequences. For example, to design and make the construct, five endogenous miRNA targets in the miR395 are replaced by five amiRNA targets for mtSSB, TWINKLE or RECA3 knock down.

### Cosuppression

Genes can suppress the expression of related endogenous genes and/or transgenes already present in the genome, a phenomenon termed homology-dependent gene silencing. Most of the instances of homology dependent gene silencing fall into two classes—those that function at the level of transcription of the transgene, and those that operate post-transcriptionally.

Post-transcriptional homology-dependent gene silencing (i.e., cosuppression) describes the loss of expression of a transgene and related endogenous or viral genes in transgenic plants. Cosuppression often, but not always, occurs when transgene transcripts are abundant, and it is generally thought to be triggered at the level of mRNA processing, localization, and/or degradation. Several models exist to explain how cosuppression works (see in Taylor, 1997).

Cosuppression involves introducing an extra copy of a gene or a fragment thereof into a plant in the sense orientation with respect to a promoter for its expression. The size of the sense fragment, its correspondence to target gene regions, and its degree of sequence identity to the target gene can be determined by those skilled in the art. In some instances, the additional copy of the gene sequence interferes with the expression of the target plant gene. Reference is made to WO 97/20936 and EP 0465572 for methods of implementing co-suppression approaches.

### Antisense Polynucleotides

The term “antisense polynucleotide” shall be taken to mean a DNA or RNA molecule that is complementary to at least a portion of a specific mRNA molecule encoding an endogenous polypeptide and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque (1995) and Senior (1998). Bourque (1995) lists a large number of examples of how antisense sequences have been utilized in plant systems as a method of gene inactivation. Bourque also states that attaining 100% inhibition of any enzyme activity may not be necessary as partial inhibition will more than likely result in measurable change in the system. Senior (1998) states that antisense methods are now a very well established technique for manipulating gene expression.

In one embodiment, the antisense polynucleotide hybridises under physiological conditions, that is, the antisense polynucleotide (which is fully or partially single stranded) is at least capable of forming a double stranded polynucleotide with mRNA encoding an endogenous mtSSB, TWINKLE or RECA3 polypeptide under normal conditions in a cell.

Antisense molecules may include sequences that correspond to the structural genes or for sequences that effect control over the gene expression or splicing event. For example, the antisense sequence may correspond to the targeted coding region of endogenous gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 30 or at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%, typically 100% identical. The antisense RNA molecule may of course comprise unrelated sequences which may function to stabilize the molecule.

### Nucleic Acid Constructs

The present invention includes nucleic acid constructs comprising the polynucleotides of or useful for the invention, and vectors and host cells containing these, methods of their production and use, and uses thereof.

The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in grain of a cereal plant such as a rice plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages. Compartmentation in specific subcellular locations such as the plastid, cytosol, vacuole, or apoplastic space may be achieved by the inclusion in the structure of the gene product of appropriate signals, e.g. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

Seed specific promoters for the invention which are suitable and include promoters which lead to the seed-specific expression in cereals well known in the art. Notable promoters which are suitable are the barley LPT2 or LPT1 gene promoters (WO 95/15389 and WO 95/23230) or the promoters described in WO 99/16890 (promoters from the barley hordein gene). Other promoters include those described by Broun et al. (1998), Potenza et al. (2004), US 20070192902 and US 20030159173. In an embodiment, the seed specific promoter is preferentially expressed in defined parts of the seed such as the endosperm, preferably the developing aleurone. In a further embodiment, the seed specific promoter is not expressed, or is only expressed at a low level, after the seed germinates.

In an embodiment, the promoter is at least active at a time point between the time of anthesis and 30 days post-anthesis, or active entirely during this period. An example of such a promoter is an OsmtSSB-1a gene promoter, a TWINKLE promoter, a RECA3 promoter or TA2 promoter (WO 2017/083920).

The promoters contemplated by the present invention may be native to the host plant to be transformed or may be derived from an alternative source, where the region is functional in the host plant. Numerous promoters that are functional in monocotyledonous plants are well known in the art. Non-limiting methods for assessing promoter activity are disclosed by Medberry et al. (1992 and 1993), Sambrook et al. (1989, supra) and U.S. Pat. No. 5,164,316.

Alternatively or additionally, the promoter may be an inducible promoter or a developmentally regulated promoter which is capable of driving expression of the introduced polynucleotide at an appropriate developmental stage of the cereal plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “chimeric vector” is meant a nucleic acid molecule, preferably a DNA molecule derived, for example, from a plasmid, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolpyruvylshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (Niedz et al., 1995) or derivatives thereof a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant or cell of the invention. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

The level of a mtSSB, TWINKLE or RECA3 polypeptide may be modulated by decreasing the level of expression of a gene encoding the protein in the cereal plant, leading to increased aleurone thickness. The level of expression of a gene may be modulated by altering the copy number per cell, for example by introducing a synthetic genetic construct comprising the coding sequence and a transcriptional control element that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in increased aleurone thickness. Alternatively, a population of mutagenized seed or a population of plants from a breeding program may be screened for individual lines with increased aleurone thickness.

### Recombinant Cells

Another embodiment of the present invention includes a recombinant cell comprising a mutated endogenous gene, for example a gene modified by gene editing, or a host cell transformed with one or more recombinant molecules of the present invention, or progeny cells thereof. Various types of mutagenesis including gene editing are described above and can be used to generate the recombinant cell. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism, for example a transgenic plant. Transformed nucleic acid molecules of the present invention can remain extrachromosomal or can integrate into one or more sites within a chromosome of the transformed cell in such a manner that their ability to be expressed is retained. Preferred host cells are plant cells, more preferably a cereal grain cell.

Plants with Genetic Variations

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture i.e. in vitro. By “plant tissue” is meant differentiated tissue in a plant or obtained from a plant (“explant”) or undifferentiated tissue derived from immature or mature embryos, seeds, roots, shoots, fruits, tubers, pollen, and various forms of aggregations of plant cells in culture, such as calli. Exemplary plant tissues in or from seeds are cotyledon, embryo and embryo axis, or scutellum, endosperm or aleurone. The invention accordingly includes plants and plant parts and products comprising these.

The terms “grain” and “seed” are used interchangeably herein. “Grain” can refer to mature grain in the plant, developing seed in the plant, harvested grain or to grain after processing such as, for example, milling or polishing, where most of the grain stays intact, or after imbibition or germination, according to the context. Mature grain commonly has a moisture content of less than about 18-20%, typically about 8-12% by weight. In an embodiment, developing grain of the invention is at least about 10 days after pollination (DAP). In an embodiment, developing seed of the invention is seed between the time of pollination and 30 days post-pollination, which may be comprised in a plant or excised.

A “transgenic plant” as used herein refers to a plant with one or more genetic variations as defined herein which contains one or more exogenous polynucleotide(s) not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The transgene is preferably stably integrated into the nuclear genome of the plant. The transgene may comprise sequences that naturally occur in the same species but in a rearranged order or in a different arrangement of elements, for example an antisense sequence. Plants containing such sequences are included herein in “transgenic plants”.

A “non-transgenic plant” is one which has not been genetically modified by the introduction of genetic material by recombinant DNA techniques.

“Wild-type”, as used herein, refers to a cell, polypeptide, gene, tissue, grain or plant that has not been modified according to the invention. Wild-type cells, tissue or plants may be used as controls to compare levels of expression of an endogenous gene, the amount of a polypeptide of the invention or other polypeptide, an exogenous nucleic acid or the extent and nature of trait modification with cells, tissue, grain or plants modified as described herein, in particular the phenotype of aleurone thickness.

As used herein, the term “corresponding wild-type” cereal plant or grain, or similar phrases, refers to a cereal plant or grain which comprises at least 75%, more preferably at least 95%, more preferably at least 97%, more preferably at least 99%, and even more preferably 99.5% of the genotype of a cereal plant or grain of the invention, but does not comprise the genetic variation(s) (such as an introduced mutation(s)) which each reduce the activity of the polypeptide in the plant or grain, and/or a thickened aleurone. In a preferred embodiment, a cereal grain or plant of the invention is isogenic relative a wild-type cereal grain or plant apart from the one or more genetic variations (such as an introduced mutation). Preferably, the corresponding wild-type plant or grain is of/from the same cultivar or variety as the progenitor of the plant/grain of the invention, or a sibling plant line which lacks the one or more genetic modifications and/or does not have a thickened aleurone, often termed a “segregant”. In an embodiment, a rice plant or grain of the invention has a genotype that is less than 50% identical to the genotype of wild-type rice cultivar Zhonghua 11 (ZH11). ZH11 has been commercially available since 1986.

Transgenic plants, as defined in the context of the present invention include progeny of the cereal plants which have been genetically modified according to the invention, wherein the progeny comprise the genetic variation(s) e.g. the mutation(s) or transgene of interest. Such progeny may be obtained by self-fertilisation of the primary transgenic plant or by crossing such plants with another cereal plant. This would generally be to modulate the production of at least one protein defined herein in the desired plant or grain thereof. Transgenic plant parts include all parts and cells of said plants comprising the transgene such as, for example, cultured tissues, callus and protoplasts, preferably the grain.

As used herein, “cereal” refers to any plant of the family Poaceae, commonly called the grasses, which is cultivated for the edible components of its grain. Cereals are therefore one family of monocotyledonous flowering plants. Examples of cereal plants/grain of the invention include, but are not limited to, rice, wheat, barley, rye, maize, sorghum, oat and triticale.

As used herein, the term “rice” refers to any plant or part thereof, including strains, varieties or cultivars of the plants, of the Genus Oryza which is cultivated for the edible components of its grain. It is preferred that the rice plant is of the species Oryza sativa.

As used herein, “brown rice” means the whole grain of rice including the bran layer and embryo (germ) but not the hull which has been removed, usually during harvesting. That is, brown rice has not been polished to remove the aleurone and embryo. The “brown” refers to the presence of brown or yellow-brown pigments in the bran layer. Brown rice is considered a wholegrain. As used herein “white rice” (milled rice) means rice grain from which the bran and germ have been removed i.e. essentially the remaining starchy endosperm of the whole rice grain. Both of these classes of rice grain may come in short, medium or long grain forms. Compared with white rice, brown rice has a higher content of protein, minerals and vitamins and a higher lysine content in its protein content.

As used herein, the term “wheat” refers to any plant or part thereof, including strains, varieties or cultivars of the plants, of the Genus Triticum which is cultivated for the edible components of its grain, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the term “barley” refers to any plant or part thereof, including strains, varieties or cultivars of the plants, of the Genus Hordeum which is cultivated for the edible components of its grain, including progenitors thereof, as well as progeny thereof produced by crosses with other species. It is preferred that the plant is of the species Hordeum vulgare.

As used herein, “pigmented” refers to grain which comprises an atypical colour. Examples include black rice, red rice, red wheat, purple rice, purple barley, and purple maize. Black rice and red rice, each of which contain pigments in the aleurone layer, in particular anthocyanidins and anthocyanins. Pigmented rice has a higher riboflavin content than non-pigmented rice, but similar thiamine content. “Black rice” has a black or almost black coloured bran layer due to a concentration of anthocyanins, and may turn a deep purple colour upon cooking. “Purple rice” (also known as “forbidden rice”) is a short grain variant of black rice and is included in black rice as defined here. It is purple in colour in the uncooked state and deep purple when cooked. “Red rice” contains a variety of anthocyanins that gives the bran a red/maroon colour, including cyanidin-3-glucoside (chrysanthemin) and peonidin-3-glucoside (oxycoccicy-anin).

Each of these types of cereal grain may be treated to prevent germination, for example by cooking (boiling) or by dry heating (roasting). For instance, brown and pigmented rice is typically cooked for 10-40 min, depending on the desired texture, whereas white rice is typically cooked for 12-18 min. Cooking or heating reduces the levels of antinutritional factors in grain such as trypsin inhibitor, oryzacystatin and haemagglutinins (lectins) by denaturation of these proteins, but not of the phytate content. Cereal grain may also be soaked in water before cooking, or slow-cooked for longer times, as known in the art. Cereal grain may also be cracked, parboiled, or heat-stabilised. Each of these forms of the cereal grain are included in the class of processed grains which are unable to germinate and which cannot be used to generate a viable plant. Bran may be steam treated to stabilise it, for example for about 6 min at 100° C.

In an embodiment, grain of the invention has delayed grain maturation when compared to corresponding wild-type cereal grain. The plant of the invention may have a reduced seed setting rate (%) relative to the wild-type plant, which entails calculating the percentage of florets in the plant that were filled by a seed at the mature grain stage of growth.

In an embodiment, grain of the invention has a decreased germination capacity when compared corresponding wild-type cereal grain. For example, the grain has about 50% of the germination capacity of corresponding wild-type cereal grain when cultured at 28° C. under 12 h light/12 h dark cycles without humidity control in a growth chamber. The term “germination” as used herein is defined as when the radicle had visibly emerged through the seed coat.

In an embodiment, plants of the invention have one or more or all of normal plant height, fertility (male and female), seed length, seed width and seed thickness relative to the wild-type parental variety, such as an isogenic plant comprising a OsmtSSB-1a polypeptide with a sequence of amino acids provided as SEQ ID NO: 3. In an embodiment, grain of the invention is capable of producing a cereal plant which has one or more or all of: normal plant height, fertility (male and female), seed length, seed width and seed thickness relative to the wild-type parental variety. As used herein, the term “normal” can be determined by measuring the same trait in the wild-type parental variety grown under the same conditions as a plant of the invention. In an embodiment, to be normal a plant of the invention has +/−20%, more preferably +/−10%, more preferably +/−5%, even more preferably +/−2.5%, of the level/number etc of the defined feature when compared to the wild-type parental variety.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the plants of the invention are homozygous for each and every genetic variation, gene or nucleic acid construct that has been introduced (transgene), or mutation(s), so that their progeny do not segregate for the desired phenotype. The transgenic plants may also be heterozygous for the introduced genetic variation, gene or nucleic acid construct, such as, for example, in F1 progeny which have been grown from hybrid seed. Such plants may provide advantages such as hybrid vigour, well known in the art.

In an embodiment, a method of selecting a plant of the invention further comprises analysing a DNA sample from the plant for at least one “other genetic marker”. As used herein, the “other genetic marker” may be any molecules which are linked to a desired trait of a plant. Such markers are well known to those skilled in the art and include molecular markers linked to genes determining traits such disease resistance, yield, plant morphology, grain quality, dormancy traits, grain colour, gibberellic acid content in the seed, plant height, flour colour and the like. Examples of such genes are the Rht genes that determine a semi-dwarf growth habit and therefore lodging resistance.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Marker Assisted Selection

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis of at least one genetic variation that alters mtSSB, TWINKLE or RECA3 activity and that confers upon the plant increased aleurone thickness, allows rapid selection of plants carrying the desired trait, which may be nurtured to maturity in the greenhouse or field for subsequent further backcrossing to the recurrent parent.

Any molecular biological technique known in the art can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of a (for example) mtSSB, TWINKLE or RECA3 gene which alters mtSSB, TWINKLE or RECA3 activity, respectively, and that confers upon the plant increased aleurone thickness. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al. (2001).

In an embodiment, a linked locus for marker assisted selection is at least within 1 cM, or 0.5 cM, or 0.1 cM, or 0.01 cM from a gene encoding a polypeptide of the invention.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G Moller (editors), BIOS Scientific Publishers Ltd, Oxford, (2000)). PCR can be performed on cDNA obtained from reverse transcribing mRNA isolated from plant cells expressing a mtSSB, TWINKLE or RECA3 gene or allele which upon the plant increased aleurone thickness. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al., (supra) and Sambrook et al., (supra). Sequencing can be carried out by any suitable method, for example, dideoxy sequencing, chemical sequencing or variations thereof. Direct sequencing has the advantage of determining variation in any base pair of a particular sequence.

### Grain Processing

Due to the thickened aleurone, cereal grain of the invention, and flour and bran therefrom, has an improved nutritional content. Isolated aleurone tissue should contain low levels of starch and pericarp, and represents a major portion of the grain's physiologically beneficial substances for human nutrition. For instance, grain of the invention and/or flour produced therefrom comprises, when compared to a corresponding wild-type cereal grain and/or flour produced therefrom, one or more or all of the following, each on a weight basis,
i) a higher fat content such as at least about 10%, at least about 20% or at least about 30%, or about 30%, higher, to a maximum about 50% higher than the wild-type,
ii) a higher ash content such as at least about 5%, at least about 10%, at least about 15% or at least about 20%, or about 20%, higher, to a maximum about 25% higher than the wild-type,
iii) a higher fiber content such as at least about 70%, at least about 80% or at least about 94%, or about 94%, higher total fibre, to a maximum about 150% higher than the wild-type,
iv) a lower starch content, such as reduced by between about 1% and about 10% by weight relative to the starch content of the corresponding wild-type cereal grain,
v) a higher mineral content such as about at least 10%, at least 15%, at least 20% or at least about 25% higher to a maximum of about 40% higher than the wild-type, preferably the mineral content is the content of one or more or all of zinc (such as at least about 10% or at least about 15% higher to a maximum of about 30% higher than the wild-type), iron (such as at least about 2.5% or at least about 5% higher to a maximum of about 10% higher than the wild-type), potassium (such as at least about 20% or at least about 25% higher to a maximum of about 40% higher than the wild-type), magnesium (such as at least about 18% or at least about 23% higher to a maximum of about 40% higher than the wild-type), phosphorus (such as at least about 17% or at least about 22% higher to a maximum of about 40% higher than the wild-type) and sulphur (such as at least about 5% or at least about 10% higher to a maximum of about 20% higher than the wild-type),
vi) a higher antioxidant content such as at least about 20%, at least about 25%, or at least about 30%, more total phenolic compounds, and/or at least about 35%, or at least about 46%, more antioxidant capacity, to a maximum of about 60% higher than the wild-type,
vii) a higher phytate content such as at least about 10% or at least about 15% higher, to a maximum of about 25% higher than the wild-type,
viii) a higher content of one or more or all of vitamins B3 (such as at least 5% or at least 9%), B6 (such as at least 100% or at least 120% to a maximum of about 200% higher than the wild-type) and B9 (such as at least 50% or at least 61% to a maximum of about 100% higher than the wild-type),
ix) a higher sucrose content such as at least 60% or at least 76%, to a maximum of about 100% higher than the wild-type,
x) a higher neutral non-starch polysaccharide content such as at least about 38% or at least about 48% higher, to a maximum about 80% higher than the wild-type,
xi) a higher monosaccharide content (for example arabinose, xylose, galactose, glucose content) such as at least about 42% or at least about 59% higher, to a maximum of about 100% higher than the wild-type, and
xii) similar nitrogen levels.

Each of these nutritional components of grain can be determined using routine techniques such as outlined in Examples 1 and 4.

In an embodiment, the grain comprises an increased proportion of amylose in its total starch content compared to the corresponding wild-type cereal grain. Methods of producing such grain are described in, for example, WO2002/037955, WO2003/094600, WO2005/040381, WO2005/001098, WO2011/011833 and WO2012/103594.

In an embodiment, grain of the invention comprises an increased proportion of oleic acid and/or a decreased proportion of palmitic acid in its total fatty acid content compared to the corresponding wild-type cereal grain. Methods of producing such grain are described in, for example, WO2008/006171 and WO2013/159149.

In a further embodiment, the grain or plant of the invention further comprises a ROS1a gene encoding a ROS1a polypeptide and one or more genetic variations which each reduce the activity of at least one ROS1a gene in the plant when compared to a corresponding wild-type plant. Such grain and plants are described in WO2017/083920. Preferably, such grain or plant is rice grain or a rice plant respectively.

Grain/seed of the invention, or other plant parts of the invention, can be processed to produce a food ingredient, food or non-food product using any technique known in the art.

As used herein, the term “other food or beverage ingredient” refers to any substance suitable for consumption by an animal, preferably any substance suitable for consumption by a human, when provided as part of a food or beverage. Examples include, but are not limited to, grain from other plant species, sugar, etc, but excluding water.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain. The particle size of refined flour is described as flour in which not less than 98% passes through a cloth having openings not larger than those of woven wire cloth designated “212 micrometers (U.S. Wire 70)”. The coarse fraction includes at least one of: bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e.-ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food. Preferred food products are cooked rice, rice porridge, rice noodles and bars containing processed rice grain, and a preferred beverage is rice tea. See Example 19 herein.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively, bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### EXAMPLES

### Example 1. General Materials and Methods

Observation of Aleurone by Staining with Sudan Red Solution

Stain solution was prepared by adding 1 g of Sudan Red IV to 50 ml of polyethylene glycol solution (average molecular weight 400, Sigma, Cat. No. 202398), incubated at 90° C. for 1 h, and mixed with equal volume of 90% glycerol. After removing the fruit coat (palea and lemma) of each grain, mature rice grains were incubated in distilled water for 5 h and then sectioned transversely or longitudinally using a razor blade. Sections were stained in Sudan Red solution at room temperature for 24 to 72 h. The sections were then counter-stained with Lugol staining solution (Sigma, 32922) at room temperature for 20 min and observed under a dissecting microscope (Sreenivasulu, 2010).

Staining of Aleurone with Evans Blue

Evans Blue stain solution was prepared by dissolving 0.1 g of Evans Blue (Sigma, E2129) in 100 ml distilled water. After removing the fruit coat of each grain, mature rice grains were sectioned transversely using a razor blade. Sections were incubated in distilled water at room temperature for 30 min, the stain added and left at room temperature for 2 min. The stain solution was then discarded, the sections washed twice with distilled water and observed under a dissecting microscope.

### Light Microscopic Observation of Rice Endosperm

Rice grains were fixed in formalin-acetic acid-alcohol (FAA) solution (60% ethanol, 33% H2O, 5% glacial acetic acid and 2% formaldehyde; v/v/v/v), degassed for one hour, dehydrated in a series of alcohol solutions containing 70%, 80%, 95% and then 100% ethanol, infiltrated by LR white resin (Electron Microscopy Sciences, 14380) and polymerized for 24 h at 60° C. Microtome sectioning was done using a Leica UC7 microtome. Sections were stained in 0.1% toluidine blue solution (Sigma, T3260) at room temperature for 2 min, then washed twice with distilled water and examined by light microscopy. Alternatively, sections were stained in 0.01% Calcofluor White solution (Sigma, 18909) at room temperature for 2 min and examined by light microscopy with UV-excitation (excitation 365 nm and emission ˜440 nm).

Staining with Periodic acid-Schiff (PAS) reagent and Coomassie Blue

The fixed sections on slides were incubated in preheated 0.4% periodic acid (Sigma, 375810) at 57° C. for 30 min, then rinsed three times in distilled water. Schiff reagent (Sigma, 3952016) was applied and the slides incubated at room temperature for 15 min, then rinsed three times in distilled water. The sections were then incubated in 1% Coomassie blue (R-250, Thermo Scientific, 20278) at room temperature for 2 min, and rinsed three times in distilled water. Dehydration of the sections was achieved using a series of alcohol solutions having 30%, 50%, 60%, 75%, 85%, 95% and 100% ethanol for 2 min each, followed by clearing of each slide in 50% xylene and 100% xylene solution (Sigma, 534056) for 2 mM each. Coverslips were then mounted with Eukitt® quick hardening mounting medium (Fluka, 03989) and the sections observed under a light microscope.

### DNA Extraction and PCR Conditions

Two methods were used for DNA extraction from plant leaf samples: a rapid DNA extraction method to provide less pure DNA samples and a more extensive DNA extraction method for purer DNA, modified from Kim et al. (2016). In the first method, four glass beads with diameter of 2 mm (Sigma, 273627), 1 to 2 mg of rice leaf tissue and 150 μl of extraction buffer (10 mM Tris, pH 9.5, 0.5 mM EDTA, 100 mM KCl) were added to each well of a 96-well PCR plate. The plate was sealed and the mixtures homogenized using a Mini-Beadbeater-96 mixer (GlenMills, 1001) for 1 min. After centrifugation of the plate at 3000 rpm for 5 min, aliquots of the supernatants containing extracted DNA were used in PCR reactions.

In the second method, 0.2 g leaf samples each with two 2 mm diameter glass beads in 1.5 ml Eppendorf tubes were cooled in liquid nitrogen for 10 min. The samples were then homogenized for 1 min in a Mini-Beadbeater-96, then 600 μl DNA extraction buffer (2% SDS, 0.4 M NaCl, 2 mM EDTA, 10 mM Tris-HCl, pH 8.0) was added to each tube and the mixtures incubated at 65° C. for one hour. After cooling the mixtures, 450 μl of 6 M NaCl was added to each tube. The tubes were vortexed and centrifuged at 12000 rpm for 20 min. Each supernatant was transferred to a new tube and the DNA precipitated using an equal volume of 2-propanol at −20° C. for 1 h. DNA was recovered by centrifugation at 2400 rpm at 4° C. for 20 min and the pellets washed twice with 75% ethanol. The pellets were air-dried at room temperature and each resuspended in 600 μl distilled water containing 10 ng/μl RNAse (Thermo Scientific, EN0201) and used in PCR reactions.

The PCR reactions used 5 μl of 2×PCR buffer containing Taq Polymerase (Thermo Scientific, K0171), 5′ and 3′ oligonucleotide primers and 1 μl of DNA sample in a total volume of 10 μl Amplification was performed using 35 cycles of 94° C. for 30 sec, 55° C. for 30 sec and 72° C. for 30 sec. Amplification products were analyzed by gel electrophoresis using 3% agarose gels. Control PCR reactions used DNA preparations from homozygous Zhonghua11 (ZH11) plants or grain, which was the parental wild-type Japonica rice variety, from homozygous NJ6 plants as the wild-type Indica rice variety, and a mixture of DNA from ZH11 and NJ6.

For genetic mapping of the ta1 allele, PCR amplifications for the genetic markers used the following primer pairs (5′ to 3′ sequences): Indel 559 (position 25,149,249 on Chromosome 5), forward primer TTATCAGCTTCTCGGTTCATCCAA (SEQ ID NO:50), reverse primer AAACAGGGTTAGCAATTTCGTTTT (SEQ ID NO:51); Indel 548 (position 25,265,533 on Chromosome 5, forward primer TCTGTCCCATATATACAACCG (SEQ ID NO:52), reverse primer ATTTGTGTTTCAATCCTATATA (SEQ ID NO:53); Indel 562 (position 25,237,218 on Chromosome 5), forward primer GGTCCTTTCAAACACTCCACA (SEQ ID NO:54), reverse primer CCAACTTTGCCTTAGCATTCA (SEQ ID NO:55); Indel 583 (position 25,265,326 on Chromosome 5), forward primer ACTACTCCCTATTTCTGTATTCACT (SEQ ID NO:56), reverse primer GAGCGTCATGGTTCACCTCTT (SEQ ID NO:57).

### TILLING Assays

The primers that were used in the TILLING assays had the nucleotide sequences: TA1-1F: CATTCATAATGACCAGCTAGGTGCT (SEQ ID NO:98) and TA1-1R: GAGTTGAAACATGCCTGTACTCCTG (SEQ ID NO:99). The PCR amplifications with ExTaq were performed with the following reaction conditions: 95° C. for 2 min; 8 cycles of 94° C. for 20 sec, 68° C. for 30 sec (1° C. decrease per cycle), and 72° C. for 60 sec for every 1 kb of amplicon length, followed by 35 cycles of 94° C. for 20 sec, 60° C. for 30 sec, and 72° C. for 60 sec for each 1 kb of amplicon length, and a final extension at 72° C. for 5 min. PCR products from the wild-type and test samples were mixed and subjected to a complete denaturation-slow annealing program to form heteroduplexes under the following conditions: 99° C. for 10 min for denaturation, followed by 70 cycles of decrements, starting at 70° C., 20 sec each, with a 0.3° C. decrease per cycle, and then holding at 15° C. to reanneal the denatured PCR products to form heteroduplexes. Cell digestion of annealed PCR products was performed in 15 μL reaction mixtures containing Cell buffer (10 mM HEPES, pH 7.5, 10 mM KCl, 10 mM MgSO₄, 0.002% Triton X-100, and 0.2 jug/mL bovine serum albumin (BSA), 4 μL of PCR product, and 1 unit Cell (10 units/μL) if PCR products were polymerized by Ex Taq, or 20 units Cell if the PCR products were polymerized by KOD, at 45° C. for 15 min, followed by adding 2 μL of 0.5 M EDTA (pH 8.0) to stop the reaction. Alternatively, the digestions were performed in 15-4, reaction mixtures containing 4 μL of PCR products and 2 units of mung bean nuclease (MBN, 10 units/μL, Cat. No. M0250S; New England Biolabs, USA) in MBN buffer (20 mM Bis-Tris, pH 6.5, 10 mM MgSO₄, 0.2 mM ZnSO₄, 0.002% Triton X-100, and 0.2 μg/mL BSA) at 60° C. for 30 min, followed by adding 2 μL of 0.2% SDS to stop the reaction.

The total Ce/I-digested PCR products were electrophoresed in a 2% agarose gel to detect mutations.

### High-Resolution DNA Melting Curve (FIRM) Analysis

Young leaf tissue (2 mm×2 mm) was homogenised with 150 μL lysis buffer containing 10 mM Tris (pH 9.5), 0.5 mM EDTA, 100 mM KCl using glass beads (2 mm diameter) in a well of a 96-well plate, homogenised as described above for DNA extraction. PCR reactions were performed in 10 μL, volumes: 5 μL of 2×master mix (Roche, Cat No: 04909631001), Forward primer at 10 μM, reverse primer at 10 μM, 1.0 μL of 25 mM MgCl₂, 1.5 μL DNA template. The mixtures were cycled at 95° C. for 10 min, then 45 cycles of 95° C. for 10 sec, annealing at 53° C. for 15 sec and 72° C. for 15 sec. Then a denaturing program: 95° C. 60 sec, 40° C. 60 sec, 65° C. 1 sec, 97° C. 1 sec; Ramp 4.4° C./sec, duration for 60 sec, target at 95° C.

### Single-Stranded DNA Binding Function Study of TA1

As described below, the TA1 gene product was identified as a mitochondrial targeted single-stranded DNA binding (mtSSB) protein. In order to study the function of TA1 polypeptide in binding single-stranded DNA (ssDNA), purified recombinant TA1 protein fused with a 6×His tag was analyzed by Electrophoretic Mobility Shift Assay (EMSA) to determine its affinity for ssDNA.

Expression of a His-Tagged TA1 Protein in E. coli

The protein coding region of 621 bp (SEQ ID NO:2) for the mature TA1 protein was amplified by PCR, cloned into the pET-30a vector (Novagen Cat. No. 69909-3) and the desired orientation of the gene insert relative to the promoter on pET-30a was confirmed. This construct was designated pET-30a-TA1. It contained a 6×His tag derived from the pET-30a vector, translationally fused at the N-terminus of the expressed TA1 polypeptide, to allow for binding to a nickel column. The recombinant TA1 was expressed in Transetta (DE3) chemically component E. coli cells (Transgen Cat. No. CD801) and grown overnight in LB medium. Cells were lysed by heating at 100° C. for 5 min in 1×SDS loading buffer (12 mM Tris pH 6.8, 5% glycerol, 0.4% SDS, 1% β-mercaptoethanol, 0.02% bromophenol blue). Soluble proteins were fractionated by electrophoresis on an SDS-PAGE gel (Bio-Rad) and transferred to a PVDF membrane (Bio-Rad). Recombinant protein was then visualized by Western analysis using a monoclonal antibody directed against the 6×His tag (Marine Biological Laboratory).

### Analytical Methods

Proximates and other major constituents in grain, food ingredients and food samples were determined using standard methods, for example as described below.

Grain moisture content was measured according to the Association of Official Analytical Chemists (AOAC) Method 925.10. Briefly, grain samples of about 2 g were dried to constant weight in an oven at 130° C. for about 1 h.

Ash content was measured according to AOAC Method 923.03. Samples used for moisture determination were ashed in a muffle furnace at 520° C. for 15 h.

### Protein Content of Grain, Food Ingredients and Food Samples

Protein content was measured according to AOAC Method 992.23. Briefly, total nitrogen was analysed by the Dumas combustion method using an automated nitrogen analyser (Elementar Rapid N cube, Elementar Analysensysteme GmbH, Hanau, Germany). The protein content of grain or food samples (g/100 g) was estimated by multiplying nitrogen content by 6.25.

### Sugars, Starch and Other Polysaccharides

Total starch content was measured according to AOAC Method 996.11 which uses the enzymatic method of McCleary et al. (1997).

The amount of sugars was measured according to AOAC Method 982.14. Briefly, simple sugars were extracted with aqueous ethanol (80% ethanol) and then quantified by HPLC using a polyamine-bonded polymeric gel column, using acetonitrile:water (75:25 v/v) as the mobile phase and an evaporative light scattering detector.

Total neutral non-starch polysaccharides (NNSP) were measured by the gas chromatographic procedure of Theander et al. (1995) with a slight modification which involved a 2 h hydrolysis with 1 M sulfuric acid followed by centrifugation.

Fructans (fructo-oligosaccharides) were analyzed by the method detailed by AOAC Method 999.03. Briefly, the fructo-oligosaccharides were extracted into water followed by digestion with a sucrase/maltase/invertase mixture. The resultant free sugars were then reduced with sodium borohydride and digested to fructose/glucose with fructanose. The released fructose/glucose was measured using p-hydroxybenzioc acid hydrazine (PAHBAH).

### Fibre Content

Total Dietary Fibre (TDF) was measured according to AOAC Method 985.29 and Soluble and Insoluble Fibre (SIF) according to AOAC Method 991.43. Briefly, TDF was determined by the gravimetric technique of Prosky et al. (1985), as detailed in the AOAC Method 985.29, and SIF was determined by a gravimetric technique as described in AOAC 991.43.

### Total Lipid

Samples of 5 g flour were incubated with 1% Clarase 40000 (Southern Biological, MC23.31) at 45° C. for 1 h. Lipids were extracted from the samples into chloroform/methanol by multiple extractions. After centrifugation to separate phases, the chloroform/methanol fraction was removed and dried at 101° C. for 30 min to recover the lipid. The mass of residue left represented the total lipid in the sample (AOAC Method 983.23).

### Fatty Acid Profile of Lipids

Lipid was extracted from milled flours into chloroform according to AOAC Method 983.23. A portion of the chloroform fraction containing the lipid was evaporated under a stream of nitrogen after addition of an aliquot of hepta-decanoic acid as an internal standard. The residue was suspended in 1% sulfuric acid in dry methanol and the mixture heated at 50° C. for 16 h. The mixture was diluted with water and extracted twice with hexane. The combined hexane solution was loaded onto a small column of Florisil and the column washed with hexane and the fatty acid methyl esters then eluted with 10% ether in hexane. The eluent was evaporated to dryness and the residue dissolved in iso-octane for injection onto the GC. Fatty acid methyl esters were quantified against a mixture of standard fatty acids. GC conditions: Column SGE BPX70 30 m×0.32 mm×0.25 μm; Injection 0.5 μL; Injector 250° C.; 15:1 split; Flow 1.723 ml/min constant flow; Oven 150° C. for 0.5 min, 10° C./min to 180° C., 1.5° C./min to 220° C., 30° C./min to 260° C., total run-time 33 min; Detector FID at 280° C.

### Antioxidant Activity (ORAC-H)

The hydrophilic antioxidant activity (ORAC-H) was determined following the method of Huang (2002a and 2002b) with modification as described by Wolbang et al. (2010). The samples were extracted for lipophilic antioxidants followed by hydrophilic antioxidants as follows: 100 mg of sample weighed in triplicate into 2 mL microtubes. 1 mL hexane:dichloromethane (50:50) was then added and mixed vigorously for 2 min and centrifuged at 13,000 rpm for 2 min at 10° C. The supernatant was transferred to a glass vial and the pellet re-extracted with a further 2 mL of hexane:dichloromethane mix. The mixing and centrifuge steps were then repeated, and the supernatant transferred to the same glass vial. Residual solvent from the pellet was evaporated under a gentle stream of nitrogen. 1 mL of acetone:water:acetic acid mix (70:29.5:0.5) was then added and the samples mixed vigorously for 2 min. The mixture was then centrifuged as before and the supernatant used in the ORAC-H plate assay. Samples were diluted as required with phosphate buffer. The area under the curve (AUC) was calculated and compared against AUC values for Trolox standards. The ORAC value is reported as μISA Trolox equivalents/g of sample.

### Phenolics

Total phenolics content as well as phenolics in the free, conjugated and bound states were determined following extraction according to the method described by Li et al. (2008) with minor modifications. Briefly, the free phenolics were determined in 100 mg samples following extraction into 2 mL 80% methanol by sonication for 10 min in a glass vial (8 ml capacity). The supernatant was transferred to a second glass vial and the extraction of the residue repeated. The combined supernatants were evaporated to dryness under nitrogen. 2 mL of 2% acetic acid was added to adjust the pH to about 2 and then 3 mL ethyl acetate added to extract the phenolics with shaking for 2 min. The vials were centrifuged at 2000×g for 5 min at 10° C. Supernatants were transferred to a clean glass vial and the extraction repeated twice more. Combined supernatants were evaporated under nitrogen at 37° C. Residues were dissolved in 2 mL 80% methanol and refrigerated.

Samples for the conjugated phenolics were treated as for the free phenolic assay for the initial 80% methanol extraction. At this point 2.5 mL 2M sodium hydroxide and a magnetic bar were added to the evaporated supernatants in the glass vial which was then filled with nitrogen and capped tightly. The vials were mixed and heated at 110° C. for 1 h with stirring. Samples were cooled on ice before extraction with 3 mL ethyl acetate by shaking for 2 min. The vials were centrifuged at 2000×g for 5 min at 10° C. Supernatants were discarded and pH adjusted to about 2 with 12 M HCl. Phenolics were extracted using 3×3 mL aliquots of ethyl acetate as described for the free phenolics. The supernatants were combined and evaporated to dryness under nitrogen at 37° C. and the residue was taken up in 2 mL 80% methanol and refrigerated.

Bound phenolics were measured from the residues following methanolic extraction of the free phenolics. 2.5 mL of 2M sodium hydroxide and a magnetic bar were added to the residue before filling the vial with nitrogen and capping it tightly. The vials were mixed and heated at 110° C. for 1 h with stirring. Samples were cooled on ice before extraction with 3 mL ethyl acetate by shaking for 2 min. The vials were centrifuged at 2000×g for 5 min at 10° C. The supernatant was discarded and pH adjusted to about 2 with 12 M HCl. Phenolics were extracted with 3×3 mL aliquots of ethyl acetate as described for the free phenolics. The supernatants were combined and evaporated to dryness under nitrogen at 37° C. and the residue was taken up in 2 mL 80% methanol and refrigerated.

Total phenolics were determined using 100 mg of samples by adding 200 μL 80% methanol to wet the samples prior to hydrolysis. 2.5 mL of 2 M sodium hydroxide and a magnetic bar were added before filling the vial with nitrogen and capping tightly. The vials were mixed and heated at 110° C. for 1 h with stirring. Samples were cooled on ice before extraction with 3 mL ethyl acetate by shaking for 2 min. The vials were centrifuged at 2000×g for 5 min at 10° C. The supernatant was discarded and pH adjusted to about 2 with 12 M HCl. Phenolics were extracted with 3×3 mL aliquots of ethyl acetate as described for the free phenolics. The supernatants were combined and evaporated to dryness under nitrogen at 37° C. and the residue was taken up in 4 mL 80% methanol and refrigerated.

The amount of phenolics in the treated/extracted samples was measured using Folin Ciocalteau's assay for determination of phenolics. Gallic acid standards at 0, 1.56, 3.13, 6.25, 12.5, and 25 μg/mL were used to prepare a standard curve. 1 mL of standards were added to 4 mL glass tubes. For test samples, 100 μL aliquots of thoroughly mixed samples were added to 900 μL water in 4 mL glass tubes. 100 mL of Folin Ciocalteau reagent was then added to each tube which was vortexed immediately. 700 μL of 1 M sodium bicarbonate solution was added after 2 min and then mixed by vortexing. Each solution was incubated at room temperature in the dark for 1 h and then absorbance read at 765 nm. Results were expressed in μg gallic acid equivalents/g sample.

### Phytate

Determination of the phytate content of flour samples was based on the method of Harland and Oberleas, as described in AOAC Official Methods of Analysis (1990). Briefly, a 0.5 g flour sample was weighed and extracted with 2.4% HCl using a rotating wheel (30 rpm) for 1 h at room temperature. The mixture was then centrifuged at 2000×g for 10 min and the supernatant extracted and diluted 20-fold with milli-Q water. An anion exchange column (500 mg Agilent Technologies) was placed on a vacuum manifold and conditioned for use following the manufacturer's instructions. The diluted supernatant was then loaded onto a column and non-phytate species removed by washing with 0.05 M HCl. Phytate was then eluted with 2 M HCl. The collected eluate was digested using a heating block. The sample was cooled and the volume made up to 10 mL with milli-Q water. Phosphorous levels were determined by spectrophotometry using the molybdate, sulphonic acid colouring method with absorbance readings at 640 nm. Phytate was calculated using the following formula:

Phytate (mg/g)=P conc*V1*V2/(1000*sample weight*0.282)

where P conc is the concentration of phosphorous (μg/mL), as determined by spectrophotometry, V1 is the volume of the final solution, V2 is the volume of the extracted phytate solution, and 0.282 is the phosphorus to phytate conversion factor.

### Total Mineral Content Estimation

Total mineral content of samples was measured by ash assay using AOAC Methods 923.03 and 930.22. About 2 g of flour was heated at 540° C. for 15 h and the mass of ash residue was then weighed. Wholemeal flour samples of 0.5 g were digested using tube block digestion with 8 M nitric acid at 140° C. for 8 h. Zinc, iron, potassium, magnesium, phosphorus and sulphur contents were then analysed using inductively coupled plasma atomic emission spectrometry (ICP-AES) according to Zarcinas (1983a and 1983b).

Minerals were analysed at CSIRO, Urrbrae, Adelaide South Australia, at Waite Analytical Service (University of Adelaide, Waite, South Australia) and at Dairy Technical Services (DTS, North Melbourne, Victoria.). Elements were determined at CSIRO by Inductively Coupled Plasma-Optical Emission Spectroscopy (ICP-OES) after digestion with nitric acid solution or at DTS after digestion with dilute nitric acid and hydrogen peroxide or by ICP-AES after digestion with nitric/perchloric acid solution.

### Vitamins

Vitamins B3 (Niacin), B6 (Pyridoxine) and total folate analyses were performed by DTS as well as National Measurement Institute (NMI). Niacin was measured by AOAC Methods 13th Ed (1980) 43.045, according to Lahey, et al. (1999). Pyridoxine was measured according to Mann et al. (2001). The method incorporated a pre-column transformation of phosphorylated and free vitamin B6 forms into pyridoxine (pyridoxol). Acid phosphatase hydrolysis was used for dephosphorylation followed by de-amination with glyoxylic acid in the presence of Fe2+ to convert pyridoxamine to pyridoxal. Pyridoxal was then reduced by sodium borohydride to pyridoxine.

Folic acid was measured either according to VitaFast Folic acid kit using the manufacturer's instructions, or according to AOAC method 2004.05.

### Example 2. Isolation and Characterisation of Thick-Aleurone (Ta) Mutants

### Establishment and Cultivation of a Mutagenised Rice Population

About 8000 grains (designated M0 grains) from wild-type rice cultivar Zhonghua11 (ZH11) were mutagenized by treatment with 60 mM ethyl methane sulfonate (EMS) using standard conditions. Mutagenised grains were sown in the field and the resultant plants cultivated to produce M1 grains. M1 grains were harvested and then sown in the field to produce M1 plants. 8925 panicles were harvested from 1327 individual M1 plants. From these plants 36,420 M2 grains were screened, including at least 4 grains from each panicle.

### Mutant Screening by Staining of Half Grains

The fruit coat (palea and lemma) of M2 rice grains were removed. Each of the 36,420 grains was transverse bisected. The halves containing an embryo were saved in 96-well plates for subsequent germination, while each half grain without an embryo was stained with Evans Blue and observed under a dissecting microscope to detect mutant grains having thickened aleurones relative to the wild-type. The staining was based on the principle that Evans Blue could only penetrate and stain non-viable cells such as the cells of the starchy endosperm while no colour change was observed in the viable aleurone layer. From initial Evans Blue staining and histological analyses, individual grains exhibiting significant increases in aleurone thickness as well as grains showing a significant thickening in the ventral side aleurone of the seed were observed. Other grains showed an increase in aleurone thickness but to a lesser extent. The unstained region of the ventral side of each seed was especially examined for thickness of the aleurone layer. Variants with increases in thickness of the aleurone layer on the dorsal side of the grains were also observed. Only variants with significant increases in the thickness of the aleurone layer across the entire cross-section were chosen for further analysis.

Compared with wild-type half-grains, the half-grains having a thicker unstained region with Evans Blue were selected. Amongst the 36,420 grains examined, 219 grains (0.60%) having differences in aleurone thickness were identified and selected. These had been obtained from 162 panicles from 140 individual M1 plants, and therefore most represented independent mutants. One mutant grain in particular was identified and characterised further as described below, having a mutation in a gene designated as thick-aleurone 1 (ta1). The corresponding wild-type gene was designated TA1; that designation is used herein. The mutant allele was designated ta1-1 as the first identified mutation in the TA1 gene.

To maintain the putative mutant lines, each corresponding embryonated half grain was germinated on medium containing half-strength MS salts medium (Murashige and Skoog, 1962) solidified with 1% Bacto agar (Bacto, 214030) and cultured at 25° C. under light of intensity 1500˜2000 Lux with 16 h light/8 h dark cycles. The plantlets were transferred to soil at the two to three leaflet stage and the resultant plants grown to maturity. Upon the germination and cultivation of the corresponding embryonated half grains, 115 seeds (52.5% survival) were grown up to produce mature and fertile plants.

Candidate mutant plants which exhibited little or no defects in general agronomical traits such as those that were of normal plant height, fertility (male and female fertility), grain size and 1,000 grain weight relative to the wild-type parental variety as well as showing stable inheritance of the thickened aleurone trait were identified, selected and further analysed. Among them, grain of the M3 generation which exhibited three to five cell layers in at least a part of the aleurone was selected and analysed in detail, having the ta1-1 mutant allele. Wild-type ZH11 grain exhibited an aleurone of 1-2 cell layers around most of the grain, as expected.

### Histological Analyses of the Ta1-1 Mutant Grain

Developing grain from wild-type ZH11 and homozygous ta1-1 mutant plants were studied and compared for morphological changes from 1 to 30 days after pollination (DAP). The ripening phase of rice grain can be said to have three stages: a milk grain stage, a dough grain stage and a mature grain stage. In the dough grain stage, the grains in wild-type panicles began to change in colour from green to yellow, following by a gradual destruction of vesicular tissue connecting the stalk and caryopsis. Grains in the ta1-1 panicles were delayed in the colour change. Microscopic examination of the transverse sections of the rice grains also showed an increase in the degree of chalkiness (opaqueness) in ta1-1 mutant grains. Scanning electron microscopy (SEM) was then used to study the structure of the starch granule organization in the middle part of the starchy endosperm. In wild-type grains, starch granules were tightly packed and showed a smooth surface and regular shape, while in ta1-1 grains a looser packing of irregular-shaped starch granules was observed. In summary, at least three changes were observed in the plants and grain having the ta1-1 mutation: a delay in grain maturation, an increase in the degree of chalkiness of the grain and in starch granule structure.

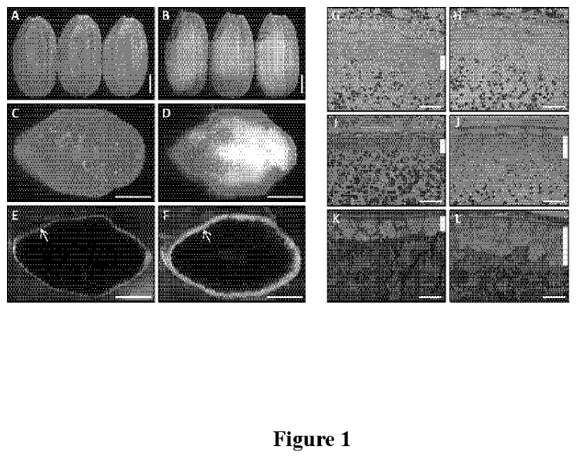

Developing mutant and wild-type grains at 6, 7, 8, 9, 10, 12, 15, 18, 21, 24, 27 and 30 DAP were stained with Evans Blue and the aleurone layers examined by light microscopy. No significant difference was observed in the thickness of the developing aleurone layers between wild-type and ta1-1 mutant grains up until 10 DAP. After 10 DAP, the aleurone layers of ta1-1 mutants were thicker than in the wild-type grains, and the difference reached a maximum at around 20 DAP. FIG. 1 shows the staining of seed sections with Lugol's reagent emphasizing the starchy endosperm (FIG. 1, panels E and F) as well as PAS and Coomassie brilliant blue (G-250) staining of semi-thin sections of seeds to demonstrate the development of the aleurone in the wild-type (ZH11) and ta1-1 mutant at three developmental time points.

The wild-type and ta1-1 mutant grains (30 DAP) were further examined for histological differences by sectioning (1 μm), staining and light microscopy. After staining with 0.1% toluidine blue which stains nucleic acid blue and polysaccharide purple, a single layer of large, regularly oriented, rectangular cells was observed in wild-type aleurones. In contrast, sections of ta1-1 mutant grains had aleurone layers of three to five cell layers, the cells also being of varying sizes and irregular orientation. These observations indicated that the thickened aleurones in the ta1-1 grains were mainly caused by the increase in the number of cell layers rather than the enlargement of individual aleurone cells.

Further staining with 0.01% Calcofluor White, a fluorescent cell wall stain, showed no difference in cell wall thickness between wild-type and ta1-1 mutant grains in the aleurone layer. The cell walls of aleurone cells were thicker than cell walls in the starchy endosperm for both wild-type and ta1-1 grains.

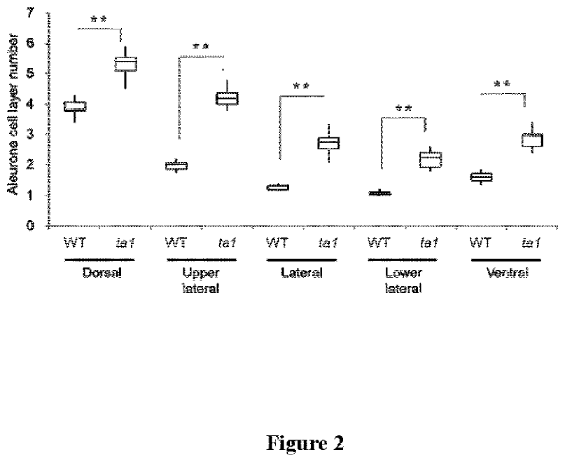

Mature wild-type and ta1-1 grains were transverse sectioned and stained with PAS as described in Example 1. The thickness of the aleurones in the dorsal, upper lateral, lateral, lower lateral and ventral positions of the grain, calculated as the number of cells layers, was counted and statistically analyzed for 15 grains of each genotype. The results are shown in FIG. 2. It was observed that the number of cell layers was not uniform in the five different positions of the mature grain for both the wild-type and mutant grain, but in every position, the number of cell layers for the mutant grain was significantly greater than for the wild-type grain (P<0.01).

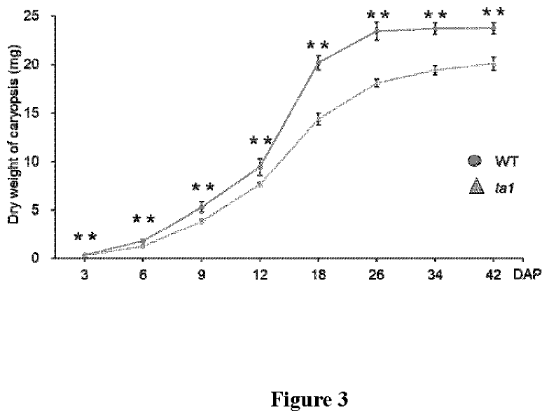

Grain filling during plant growth and development was examined by measuring the grain dry weight at 3, 6, 9, 12, 18, 26, 34 and 42 DAP, and compared to wild-type grain. The results (FIG. 3) showed reduced grain filling for the ta1-1 grain.

### Analysis of the Agronomical Characteristics of Ta1 Mutant Plants and Grains

After backcrossed to wild-type (ZH11) plants for three generations in the field to yield the BC3F3 generation, thereby removing additional, unlinked mutations that might have arisen from the mutagenic treatment, ta1-1 mutant plants were analysed for some agronomical traits. The ta1-1 mutant plants and grain were not significantly different, compared to wild-type plants and grain, in plant height, grain size in terms of length, width and thickness, and caryopsis morphology (Table 2). In contrast, wild-type plants showed a seed setting rate of 98.90% whereas homozygous ta1-1 mutant plants showed a decrease in seed setting rate at 89.32%. The seed setting rate was calculated as the percentage of florets in the plant that were filled by a seed by the mature grain stage. Moreover, the ta1-1 mutant grains showed a decrease in germination capacity of 49.8% in comparison with wild-type grain of 97.3% when cultured at 28° C. under 12 h light/12 h dark cycles without humidity control in a growth chamber. Germination was defined as when the radicle had visibly emerged through the seed coat.

TABLE 2
Comparison of wild-type (ZH11) and ta1-1 mutant plants for agronomical
traits (Means ± SE).

| Trait | ZH11 | ta1-1 |
|---|---|---|
| Plant Height (cm) | 103.7 (±3.24) | 101.5 (±5.62) |
| Seed setting rate (%) | 98.9 (±3.41) | 89.32 (±5.15) |
| 1000 seeds weight (g) | 22.73 (±0.17) | 19.99 (±0.33) |
| Seed length (mm) | 7.46 (±0.26) | 7.38 (±0.25) |
| Seed width (mm) | 3.27 (±0.12) | 3.37 (±0.13) |
| Seed thickness (mm) | 2.35 (±0.10) | 2.24 (±0.11) |

### Example 3. Genetic Analysis of the Ta1-1 Mutation

Crosses were made to determine the genetic control of the ta1 mutation, in particular whether the mutation was dominant or recessive, and whether it was inherited from one or both of male and female parents. Two genetic experiments were performed to determine whether the thick aleurone phenotype was maternally determined. Firstly, a test cross was performed between a maternal, homozygous ta1-1 plant and pollen from a wild-type ZH11 plant. F2 progeny grains were obtained. Of the F2 grains, 24.1% (n=1093) showed the thick aleurone phenotype, which accorded with a 3:1 (wild-type:mutant) ratio predicted for Mendelian inheritance of a recessive, nuclear encoded mutation in an F2 population. It was observed that 98.7% of the F1 seeds (n=218) had the wild-type aleurone phenotype, while the remaining 1.3% of seeds were difficult to score but proved to have wild-type aleurone in the F2 generation. Secondly, a reciprocal cross was performed using pollen from a ta1-1 plant applied to a wild-type plant as the female parent. From the reciprocal cross, 99.7% F1 seeds (n=325) exhibited the wild-type aleurone phenotype. These data indicated that the thick aleurone phenotype of ta1-1 was controlled by a single nuclear gene where the mutant allele was recessive to the wild-type allele.

### Example 4. Analysis of Nutritional Components in Ta1-1 Mutant Grain

To measure the composition of mutant grain, particularly for nutritionally important components, ZH11 and homozygous ta1-1 plants were grown in the field at the same time and under the same conditions. Samples of whole grain flour, also referred to herein as wholemeal or brown rice flour, were prepared from grain harvested from the plants and used for the compositional analysis, using the methods described in Example 1. The results of the proximate analyses of the flours are given in Table 3, showing the means of duplicate measurements.

The proximate analyses indicated an increase of about 30% in the total fat content in the ta1-1 mutant flour. Total nitrogen analyses showed no significant change in the protein levels between the ta1-1 mutant and wild-type grains. Ash assays, which measured the amount of materials left behind after combustion of dehumidified flour samples, demonstrated an increase of 20% in ta1-1 grain relative to wild-type. The total fibre level increased by about 94% in ta1-1 grain. The starch content decreased by 5% in ta1-1 grain relative to wild-type. These data demonstrated that the increase in thickness of the aleurone layer in the ta1-1 mutant caused an increase in the level of aleurone-rich nutrients such as lipid, minerals and total dietary fibre without changing the size of the seed. In order to understand these and other changes in greater detail, more extensive analyses were done as follows.

TABLE 3
Compositional analysis of rice grain (in g/100 g of grain)

| | | | | Total | Total | Total | Total | Soluble | Insoluble |
|---|---|---|---|---|---|---|---|---|---|
| Sample | Moisture | Ash | Protein | Fat | Starch | Sugars | Fibre | Fibre | Fibre |
| ZH 11 | 9.54 | 1.79 | 15.3 | 3.30 | 66.0 | 1.02 | 1.6 | 0.7 | 2.9 |
| ta1-1 | 9.19 | 2.14 | 15.8 | 4.28 | 62.7 | 1.84 | 3.1 | 0.6 | 3.7 |

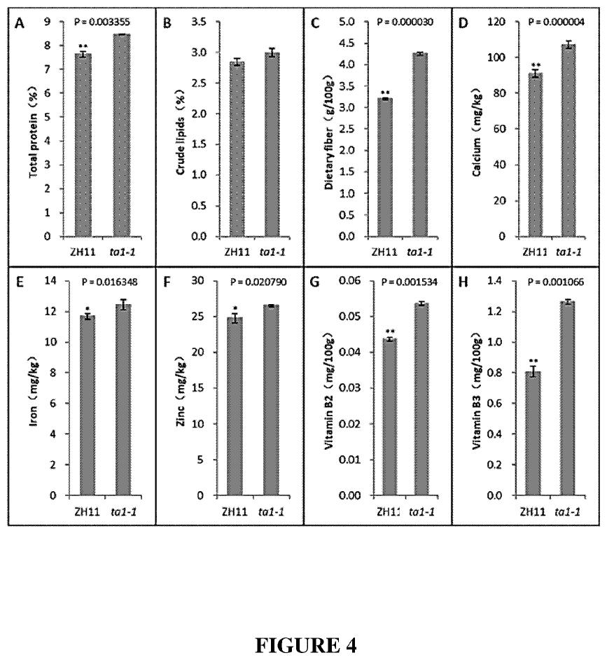

Further compositional analyses are recorded in FIG. 4, confirming statistically significant increases in brown rice flour in protein, dietary fibre, calcium, iron, zinc, vitamin B2, and vitamin B3.

### Minerals

To measure mineral contents, ICP-AES was used which combined inductively coupled plasma (ICP) with atomic emission spectrometry (AES) techniques (Example 1). This was a standard method for measuring mineral content, providing a sensitive and high throughput quantitation of a large number of elements in a single analysis. The data obtained from the analysis showed that the ta1-1 mutant grain had levels of zinc increased by about 15% from 13.9 mg/kg to 15.68 mg/kg. In the same sample of field grown ta1-1 wholegrain flour, iron increased by 5%, from 12.43 mg/kg to 13.03 mg/kg.

Increases in potassium, magnesium, phosphorus and sulphur were also observed, being increased by about 26%, 23%, 22% and 10%, respectively. These results were consistent with the increase in ash content in ta1-1 grain, which measured mostly minerals.

### Antioxidants

Antioxidants are biomolecules capable of counteracting the negative effects of oxidation in animal tissues, thus protecting against oxidative stress-related diseases such as inflammation, cardiovascular disease, cancer and aging-related disorders (Huang, 2005).

The antioxidant capacity in wholemeal flour obtained from the ta1-1 mutant and wild-type rice grain was measured by an oxygen radical absorbance capacity (ORAC) assay as described in Example 1. In the ORAC assay, the antioxidant capacity is represented by the competition kinetics between endogenous radical scavenging biomolecules and the oxidisable molecular fluorescent probe fluorescein, against the synthetic free radicals generated by AAPH (2,2′-azobis(2-amidino-propane) dihydrochloride). The capacity was calculated by comparison of the area under the kinetic curve (AUC), representing the fluorescence degradation kinetics of the molecular probe fluorescein for the grains with the AUC generated by Trolox standards (Prior, 2005). An alternative approach to quantifying antioxidant capacity was through the use of the Folin-Ciocalteau reagent (FCR); this represented the antioxidant capacity by measuring the reducing capacity of the total phenolic compounds in the food sample. The FCR assay was relatively simple, convenient and reproducible. However, the more time-consuming ORAC assay measured more biologically relevant activity. Since antioxidants include a wide range of polyphenols, reducing agents and nucleophiles, measurement by both FCR and ORAC can provide a better coverage and more comprehensive representation of the total antioxidant capacity. As reported by Prior (2005), the results of FCR assay and ORAC measurement are usually consistent.

Both of the FCR and ORAC assays showed increased antioxidant capacity of flour from the ta1-1 mutant whole grain flour relative to wild-type, ZH11 whole grain flour. ORAC demonstrated an increase of about 46% in antioxidant capacity and 30% in total phenolics in the ta1-1 mutant grain.

### Phytate

When grown under conditions with adequate phosphorus, about 70% of total phosphorus content in rice grain is in the form of phytate or phytic acid (myo-inostitol-1,2,3,4,5,6-hexakisphosphate). Dietary phytate may also have beneficial roles for health as a strong antioxidant (Schlemmer, 2009). Total phytate analyses showed an increase in phytate content by about 15% in ta1-1 grain as compared to the wild-type ZH11 grain, increasing from about 10.8 mg/g to about 12.4 mg/g.

### B Vitamins

Levels of the vitamins B3 (niacin), B6 (pyridoxine) and B9 (folate) in the ta1-1 flour were higher than those in wild-type flour by about 9%, 120% and 61%, respectively. Aleurone is known to be richer in vitamins B3, B6 and B9 than endosperm (Calhoun, 1960), so the increase in aleurone thickness in the ta1-1 mutant grain was likely to have created a stronger sink for these vitamins and probably others.

### Carbohydrates

There was a 6% decrease in the starch content of the ta1-1 grain on a weight basis. In contrast, sucrose levels increased by 76% in the mutant grain. Neutral non-starch polysaccharides (NNSP) increased by 48%, and the monosaccharide components of the NNSP (arabinose, xylose, galactose, glucose) all increased from 42% to 59% relative to the wild-type.

### Conclusions

The nutritional analyses showed that wholegrain flour produced from field-grown ta1-1 grain and therefore of the whole grain was significantly increased relative to wild-type flour in most of the aleurone-rich nutrients including the macro-nutrients such as lipid and fibre, micro-nutrients such as minerals including at least the iron, zinc, potassium, magnesium, phosphorus and sulphur levels, B vitamins such as B3, B6 and B9, antioxidants, and aleurone-associated biomolecules such as phenolic compounds and phytate. There was also a substantial increase in free sucrose. Concomitant with the increase in these nutrients and micronutrients was a small decrease in starch content in the ta1-1 mutant on a weight basis, as a relative percentage.

### Example 5. α-Amylase Activity Assay of Ta1-1 Mutant Grain

During cereal seed germination, α-amylase produced from the aleurone layer after imbibition plays an important role in hydrolysing the endosperm starch into metabolizable sugars, which provide the energy for the growth of roots and shoots (Akazawa and Hara-Mishimura, 1985; Beck and Ziegler, 1989). Because ta1 mutant grain had increased aleurone cell layers, α-amylase activity of germinated rice grains was assayed over a number of days. The Amylase Activity Assay Kit (Sigma, MAK009) was used to do the assay and the detailed method was as follows.

### Sample Preparation

100 mg germinated rice grains were each dissected to remove the seedling. The remaining endosperm and aleurone tissues were homogenized in 0.5 mL of Amylase Assay Buffer and mixtures centrifuged at 13,000 g for 10 min to remove insoluble material. 1-50 μL of sample was added per well of a 96 well plate, and each sample brought to 50 μL volume with Amylase Assay Buffer. For positive controls, 5 μL of the Amylase Positive Control solution was used and adjusted to 50 μL with the Amylase Assay Buffer. Standards comprised of 0, 2, 4, 6, 8, 10 μL of a 2 mM Nitrophenol Standard per well and were brought to 50 μL volume, generating 0, 4, 8, 12, 16, and 20 nmole/well standards.

### Assay Reaction

1. A Master Reaction Mix was prepared by mixing 50 mL of Amylase Assay Buffer and 50 mL of Amylase Substrate Mix.

2. 100 μL of the Master Reaction Mix was added to each of the samples, standards, and positive control wells, mixing each sample by pipetting.

3. After 2-3 minutes (Tinitial), the absorbance was measured at 405 nm, called (A405)initial.

4. The plate was incubated at 25° C., measuring the absorbance (A405) every 5 minutes. The plate was protected from light during the incubation.

5. Measurements were taken until the value of the most active sample was greater than the value of the highest standard (20 nmole/well). At this time, the most active sample was near the end of the linear range of the standard curve.

6. The final absorbance measurement [(A405)final] used for calculating the enzyme activity was the value at the time point immediately before the most active sample was near the end of the linear range of the standard curve. The time of the penultimate reading was designated Tfinal.

Correction was made for the background by subtracting the final measurement (A405)final obtained for the 0 nitrophenol standard from the (A405)final measurement of the standards and samples. The change in absorbance from Tinitial to Tfinal was calculated: ΔA405=(A405)final—(A405)initial

The ΔA405 of each sample was compared to the standard curve to determine the amount of nitrophenol (B) generated by the amylase between Tinitial to Tfinal. The amylase activity of a sample was determined by the following equation:

Amylase Activity=B×Sample Dilution Factor

### (Reaction Time)×V

B=Amount (nmole) of nitrophenol generated between Tinitial and Tfinal
Reaction Time=Tfinal—Tinitial (minutes)
V=sample volume (mL) added to well

The amylase activity for each sample was reported as nmole/min/mL (milliunits) One unit of amylase was the amount of amylase that cleaved ethylidene-pNP-G7 to generate 1.0 μmole of p-nitrophenol per minute at 25° C.

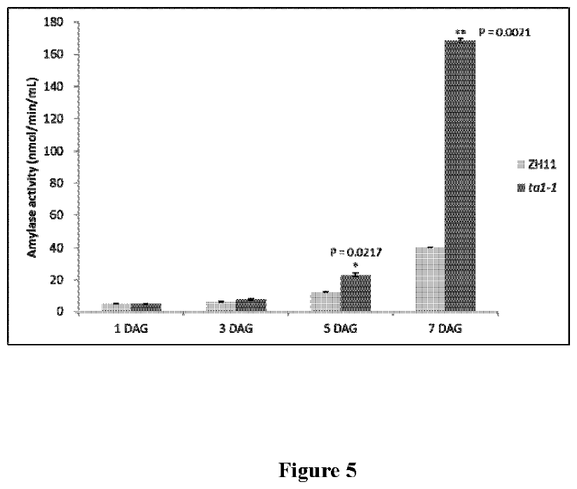

The results of the assays (FIG. 5) showed that germinated ta1-1 grains had higher α-amylase activity compared with ZH11 from 3 days onward, with the extent of increase increasing with time, greatest at 7 days after the start of imbibition (DAG).

### Example 6. Identification of the TA1 Gene and Ta1-1 Mutation by Genetic Mapping and Sequence Analysis

### Identification and Use of SSR and Indel Markers for Gene Mapping

For gene mapping, an F2 population of plants was produced from the genetic cross between a plant containing the ta1-1 mutation in the genetic background of ZH11, a Japonica variety, and a plant of the Indica variety, NJ6. To identify genetic markers which were polymorphic between ZH11 and NJ6 and which could then be used in the gene mapping, a set of PCR experiments was performed on leaf DNA samples from homozygous ZH11 plants, homozygous NJ6 plants and a 1:1 mixture of the DNAs. Analysis of the PCR products by gel electrophoresis allowed comparison of the products from ZH11, NJ6 and the mixtures to identify polymorphic markers. Primer pairs were selected for the gene mapping only if the amplifications with separate ZH11 and NJ6 DNAs showed discrete and different amplified products and the mixed DNA showed the combination of both products. A total of 124 primer pairs were thereby selected including 54 insertion-deletion polymorphisms (INDEL) and 70 short sequence repeats (SSR) polymorphisms. These genetic markers were distributed at approximately 3-4 Mbp intervals along the rice genome and gave good coverage for gene mapping.

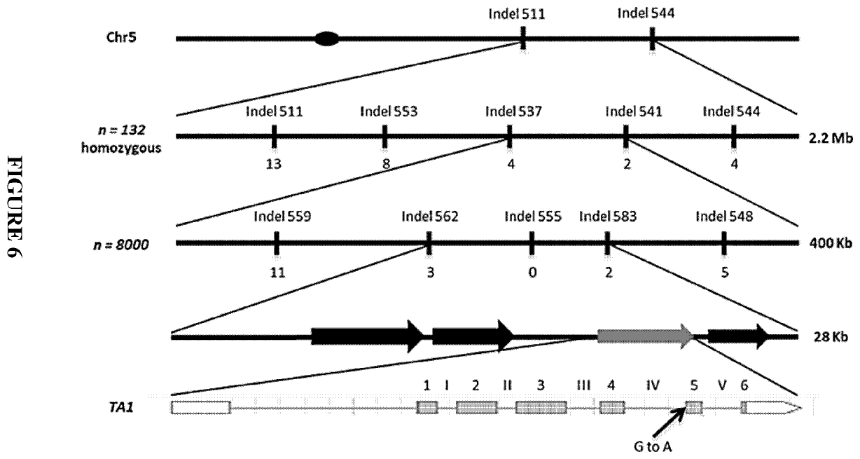

For genetic mapping of the ta1-1 allele, 132 plants from the F2 population were scored with the 124 polymorphic markers. Homozygosity or heterozygosity of the individual F2 plants in the mapping population for the aleurone phenotype was assessed carefully by phenotyping of F3 progeny grains obtained from each F2 plant. Leaf DNA was extracted as described in Example 1. PCR amplifications were done as described in Example 1 and the products separated by gel electrophoresis through 3% agarose. It was concluded from the results that the ta1 locus was located between markers Indel 537 and Indel 541 on Chromosome 5 (FIG. 6), which from the genome sequence of rice corresponding to a physical distance of approximately 400 kb.

Another 8000 F2 plants were screened with this pair of markers. 1076 individuals were identified and selected which exhibited a recombination between the Indel 537 and Indel 541 markers. When these recombinant plants were phenotyped, the ta1 locus was thereby mapped to a 28 kb region which lay between the Indel 562 and Indel 583 markers (FIG. 6).

To obtain the nucleotide sequence of this region in the ta1-1 mutant plants and compare it to the wild-type sequence and thereby identify a mutation corresponding to the ta1-1 allele, primers flanking the genomic region were designed and DNA sequencing was carried out. The comparison of the genomic DNA sequences identified a single-nucleotide polymorphism (SNP) in the sequenced region in the gene annotated on the rice genome sequence (EnsemblPlants) as Os05g0509700. This gene was in position Chromosome 5: 25,254,990-25,259,747, with the reverse strand annotated as a mitochondrial ssDNA binding protein. The same gene was annotated in the UniPARC genome sequence as LOC Os05g43440 for a protein which linked to Japonica gene Os05g0509700. The single nucleotide G (wild-type) to A polymorphism was identified at nucleotide position Chr5:25257622 with reference to the EnsemblPlants rice genome sequence of the Japonica variety. This nucleotide substitution G2126A with reference to SEQ ID NO:4 was the last nucleotide of intron IV, between exon 4 and exon 5 of the gene Os05g0509700 in chromosome 5 (FIG. 6, lowest line). The intron numbering was taken from the protein coding region annotation of 0505g0509700 and does not include a long intron in the 5′-UTR of the gene. Since the mutation involved the last nucleotide of the intron, within a splice site, the inventors suspected that the mutation involved disruption of splicing of the TA1 gene. Subsequent experiments described below confirmed that this polymorphism was the ta1-1 mutation.

This experiment also showed that the ta1-1 mutation could be introduced into different genetic backgrounds such as, for example, an Indica variety, and still conferred the thick aleurone phenotype.

### Example 7. Nature of the Ta1-1 Mutation

To analyze expression and splicing of the ta1-1 gene, RNA was extracted from leaves and developing grain from ta1-1 plants as described in Example 1. The RNAs were combined, reverse transcribed and the nucleotide sequences of individual cDNAs obtained. Three different cDNA sequences were obtained from the ta1-1 cDNA, designated ta1-1 cDNAs I, II and III, each having a different sequence to the wild-type cDNA sequence from ZH11. Comparison of the ta1-1 cDNA nucleotide sequences with the wild-type cDNA (SEQ ID NO:2) showed that the G to A polymorphism in intron IV at nucleotide position 2126 (G2126A) was associated with three different mature mRNAs in the ta1-1 grain. From this and the further analysis described below, the inventors concluded that these variants arose because of alternative splicing of the ta1 transcripts.

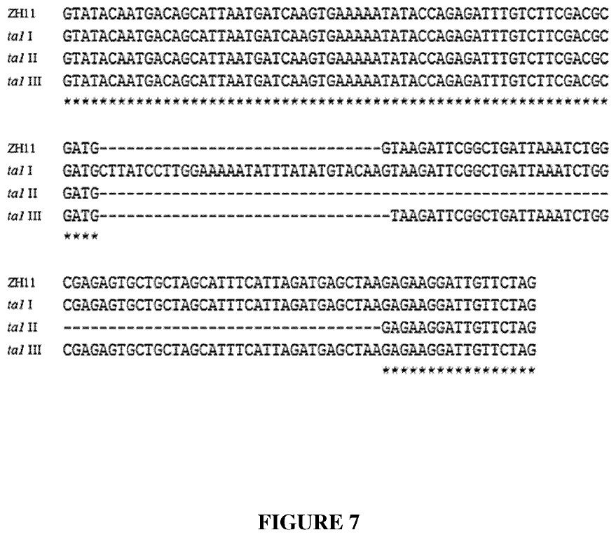

The three ta1-1 transcripts were analyzed further by RT-PCR using oligonucleotide primers (SEQ ID NO:58 and SEQ ID NO:59) spanning the mutation site and therefore the alternative splicing region. The PCR products were then recovered and cloned into a Blunt vector, followed by nucleotide sequencing of individual clones. FIG. 7 shows aligned sequences of a region of the cDNAs spanning the mutation site, aligned with the wild-type cDNA sequence. It was observed that the cDNAs I, II and III had identical nucleotide sequences with the wild-type cDNA until the end of Exon 4, i.e. the beginning of intron IV, but then diverged with either an insertion of the last 31 nucleotides of intron IV in cDNA I or deletion of the exon 5 sequence (cDNA II) or of a single nucleotide from the beginning of exon 5 (cDNA III). Since ta1-1 cDNA I (SEQ ID NO:12) had a 31 bp insertion just before exon 5, that cDNA encoded a polypeptide (SEQ ID NO:8) resulting from a translational frameshift at amino acid position 182 and leading to a premature translation stop codon at position 193 relative to the amino acid sequence of wild-type TA1 protein (SEQ ID NO:3). Since ta1-1 cDNA II (SEQ ID NO:13) had a deletion of the entire exon 5, that cDNA encoded a polypeptide (SEQ ID NO:9) resulting from a translational frameshift at amino acid position 182 and leading to a premature translational stop at position 186. Since ta1-1 cDNA III (SEQ ID NO:14) had a deletion of the first G nucleotide in exon 5, that cDNA encoded a polypeptide (SEQ ID NO:10) resulting from a translational frameshift at position 182 and leading to a premature translational stop at 185 amino acid relative to the wild-type TA1 protein. The predicted full-length amino acid sequences were compared to the wild-type TA1 sequence in FIG. 8.

The relative abundance of the ta1-1 I, II, and III cDNAs was 67%, 29% and 4% of the total transcripts, respectively. No wild-type transcript was detected in the leaves and developing grain of the ta1-1 mutant plant by the cDNA analysis; the inventors concluded from the absence of wild-type mRNA in the mutant plants that wild-type splicing was abolished. It was concluded from these data, confirmed by data from following experiments described below, that the polymorphism in intron IV of the ta1-1 gene was the causative change, i.e. the ta1-1 mutation in that plant and its grain. It was also concluded that the mutation led to a change in the splicing pattern of the RNA transcript of the ta1-1 gene relative to the wild-type TA1 gene, thereby causing the ta1 mutant phenotype for the ta1-1 allele.

The gene at position Os05g0509700 in chromosome 5 of the rice genome has been annotated as a rice mtSSB-1a gene (OsmtSSB-1a), a homolog of the Arabidopsis thaliana mtSSB-1 gene (AtmtSSB-1) which encodes a mitochondrial targeted single-stranded DNA binding protein (Edmondson et al., 2005).

The nucleotide sequence of the wild-type rice mtSSB-1a (TA1) gene is provided as SEQ ID NO:1, including a promoter and 5′-UTR (untranslated region) of 1024 nucleotides, a protein coding region from nucleotides 1025-2369 including 5 introns, and a 411 nucleotide 3′-UTR. The nucleotide positions of the S introns are provided in the legend to SEQ ID NO:1. The nucleotide sequence of the cDNA corresponding to wild-type TA1 (OsmtSSB-1a) gene is provided as SEQ ID NO:2, and the amino acid sequence of the encoded wild-type TA1 polypeptide of 206 amino acids is provided as SEQ ID NO:3.

### Description of the Structural Features in the Wild-Type Rice TA1 Polypeptide

After finding that the rice TA1 gene was the same as OsmtSSB-1a, the OsTA1 (OsmtSSB-1a) polypeptide amino acid sequence was examined. One typical single-stranded DNA binding domain was identified (FIG. 9), corresponding to amino acids 73-184 of SEQ ID NO:3. The amino acid sequence was compared to the most homologous sequences encoded by the genomes of seven other plant species (see Example 12) which are broadly representative of angiosperms, namely Zea mays (SEQ ID NO:16), Sorghum bicolor (SEQ ID NO:17), Hordeum vulgare (SEQ ID NO:18), Triticum aestivum (SEQ ID NO:19) and Brachypodium distachyon (SEQ ID NO:20) which is a model monocotyledonous plant related to cereals, Arabidopsis thaliana (SEQ ID NO:15) which is a model dicotyledonous plant, and poplar (SEQ ID NO:21) which is a model tree. Well assembled and annotated genome sequences were available for these species. When the mtSSB amino acid sequences were aligned (FIG. 9), it was apparent that the SSB domain was more highly conserved, having at least 80% identity over the 112 amino acids of the SSB domain of OsTA1, than the N-terminal and C-terminal flanking sequences. The amino acid sequence identity overall to the full length of SEQ ID NO:3 was at least 60%, including at least 79% for the monocot TA1 sequences. The SSB domain for the nine TA1 polypeptides shown in FIG. 9 included the conserved amino acid motifs FRGVHRAI(I/L)CGKVGQ(V/A)P(V/L)QKILRNG(R/H)T(V/I)T(V/I)FT(V/I)GTGG MFDQR (Motif I, SEQ ID NO:45) corresponding to amino acids 73-114 of SEQ ID NO:3, P(K/M)PAQWHRI(A/S)(V/I)H(N/S)(D/E) (Motif II, SEQ ID NO:46) corresponding to amino acids 122-135 of SEQ ID NO:3, AVQ(K/Q)L(V/T)KNS(A/S)VY(V/I)EG(D/E)IE(T/I)R(V/I)YND (Motif III, SEQ ID NO:47) corresponding to amino acids 141-164 of SEQ ID NO:3 and 184 of SEQ ID NO:3. These four motifs were completely conserved in all nine of the aligned TA1 sequences, as was the spacing between each motif except for the poplar sequence (FIG. 9). It was concluded that the four motifs and their spacing were likely to be required for full function of TA1 polypeptides in plants.

When the OsmtSSB-1a amino acid sequence (SEQ ID NO:3) was analysed with Mitofates software using the plant settings, a mitochondrial protease cleavage site was predicted between amino acids 28 and 29 of SEQ ID NO:3 (FIG. 9), predicting the presence of a mitochondrial transit peptide of 28 amino acids at the N-terminal end of the polypeptide which would be removed upon entry into the mitochondria in rice cells to produce a mature polypeptide of 178 amino acids. This predicted site aligned within one amino acid residue compared to the prediction for the Arabidopsis sequence of AtmtSSB-1 (Edmondson et al., 2005).

It was noted that the last three amino acids (GKI) of the DNA binding domain of the wild-type OsTA1 (OsmtSSB-1a) polypeptide were missing from the predicted translation products of the ta1-1 cDNAs I, II and III (FIG. 6), as well as the C-terminal region of TA1 polypeptide after the DNA binding domain.

### Example 8. Expression of the TA1 Gene in Rice

Experiments were carried out to analyze expression of the TA1 gene in different rice tissues, including in parts of the developing grain, specifically the embryo, starchy endosperm and aleurone. In a first experiment, TA1 mRNA was detected in rice tissue sections by in situ hybridisation as described by Brewer et al. (2006). Briefly, various rice tissues were fixed in FAA fixative for 8 h at 4° C. after vacuum infiltration, dehydrated using a graded ethanol series followed by a xylene series, and embedded in Paraplast Plus (Sigma-Aldrich) as described in Example 1. Microtome sections 8 μm thick were mounted on Probe-On Plus microscope slides (Fisher).

From the hybridisation signals, it was concluded that the TA1 gene is expressed in the testa, aleurone tissues and in the embryo of developing grain, but not in the vascular bundle. TA1 was also expressed in the developing inflorescence, but not in leaf and stem.

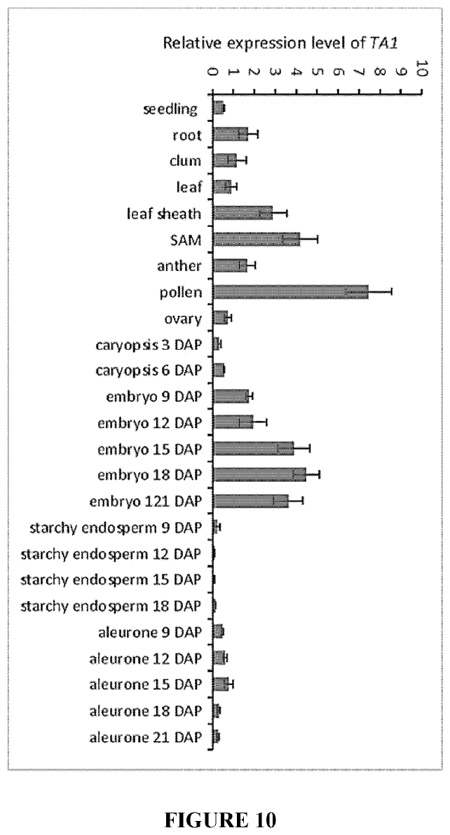

Real time reverse transcription polymerase chain reaction (RT-PCR) was used to assay relative expression levels in different plant tissues. Expression levels were normalized to expression of an actin gene, a constitutively expressed gene in the same tissue. Surprisingly, the results indicated highest relative expression in pollen, followed by embryo, shoot apical meristem and aleurone tissue (FIG. 10). It was considered that the specific expression of TA1 in active growing tissues might be involved in cell division. It was considered that as a predicted SSB protein, TA1 protein functions in protecting single strand DNA and recruiting other related proteins during DNA replication, recombination, transcription and DNA repair in these active growing tissues.

### Example 9. ssDNA Binding Activity of TA1 Protein

### Purification of a TA1 Fusion Protein

In order to test the DNA binding activity of TA1 polypeptide in vitro, a recombinant TA1 fusion protein having a 6×His tag at its N-terminal end (6×His-TA1) was produced from an expression vector in E. coli as described in Example 1. The 6×His tag was incorporated to allow for rapid and simple purification of the fusion protein by bonding to a nickel column. E. coli cells expressing the fusion protein were disrupted by freezing in liquid nitrogen, thawing on ice and sonicating for 30 min (5 s pulses at 10 s intervals). The 6×His-TA1 protein was enriched by 40% ammonium sulfate salt precipitation in potassium phosphate buffer. The precipitated protein was resuspended and dialyzed overnight against sodium phosphate buffer (50 mM NaPO4, 500 mM NaCl, pH 7.8). The protein was then loaded onto a nickel column (Bio-Rad) and washed extensively with sodium phosphate buffer containing 10 mM imidazole to remove non-binding proteins. Purified 6×His-TA1 protein was isolated from the column using a 10-500 mM linear gradient of imidazole in sodium phosphate buffer. The protein concentration in each fraction was determined by spectrophotometry and samples were separated by SDS-PAGE, followed by staining by Coomassie Blue. The purified 6×His-TA1 protein was transferred to storage buffer (50 mM Tris-HCl pH 7.4, 1 mM EDTA, 1 mM DTT, 0.2 M NaCl, 50% (v/v) glycerol) and stored at −80° C.

### Electrophoretic Mobility Shift Assay (EMSA)

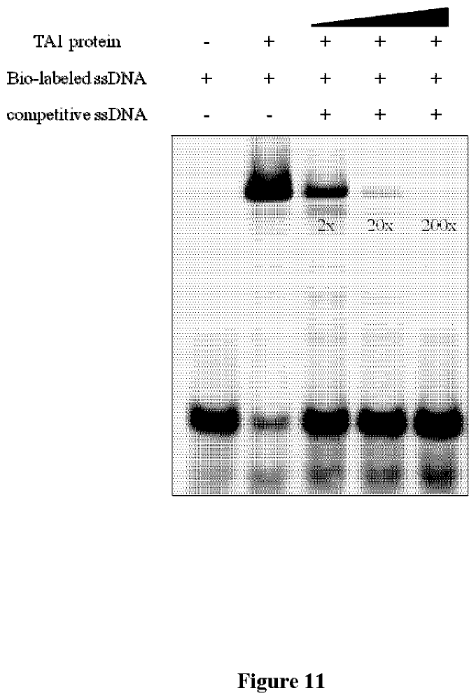

The affinity of purified 6×His-TA1 protein for ssDNA was determined using an EMSA (Thermo Cat. No. 20148X) as described in general terms by Reddy et al. (2001). A 45-nucleotide single-stranded oligonucleotide (SEQ ID NO:60) was labelled at the 3′ end with biotinylated CTP (Thermo Cat. No. 89818) according to the manufacturer's instructions. Recombinant 6×His-TA1 protein (2 μg) was added to 750 attomoles of biotin-labelled oligonucleotide and the mixture was incubated at room temperature for 10 min. A control sample lacked the 6×His-TA1 protein was applied to lane 1 in FIG. 11. Separate reactions were carried out in the presence of a 60-bp double-stranded DNA (dsDNA; control fragment from Thermo EMSA kit) to determine if the mobility shift was specific for ssDNA. Binding reaction products were separated by electrophoresis in a 5% native polyacrylamide gel in 0.5×TBE (Bio-Rad) and transferred to a nylon membrane (Bio-Rad). DNA bands were visualized using streptavidin-conjugated horseradish peroxidase (Thermo). As showed in FIG. 11, the 6×His-TA1 protein bound to ssDNA and the binding was diminished by competition with unlabeled ssDNA. The dsDNA did not compete for the binding of TA1 protein to the single-stranded DNA (ssDNA), showing that the protein was specific for ssDNA, consistent with its designation as a single-stranded DNA binding (SSB) protein.

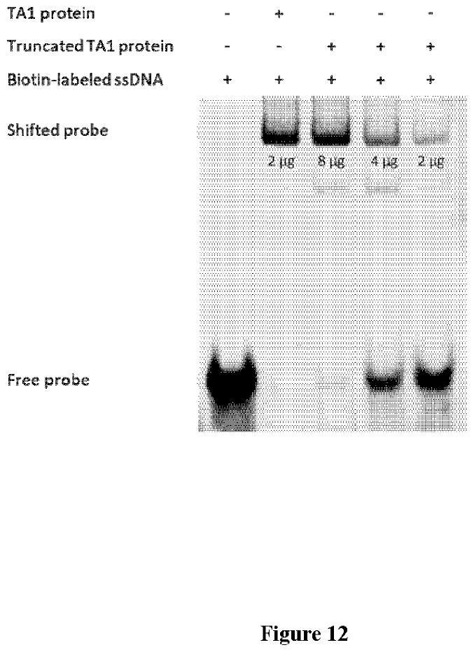

To examine whether the mutant ta1-1 I polypeptide retained an ability to bind to ssDNA, an analogous genetic construct was made that encoded the predicted polypeptide encoded by ta1-1 cDNA I, with the 6×His tag at the N-terminal end, and expressed in E. coli. The cDNA I sequence was chosen because the corresponding mRNA was the predominant, spliced transcript from the mutant ta1-1 gene, representing about 70% of the total transcription from the ta1-1 gene. The mutant 6×His-ta1-1 fusion polypeptide was purified by the same method as for the wild-type 6×His-TA1 protein. The purified 6×His-ta1-1 I fusion polypeptide was used in an EMSA experiment as described above for the corresponding wild-type protein, to compare the binding of the wild-type and mutant polypeptides to a ssDNA. The binding experiment showed that the mutant (truncated) polypeptide bound to ssDNA, but only with reduced efficiency, at about 3- to 4-fold decreased efficiency compared with the wild-type TA1 protein (FIG. 12). Both TA1 and its truncated form (6×His-ta1-1 I) were able to bind to the ssDNA probe, but the binding activity of 6×His-TA1 was higher than its truncated form (6×His-ta1-1 I), in that 2 μg 6×His-TA1 had a similar intensity of shifted probe to 8 μg truncated 6×His-ta1-1 I polypeptide. The inventors concluded from this data that at least the ta1-1 I polypeptide retained some SSB activity and that the ta1-1 allele was not a null allele. It was considered that the ta1-1 gene was reduced in activity by at least 67% relative to the wild-type TA1 gene.

### Example 10. Complementation Analysis of the Ta1-1 Mutant

In order to strengthen the conclusion that a mutation in the TA1 gene was responsible for the thickened aleurone and associated phenotypes seen in the ta1-1 mutant, two complementation experiments were performed. In a first experiment, a wild-type copy of the TA1 gene including the native TA1 promoter was introduced into ta1-1 mutant plants by Agrobacterium-mediated transformation. The DNA sequence to be introduced was amplified from ZH11 genomic DNA using a series of oligonucleotide primers, and then assembled into the binary vector pPLV15. That vector also contained a hygromycin resistance gene as a selectable marker gene. The plasmid for transformation and a control plasmid (empty vector) were each introduced into Agrobacterium tumefaciens strain EHA105 and used to transform ta1-1 rice recipient cells using the method as described by Nishimura et al. (2006). A total of 53 TO transgenic plants were regenerated from the transformation with the wild-type TA1 gene. These plants were transferred to soil and grown to maturity in a growth chamber. When PCR was used to test for the presence of the hygromycin resistance gene, 34 transformant lines were confirmed and selected which carried the hygromycin gene. These were grown to maturity and grain (T1 seed) harvested from each plant. Each of these plants contained the T-DNA from the vector containing the wild-type TA1 gene as demonstrated by PCR assays.

Grain harvested from these plants was examined for the aleurone phenotype by staining with Evans Blue. Eight independent transformed plants were tested and all of them produced grain with normal aleurones like the wild-type, indicating positive expression of the introduced gene and therefore complementation of the ta1-1 mutation. This conclusively proved that the mutation in the TA1 gene caused the mutant phenotype.

In a second complementation experiment and to allow for observation of the expression from the TA1 promoter, a second genetic construct was prepared and used for transformation. This construct included a GUS reporter sequence translationally fused after the TA1 protein coding sequence isolated from the wild-type rice genome. The genetic construct included a 4,550 nucleotide DNA fragment (nucleotide sequence provided as SEQ ID NO:49) which contained, in order, a 3208-bp upstream sequence which was considered to contain the promoter of the TA1 gene, the entire TA1 protein coding region including all of the introns, then the GUS reporter coding region translationally fused to the C-terminus of the TA1 sequence, and finally the nos 3′ polyadenylation/transcription termination region. This GUS translational fusion was designed to test whether the TA1-GUS fused protein was expressed and in which tissues of the transformed plants.

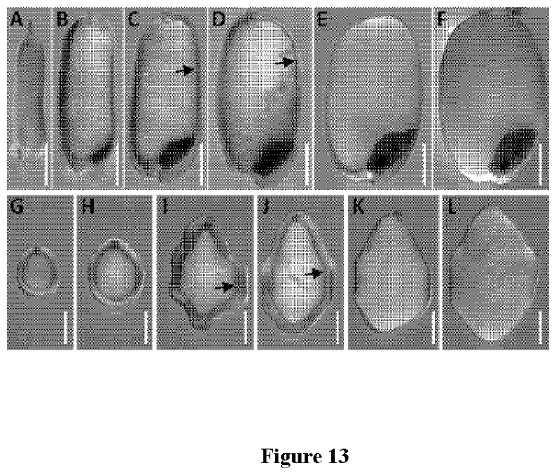

This second construct was introduced into tat-1 plants by Agrobacterium-mediated transformation, as before. Various tissues of the transformed plants including developing seeds were stained for GUS activity. The expression pattern of the TA1-GUS fusion gene in aleurone and embryos of the transgenic plants is shown in FIG. 13. GUS activity was observed in the aleurone and embryo of developing rice grain of the transgenic plants, particularly at 5-11 days DAP. Strong expression of GUS was still observed in embryos at 18 and 24 DAP, and weaker expression in aleurone at these time points. Only very low levels of expression were seen in the endosperm.

The genetic construct encoding the TA1-GUS fusion polypeptide under the control of the native TA1 promoter also complemented the ta1-1 mutation, giving plants with the normal, wild-type aleurone phenotype. This further confirmed that the mutation identified in the ta1-1 plants was responsible for the thick aleurone phenotype. It was also concluded that the GUS fusion C-terminal to the TA1 polypeptide did not compromise TA1 function.

### Example 11. Subcellular Localization of TA1 Protein

A genetic construct was made which translationally fused the protein coding regions of the wild-type rice TA1 polypeptide (SEQ ID NO:3) and a green fluorescent protein (GFP) in a vector under the control of a CaMV 35S promoter and a nos 3′ polyadenylation region/transcription terminator, designed for transient expression in plant cells. This construct was then introduced into tobacco leaves to determine the subcellular localization of the fusion protein through its GFP fluorescence. After several days to allow for expression of the introduced fusion gene, the localization of the GFP signal was compared to that from a mitochondria-specific stain, MitoTracker. By confocal-fluorescence microscopy, it was observed that the TA1-GFP fusion protein expressed from the construct in leaf epidermal cells co-localized with the MitoTracker stain. It was concluded that the TA1 protein had an N-terminal mitochondrial transit peptide (MTP) which provided for mitochondrial localization, consistent with its alternate designation as OsmtSSB-1a. These data and the conclusion were consistent with the analysis described in Example 7 regarding the presence of an MTP sequence.

### Example 12. Mitochondrial Function and Energy Homeostasis in Ta1 Rice Aleurone

In view of the mitochondrial localization of the wild-type TA1 protein and its apparent function as an SSB protein in mitochondria of cells in developing grain, the inventors examined mitochondria in the aleurone of developing ta1-1 grain. Examination of aleurone cells at 15 DAP by Transmission Electron Microscopy showed that mitochondria in the mutant grain had an abnormal shape and morphology. The mitochondria were more circular in shape compared to the more elongated shape in the wild-type aleurone, and did not have the same internal structure with distinct and well developed cristae.

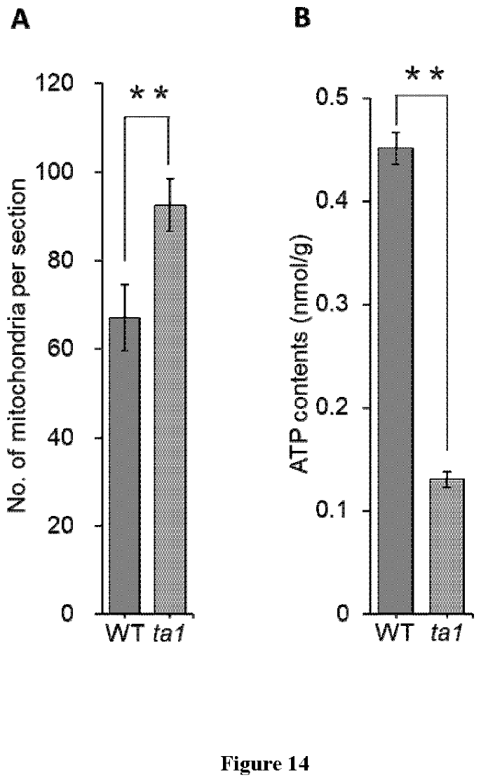

The numbers of mitochondria were counted in each section of aleurone cells of DAP caryopses of ta1-1 and wild-type grain. The ATP contents in the wild-type and ta1-1 aleurone at 11 DAP were also measured. The results (FIG. 14) showed that the number of mitochondria in ta1 aleurone cells increased compared to the wild-type, but the ATP contents were significantly reduced.

Furthermore, when the transcriptome of the ta1-1 grain was compared to the wild-type, numerous genes involved in glycolysis, oxidative phosphorylation and the citrate cycle were observed to be upregulated in the ta1-1 grain.

The inventors concluded from these data that mutation of the TA1 gene led to defective mitochondrial function, associated with increased numbers of mitochondria and upregulated genes related to mitochondrial function as the cells of the developing grain tried to compensate for the mutant ta1 gene and the associated reduced function of the mtSSB protein.

### Example 13. Screening for Additional Mutant Alleles in the TA1 Gene

To provide for further mutations including possible knock-out (null) mutations of the TA1 gene, CRISPR-Cas9 gene editing methods were used, as follows. Four separate CRISPR constructs were made by modifying the vector pYLCRISPR/Cas9-MH, provided by Dr. Yaoguang Liu (School of Life Science, South China Agricultural University, China). First, four 20 nucleotide regions each followed immediately by a 3-nucleotide PAM (protospacer adjacent motif) sequence were identified from the TA1 cDNA nucleotide sequence as potential guide RNA (gRNA) target sequences. The gRNA targets, designated Casg1, Casg2, Casg3 and Casg4, were selected with the aim of producing mutations in the second (g1 and g2), third (g3) and fourth (g4) exons respectively. The gRNA target constructs, with the gRNA target integrating into the gRNA backbone and driven by a U3 promoter from rice, were then synthesized. The synthesized DNAs were excised using restriction enzyme BsaI and inserted into the pYLCRISPR/Cas9-MH vector. The synthetic gene constructs were then isolated and transformed into Agrobacterium strain EHA105. Transformation of rice was performed by the method by Nishimura et al. (2006). Transgenic rice plants are regenerated from transformed calli. Seeds of mutant plants were examined for their aleurone phenotypes by thin-sectioning mature caryopses to analyze the aleurone layer cell number.

Table 4 lists the TA1 mutations in 28 lines including at least 13 independent lines produced using the CRISPR/Cas9 method. All of these lines had frameshift mutations within the protein coding regions that resulted in stop codons which caused premature translation termination and so would have been null mutations. Importantly, seeds of all of the mutant lines showed the thick aleurone phenotype. Grain of some of the mutant lines additionally had other phenotypic changes relative to the wild-type including one or more of chalky endosperm, reduced seed set and reduced thousand grain weight. It was concluded that null alleles of the TA1 gene (SEQ ID NO:1, encoding SEQ ID NO:3, and homologous TA1 genes) conferred the thick aleurone and associated phenotypes. With the current efficiency of gene editing techniques in rice and other plants including cereals, gene editing such as using CRISPR or TALENS is a preferred method to obtain the thick aleurone phenotype.

TABLE 4
TA1 knock-out mutants created using CRISPR/Cas9 method

| | | | putative ta1 | |
|---|---|---|---|---|
| target | line | mutation in TA1 | proteins | phenotype |
| g1 | 3,30 | T insertion | stop at 112 aa | thick aleurone |
| | 13, 16, 23, 24, | T deletion | stop at 80 aa | thick aleurone |
| | 26 | | | |
| | 4, 5 | 44 bp deletion | stop at 93 aa | thick aleurone |
| | 21 | C insertion | stop at 112 aa | thick aleurone |
| | 27 | G insertion | stop at 112 aa | thick aleurone |
| g2 | 1, 2, 5 | A insertion | stop at 112 aa | thick aleurone |
| | 10, 12, 16, 17, | T insertion | stop at 112 aa | thick aleurone |
| | 26, 27 | | | |
| | 15 | C insertion | stop at 112 aa | thick aleurone |
| g3 | 31 | T/A insertion | stop at 134 aa | thick aleurone |
| | 43 | T deletion | stop at 130 aa | thick aleurone |
| g4 | 3, 5, 11 | C/T insertion | stop at 183 aa | thick aleurone |
| | 8 | A insertion | stop at 183 aa | thick aleurone |
| | 16 | CG deletion | stop at 182 aa | thick aleurone |

### Example 14. TA1 Protein Associates with RECA3 and TWINKLE Polypeptides

SSB proteins have an important role in DNA replication, homologous recombination between closely related or identical chromosomes and DNA repair. In bacteria, the RecA protein catalyzes homologous strand exchange and SSB proteins enhance the reaction, likely by stabilizing DNA strand separation. Mitochondrial SSB proteins are considered to have a similar role in replication and recombination of the multiple mitochondrial genomes in plant cells (Edmondson et al., 2005). Mitochondrial DNA replication requires multiple nuclear-encoded proteins including a DNA polymerase known as plant organellar DNA polymerase or POP (Moriyama and Sato, 2014), a primase which acts to synthesize an RNA primer, a DNA topoisomerase also called a gyrase, mtSSB and a helicase which unwinds double-stranded DNA, also known as TWINKLE. The TWINKLE enzyme in plants, localized to the mitochondria, has both primase and helicase activity, being capable of synthesizing RNA primers of at least 15 nucleotides in length that were extended by E. coli DNA Polymerase I into high molecular weight DNA (Diray-Arce et al., 2013).

The multiple mitochondrial chromosomes of angiosperms often rearrange and recombine, in particular involving repeated sequences, to produce a multiplicity of sequence duplications, inversions, deletions and insertions (Gualberto and Newton, 2017). Several nuclear genes are known to control mitochondrial DNA recombination and genome stability. Usually, these genes code for proteins that affect the fidelity of DNA replication, recombination or repair, and mutants in these genes often result in the accumulation of recombined alternative configurations of the mitochondrial DNA. These proteins include recombinases that catalyze strand exchange, similar to RecA in bacteria. Arabidopsis thaliana has three RecA-like proteins (RECA1—RECA3) (Shedge et al., 2007). RECA1 is conserved in all plants and is targeted to plastids. RECA2 is present in flowering plants and mosses and is targeted to both mitochondria and plastids. In contrast, the RECA3 protein is targeted only to mitochondria. In Arabidopsis thaliana, both recA2 and recA3 mutations trigger increased ectopic recombination across intermediate-size repeats. RecA2 mutations are usually lethal at the early seedling stage, whereas recA3 knock-out plants are usually phenotypically normal in the first generation but can show effects in later generations.

The inventors therefore tested whether TWINKLE and RECA3 might be involved in the same processes as TA1 in rice. The rice TWINKLE and RECA3 sequences were identified from the rice genome sequence by searching with the Arabidopsis sequences as query. The amino acid sequences of the rice proteins, the nucleotide sequences of the genes encoding these proteins and the nucleotide sequences of the protein coding sequences of the cDNAs for these genes are provided as SEQ ID NOs:61-66.

In a series of experiments, interactions of the TWINKLE and RECA3 proteins with TA1 were shown. In one experiment, a split yellow fluorescent protein (YFP) assay was performed in tobacco leaves by co-transformation with pairs of constructs encoding fusion proteins. In each pair, one encoded the N-terminal half of YFP and the other encoded the C-terminal half of YFP. One construct had the chimeric genes p35S: TA1-nYPF and the other had either p35S:RECA3-cYFP or p35S:TWINKLE-cYPF. Transformed cells were examined under a confocal microscope. Fluorescent dots were observed in the cytoplasm. It was concluded from this and mitochondrial-specific staining that TA1 protein interacted with both RECA3 and TWINKLE, and that all three are localized in mitochondria.

In vivo interactions of TA1 with RECA3 and TWINKLE were also demonstrated by immunoprecipitation experiments to specifically recover one of the polypeptides, where the other polypeptides co-precipitated. To do this, a construct p35S:TA1-MYC was co-infiltrated into tobacco leaves with either a p35S:RECA3-HA construct or a p35S:TWINKLE-HA construct for transient expression of the tagged proteins. Total proteins were extracted from the infiltrated tissues and subjected to co-immunoprecipitation and resultant protein fractions tested using western blot. In another experiment, a split luciferase assay was carried out in infiltrated tobacco leaves to show that TA1 interacted with both RECA3 and TWINKLE.

From each of these experiments, the inventors concluded that the TA1 protein associated with the RECA3 and TWINKLE proteins and that these were localized in the mitochondria.

### Example 15. Downregulation of RECA3 or TWINKLE Genes Produces Thickened Aleurone in Rice

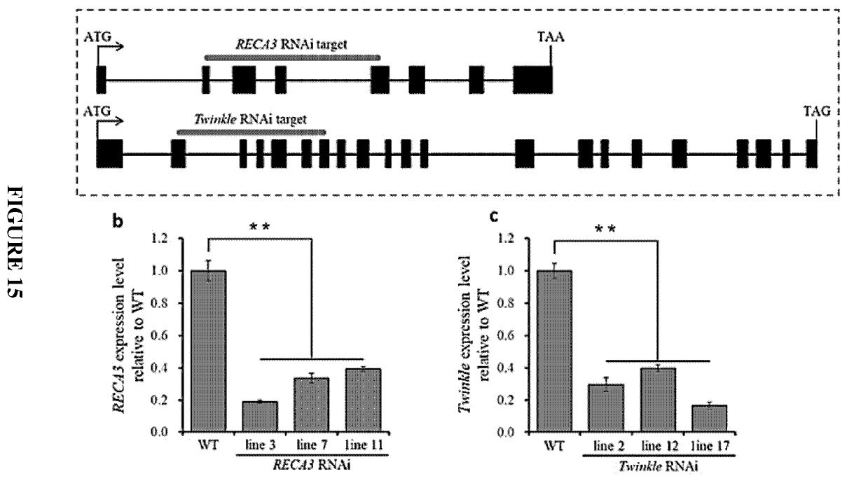

In view of the observations showing that TA1 protein associated with RECA3 and TWINKLE in mitochondria, the inventors tested whether reduction of expression of the genes encoding RECA3 and TWINKLE in rice would also result in thicker aleurone in the grain. This was tested using RNA interference (RNAi) to reduce expression of the RecA3 and TWINKLE genes. To make the RNAi constructs, a region of each of the RecA3 and TWINKLE genes was selected and used to create an inverted repeat construct to express a hairpin RNA. These constructs were used for Agrobacterium-mediated transformation of wild-type rice using standard methods, and transformants selected. Plants were selected which had reduced expression of the genes in the grain as shown by qRT-PCR (FIG. 15). Three lines were chosen for each construct.

When caryopses from the selected lines were examined by microscopy for aleurone thickness by transverse section and staining with PAS, it was observed that all of the transgenic caryopses had thickened aleurone compared to the wild-type. Lines which were more reduced for RecA3 and TWINKLE genes were more affected, with thicker aleurones, ranging from 2-8 layers in at least some zones of the aleurone. It was also observed that the starchy endosperms of the transgenic grain appeared to be chalky, or opaque.

On the basis of these experiments, the inventors developed a model describing the regulation of energy homeostasis in aleurone cell layer mediated by TA1-RECA3/TWINKLE complex, which suppresses illegitimate recombination of mtDNA during grain development, thus determining the differentiation of sub-aleurone cell fate where the cells that would form the outmost cells of starchy endosperm in wild-type grain instead retained identity as aleurone cells, resulting in the thickened aleurones.

### Example 16. Homologues of the Rice TA1 Gene in Other Cereals

Single-stranded DNA binding (SSB) proteins play an important role in DNA replication, homologous recombination and DNA repair (Moriyama and Sato, 2014). In bacteria, the RecA protein catalyzes homologous strand exchange, and SSB proteins enhance this reaction (McEntee et al. 1980; Steffen and Bryant 2001). A plant gene that encodes mitochondrially targeted single-stranded DNA binding protein (mtSSB) has been characterized in Arabidopsis thaliana and it was the earliest direct evidence that mitochondrially targeted homologues of RecA, SSB, or other proteins involved in mitochondrial DNA (mtDNA) recombination exist in plants (Edmondson et al., 2005). Using SEQ ID NO:3 as a query, the A. thaliana SSB genes At4g11060 and At3g18580, hereinafter termed AtmtSSB-1 and AtmtSSB-2 respectively, were identified by a BLAST search of the Arabidopsis genome in The Arabidopsis Information Resource (TAIR) database (http://www.arabidopsis.org). The AtmtSSB-1 polypeptide was 66% identical in amino acid sequence to the full length of SEQ ID NO:3, whereas the AtmtSSB-2 polypeptide was much less homologous, at 26% identity (Table 5). A. thaliana therefore has two mtSSB genes. At4g11060 codes for a protein of 201 amino acids (SEQ ID NO:15), including a 28-residue putative mitochondrial targeting peptide (MTP). At3g18580 codes for a protein of 217 amino acids (SEQ ID NO:31), including a 23-residue putative MTP. The model tree species Populus trichocarpa (poplar) also had two mtSSB sequences, namely PtmtSSB-1 (SEQ ID NO:21) and PtmtSSB-2 (SEQ ID NO:39).

The rice (Oryza sativa, Japonica Group) genome was observed to have two other, related genes to Os05g0509700 (encoding OsTA1=OsmtSSB-1a), namely Os04g0363700 and Os01g0642900, hereinafter termed OsmtSSB-1b and OsmtSSB-2 respectively, which were identified by a BLAST search of the rice genome and amino acid sequences in the Rice Genome Annotation Project (RGAP) database (http://rice.plantbiology.msu.edu/). OsmtSSB-1b was closer in sequence to OstmtSSB-1a at 72% identity to the full length of SEQ ID NO:3, whereas OstmtSSB-2 was less homologous at 32% identity. Ostmt-SSB-1b is also designated herein as OsTA1-like or OsTA1L. Rice therefore has three mtSSB genes. Like the AtmtSSB proteins, all three of the OsmtSSB polypeptides had a SSB domain, which for OsmtSSB-1b included Motifs I-IV present in all of the examined plant TA1 polypeptide sequences.

Table 5 provides the amino acid sequence identities of the rice TA1 (OsmtSSB-1a; SEQ ID NO:3) polypeptide to a range of homologous plant polypeptides, and for OsTA1L. FIG. 7 shows the alignment of some of these plant sequences. The two dicotyledonous species examined each had only two mtSSB sequences, as did some of the monocotyledonous species, whereas other monocotyledonous species had three mtSSB sequences including one closest to OsTA1 (at least 80% identical) and one closer in sequence to OsTA1L. Overall, the plant mtSSB-1 sequences had at least 60% identity to SEQ ID NO:3, whereas the mtSSB-2 sequences had no more than 32% identity to SEQ ID NO:3.

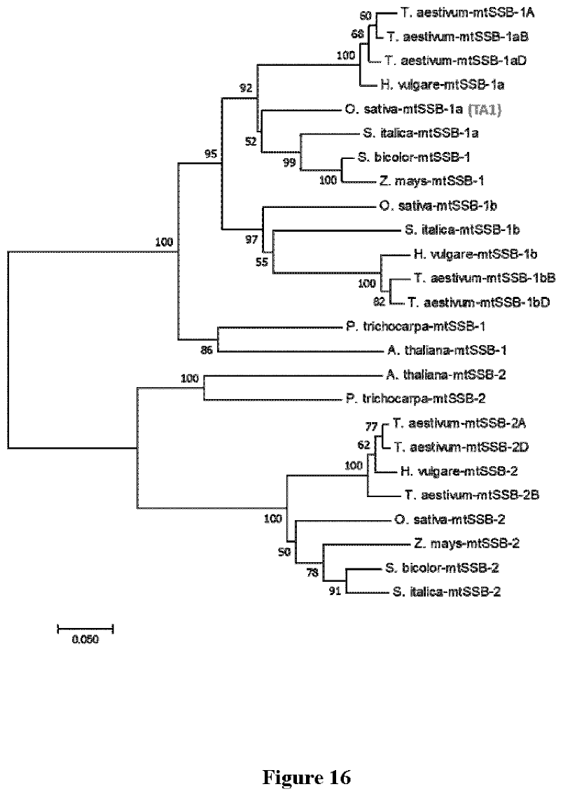

FIG. 16 provides a phylogenetic tree showing the relatedness of homologs of TA1 (OsmtSSB-1a) polypeptide from other plant species, mainly cereal species. In each case the most homologous amino acid sequence was at least 60% identical to SEQ ID NO:3 along the full length of SEQ ID NO:3. The amino acid sequences used in the analysis are provided as SEQ ID NO:15-23 for mtSSB-1 or mtSSB-1a sequences most related to OsTA1, SEQ ID NOs:24-29 for mtSSB-1b sequences which were more closely related to OsTA1L than OsTA1, and SEQ ID NOs:30-39 for mtSSB-2 sequences.

The inventors concluded that plant species have 2 or 3 mtSSB sequences, all of which would be expected to be functional, providing some degree of functional redundancy. This analysis did not address the tissue specificity of the different mtSSB genes.

TABLE 5
Amino acid sequence identity of plant homologs to rice TA1
(OsmtSSB-1a) and TA1-like (OsmtSSB-1b).

| | | | Identity to | Identity to |
|---|---|---|---|---|
| | | SEQ | OsmtSSB- | OsmtSSB- |
| | | ID | 1a | 1b |
| Protein | Accession Number | NO | (%) | (%) |
| OsmtSSB-1a | Os05t0509700-01 | 3 | 100 | 72 |
| OsmtSSB-1b | Os04t0363700-01 | 24 | 72 | 100 |
| OsmtSSB-2 | Os01t0642900-01 | 30 | 32 | |
| AtmtSSB-1 | AT4G11060.1 | 15 | 66 | 58 |
| AtmtSSB-2 | AT3G18580.1 | 31 | 26 | |
| SbmtSSB-1 | KXG22311 | 17 | 84 | 70 |
| SbmtSSB-2 | EES03342 | 32 | 32 | |
| ZmmtSSB-1 | Zm00001d000241 P001 | 16 | 79 | 70 |
| ZmmtSSB-2 | Zm00001d044086 P001 | 33 | 30 | |
| SimtSSB-1a | KQL14798 | 23 | 84 | 66 |
| SimtSSB-1b | KQL07192 | 25 | 67 | 72 |
| SimtSSB-2 | KQL06067 | 34 | 32 | |
| HvmtSSB-1a | BAK06246.1 | 18 | 82 | 66 |
| HvmtSSB-1b | HORVU 3Hr1G075580.1 | 26 | 63 | 74 |
| HvmtSSB-2 | HORVU3Hr1G057550.1 | 35 | 29 | |
| TamtSSB-1;A | TraesCS3A02G322900.1 | 27 | 63 | 73 |
| TamtSSB-2;A | TraesCS3A02G231400.1 | 36 | 25 | |
| TamtSSB-1a;B | TraesCS1B02G341000.1 | 22 | 82 | 64 |
| TamtSSB-1b;B | TraesCS3B02G342800.1 | 28 | 64 | 74 |
| TamtSSB-2;B | TraesCS3B02G260600.1 | 37 | 31 | |
| TamtSSB-1a;D | TraesCS1D02G329700.2 | 19 | 81 | 65 |
| TamtSSB-1b;D | TraesCS3D02G308600.3 | 29 | 64 | 74 |
| TamtSSB-2;D | TraesCS3D02G222000.1 | 38 | 30 | |

### Example 17. Homologues of the Rice RECA3 and TWINKLE Genes in Other Plant Species

Using SEQ ID NO:61 (rice RECA3 polypeptide) as a query, homologous amino acid sequences from various cereals and related monocotyledonous plants and several dicotyledonous plants were identified by a BLAST search of sequence databases. Homologous sequences were readily identified due the high degree of sequence conservation of the RECA3 polypeptides in different plant species. The polypeptides had a length, including the N-terminal signal sequences for mitochondrial or plastid translocation, between 425-435 amino acid residues (Table 6). Corresponding cDNA sequences for the genes encoding these RECA3 proteins were also identified (Table 6). The cereal and grass RECA3 polypeptides were at least 80% identical in amino acid sequence to the full length of SEQ ID NO:61, whereas the RECA3 polypeptides from dicot plants were less homologous, at between 67-72% identical to SEQ ID NO:61. Each of the amino acid sequences were presumed to include a mitochondrial targeting peptide (MTP) at their N-terminus which was predicted to be cleaved off as the polypeptides were translocated to the mitochondria.

The inventors concluded that plant species have a wild-type RECA3 sequence which would be functional in mitochondria, and which could be modified by mutagenesis or down-regulation by silencing RNAs.

Using SEQ ID NO:64 (rice TWINKLE polypeptide) as a query, homologous amino acid sequences from various monocotyledonous plants and dicotyledonous plants were also identified by a BLAST search of the sequence databases. Homologous sequences were readily identified, again due the high degree of sequence conservation of the TWINKLE polypeptides in different plant species. The polypeptides had a length, including the N-terminal signal sequences for mitochondrial or plastid translocation, between 695-761 amino acid residues (Table 7). Corresponding cDNA sequences for the genes encoding these RECA3 proteins were also identified (Table 7). For many of these plant species, multiple cDNA sequences were identified, probably indicating alternative splicing of transcripts from the genes. The cereal and grass RECA3 polypeptides were at least 77% identical in amino acid sequence to the full length of SEQ ID NO:64, whereas the RECA3 polypeptides from dicot plants were less homologous, at between 67-75% identical to SEQ ID NO:64. Each of the amino acid sequences were presumed to include a mitochondrial targeting peptide (MTP) at their N-terminus which was predicted to be cleaved off as the polypeptides were translocated to the mitochondria.

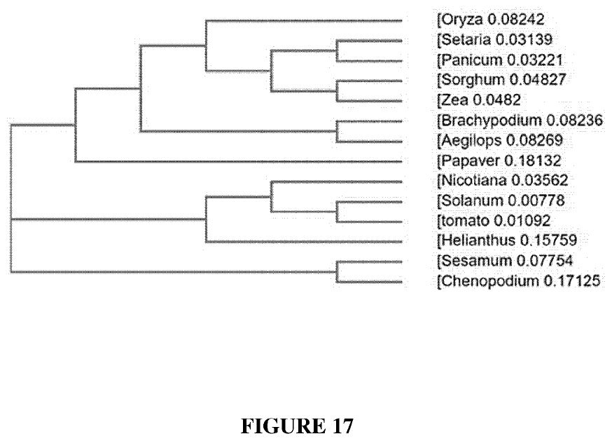

FIG. 17 provides a phylogenetic tree showing the relatedness of homologs of rice TWINKLE polypeptide from other plant species, including cereal species. In each case the most homologous amino acid sequence was at least 60% identical to SEQ ID NO:64 along the full length of SEQ ID NO:64. The inventors concluded that plant species have a wild-type TWINKLE sequence which would be functional in mitochondria.

TABLE 6
RECA3 homologous proteins identified in a range of plant species.

| | Accession No | | | | Length | Identity to |
|---|---|---|---|---|---|---|
| | of cDNA for | SEQ | Accession No. | SEQ | (amino acid | rice RECA3 |
| Plant species | homologous gene | ID NO | of protein | ID NO | residues) | (%) |
| Oryza sativa | XM_015756709.2 | 63 | XP_015612195.1 | 61 | 429 | 100 |
| Brachypodium | XM_003564806 | 68 | XP_003564854.1 | 67 | 429 | 87 |
| distachyon | | | | | | |
| Digitaria | — | — | CAB3479142.1 | — | 427 | 85 |
| exilis | | | | | | |
| Sorghum | XM_002458886.2 | 70 | XP_002458931.1 | 69 | 427 | 85 |
| bicolor | | | | | | |
| Zea mays | NM_001176740.1 | 72 | NP_001170211.1 | 71 | 425 | 82 |
| Hordeum | AK366136.1 | 74 | BAJ97339.1 | 73 | 429 | 84 |
| vulgare | | | | | | |
| Triticum | — | — | VAH82650.1 | 79 | 435 | 83 |
| turgidum | | | | | | |
| subsp. durum | | | | | | |
| Triticum | AK335548.1 | 76 | CDM84906.1 | 75 | 435 | 83 |
| aestivum | | | | | | |
| Aegilops | XM_020339638.1 | 78 | XP_020195227.1 | 77 | 433 | 83 |
| tauschii | | | | | | |
| Panicum hallii | XM_025963278.1 | — | XP_025819063. | — | 426 | 84 |
| Lupinus | XM_019607017.1 | — | XP_019462562.1 | — | 419 | 72 |
| angustifolius | | | | | | |
| Trifolium | DF973566.1 | — | GAU34790.1 | — | 411 | 73 |
| subterraneum | | | | | | |
| Nicotiana | XM_016629478.1 | — | XP_016484964.1 | — | 423 | 71 |
| tabacum | | | | | | |
| Glycine max | XM_003549185.4 | — | XP_003549233.1 | — | 459 | 68 |
| Solanum | XM_015308059.1 | — | XP_015163545.1 | — | 424 | 67 |
| tuberosum | | | | | | |
| Solanum | XM_004240132.4 | — | XP_004240180.1 | — | 418 | 69 |
| lycopersicum | | | | | | |

TABLE 7
TWINKLE homologs identified in a range of plant species.

| | Accession No | | | | Length | Identity to |
|---|---|---|---|---|---|---|
| | of cDNA for | SEQ | Accession No. | SEQ | (amino acid | rice TWINKLE |
| Plant species | homologous gene | ID NO | of protein | ID NO | residues) | (%) |
| Oryza sativa | XM_015785659.2 | 66 | XP_015641145.1 | 64 | 723 | 100 |
| Brachypodium | XM_010242321.3 | 81 | XP_010240623.1 | 80 | 723 | 79 |
| distachyon | | | | | | |
| Sorghum | XM_021450218.1 | 83 | XP_021305893.1 | 82 | 761 | 80 |
| bicolor | | | | | | |
| Zea mays | XM_008650737.2 | 85 | XP_008648959.2 | 84 | 736 | 80 |
| Aegilops | XM_020311186.1 | 87 | — | 86 | 726 | 79 |
| tauschii | | | | | | |
| Panicum hallii | XM_025957680.1 | 89 | XP_025813465.1 | 88 | 748 | 84 |
| Setaria viridis | XM_034736031.1 | 91 | XP_034591922.1 | 90 | 759 | 84 |
| Nicotiana | XM_016653437.1 | 93 | XP_016508923.1 | 92 | 699 | 71 |
| tabacum | | | | | | |
| Solanum | XM_006360255.2 | 95 | XP_006360317.1 | 94 | 695 | 75 |
| tuberosum | | | | | | |
| Solanum | XM_004231508.4 | 97 | XP_004231556.1 | 96 | 697 | 69 |
| lycopersicum | | | | | | |

### Example 18. Breeding of the Ta1 Mutation into Black Rice

“Black rice” is a term used to describe rice grain which is pigmented in its outer layer—the bran and the hull—with anthocyanins. These pigments are actually deep purple in colour, not black, but the high concentration of the pigments makes the grain look black or dark grey. The pigments are powerful antioxidants that are thought to have a variety of health benefits (Yao et al., 2013).

In order to introduce a ta1 mutant allele into a different genetic background, a black rice variety Zixiangnuo 1306 was first of all chosen as the recurrent parent. This variety is a Japonica rice and is wild-type for the TA1 gene. It is suitable for growing in more northern regions of China, having good cold tolerance, tolerance to saline soils and disease resistance. At maturity, the number of leaves of Zixiangnuo 1306 is about 15, the plant height is in the ideal range at 70-85 cm, the leaf color is dark green, and the seedlings and plants are fragrant. The total number of grains per mu is about 100, and the seed setting rate is at least 85%. The color of the palea is green at the beginning of panicle, and it is light purple when the grains reach the filled stage and silvery gray at maturity. The weight of 1000 mature grain is typically about 23 g. Generally, the yield per mu is good at 300-400 kg, under ideal growth conditions reaching more than 450 kg/mu. The amylose content of the starch of the grain is about 5-6% and the cooked grain is considered soft in texture when eaten.

To introduce the ta1-1 allele, a cross was made between a Zhonghua 11 plant carrying the ta1-1 allele as female parent and a plant of Zixiangnuo 1306 (TA1) as male parent. F1 progeny plants from the cross were then backcrossed for two generations to plants of Zixiangnuo 1306 as the recurrent parent to establish a population of BC2F1 backcross progeny plants. Several of these plants were selfed to produce BC2F2 plants and, from those, BC2F3 progeny were identified and selected that were homozygous for the ta1-1 allele. In this breeding program, the TA1 and ta1-1 alleles were detected by enzyme digestion of PCR products (years 2014-17) or by high-resolution DNA melting curve (HRM, 2018 to present) as described in Example 1. Aleurone layer thickening was monitored by observing the aleurone layer in cross-sections of the rice grain. Four genetically stable lines were obtained that were homozygous for ta1-1, named Zhongzi1, Zhongzi2, Zhongzi3 and Zhongzi4. These lines had inherited most of the favourable characteristics of Zixiangnuo 1306 mentioned above such as rice blast resistance as well as the introduced ta1-1 allele conferring the thickened aleurone.

The new varieties were grown in the field and a range of parameters were measured and compared to wild-type. The data are shown in Table 8. The wild-type variety (WT) in those trials was Zixiangnuo 1306.

The nutritional composition of the Zhongzi ta1 mutant grain was determined for a range of nutrients and compared to wild-type Zhonghua11 (ZH11) having the TA1 gene with normal aleurone, or ta1-1 in the ZH11 genetic background having thickened aleurone, wild-type Zixiangnuo 1306 (black rice, TA1) and Zhongzi (black rice, ta1). It was observed that the ta1 black rice had significantly higher amounts of all of the nutrients that were measured, including protein, dietary fibre, minerals and B vitamins. The increases in iron, zinc and vitamin B6 were particularly impressive and surprising to the inventors.

The successful breeding to produce these Zhongzi high nutrition rice varieties with a ta1 mutation in a black rice variety therefore provided new rice varieties that can be used for more nutritious rice foods and beverages.

TABLE 8
Field trial data for Zhongzi varieties containing ta1-1 allele

| | | Mean | SD | T. test (P. Value) |
|---|---|---|---|---|
| Plant height (cm) | WT | 71.3 | 0.40 | |
| | Zhongzi 1 | 99.3 | 0.76 | 0.000224 |
| | Zhongzi 2 | 79.9 | 0.13 | 0.000305 |
| | Zhongzi 4 | 82.4 | 0.50 | 0.001049 |
| Tiller number | WT | 10.8 | 0.06 | |
| | Zhongzi 1 | 9.9 | 0.26 | 0.011694 |
| | Zhongzi 2 | 10.9 | 0.44 | 0.414874 |
| | Zhongzi 4 | 12.2 | 0.26 | 0.008126 |
| Length of seed setting (cm) | WT | 18.5 | 0.19 | |
| | Zhongzi 1 | 24.4 | 0.34 | 0.001319 |
| | Zhongzi 2 | 18.9 | 0.54 | 0.189372 |
| | Zhongzi 4 | 19.5 | 0.28 | 0.002693 |
| Number of grains of per | WT | 176.3 | 0.86 | |
| seed setting | Zhongzi 1 | 171.5 | 4.00 | 0.060319 |
| | Zhongzi 2 | 185.3 | 2.91 | 0.026657 |
| | Zhongzi 4 | 241.1 | 4.70 | 0.001026 |
| Grains of per seed setting | WT | 89.3 | 0.55 | |
| (%) | Zhongzi 1 | 94.1 | 0.21 | 0.000852 |
| | Zhongzi 2 | 91.9 | 0.11 | 0.007130 |
| | Zhongzi 4 | 93.1 | 0.23 | 0.005084 |
| Weight of 1000 grains (g) | WT | 19.6 | 0.20 | |
| | Zhongzi 1 | 23.6 | 0.09 | 0.000577 |
| | Zhongzi 2 | 20.9 | 0.14 | 0.007057 |
| | Zhongzi 4 | 19.2 | 0.04 | 0.054976 |
| Average length of grains | WT | 6.3 | 0.05 | |
| (mm) | Zhongzi 1 | 7.1 | 0.09 | 4.770523E−20 |
| | Zhongzi 2 | 6.2 | 0.08 | 0.001368 |
| | Zhongzi 4 | 6.4 | 0.04 | 0.000037 |
| Average width of grains | WT | 2.5 | 0.02 | |
| (mm) | Zhongzi 1 | 2.6 | 0.04 | 1.628774E−09 |
| | Zhongzi 2 | 2.6 | 0.05 | 2.256365E−08 |
| | Zhongzi 4 | 2.4 | 0.03 | 0.000001 |
| Average thickness of grains | WT | 1.8 | 0.05 | |
| (mm) | Zhongzi 1 | 1.9 | 0.10 | 0.002619 |
| | Zhongzi 2 | 2.0 | 0.07 | 0.000012 |
| | Zhongzi 4 | 1.9 | 0.07 | 0.044645 |

The nutritional composition of the Zhongzi ta1 mutant grain was determined for a range of nutrients and compared to wild-type Zhonghua11 (ZH11) having the TA1 gene with normal aleurone, or ta1-1 in the ZH1 genetic background having thickened aleurone, wild-type Zixiangnuo 1306 (black rice, TA1) and Zhongzi (black rice, ha). It was observed that the ta1 black rice had significantly higher amounts of all of the nutrients that were measured, including protein, dietary fibre, minerals and B vitamins (Table 9). The increases in iron, zinc and vitamin B6 were particularly impressive and surprising to the inventors.

The successful breeding to produce these Zhongzi high nutrition rice varieties with a ta1 mutation in a black rice variety therefore provided new rice varieties that can be used for more nutritious rice foods and beverages.

TABLE 9
Nutrient composition of ta1 black rice grain.

| Nutrient | ZH11 | ta1 | TA1 black rice | ta1 black rice |
|---|---|---|---|---|
| total protein | 7.63 ± 0.09 | 8.48 ± 0.10 | 9.48 ± 0.08 | 10.03 ± 0.10 |
| (%) | | | | |
| total lipids | 2.84 ± 0.03 | 3.00 ± 0.04 | 2.75 ± 0.09 | 3.42 ± 0.08 |
| (%) | | | | |
| iron (mg/kg) | 11.69 ± 0.36 | 12.45 ± 0.35 | 15.11 ± 0.31 | 18.16 ± 0.38 |
| zinc (mg/kg) | 16.02 ± 0.55 | 23.42 ± 0.52 | 23.18 ± 0.15 | 26.14 ± 0.42 |
| dietary fiber | 3.20 ± 0.06 | 4.25 ± 0.07 | 5.03 ± 0.12 | 6.23 ± 0.21 |
| (g/100g) | | | | |
| Vitamin B2 | 0.044 ± 0.002 | 0.054 ± 0.003 | 0.065 ± 0.003 | 0.072 ± 0.003 |
| (mg/100g ) | | | | |
| Vitamin B6 | 0.090 ± 0.003 | 0.102 ± 0.004 | 0.377 ± 0.009 | 0.822 ± 0.012 |
| (mg/100 g) | | | | |

### Example 19. Production of Food and Beverages from Ta1 Mutant Rice

The rice grain having thickened aleurone was considered useful for production of food ingredients, beverage ingredients, and foods and beverages having increased nutrient amounts relative to the corresponding ingredients, food or beverage made with an equivalent amount of wild-type rice. Therefore, a variety of foods and beverages were prepared and tested for quality, aroma and taste by human volunteers. The rice grain used for this were varieties Zhongzi1, Zhongzi2, Zhongzi3 and Zhongzi4 (Example 18). The rice grain after harvest had a moisture content of 11.65% and a density of 760 g/L.

### Roasted Rice Grain and Black Rice Tea

Rice tea was prepared by soaking whole grain, roasting (baking) it for a period of time, and then soaking the grain in boiling water (steeping or brewing). In one experiment to test roasting temperatures and times, samples of 50 g of grain were soaked in water for 14 h, drained, and the rice grain roasted under different conditions (Table 10). The results showed that when the roasting temperature was kept at about 210° C. for more than 6 min, the aroma of the roasted grain was strong and pleasant. The aroma was considered by the human volunteers to be reminiscent of fermented sauce. When the roasting temperature was above 180° C. and the time was more than 3.5 min, the granule aroma was more obvious than at lower temperatures or for less time. Volunteers described the aroma as having a distinctive “special burnt” aroma with a strong sense of fried rice, according to particle integrity and aroma.

TABLE 10
Rice roasting (baking) experiment.

| Samples | Baking temperature | Time | |
|---|---|---|---|
| T1 | 195° C. | 5.5 | min |
| T2 | 195° C. | 9 | min |
| T3 | 210° C. | 6.5 | min |
| T4 | 210° C. | 7 | min |
| T5 | 220° C. | 5 | min |
| T6 | 230° C. | 4.5 | min |

To make black rice tea, the black rice grain was soaked in boiling water for 10 min and then drained. 50 g was weighed each time for the roasting test under different conditions. The aroma of the roasted black rice grains was described as soft and without a burnt aroma. According to the evaluation of particle integrity and aroma, the rice grain of sample T2 was the best.

Grain samples of Zhongzi1, Zhongzi2, Zhongzi3 and Zhongzi4 were roasted at 180° C. for 3.5 min. For brewing the tea, samples of 6 g of the roasted rice grain were added to 150 mL boiling water for 10 min. The colour and taste of the grain before and after brewing, and of the resultant beverages, were evaluated by a sensory panel of 7 volunteers. Each of the Zhongzi1, Zhongzi2 and Zhongzi3 grain samples provided strong aroma and flavor before brewing, and the best brown colour after brewing. The taste and particle integrity of the Zhongzi1 grain was similar to Zhongzi2. The aroma of Zhongzi4 was less attractive before and after brewing, described by some volunteers as “poor”, and the colour of the resultant tea was the lightest after brewing.

### Rice Porridge

Rice porridge was prepared by soaking 100 g of black rice grain in water for at least 1 h, up to 16 h. Soaking for 16 h was recommended compared to 1 h. The rice grain was rinsed twice with water and then cooked in 1 L of water for 90 min in an electric cooker. Volunteers who tasted the porridge described it as neither too hard nor too soft, with a pleasant taste and a “special aroma”, and suitable for children and adults, for example adults suffering from intestinal upset.

To make a black rice paste suitable as a food for infants, 500 g of rice grain was roasted at 90° C. for 60 min. The roasted grain was ground and then sieved (100 mesh) to produce a fine milled flour. Samples of 20 g of the black rice flour were soaked in 100 mL of boiling water, with mixing until a black rice paste was produced. Volunteers who tasted the rice paste considered it a convenient fast food for eating as well as suitable for infants. It was a little sweeter than they had expected.

### Cooked Rice Grain

50 g of black rice grain was mixed with 200 g of white rice, rinsed twice with water twice, and cooked for 60 min with 300-350 mL of water in an electric cooker. The black rice grains cooked as evenly as the white rice. The black rice remained deeply coloured after cooking, with a soft texture and a taste or aroma described as “the special aroma”.

### Rice Bars

500 g of black rice grain is roasted at 90° C. for at least than 60 min, ground and sieved (100 mesh), and shaped into bars using a chocolate bar making machine. This produces bars as a food suitable for consumption, having a unique taste.

### Black Rice Oil

Black rice grain is milled and the bran, including the pericarp and aleurone layer, produced from the milling is separated from the polished grain. Oil is produced from the bran by cold pressing, producing rice bran oil. The oil contains relatively high levels of antioxidants and oryzanols, relative to rice bran oil from wild-type rice.

The present application claims priority from AU 2020904452 filed 1 Dec. 2020, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Akazawa and Haranishimura (1985) Ann. Rev. Plant Physiol. Mol. Biol. 36:441-472.
Anzalone et al. (2019) Nature 576:149-157.
Beck and Ziegler (1989) Ann. Rev. Plant Physiol. Mol. Biol. 40:95-117.
Becraft et al. (2001a) In Bhojwani and Soh (eds). Current Trends in the Embryology of
Angiosperms, Kluwer Academic Publishers, pp353-374.
Becraft et al. (2001b) Plant Physiol. 127:4039-4048.
Becraft et al. (2002) Development 129:5217-5225.
Becraft and Yi (2011) J. Exp. Botany 62:1669-1675.
Brewer et al. (2006) Nature Protocols. 1:1462-1467.
Buttrose et al. (1963) Aust. J. Biol. Sci. 16:768-774.
Calhoun (1960) Cereal Chemistry. 37:755.
Ciesielski et al. (2016) Methods Mol. Biol. 1351:211-222.
Comai et al. (2004) Plant J 37: 778-786.
Diray-Arce et al. (2013) BMC Plant Biol. 13:36. doi:10.1186/1471-2229-13-36.
Durai et al. (2005) Nucleic Acids Research 33:5978-5990.
Edmondson et al. (2005) Molecular Genetics and Genomics 273, 115-122.
Farr et al. (2004) J. Biol. Chem. 279:17047-17053.
Flynn and Zou (2010) Crit. Rev. Biochem. Mol. Biol. 45 266-275.
Gualberto and Newton (2017) Annu. Rev. Plant Biol. 68:225-52.
Hayashi et al. (2004) J Eukaryot Microbiol. 51:321-324.
Henikoff et al. (2004) Plant Phys 135:630-636.
Hoshikawa (1993) in Matsuo and Hoshikawa (eds), Science of the Rice Plant:
Morphology. Nobunkyo, Tokyo, pp 339-376.
Hu et al. (2018) Nature 556:57-63.
Huang (2002a) Journal of Agricultural and Food Chemistry. 50:1815-1821.
Huang (2002b) Journal of Agricultural and Food Chemistry. 50:4437-4444.
Huang (2005) Journal of Agricultural and Food Chemistry. 53:1841-1856.
Jayathilaka et al. (2008) Proc. Natl. Acad. Sci. USA 105:15848-15853.
Jones (1969) Planta 85:359-375.
Kawakatsu et al. (2009) The Plant J. 59:908-920.
Kazama et al. (2008) Plant Biotechnology 25: 113-117.
Kessler et al. (2002) Development 129:1859-1869.
Kim et al. (2016) Rice 9:12.
Korhonen et al. (2003) J. Biol. Chem. 278:48627-48632.
Lahey et al. (1999) Food Chemistry 65:129-133.
Le Provost et al. (2009) Trends in Biotechnology 28:134-141.
Lewis et al. (2009) Plant and Cell Physiol. 50:554-571.
Li et al. (2008) J. Agric. Food Chem. 56:9732-9739.
Lid et al. (2004) Planta 218:370-378.
Liu et al. (2010) Biotechnology and Bioengineering 106:97-105.
Mann et al. (2001) J. AOAC Int. 84:1593.
McCleary et al. (1997) J AOAC Int. 80:571-580.
McEntee et al. (1980) Proc. Natl. Acad. Sci. USA 77:857-861.
Moriyama and Sato (2014) Frontiers in Plant Science Vol 5, article 480.
Murashige and Skoog (1962) Physiologia Plantarum 15:473-497.
Nishimura et al. (2006) Nature Protocols. 1:2796-2802.
Osakabe et al. (2020) Communications Biol 3:648.
Prior (2005) Journal of Agricultural and Food Chemistry. 53:4290-4302.
Prosky et al. (1985) AOAC Official method 991.
Reddy et al. (2001) J. Biol. Chem. 276:45959-45968.
Schlemmer (2009) Molecular Nutrition and Food Research. 55(Supplement 2):5330-S375.
Shedge et al. (2007) Plant Cell 19:1251-64.
Shen et al. (2003) Proc. Natl. Acad. Sci. USA 100:6552-6557.
Silva et al. (2017) DNA Repair 60:64-76.
Slade and Knauf (2005) Transgenic Res. 14:109-115.
Sreenivasulu (2010) The Plant Journal. 64:589-603.
Steffen and Bryant (2001) Archives of Biochemistry and Biophysics 388:165-170.
Theander et al. (1995) J AOAC Int. 78:1030-1044.
Toseland et al. (2010) Methods 51:259-268.
Wolbang (2010) J. Agric Food Chem. 58, 1732-1740.
Yao et al. (2013) Food & Function. 4: 1602-1608.
Yi et al. (2011) Plant Physiol. 156:1826-1836.
Zarcinas (1983a) CSIRO Division of Soils Technical Paper. 1-36.
Zarcinas (1983b) Communications in Soil Science and Plant Analysis. 18:131-146.
Zwar and Chandler (1995) Planta 197: 39-48.

1. Cereal grain comprising an aleurone, an embryo, a starchy endosperm and a reduced level and/or activity of at least one mitochondrial polypeptide relative to a corresponding wild-type cereal grain, wherein the mitochondrial polypeptide which is reduced in its level and/or activity is at least one of a mitochondrial single-stranded DNA binding (mtSSB) polypeptide, a RECA3 polypeptide and a TWINKLE polypeptide.

2. The grain of claim 1 which comprises a genetic variation which reduces the level and/or activity of the at least one mitochondrial polypeptide in the grain relative to the corresponding wild-type cereal grain.

3. The grain of claim 2, wherein the genetic variation comprises
(i) a mutation in an endogenous gene which encodes the mitochondrial polypeptide, whereby the mutation results in the reduced level and/or activity of the mitochondrial polypeptide, or
(ii) an exogenous polynucleotide which encodes a silencing RNA molecule, wherein the silencing RNA molecule, and/or a processed RNA molecule produced from the silencing RNA molecule, reduces the expression of the endogenous gene, preferably wherein the exogenous polynucleotide comprises a DNA region encoding the silencing RNA molecule operably linked to a promoter which is expressed in developing grain of a cereal plant, wherein the promoter is preferably expressed at least at a time point between the time of pollination and 30 days post-pollination.

4. The grain of claim 2 or claim 3, wherein the genetic variation comprises:
(a) a splice site mutation which results in modified, preferably reduced, splicing of an RNA transcript of an endogenous gene which encodes the mitochondrial polypeptide, wherein the splice site mutation is preferably a single nucleotide substitution at a splice site, more preferably an adenine nucleotide at a position corresponding to nucleotide number 2126 of SEQ ID NO:4,
(b) a deletion or an insertion within the endogenous gene which encodes the mitochondrial polypeptide, preferably a deletion which was introduced by mutagenesis of a progenitor cereal plant cell,
(c) a premature translational stop codon in the protein coding region of an endogenous gene which encodes the mitochondrial polypeptide,
(d) a mutation in an endogenous gene which encodes the mitochondrial polypeptide such that it encodes a polypeptide with reduced activity relative to the wild-type polypeptide, preferably wherein the mutation is a nucleotide substitution in the endogenous gene whereby the endogenous gene comprising the mutation encodes a polypeptide with an amino acid substitution relative to SEQ ID NO:3, or
(e) a mutation in an endogenous gene which encodes the mitochondrial polypeptide such that when the gene with the mutation is expressed it produces a reduced level of the polypeptide relative to the corresponding wild-type gene.

5. The grain according to any one of claims 2 to 4, wherein the genetic variation is an introduced genetic variation.

6. The grain according to any one or more of claims 2 to 5 which is homozygous for the genetic variation.

7. The grain according to any one of claims 1 to 6 which has a thickened aleurone.

8. The grain of claim 7 which has a thickened aleurone at day 20 after pollination.

9. The grain of claim 7 or claim 8, wherein the thickened aleurone comprises at least two, at least three, at least four or at least five layers of cells, about 3, about 4, about 5 or about 6 layers of cells, or 2-8, 2-7, 2-6, 2-5 or 3-5 layers of cells, or a number of cell layers between 2 and 8, 2 and 7, or 2 and 6.

10. The grain according to any one or more of claims 1 to 9 which is further characterised by one or more of:
(a) the grain comprises, when compared to a corresponding wild-type cereal grain, one or more or all of the following, each on a weight basis,
i) a higher fat content,
ii) a higher ash content,
iii) a higher fiber content,
iv) a lower starch content,
v) a higher mineral content, preferably the mineral content is the content of one or more or all of calcium, iron, zinc, potassium, magnesium, phosphorus and sulphur,
vi) a higher antioxidant content,
vii) a higher phytate content,
viii) a higher content of one or more or all of vitamins B3, B6 and B9,
ix) a higher sucrose content,
x) a higher neutral non-starch polysaccharide content, and
xi) a higher monosaccharide content;
(b) the grain comprises, when compared to a corresponding wild-type cereal grain, a higher degree of chalkiness,
(c) the grain comprises, when compared to a corresponding wild-type cereal grain, a higher number of aleurone cells,
(d) the grain is whole grain or cracked grain;
(e) the grain has a germination rate which is between about 40 and about 100% relative to the germination rate of a corresponding wild-type cereal grain;
(f) the grain has an increased α-amylase activity during germination compared to the corresponding wild-type cereal grain;
(g) the grain has looser packing of irregular-shaped starch granules when compared to a corresponding wild-type cereal grain; and
(h) the grain has one or more or all of about the same length, width, thickness, protein level and caryopsis morphology when compared to a corresponding wild-type cereal grain.

11. The grain according to any one or more of claims 1 to 10 which has been processed so that it is no longer able to germinate, preferably by heat treatment, more preferably which has been cooked.

12. The grain according to any one of claims 1 to 11, wherein the polypeptide is expressed in one or more or all of the developing endosperm, testa, aleurone, and embryo of a developing grain.

13. The grain according to any one of claims 1 to 12 which is pigmented in its outer layer(s).

14. The grain according to any one of claims 1 to 13 which is rice grain, wheat grain, barley grain, maize grain, sorghum grain or oat grain.

15. The grain according to any one of claims 1 to 14 which is rice grain.

16. The grain of claim 15 which is brown rice grain or black rice grain.

17. The grain according to any one of claims 1 to 16, wherein the wild-type grain comprises a mtSSB polypeptide which comprises an amino acid sequence as provided in any one of SEQ ID NOs: 3 or 15 to 39, or an amino acid sequence which is at least 75% identical to any one or more of SEQ ID NOs: 3 or 15 to 39.

18. The grain of claim 17, wherein the mtSSB polypeptide is a mtSSB-1a polypeptide.

19. The grain of claim 18, wherein the wild-type grain comprises mtSSB-1a polypeptide which comprises an amino acid sequence as provided in any one of SEQ ID NOs: 3 or 15 to 23, or an amino acid sequence which is at least 75% identical to any one or more of SEQ ID NOs: 3 or 15 to 23.

20. The grain of claim 18 or claim 19, wherein the wild-type grain comprises a mtSSB-1a polypeptide which comprises an amino acid sequence as provided in SEQ ID NO: 3, or an amino acid sequence which is at least 75% identical to SEQ ID NO: 3.

21. The grain according to any one of claims 1 to 20 which comprises an endogenous gene which encodes a truncated mtSSB polypeptide, preferably a truncated mtSSB-1a polypeptide.

22. The grain of claim 21, wherein the truncated mtSSB polypeptide consists of an amino acid sequence selected from SEQ ID NO:8, SEQ ID NO:9, SEQ ID NO:10, or a truncated version of any one thereof.

23. The grain according to any one of claims 1 to 22, wherein the reduced mtSSB activity is one or more or all of:
i) reduced ability to bind single stranded DNA,
ii) reduced ability to bind a RECA3 polypeptide, and
iii) reduced ability to bind a TWINKLE polypeptide.

24. The grain according to any one of claims 1 to 23, wherein the wild-type grain comprises a RECA3 polypeptide which comprises an amino acid sequence as provided in any one of SEQ ID NOs: 61, 67, 69, 71, 73, 75, 77 or 79, or an amino acid sequence which is at least 75% identical to any one or more of SEQ ID NOs: 61, 67, 69, 71, 73, 75, 77 or 79.

25. The grain according to any one of claims 1 to 24, wherein the reduced RECA3 polypeptide activity is one or both of:
i) reduced ability to bind a mtSSB polypeptide, and
ii) reduced recombinase activity.

26. The grain according to any one of claims 1 to 25, wherein the wild-type grain comprises a TWINKLE polypeptide which comprises an amino acid sequence as provided in any one of SEQ ID NOs: 64, 80, 82, 84, 86, 88, 90, 92, 94 or 96, or an amino acid sequence which is at least 75% identical to any one or more of SEQ ID NOs: 64, 80, 82, 84, 86, 88, 90, 92, 94 or 96.

27. The grain according to any one of claims 1 to 26, wherein the reduced TWINKLE polypeptide activity is one or both of:
i) reduced ability to bind a mtSSB polypeptide, and
ii) reduced helicase activity.

28. A mutant mitochondrial single-stranded DNA binding (mtSSB) polypeptide, a mutant RECA3 polypeptide or a mutant TWINKLE polypeptide whose amino acid sequence is different to the amino acid sequence of a corresponding wild-type mitochondrial single-stranded DNA binding (mtSSB) polypeptide, RECA3 polypeptide or TWINKLE polypeptide, respectively, and which has reduced activity when compared to the corresponding wild-type polypeptide, preferably which is encoded by an endogenous gene of a cereal plant comprising a genetic variation according to any one of claims 3 to 5.

29. A polynucleotide encoding the polypeptide of claim 28, preferably an endogenous gene of a cereal plant comprising a genetic variation according to any one of claims 3 to 5.

30. An isolated and/or exogenous polynucleotide which, when present in a grain of a cereal plant, reduces the expression of a gene encoding a mitochondrial single-stranded DNA binding (mtSSB) polypeptide, a RECA3 polypeptide or a TWINKLE polypeptide.

31. The polynucleotide of claim 30 when used for reducing the expression of the gene in developing grain of a cereal plant at least at a time point between the time of pollination and 30 days post-pollination.

32. A nucleic acid construct and/or vector encoding a polynucleotide according to any one of claims 29 to 31, wherein the nucleic acid construct or vector comprises a DNA region encoding the polynucleotide operably linked to a promoter which is expressed in developing grain of a cereal plant at least at a time point between the time of pollination and 30 days post-pollination.

33. A cell, tissue, organ, plant part or plant comprising a polynucleotide according to any one of claims 29 to 31, or a nucleic acid construct and/or vector of claim 32.

34. The plant cell, tissue, organ, plant part or plant of claim 33, wherein the polynucleotide, nucleic acid construct or vector is integrated into the genome of the cell, tissue, organ, plant part or plant, preferably into the nuclear genome.

35. A cereal plant cell, seed or tissue therefrom, the cell, seed or tissue therefrom comprising a reduced level and/or activity of at least one mitochondrial polypeptide relative to a corresponding wild-type cereal plant cell, wherein the mitochondrial polypeptide which is reduced in its level and/or activity is at least one of a mitochondrial single-stranded DNA binding (mtSSB) polypeptide, a RECA3 polypeptide and a TWINKLE polypeptide.

36. The cell, seed or tissue therefrom of claim 35 which is or comprises an endosperm, testa, aleurone or embryo cell.

37. The cell, seed or tissue therefrom of claim 35 which is an aleurone cell.

38. A cereal plant which produces grain according to any one of claims' to 10 or 12 to 27, and/or which comprises one or more or all of a polypeptide of claim 28, a polynucleotide according to any one of claims 29 to 31, a nucleic acid construct and/or vector of claim 32 or a cell, seed or tissue according to any one of claims 33 to 37.

39. The cereal plant of claim 38 which is characterised by one or more or all of:
(a) has about the same height as a corresponding wild-type plant;
(b) the plant is male and female fertile; and
(c) the plant exhibits delayed grain maturation.

40. A population of at least 100 cereal plants of claim 38 or claim 39 growing in a field.

41. A method of producing the cell according to any one of claims 33 to 37, the method comprising a step of introducing a genetic variation as defined in any one of claims 2 to 5, a polynucleotide according to any one of claims 29 to 31, or a nucleic acid construct and/or vector of claim 32, into a cell, preferably a cereal plant cell.

42. A method of producing a cereal plant of claim 38 or claim 39, or grain therefrom, the method comprising the steps of
i) introducing into a cereal plant cell, a genetic variation as defined in any one of claims 2 to 5, a polynucleotide according to any one of claims 29 to 31, or a nucleic acid construct and/or vector of claim 32,
ii) obtaining a cereal plant comprising the genetic variation or polynucleotide from a cell obtained from step i), and
iii) optionally harvesting grain from the plant of step ii), the grain comprising the genetic variation or being transgenic for the polynucleotide, nucleic acid construct or vector, and
iv) optionally producing one or more generations of progeny plants from the grain, the progeny plants comprising the genetic variation or being transgenic for the polynucleotide, nucleic acid construct or vector, thereby producing the cereal plant or grain.

43. A method of producing a cereal plant of claim 38 or claim 39, or grain therefrom, the method comprising the steps of
i) introducing into a cereal plant cell, a mutation to an endogenous gene which encodes a mitochondrial single-stranded DNA binding (mtSSB) polypeptide, a RECA3 polypeptide or a TWINKLE polypeptide, such that the cell has reduced level and/or activity of the mitochondrial single-stranded DNA binding (mtSSB) polypeptide, RECA3 polypeptide or TWINKLE polypeptide relative to a corresponding wild-type cereal plant cell,
ii) obtaining a cereal plant from a cell of step i), the cereal plant comprising the mutation of the endogenous gene, and
iii) optionally harvesting grain from the plant of step ii), the grain comprising the mutation, and
iv) optionally producing one or more generations of progeny plants from the grain, the progeny plants comprising the mutation,
thereby producing the cereal plant or grain.

44. A method of selecting a cereal plant of claim 38 or claim 39 or grain according to any one of claims 1 to 27, the method comprising the steps of
i) screening a population of cereal plants or grain each of which were obtained from a mutagenic treatment of progenitor cereal plant cells, grain or plants, for the production of grain according to any one of claims 1 to 10 or 12 to 27 or for the presence of a mutation in a gene encoding a mitochondrial single-stranded DNA binding (mtSSB) polypeptide, a RECA3 polypeptide or a TWINKLE polypeptide, and
ii) selecting from the population of step (i) a cereal plant which produces grain according to any one of claims 1 to 10 or 12 to 27 or which comprises a mutation in the gene,
thereby selecting the cereal plant or grain.

45. A method of selecting a cereal plant of claim 38 or claim 39, the method comprising the steps of
i) producing one or more progeny plants from cereal grain, the cereal grain having been derived from a cross of two parental cereal plants,
ii) screening the one or more progeny plants of step i) for the production of grain according to any one of claims 1 to 10 or 12 to 27, and
iii) selecting a progeny plant which produces the grain,
thereby selecting the cereal plant.

46. The method of claim 45, wherein step ii) comprises one or more or all of:
i) analysing a sample comprising DNA from a progeny plant for the genetic variation,
ii) analysing the thickness of aleurone of grain obtained from a progeny plant, and
iii) analysing the nutritional content of the grain or a part thereof.

47. The method of claim 45 or claim 46, wherein step iii) comprises one or more or all of:
i) selecting a progeny plant which is homozygous for the genetic variation,
ii) selecting a progeny plant whose grain has an increased aleurone thickness compared to a corresponding wild-type cereal grain,
iii) selecting a progeny plant whose grain or a part thereof has an altered nutritional content compared to a corresponding wild-type cereal grain or part thereof.

48. The method according to any one of claims 45 to 47 which further comprises
i) crossing two parental cereal plants, preferably wherein one of the parental cereal plants produces grain according to any one of claims 1 to 10 or 12 to 27, or
ii) backcrossing one or more progeny plants from step i) with plants of the same genotype as a first parental cereal plant which does not produce grain according to any one of claims 1 to 10 or 12 to 27 for a sufficient number of times to produce a plant with a majority of the genotype of the first parental cereal plant but which produces grain according to any one of claims 1 to 10 or 12 to 27, and
iii) selecting a progeny plant which produces grain according to any one of claims 1 to 10 or 12 to 27.

49. A cereal plant produced using the method according to any one of claims 42 to 48.

50. Use of a polynucleotide according to any one of claims 29 to 31, or a nucleic acid construct and/or vector of claim 32, to produce a cell, a cereal plant or plant part such as cereal grain.

51. The use of claim 50 when used to produce grain according to any one of claims 1 to 27.

52. A method for identifying a cereal plant which produces grain according to any one of claims 1 to 10 or 12 to 27, the method comprising the steps of
i) obtaining a nucleic acid sample from a cereal plant, and
ii) screening the sample for the presence or absence of a genetic variation which reduces the level of activity of a mitochondrial single-stranded DNA binding (mtSSB) polypeptide, a RECA3 polypeptide or a TWINKLE polypeptide in the plant when compared to a corresponding wild-type cereal plant.

53. The method of claim 52, wherein the genetic variation is one or both of
a) a nucleic acid construct expressing a polynucleotide, or the polynucleotide encoded thereby, which when present in a cereal plant reduces the expression of a gene encoding a mitochondrial single-stranded DNA binding (mtSSB) polypeptide, a RECA3 polypeptide or a TWINKLE polypeptide, and
b) a gene, or mRNA encoded thereby, which expresses a mutant, preferably truncated, mitochondrial single-stranded DNA binding (mtSSB) polypeptide, RECA3 polypeptide or TWINKLE polypeptide with reduced activity.

54. The method of claim 52 or claim 53, wherein the presence of the genetic variation indicates that grain of the cereal plant has a thickened aleurone when compared to a corresponding cereal plant lacking the genetic variation(s).

55. A method for identifying a cereal plant which produces grain according to any one of claims 1 to 10 or 12 to 27, the method comprising the steps of
i) obtaining grain from a cereal plant, and
ii) screening the grain or a portion thereof for one or more of
a) a thickened aleurone,
b) the amount of mitochondrial single-stranded DNA binding (mtSSB) polypeptide, RECA3 polypeptide or TWINKLE polypeptide and/or activity in the grain, and
c) the amount of mRNA encoded by a gene encoding a mitochondrial single-stranded DNA binding (mtSSB) polypeptide, a RECA3 polypeptide or a TWINKLE polypeptide in the grain.

56. The method of according to any one of claims 52 to 55 which identifies a cereal plant according to any one of claim 28, 38 or 39.

57. A method of producing a cereal plant part, the method comprising,
a) growing a cereal plant, or at least 100 such cereal plants in a field, according to any one of claim 28, 38 or 39, and
b) harvesting the cereal plant part from the cereal plant or cereal plants.

58. A method of producing processed grain, or flour, bran, wholemeal, malt, starch or oil obtained from grain, the method comprising;
a) obtaining grain according to any one of claims 1 to 27, and
b) processing the grain to produce the processed grain, flour, bran, wholemeal, malt, starch or oil.

59. A product produced from grain according to any one of claims 1 to 27, or a cereal plant according to any one of claim 28, 38 or 39, or from a part of said grain or cereal plant comprising the genetic variation.

60. The product of claim 59 comprising one or more or all of the genetic variations, the polynucleotide, the nucleic acid construct or the vector, and the thickened aleurone.

61. The product of claim 59 or claim 60, wherein the part is cooked, boiled, parboiled, roasted, baked, polished, cracked, puffed, milled or flaked grain, or bran.

62. The product according to any one of claims 59 to 61, wherein the product is a food ingredient, beverage ingredient, food product or beverage product.

63. The product of claim 62, wherein
i) the food ingredient or beverage ingredient is selected from the group consisting of roasted grain, polished grain, cracked grain, puffed grain, milled grain, flaked grain, wholemeal, flour, bran, starch, malt and oil,
ii) the food product is selected from the group consisting of: processed grain, cooked grain, boiled grain, porridge, leavened or unleavened breads, pasta, noodles, animal fodder, breakfast cereals, snack foods, cakes, pastries and foods containing a flour-based sauce, or
iii) the beverage product is a tea, a packaged beverage or a beverage comprising ethanol.

64. A method of preparing a food or beverage ingredient of claim 62 or claim 63, the method comprising processing grain according to any one of claims 1 to 27, or bran, flour, wholemeal, malt, starch or oil from the grain, to produce the food or beverage ingredient.

65. A method of preparing a food or beverage product of claim 62 or claim 63, the method comprising processing grain, preferably by cooking, boiling, roasting or flaking grain, or mixing grain according to any one of claims 1 to 27, or bran, flour, wholemeal, malt, starch or oil from the grain, with another food or beverage ingredient.

66. Use of grain according to any one of claims 1 to 27 or part thereof, or a cereal plant according to any one of claim 28, 38 or 39 or part thereof, as animal feed or food, or to produce feed for animal consumption or food for human consumption.

67. A composition comprising one or more of a polypeptide of claim 28, a polynucleotide according to any one of claims 29 to 31, a nucleic acid construct and/or vector of claim 32, or a cell according to any one of claims 33 to 37, and one or more acceptable carriers, preferably another food ingredient.
